(19) United States
(12) Patent Application Publication
(10) Pub. No.: US 2025/0250304 A1
(43) Pub. Date: Aug. 7, 2025

(54) RECOMBINANT NEWCASTLE DISEASE VIRUS EXPRESSING LASSA VIRUS GP OR NP, AND USES THEREOF

(71) Applicants: Icahn School of Medicine at Mount Sinai, New York, NY (US); BERNHARD-NOCHT-INSTITUT FÜR TROPENMEDIZIN, Hamburg (DE)

(72) Inventors: Adolfo GARCIA-SASTRE, New York, NY (US); César MUÑOZ-FONTELA, Hamburg (DE); Peter PALESE, New York, NY (US); Florian KRAMMER, New York, NY (US); Ignacio MENA, New York, NY (US); Arantza COBELA-GARCÍA, New York, NY (US)

(21) Appl. No.: 18/854,935

(22) PCT Filed: Apr. 7, 2023
(86) PCT No.: PCT/US2023/065491

§ 371 Date: Oct. 7, 2024

Related U.S. Application Data
(60) Provisional application No. 63/329,322, filed on Apr. 8, 2022.

(51) Int. Cl. C07K 14/005; A61K 39/00; A61K 39/12; A61P 31/14; C12N 7/00

(52) U.S. Cl. CPC C07K 14/005; A61K 39/12; A61P 31/14; C12N 7/00; A61K 2039/5252; A61K 2039/5256; C12N 2760/10022; C12N 2760/10034; C12N 2760/18121

(57) ABSTRACT

Provided herein are polynucleotides encoding Lassa virus glycoprotein or Lassa virus nucleoprotein, or a chimeric protein comprising the ectodomain of Lassa virus glycoprotein and the transmembrane and cytoplasmic domains of NDV F protein. Also, provided herein are recombinant Newcastle disease virus (NDV) comprising such a polynucleotide, and immunogenic compositions comprising such recombinant NDV. Further, provided herein are methods for immunizing against Lassa virus comprising administering the recombinant NDV or an immunogenic composition thereof.

### CROSS-REFERENCE TO RELATED APPLICATIONS

This application claims the benefit of U.S. Provisional Patent Application No. 63/329,322, filed Apr. 8, 2022, the disclosure of which is incorporated by reference herein in its entirety.

### STATEMENT REGARDING FEDERALLY SPONSORED RESEARCH

This invention was made with government support under grant 75N93019C00045 awarded by the National Institutes of Health. The government has certain rights in the invention.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “06923-392-228_SEQ_LISTING.xml”, was created on Apr. 3, 2023 and is 125,106 bytes in size.

### 1. INTRODUCTION

Provided herein are polynucleotides encoding Lassa virus glycoprotein or Lassa virus nucleoprotein, or a chimeric protein comprising the ectodomain of Lassa virus glycoprotein and the transmembrane and cytoplasmic domains of NDV F protein. Also, provided herein are recombinant Newcastle disease virus (NDV) comprising such a polynucleotide, and immunogenic compositions comprising such recombinant NDV. Further, provided herein are methods for immunizing against Lassa virus comprising administering the recombinant NDV or an immunogenic composition thereof.

### 2. BACKGROUND

Lassa virus (LASV, species Lassa mammarenavirus) is an Old World arenavirus and the causative agent of Lassa fever (LASF) outbreaks [1]. LASV infection in humans causes severe hemorrhagic fever, which in fatal cases leads to multiorgan failure. LASV is responsible for more than 300,000 infections and 5,000 deaths in West Africa every year, with a case fatality ratio of around 18%. Currently there is no approved vaccine [2] nor specific antiviral treatment [3] available. The virus is categorized under the highest biosafety level (BSL-4) which hinders LASV diagnostics and vaccine development.

Thus, there is a need for LASV vaccines.

### 3. SUMMARY

In one aspect, provided herein are recombinant proteins encoding a Lassa virus glycoprotein or a derivative thereof, or the ectodomain of a Lassa virus glycoprotein or a derivative thereof. In some embodiments, a recombinant protein comprises an amino acid sequence provided in Table 3, infra. In some embodiments, provided herein is a recombinant protein comprising a derivative of the ectodomain of a Lassa virus glycoprotein, wherein the derivative of the ectodomain comprises an amino acid sequence that is at least 90% (e.g., at least 91%, at least 92%, at least 93%, or at least 94%) identical to the amino acid sequence of SEQ ID NO:42 or 36. In some embodiments, the derivative of the ectodomain comprises an amino acid sequence that is at least 95% identical to the amino acid sequence of SEQ ID NO:42 or 36. In some embodiments, the derivative of the ectodomain comprises an amino acid sequence that is at least 96%, at least 97%, at least 98%, or at least 99% identical to the amino acid sequence of SEQ ID NO:42 or 36. In some embodiments, the recombinant protein further comprises the transmembrane and cytoplasmic domains of NDV F protein. In some embodiments, the NDV F protein is of the LaSota strain. In some embodiments, the transmembrane and cytoplasmic domains of NDV F protein comprise the amino acid sequence of SEQ ID NO:5. In some embodiments, the derivative of the ectodomain is linked directly to the transmembrane of the NDV F protein. In some embodiments, the derivative of the ectodomain is linked to the transmembrane of the NDV F protein by a linker.

In some embodiments, provided herein is a recombinant protein comprising a derivative of the ectodomain of a Lassa virus glycoprotein, wherein the derivative of the ectodomain comprises an amino acid sequence that is at least 90% (e.g., at least 91%, at least 92%, at least 93%, or at least 94%) identical to the amino acid sequence of SEQ ID NO:42 or 36, and wherein the derivative of the ectodomain comprises: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP. In some embodiments, the derivative of the ectodomain comprises an amino acid sequence that is at least 95% identical to the amino acid sequence of SEQ ID NO: 42 or 36. In some embodiments, the derivative of the ectodomain comprises an amino acid sequence that is at least 96%, at least 97%, at least 98%, or at least 99% identical to the amino acid sequence of SEQ ID NO: 42 or 36. In some embodiments, the derivative of the ectodomain comprises the amino acid sequence of SEQ ID NO: 42 or 36. In some embodiments, the recombinant protein further comprises the transmembrane and cytoplasmic domains of NDV F protein. In some embodiments, the NDV F protein is of the LaSota strain. In some embodiments, the transmembrane and cytoplasmic domains of NDV F protein comprise the amino acid sequence of SEQ ID NO:5. In some embodiments, the derivative of the ectodomain is linked directly to the transmembrane of the NDV F protein. In some embodiments, the derivative of the ectodomain is linked to the transmembrane of the NDV F protein by a linker.

In some embodiments, provided herein is a recombinant protein comprising a derivative of the ectodomain of a Lassa virus glycoprotein, wherein the derivative of the ectodomain comprises the amino acid sequence of a Lassa virus glycoprotein ectodomain and amino acid substitutions resulting in: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP. In some embodiments, the recombinant protein further comprises the transmembrane and cytoplasmic domains of NDV F protein. In some embodiments, the NDV F protein is of the LaSota strain. In some embodiments, the transmembrane and cytoplasmic domains of NDV F protein comprise the amino acid sequence of SEQ ID NO:5. In some embodiments, the derivative of the ectodomain is linked directly to the transmembrane of the NDV F protein. In some embodiments, the derivative of the ectodomain is linked to the transmembrane of the NDV F protein by a linker.

In some embodiments, provided herein is a recombinant protein comprising an amino acid sequence that is at least 90% (e.g., at least 91%, at least 92%, at least 93%, or at least 94%) identical to the amino acid sequence of SEQ ID NO:39, 40, 12, or 13. In some embodiments, the recombinant protein comprises an amino acid sequence that is at least 95% identical to the amino acid sequence of SEQ ID NO:39, 40, 12, or 13. In some embodiments, the recombinant protein comprises an amino acid sequence that is at least 96%, at least 97%, at least 98%, or at least 99% identical to the amino acid sequence of SEQ ID NO:39, 40, 12, or 13. In some embodiments, the recombinant protein comprises the amino acid sequence of SEQ ID NO:39, 40, 12, or 13.

In some embodiments, provided herein is a recombinant protein comprising an amino acid sequence that is at least 90% (e.g., at least 91%, at least 92%, at least 93%, or at least 94%) identical to the amino acid sequence of SEQ ID NO:39, 40, 12, or 13, wherein the protein comprises: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP. In some embodiments, the recombinant protein comprises an amino acid sequence that is at least 95% identical to the amino acid sequence of SEQ ID NO:39, 40, 12, or 13. In some embodiments, the recombinant protein comprises an amino acid sequence that is at least 96%, at least 97%, at least 98%, or at least 99% identical to the amino acid sequence of SEQ ID NO:39, 40, 12, or 13. In some embodiments, the recombinant protein comprises the amino acid sequence of SEQ ID NO:39, 40, 12, or 13.

In another aspect, provided herein are recombinant proteins encoding a Lassa virus nucleoprotein or a derivative thereof. In some embodiments, a recombinant protein comprises an amino acid sequence provided in Table 4, infra. In some embodiments, provided herein is a recombinant protein comprising a derivative of a Lassa virus nucleoprotein, wherein the derivative comprises an amino acid sequence that is at least 90% (e.g., at least 91%, at least 92%, at least 93%, or at least 94%) identical to the amino acid sequence of SEQ ID NO:17, wherein the derivative comprises alanine at amino acid positions 389 and 392 of Lassa virus/H. sapiens-wt/NGA/2018/IRR 013 nucleoprotein (NP). In some embodiments, the derivative comprises an amino acid sequence that is at least 95% identical to the amino acid sequence of SEQ ID NO:17. In some embodiments, the derivative comprises an amino acid sequence that is at least 96%, at least 97%, at least 98%, or at least 99% identical to the amino acid sequence of SEQ ID NO:17. In some embodiments, the derivative comprises the amino acid sequence of SEQ ID NO:17.

In another aspect, provided herein are polynucleotides comprising a nucleotide sequence encoding a Lassa virus glycoprotein or a derivative thereof, a Lassa virus nucleoprotein or a derivative thereof, or a protein comprising a Lassa virus glycoprotein ectodomain or a derivative thereof. In some embodiments, provided herein is a polynucleotide comprising a nucleotide sequence encoding a recombinant protein described herein. In some embodiments, provided herein is a polynucleotide comprising the nucleotide sequence of SEQ ID NO:6, 7, 8, 9, 14, 15, 43, 44, 45, 46, 47, or 48. In some embodiments, provided herein is a polynucleotide comprising the nucleotide sequence that is at least 80% (e.g., at least 81%, at least 82%, at least 83%, or at least 84%) identical to the nucleotide sequence of SEQ ID NO: 6, 7, 8, 9, 14, 15, 43, 44, 45, 46, 47, or 48. In some embodiments, provided herein is a polynucleotide comprising the nucleotide sequence that is at least 85% (e.g., at least 86%, at least 87%, at least 88%, or at least 89%) identical to the nucleotide sequence of SEQ ID NO: 6, 7, 8, 9, 14, 15, 43, 44, 45, 46, 47, or 48. In some embodiments, provided herein is a polynucleotide comprising the nucleotide sequence that is at least 90% (e.g., at least 91%, at least 92%, at least 93%, or at least 94%) identical to the nucleotide sequence of SEQ ID NO: 6, 7, 8, 9, 14, 15, 43, 44, 45, 46, 47, or 48. In some embodiments, provided herein is a polynucleotide comprising the nucleotide sequence that is at least 95% (e.g., at least 96% or at least 97%) identical to the nucleotide sequence of SEQ ID NO: 6, 7, 8, 9, 14, 15, 43, 44, 45, 46, 47, or 48. In some embodiments, provided herein is a polynucleotide comprising the nucleotide sequence that is at least 98% or 99% identical to the nucleotide sequence of SEQ ID NO: 6, 7, 8, 9, 14, 15, 43, 44, 45, 46, 47, or 48.

In some embodiments, provided herein is a polynucleotide comprising the corresponding negative RNA sense of the nucleotide sequence of SEQ ID NO: 6, 7, 8, 9, 14, 15, 43, 44, 45, 46, 47, or 48. In some embodiments, provided herein is a polynucleotide comprising the corresponding negative RNA sense of a nucleotide sequence that is at least 80% (e.g., at least 81%, at least 82%, at least 83%, or at least 84%) identical to the nucleotide sequence of SEQ ID NO: 6, 7, 8, 9, 14, 15, 43, 44, 45, 46, 47, or 48. In some embodiments, provided herein is a polynucleotide comprising the corresponding negative RNA sense of a nucleotide sequence that is at least 85% (e.g., at least 86%, at least 87%, at least 88%, or at least 89%) identical to the nucleotide sequence of SEQ ID NO: 6, 7, 8, 9, 14, 15, 43, 44, 45, 46, 47, or 48. In some embodiments, provided herein is a polynucleotide comprising the corresponding negative RNA sense of a nucleotide sequence that is at least 90% (e.g., at least 91%, at least 92%, at least 93%, or at least 94%) identical to the nucleotide sequence of SEQ ID NO: 6, 7, 8, 9, 14, 15, 43, 44, 45, 46, 47, or 48. In some embodiments, provided herein is a polynucleotide comprising the corresponding negative RNA sense of a nucleotide sequence that is at least 95% (e.g., at least 96% or at least 97%) identical to the nucleotide sequence of SEQ ID NO: 6, 7, 8, 9, 14, 15, 43, 44, 45, 46, 47, or 48. In some embodiments, provided herein is a polynucleotide comprising the corresponding negative RNA sense of a nucleotide sequence that is at least 98% or 99% identical to the nucleotide sequence of SEQ ID NO: 6, 7, 8, 9, 14, 15, 43, 44, 45, 46, 47, or 48.

In some embodiments, the polynucleotide further comprises an NDV regulatory sequence. In some embodiments, the polynucleotide further comprises a Kozak sequence. In some embodiments, the polynucleotide further comprises a restriction site. In some embodiments, the polynucleotide further comprises an NDV regulatory sequence, a Kozak sequence, a restriction site, or a combination thereof.

In some embodiments, provided herein is a transgene comprising a polynucleotide described herein or nucleotide sequence described herein. In some embodiments, provided herein is a nucleotide sequence comprising a transgene and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV transcription unit encodes a NDV F protein comprising a leucine to alanine amino acid substitution at the amino acid residue corresponding to amino acid residue 289 of the LaSota NDV strain F protein. In some embodiments, the transgene is incorporation between two transcription units. In some embodiments, the two transcription units are the transcription units for the NDV P gene and the NDV M gene. In some embodiments, the two transcription units are the transcription units for the NDV NP gene and the NDV P gene.

In some embodiments, provided herein is a vector comprising a polynucleotide described herein or a nucleotide sequence described herein. In some embodiments, provided herein is a vector comprising a transgene described herein. In some embodiments, provided herein is a vector comprising a polynucleotide encoding a protein described herein. In some embodiments, the vector is a plasmid. In some embodiments, the vector is a viral vector.

In another aspect, provided herein are recombinant NDV comprising a polynucleotide or transgene described herein. In a specific embodiment, provided herein is a recombinant Newcastle disease virus (NDV) comprising a packaged genome, wherein the package genome comprises the polynucleotide or transgene described herein. In some embodiments, provided herein is a recombinant NDV comprising a packaged genome, wherein the package genome comprises a polynucleotide sequence encoding the recombinant protein described herein.

In some embodiments, provided herein is a recombinant NDV comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein or a derivative thereof and the transmembrane and cytoplasmic domains of NDV F protein. In some embodiments, the ectodomain comprises the amino acid sequence of SEQ ID NO:35 or 41. In some embodiments, the ectodomain comprises an amino acid sequence that is at least 80%, at least 81%, at least 82%, at least 83% or at least 84% identical to the amino acid sequence of SEQ ID NO:35 or 41. In some embodiments, the ectodomain comprises an amino acid sequence that is at least 85%, at least 86%, at least 87%, at least 88% or at least 89% identical to the amino acid sequence of SEQ ID NO:35 or 41. In some embodiments, the ectodomain comprises an amino acid sequence that is at least 90%, at least 91%, at least 92%, at least 93% or at least 94% identical to the amino acid sequence of SEQ ID NO:35 or 41. In some embodiments, the ectodomain comprises an amino acid sequence that is at least 80%, at least 85%, at least 90%, at least 95% or at least 98% identical to the amino acid sequence of SEQ ID NO:35 or 41. In some embodiments, the ectodomain is encoded by the nucleotide sequence that is at least 95%, at least 96%, at least 97%, at least 98% or at least 99% identical to the nucleotide sequence of SEQ ID NO:35 or 41. In some embodiments, the ectodomain is encoded by the nucleotide sequence of SEQ ID NO:33 or 47. In some embodiments, the ectodomain is encoded by the nucleotide sequence that is at least 80%, at least 81%, at least 82%, at least 83% or at least 84% identical to the nucleotide sequence of SEQ ID NO: 33 or 47. In some embodiments, the ectodomain is encoded by the nucleotide sequence that is at least 85%, at least 86%, at least 87%, at least 88% or at least 89% identical to the nucleotide sequence of SEQ ID NO: 33 or 47. In some embodiments, the ectodomain is encoded by the nucleotide sequence that is at least 90%, at least 91%, at least 92%, at least 93% or at least 94% identical to the nucleotide sequence of SEQ ID NO: 33 or 47. In some embodiments, the ectodomain is encoded by a nucleotide sequence that is at least 80%, at least 85%, at least 90%, at least 95% or at least 98% identical to the nucleotide sequence of SEQ ID NO:33 or 47. In some embodiments, the ectodomain is encoded by the nucleotide sequence that is at least 95%, at least 96%, at least 97%, at least 98% or at least 99% identical to the nucleotide sequence of SEQ ID NO: 33 or 47. In some embodiments, the derivative of the Lassa virus glycoprotein ectodomain comprises the amino acid sequence of SEQ ID NO:36 or 42. In some embodiments, the ectodomain comprises an amino acid sequence that is at least 80%, at least 81%, at least 82%, at least 83% or at least 84% identical to the amino acid sequence of SEQ ID NO:36 or 42. In some embodiments, the ectodomain comprises an amino acid sequence that is at least 85%, at least 86%, at least 87%, at least 88% or at least 89% identical to the amino acid sequence of SEQ ID NO:36 or 42. In some embodiments, the derivative of the Lassa virus glycoprotein ectodomain comprises an amino acid sequence that is at least 90% identical to the amino acid sequence of SEQ ID NO:36 or 42. In some embodiments, the ectodomain comprises an amino acid sequence that is at least 95%, at least 96%, at least 97%, at least 98% or at least 99% identical to the amino acid sequence of SEQ ID NO:36 or 42. In some embodiments, the derivative of the Lassa virus glycoprotein ectodomain is encoded by the nucleotide sequence of SEQ ID NO:34 or 48. In some embodiments, the derivative of the Lassa virus glycoprotein ectodomain is encoded by a nucleotide sequence that is at least 80%, at least 81%, at least 82%, at least 83% or at least 84% identical to the nucleotide sequence of SEQ ID NO:34 or 48. In some embodiments, the derivative of the Lassa virus glycoprotein ectodomain is encoded by a nucleotide sequence that is at least 85%, at least 86%, at least 87%, at least 88% or at least 89% identical to the nucleotide sequence of SEQ ID NO:34 or 48. In some embodiments, the derivative of the Lassa virus glycoprotein ectodomain is encoded by a nucleotide sequence that is at least 90%, at least 91%, at least 92%, at least 93% or at least 94% identical to the nucleotide sequence of SEQ ID NO:34 or 48. In some embodiments, the derivative of the Lassa virus glycoprotein ectodomain is encoded by a nucleotide sequence that is at least 95%, at least 96%, at least 97%, at least 98% or at least 99% identical to the nucleotide sequence of SEQ ID NO:34 or 48. In some embodiments, the derivative of the Lassa virus glycoprotein ectodomain is encoded by a nucleotide sequence that is at least 80%, at least 85%, at least 90%, at least 95% or at least 98% identical to the nucleotide sequence of SEQ ID NO:34 or 48. In some embodiments, the NDV F protein is of the LaSota strain. In some embodiments, the transmembrane and cytoplasmic domain of NDV F protein comprise the amino acid sequence of SEQ ID NO:5. In some embodiments, the ectodomain or derivative of the ectodomain is linked directly to the transmembrane of the NDV F protein. In some embodiments, the ectodomain or derivative of the ectodomain is linked to the transmembrane of the NDV F protein by a linker.

In some embodiments, provided herein is a recombinant NDV comprising a LASV glycoprotein or derivative thereof, a chimeric Lassa virus glycoprotein, or a LASV NP described herein. In some embodiments, provided herein is a recombinant NDV comprising a protein described herein. In some embodiments, provided herein is a recombinant NDV comprising a protein encoded by a polynucleotide or transgene described herein.

In some embodiments, provided herein is a recombinant Newcastle disease virus (NDV) comprising a protein comprising the amino acid sequence of SEQ ID NO:10, 12, 37 or 39. In some embodiments, provided herein is a protein comprising an amino acid sequence that is at least 80%, at least 81%, at least 82%, at least 83%, or at least 84% identical to the amino acid sequence of SEQ ID NO:10, 12, 37, or 39. In some embodiments, provided herein is a protein comprising an amino acid sequence that is at least 85%, at least 86%, at least 87%, at least 88%, or at least 89% identical to the amino acid sequence of SEQ ID NO:10, 12, 37, or 39. In some embodiments, provided herein is a protein comprising an amino acid sequence that is at least 90%, at least 91%, at least 92%, at least 93%, or at least 94% identical to the amino acid sequence of SEQ ID NO:10, 12, 37, or 39. In some embodiments, provided herein is a protein comprising an amino acid sequence that is at least 95%, at least 96%, at least 97%, at least 98%, or at least 99% identical to the amino acid sequence of SEQ ID NO:10, 12, 37, or 39. In some embodiments, provided herein is a protein comprising an amino acid sequence that is at least 80%, at least 85%, at least 90%, at least 95%, or at least 98% identical to the amino acid sequence of SEQ ID NO:10, 12, 37, or 39.

In some embodiments, provided herein is a recombinant Newcastle disease virus (NDV) comprising a protein comprising the amino acid sequence of SEQ ID NO: 11, 13, 38, or 40. In some embodiments, provided herein is a protein comprising an amino acid sequence that is at least 80%, at least 81%, at least 82%, at least 83%, or at least 84% identical to the amino acid sequence of SEQ ID NO: 11, 13, 38, or 40. In some embodiments, provided herein is a protein comprising an amino acid sequence that is at least 85%, at least 86%, at least 87%, at least 88%, or at least 89% identical to the amino acid sequence of SEQ ID NO: 11, 13, 38, or 40. In some embodiments, provided herein is a protein comprising an amino acid sequence that is at least 90%, at least 91%, at least 92%, at least 93%, or at least 94% identical to the amino acid sequence of SEQ ID NO: 11, 13, 38, or 40. In some embodiments, provided herein is a protein comprising an amino acid sequence that is at least 95%, at least 96%, at least 97%, at least 98%, or at least 99% identical to the amino acid sequence of SEQ ID NO: 11, 13, 38, or 40. In some embodiments, provided herein is a protein comprising an amino acid sequence that is at least 80%, at least 85%, at least 90%, at least 95%, or at least 98% identical to the amino acid sequence of SEQ ID NO: 11, 13, 38, or 40.

In some embodiments, provided herein is a recombinant Newcastle disease virus (NDV) comprising a protein comprising the amino acid sequence of SEQ ID NO:16 or 17. In some embodiments, provided herein is a protein comprising an amino acid sequence that is at least 80%, at least 81%, at least 82%, at least 83%, or at least 84% identical to the amino acid sequence of SEQ ID NO:16 or 17. In some embodiments, provided herein is a protein comprising an amino acid sequence that is at least 85%, at least 86%, at least 87%, at least 88%, or at least 89% identical to the amino acid sequence of SEQ ID NO:16 or 17. In some embodiments, provided herein is a protein comprising an amino acid sequence that is at least 90%, at least 91%, at least 92%, at least 93%, or at least 94% identical to the amino acid sequence of SEQ ID NO:16 or 17. In some embodiments, provided herein is a protein comprising an amino acid sequence that is at least 95%, at least 96%, at least 97%, at least 98%, or at least 99% identical to the amino acid sequence of SEQ ID NO:16 or 17. In some embodiments, provided herein is a protein comprising an amino acid sequence that is at least 80%, at least 85%, at least 90%, at least 95%, or at least 98% identical to the amino acid sequence of SEQ ID NO:16 or 17.

In some embodiments, provided herein is a recombinant Newcastle disease virus (NDV) comprising a chimeric Lassa virus glycoprotein that comprises the ectodomain of a Lassa virus glycoprotein and the transmembrane and cytoplasmic domains of NDV F protein, wherein the ectodomain comprises the amino acid sequence of SEQ ID NO:35 or 41. In some embodiments, the ectodomain comprises an amino acid sequence that is at least 80%, at least 81%, at least 82%, at least 83%, or at least 84% identical to the amino acid sequence of SEQ ID NO:35 or 41. In some embodiments, the ectodomain comprises an amino acid sequence that is at least 85%, at least 86%, at least 87%, at least 88%, or at least 89% identical to the amino acid sequence of SEQ ID NO:35 or 41. In some embodiments, the ectodomain comprises an amino acid sequence that is at least 90%, at least 91%, at least 92%, at least 93%, or at least 94% identical to the amino acid sequence of SEQ ID NO:35 or 41. In some embodiments, the ectodomain comprises an amino acid sequence that is at least 90%, at least 95%, at least 96%, at least 97%, at least 98%, or at least 99% identical to the amino acid sequence of SEQ ID NO:35 or 41. In some embodiments, provided herein is a recombinant Newcastle disease virus (NDV) comprising a chimeric Lassa virus glycoprotein that comprises the ectodomain of a Lassa virus glycoprotein and the transmembrane and cytoplasmic domains of NDV F protein, wherein the ectodomain comprises an amino acid sequence that is at least 80%, at least 85%, at least 90%, at least 95%, or at least 98% identical to the amino acid sequence of SEQ ID NO:35 or 41. In some embodiments, the NDV F protein is of the LaSota strain. In some embodiments, the transmembrane and cytoplasmic domain of NDV F protein comprise the amino acid sequence of SEQ ID NO:5. In some embodiments, the ectodomain is linked directly to the transmembrane of the NDV F protein. In some embodiments, the ectodomain is linked to the transmembrane of the NDV F protein by a linker.

In some embodiments, provided herein is a recombinant Newcastle disease virus (NDV) comprising a chimeric Lassa virus glycoprotein, wherein the chimeric Lassa virus glycoprotein comprises a derivative of the ectodomain of a Lassa virus glycoprotein and the transmembrane and cytoplasmic domains of NDV F protein, wherein the derivative comprises the amino acid sequence of SEQ ID NO:36 or 42. In some embodiments, the derivative of the ectodomain comprises an amino acid sequence that is at least 80%, at least 81%, at least 82%, at least 83%, or at least 84% identical to the amino acid sequence of SEQ ID NO: 36 or 42. In some embodiments, the derivative of the ectodomain comprises an amino acid sequence that is at least 85%, at least 86%, at least 87%, at least 88%, or at least 89% identical to the amino acid sequence of SEQ ID NO: 36 or 42. In some embodiments, the derivative of the ectodomain comprises an amino acid sequence that is at least 90%, at least 91%, at least 92%, at least 93%, or at least 94% identical to the amino acid sequence of SEQ ID NO: 36 or 42. In some embodiments, the derivative of the ectodomain comprises an amino acid sequence that is at least 90%, at least 95%, at least 96%, at least 97%, at least 98%, or at least 99% identical to the amino acid sequence of SEQ ID NO: 36 or 42. In some embodiments, provided herein is a recombinant Newcastle disease virus (NDV) comprising a chimeric Lassa virus glycoprotein, wherein the chimeric Lassa virus glycoprotein comprises a derivative of the ectodomain of a Lassa virus glycoprotein and the transmembrane and cytoplasmic domains of NDV F protein, wherein the derivative comprises an amino acid sequence that is at least 80%, at least 85%, at least 90%, at least 95%, or at least 98% identical to the amino acid sequence of SEQ ID NO:36 or 42. In some embodiments, the NDV F protein is of the LaSota strain. In some embodiments, the transmembrane and cytoplasmic domain of NDV F protein comprise the amino acid sequence of SEQ ID NO:5. In some embodiments, the derivative of the ectodomain is linked directly to the transmembrane of the NDV F protein. In some embodiments, the derivative of the ectodomain is linked to the transmembrane of the NDV F protein by a linker.

In some embodiments, provided herein are cells comprising a polynucleotide herein, a nucleic acid sequence, or a transgene described herein. In some embodiments, provided herein are cells comprising a vector described herein. In some embodiments, provided herein are cells comprising a recombinant NDV described herein. In some embodiments, provided herein are cells expressing a recombinant protein described herein. In some embodiments, the cells are cell lines. In some embodiments, the cells are primary cells. In some embodiments, the cells are in vitro or ex vivo.

In some embodiments, provided herein is an embryonated egg comprising a polynucleotide herein or a transgene described herein. In some embodiments, provided herein is an embryonated egg comprising a vector described herein. In some embodiments, provided herein is an embryonated egg comprising a recombinant NDV described herein. In some embodiments, provided herein is an embryonated egg expressing a recombinant protein described herein. In some embodiments, the embryonated egg is a non-human egg. In some embodiments, the embryonated egg is ex vivo. In some embodiments, the embryonated egg is a non-human egg that is ex vivo. In some embodiments, the embryonated egg is a chicken egg or other avian egg. In some embodiments, the embryonated egg is a chicken egg that is about 8 to about 12 days old (e.g., 8, 9, 10 or 11 days old).

In another aspect, provided herein are immunogenic compositions. In some embodiments, provided herein is an immunogenic composition comprising a recombinant protein described herein. In some embodiments, provided herein is an immunogenic composition comprising a polynucleotide described herein or a transgene described herein. In some embodiments, provided herein is an immunogenic composition comprising a vector described herein. In some embodiments, provided herein is an immunogenic composition comprising the recombinant NDV described herein. In some embodiments, the recombinant NDV is a live virus. In some embodiments, the recombinant NDV is inactivated.

In another aspect, provided herein is a method for immunizing against Lassa virus, comprising administering a recombinant NDV or an immunogenic composition described herein to a subject. In some embodiments, provided herein is a method for immunizing against Lassa virus, comprising administering a recombinant NDV described herein to a subject. In some embodiments, provided herein is a method for immunizing against Lassa virus, comprising administering an immunogenic composition described herein to a subject. In some embodiments, the subject is a human subject.

In another aspect, provided herein is a method for inducing an immune response against Lassa virus, comprising administering a recombinant NDV or an immunogenic composition described herein to a subject. In some embodiments, provided herein is a method for inducing an immune response against Lassa virus, comprising administering a recombinant NDV described herein to a subject. In some embodiments, provided herein is a method for inducing an immune response against Lassa virus, comprising administering an immunogenic composition described herein to a subject. In some embodiments, the subject is a human subject.

In another aspect, provided herein is a method for preventing Lassa virus disease, comprising administering a recombinant NDV or an immunogenic composition described herein to a subject. In some embodiments, provided herein is a method for preventing Lassa virus disease, comprising administering a recombinant NDV described herein to a subject. In some embodiments, provided herein is a method for preventing Lassa virus disease, comprising administering an immunogenic composition described herein to a subject. In some embodiments, the subject is a human subject.

In another aspect, provided herein are kits. In some embodiments, provided herein is a kit comprising a container containing a recombinant NDV described herein. In some embodiments, provided herein is a kit comprising a container containing a polynucleotide described herein or a nucleotide sequence described herein. In some embodiments, provided herein is a kit comprising a container containing a transgene described herein. In some embodiments, provided herein is a kit comprising a container containing a vector described herein. In some embodiments, provided herein is a kit comprising a container containing a recombinant protein described herein.

### 3.1 Terminology

As used herein, the term “about” or “approximately” when used in conjunction with a number refers to any number within 1, 5 or 10% of the referenced number, including the referenced number.

As used herein, the terms “antibody” and “antibodies” refer to molecules that contain an antigen binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the phrases “IFN deficient systems” or “IFN-deficient substrates” refer to systems, e.g., cells, cell lines and animals, such as mice, chickens, turkeys, rabbits, rats, horses etc., which do not produce one, two or more types of IFN, or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, are deficient in the activity of antiviral genes induced by one, two or more types of IFN, or induced by any type of IFN, or any combination thereof.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, bovine, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In specific embodiments, the subject is a human. In certain embodiments, the mammal (e.g., human) is 4 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the term “in combination” in the context of the administration of a therapy(ies) to a subject, refers to the use of more than one therapy. The use of the term “in combination” does not restrict the order in which therapies are administered to a subject. A first therapy can be administered prior to, concomitantly with, or subsequent to the administration of a second therapy to a subject.

As used herein, the terms “Lassa virus” and “LASV” refer to a Lassa virus known to one of skill in the art. In some embodiments, a Lassa virus is of a specific lineage (e.g., a Lassa virus lineage II). There are at least 7 different lineages of Lassa virus. In some embodiments, a Lassa virus is of a sublineage of Lassa virus lineage II (e.g., a Lassa virus of any one of clades 2a through 2g). In specific embodiments, a Lassa virus is a Lassa virus/H. sapiens-wt/NGA/2018/IRR 013. In some embodiments, a Lassa virus is a Nig08-04 strain (see, e.g., GenBank Accession No. GU481068.1 for the glycoprotein precursor (GPC) and nucleoprotein (NP) genes of Nig08-04 strain).

As used herein, the terms “Lassa virus glycoprotein”, “Lassa virus GP”, LASV glycoprotein”, and “LASV GP” refer to a glycoprotein of a Lassa virus known to one of skill in the art. See, e.g., GenBank No. MK117961.1. Typically, a Lassa virus glycoprotein is synthesized as a 76-kDa glycosylated precursor protein (GP-C). The GP-C is post-translationally cleaved twice by a signal peptidase (SPase) and a subtilisin kexin isozyme-1/site-1 (SKI-1) protease to yield three subunits, the stable signal peptide (SSP), the N-terminal 44-kDa subunit GP-1 and the membrane bound 36-kDa subunit GP-2. See, e.g., Lenz et al., PNAS 98(22): 12701-12705 and Katz et al., 2022, Nature 603:174. GP-1 of Lassa virus interacts with the cellular receptor (matriglycan, a linear carbohydrate present on α-dystroglycan). GP-2 mediates pH-dependent fusion of the viral envelope with the cellular target membrane. Id. The SSP translocates with the spike and forms part of the mature complex. The Lassa virus spike complex comprises three protomers and each protomer comprises the SSP, GP1, and GP2 subunits.

As used herein, the terms “Lassa virus nucleoprotein”, “Lassa virus NP”, LASV nucleoprotein”, and “LASV NP” refer to a nucleoprotein of a Lassa virus known to one of skill in the art. See, e.g., GenBank No. MK117961.1. The Lassa virus NP is found in both virions and LASV infected cells. Loureiro et al., 2019, Pathogens 8(1) 17, https://doi.org/10.3390/pathogens8010017. The Lassa virus NP plays associates tightly with viral genomic and antigenomic RNAs forming ribonucleoprotein (RNP) complexes called nucleocapsids. Id. The nucleocapsids bind the L polymerase, and constitute the biologically active units for transcription of subgenomic viral mRNAs and for viral genome replication. Id. The LASV NP also interacts with the Z matrix protein and contributes to the packaging of RNPs into viral particles during virion morphogenesis. Id. The LASV NP is organized in two distinct domains, the N-terminal domain and C-terminal domain. Id. The N-terminal domain has been reported to function in binding RNA, and the C-terminal domain of NP contains a functional 3′-5′ exoribonuclease activity of the DExD/H-box protein family, which has been demonstrated to oppose the host type I interferon (IFN-I)-mediated immune response during viral infection. The NP has been reported to be capable of degrading small viral doubled-stranded RNA fragments. Id.

As used herein, the terms “therapies” and “therapy” can refer to any protocol(s), method(s), agent(s) or a combination thereof that can be used in the treatment or prevention of Lassa virus disease (e.g., Lassa fever), or vaccination. In certain embodiments, the term “therapy” refers to a recombinant NDV described herein. In other embodiments, the term “therapy” refers to an agent that is not a recombinant NDV described herein.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). In one embodiment, the two sequences are the same length. In a certain embodiment, the percent identity is determined over the entire length of an amino acid sequence or nucleotide sequence. In some embodiments, the length of sequence identity comparison may be over the full-length of the two sequences being compared (e.g., the full-length of a gene coding sequence, or a fragment thereof). In some embodiments, a fragment of a nucleotide sequence is at least 25, at least 50, at least 75, or at least 100 nucleotides. Similarly, “percent sequence identity” may be readily determined for amino acid sequences, over the full-length of a protein, or a fragment thereof. In some embodiments, a fragment of a protein comprises at least 20, at least 30, at least 40, at least 50 or more contiguous amino acids of the protein. In certain embodiments, a fragment of a protein comprises at least 75, at least 100, at least 125, at least 150 or more contiguous amino acids of the protein.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe).

In certain embodiments, an “isolated” polynucleotide, nucleotide sequence or nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” polynucleotide, nucleotide sequence or nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of polynucleotides, nucleotide sequences or nucleic acid sequences in which the nucleic acid sequence is separated from cellular components of the cells from which it is isolated or recombinantly produced. Thus, polynucleotide, nucleotide sequence or nucleic acid sequence that is substantially free of cellular material includes preparations of nucleic acid sequence having less than about 30%, 20%, 10%, or 5% (by dry weight) of other nucleic acids. The term “substantially free of culture medium” includes preparations of polynucleotide, nucleotide sequence or nucleic acid sequence in which the culture medium represents less than about 50%, 20%, 10%, or 5% of the volume of the preparation. The term “substantially free of chemical precursors or other chemicals” includes preparations in which the polynucleotide, nucleotide sequence or nucleic acid sequence is separated from chemical precursors or other chemicals which are involved in the synthesis of the polynucleotide, nucleotide sequence or nucleic acid sequence. In specific embodiments, such preparations of the nucleic acid sequence have less than about 50%, 30%, 20%, 10%, 5% (by dry weight) of chemical precursors or compounds other than the polynucleotide, nucleotide sequence or nucleic acid sequence of interest.

In specific embodiments, a polynucleotide sequence described herein, a nucleic acid sequence described herein, or nucleotide sequence described herein is a recombinant polynucleotide sequence described herein, recombinant nucleic acid sequence described herein, or recombinant nucleotide sequence. In certain embodiments, a polynucleotide sequence described herein, a nucleotide sequence described herein, or nucleic acid sequence described herein may be a DNA molecule (e.g., cDNA), an RNA molecule (e.g., mRNA), or a combination of a DNA and RNA molecule. In some embodiments, a polynucleotide sequence described herein, nucleotide sequence described herein, or nucleic acid sequence described herein may comprise analogs of DNA or RNA molecules. Such analogs can be generated using, for example, nucleotide analogs, which include, but are not limited to, inosine, methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The polynucleotide sequences, nucleic acid sequences, or nucleotide sequences can be single-stranded, double-stranded, may contain both single-stranded and double-stranded portions, and may contain triple-stranded portions. In a specific embodiment, a polynucleotide sequence described herein, nucleotide sequence described herein, or nucleic acid sequence described herein is a negative sense single-stranded RNA. In another specific embodiment, a polynucleotide sequence described herein, a nucleotide sequence described herein, or nucleic acid sequence described herein is a positive sense single-stranded RNA. In another specific embodiment, a polynucleotide sequence described herein, nucleotide sequence described herein, or nucleic acid sequence described herein is a cDNA.

### 4. BRIEF DESCRIPTION OF THE FIGURES

FIG. 1: Schematic illustration representing the recombinant NDV segment containing the LASV insert.

FIGS. 2A-2B. Design of the NDV rescue system (FIG. 2A) A549 or HEp-2 cells are infected with the modified vaccinia virus Ankara expressing the bacteriophage T7 polymerase (MVA-T7). After viral infection, cells are co-transfected with the expression plasmids required for replication and transcription of the NDV viral genome (NP, P, and L), together with the full length NDV cDNA, under the T7 promoter. Twenty-four hours post-infection/transfection, 8-10 day-old chicken embryonated eggs are inoculated with the tissue culture supernatants of the transfected cells for further amplification. Three days after incubation of the 8-10 day-old eggs at 37° C., allantoic fluid from the eggs is harvested and analyzed for successful rescue of the recombinant virus by HA assay. Positive (+) rescue viruses are further characterized at genomic (RT-PCR) and protein (e.g., immunofluorescence assay (IFA) and western blot (WB) levels. Negative (−) HA results may be due to the lack of enough viruses to be detected by HA. Reinfection of fresh chicken embryonated eggs to amplify the virus is indicated. (FIG. 2B) Infection of chicken embryonated eggs: 8-10 chicken embryonated eggs are candled to mark the interface between the air sac and the allantoic cavity. With an insulin (1 ml) syringe, eggs are infected with the tissue culture supernatant inside the allantoic cavity, as indicated. See Ayllon et al., J Vis Exp. 2013; (80): 50830.

FIGS. 3A-3E. Confirmation of recombinant NDV and LASV GP protein expression by immunofluorescence (IFA). LASV GP protein expression was confirmed by immunofluorescence. The different rNDV-LASV vaccine candidates were stained using DAPI, IB3 against LASV GP (Alexa Fluor 488) and rabbit polyclonal sera against NDV (Alexa Fluor 594). FIG. 3A) NDV was used as the negative control, where only the viral vector was detected. While the expression of the GP was observed for FIG. 3B) rNDV-LASV GP, FIG. 3C) rNDV-LASV GP chimera, FIG. 3D) rNDV-LASV GP 1 Pro and FIG. 3E) rNDV-LASV GP 1 Pro chimera within the NDV backbone. LASV GP can be seen in green, NDV in red and the cell nucleus in blue.

FIG. 4. Confirmation of the presence of recombinant NDV. The presence of the viral vector, NDV, by Western Blot (WB) of the purified vaccine candidates, was detected using the monoclonal antibody 8H2 to detect the presence of the HN protein.

FIG. 5. Confirmation of LASV GP expression. The expression of the LASV GP was confirmed by WB of the purified vaccine candidates, using the monoclonal antibody IB3. Vero cells were infected at MOIs of 0.5 FFU/cell or 2.5 FFU/cell. Both the GPC and the fusion subunit of the GP (GP2) were detected. Different cleavage efficiencies were observed between the vaccine candidates for the GP2. Non-infected cells (mock) were used as a negative control (FIG. 5, left lane). As a positive control, cells were transfected with a mammalian expression vector expressing LASV GP (FIG. 5, right lane).

FIG. 6. Confirmation of LASV NP expression. The expression of the LASV NP was confirmed by WB of the purified vaccine candidates, using the polyclonal antibody PA5117437. The presence of the NP was only detected in the LASV NP vaccine candidates.

FIG. 7. Weight Variation post-immunization as an indication of toxicity of the recombinant NDV. The acute toxicity of the recombinant NDV vector was determined by vaccinating interferon-α/β receptor (IFNAR)^{-/-} mice (C57BL/6 background) with different doses of NDV, as assessed by percent weight variation. No mortality was observed for any of the doses, corroborating the vaccine safety.

FIG. 8. Characterization of the cellular host immune response against the different LASV GP rNDV vaccine candidates by IFN-γ ELISpot. C57BL/6J female mice were intranasally vaccinated following a 3-week interval prime-boost regimen. Spleens were collected to quantify LASV-specific T cells at 10 days post-immunization. The cells isolated from the spleens were stimulated with either a LASV GP-derived peptide pool or an irrelevant peptide pool. Each symbol represents the number if IFN-γ producing cells measure by ELISpot per mouse. Bars represent the mean average. n=5 mice/group, except for the unvaccinated (n=2) and mice vaccinated with rNDV-LASV GP 1 Pro (n=1).

FIGS. 9A-9C. Characterization of LASV GP-specific cytolytic response by CTL assay. C57BL/6J female mice were intranasally vaccinated following a 3-week interval prime-boost regimen. The killing potential was monitored 7 days post immunization with different vaccine candidates: I) rNDV-LASV GP (FIG. 9A), II) rNDV-LASV GP 1 Pro (FIG. 9B), and III) NDV (FIG. 9C). CFSE-labelled irrelevant peptide pool (insert E; KSFLWTQSL (SEQ ID NO: 23), QAVNNLVEL (SEQ ID NO: 24), LTYSQLMTL (SEQ ID NO: 25), YQPMSGCYI (SEQ ID NO: 26), and SGGLNIPVL (SEQ ID NO: 27)) and LASV GP-derived peptide pool (insert D; QIITFFQEV (SEQ ID NO: 28), ANLNMTMPL (SEQ ID NO: 29), IINHKFCNL (SEQ ID NO: 30), NALINDQLI (SEQ ID NO: 31), and CNYSKYWYL (SEQ ID NO:32)) targeted populations killing was evaluated by flow cytometry. A strong CTL response was generated in the mice vaccinated with LASV GP rNDV vaccine candidates, which showed 85.4% (FIG. 9A) and 80% (FIG. 9B) killing of targeted cells, for rNDV-LASV GP and rNDV-LASV GP 1 Pro, respectively.

FIG. 10. Quantification of LASV GP-specific antibodies by indirect ELISA for vaccine candidate selection. C57BL/6J female mice were intranasally vaccinated following a 3 week-interval prime-boost regimen. Blood was collected for serology to quantify total serum LASV GP IgG titers after the vaccine prime and boost. High titers of LASV GP IgG were detected in mice vaccinated with the rNDV-LASV GP chimera candidate.

FIGS. 11A-11B. Quantification of LASV GP-specific antibodies by indirect ELISA to determine time- and dose-dependent humoral responses. C57BL/6J female mice were intranasally vaccinated following a 5 week-interval prime-boost regimen. Blood was collected for serology to quantify total LASV GP IgG titers after the vaccine prime and boost. FIG. 11A shows that LASV GP IgG titers increased over time, resulting in a 2-fold increase after the prime and 6-fold increase after the boost. FIG. 11B shows that LASV GP IgG titers were also dose-dependent, as higher antibody titers were detected at the highest doses. No significant reduction was observed in the total IgG titers over time.

FIG. 12. Quantification of LASV NP-specific antibodies by indirect ELISA. 7 days after intranasal vaccination of C57BL/6J female mice, LASV NP-specific antibodies were quantified by indirect ELISA. High titers of LASV NP specific IgG were detected after only one dose of LASV NP vaccine candidates. These results corroborate the appropriate expression of the NP after vaccination.

FIG. 13. Quantification of NDV NP-specific antibodies by indirect ELISA. 7 days after intranasal vaccination of C57BL/6J female mice (with or without a previous immunization), NDV NP-specific antibodies were quantified by indirect ELISA. High titers of NDV NP specific IgG were detected after both prime and prime-boost vaccination regimens, which confirm the appropriateness of intranasal vaccination of mice. Mock vaccination results are the circles on the x-axis.

### 5. DETAILED DESCRIPTION

### 5.1 Polynucleotides

In a specific embodiment, provided herein is a polynucleotide or transgene comprising a nucleotide sequence encoding a LASV GP or a protein comprising a LASV GP ectodomain. In some embodiments, provided herein is a polynucleotide or transgene comprising a nucleotide sequence encoding a derivative of a LASV GP or ectodomain thereof. In some embodiments, the LASV GP or ectodomain thereof is from or derived from a specific lineage of Lassa virus (e.g., Lassa virus lineage II). In a specific embodiment, provided herein is a polynucleotide or transgene comprising a nucleotide sequence encoding a LASV NP or a derivative thereof. In some embodiments, the LASV NP is from or derived from a specific lineage of Lassa virus (e.g., Lassa virus lineage II). In some embodiments, the LASV NP is from or derived from Lassa virus/H. sapiens-wt/NGA/2018/IRR 013. See, e.g., Section 3.1, for types and strains of Lassa virus that may be used.

In some embodiments, a derivative of a LASV GP or ectodomain thereof comprises a certain percent identity to a Lassa virus GP known to one of skill in the art (e.g., Lassa virus lineage II GP, such as Lassa virus/H. sapiens-wt/NGA/2018/IRR 013 GP). For example, a derivative of a LASV GP or ectodomain thereof may have at least 80%, at least 85%, at least 90%, at least 95%, at least 96%, at least 97% or at least 98% identity to a Lassa virus GP known to one of skill in the art (e.g., Lassa virus lineage II GP, such as Lassa virus/H. sapiens-wt/NGA/2018/IRR 013 GP). A derivative of a LASV GP or ectodomain thereof may have a certain number of amino acid mutations (e.g., insertions, deletions, and/or substitutions) relative to a Lassa virus GP known to one of skill in the art (e.g., Lassa virus lineage II GP, such as Lassa virus/H. sapiens-wt/NGA/2018/IRR 013 GP). For example, a derivative of a LASV GP or ectodomain thereof may comprise 1, 2, 3, 4, 5, 6, 7, 8, 9, 10 or more amino acid substitutions (e.g., conservative amino acid substitutions) relative to a Lassa virus GP known to one of skill in the art (e.g.,, Lassa virus lineage II GP, such as Lassa virus/H. sapiens-wt/NGA/2018/IRR 013 GP). In some embodiments, a derivative of a LASV GP or ectodomain thereof comprises amino acid substitutions at amino acid positions corresponding to amino acid positions 206, 328, and 359 of Lassa virus/H. sapiens-wt/NGA/2018/IRR 013 GP. In some embodiments, a derivative of a LASV GP or ectodomain thereof comprises the following: (1) an amino acid substitution to cysteine at the amino acid position corresponding to amino acid position 206 of Lassa virus/H. sapiens-wt/NGA/2018/IRR 013 GP, (2) an amino acid substitution to proline at the amino acid position corresponding to amino acid position 328 of Lassa virus/H. sapiens-wt/NGA/2018/IRR 013 GP, and (3) an amino acid substitution to cysteine at the amino acid position corresponding to amino acid position 359 of Lassa virus/H. sapiens-wt/NGA/2018/IRR 013 GP. In some embodiments, the amino acid substitutions stabilize the pre-fusion conformation of the GP. Techniques known to one of skill in the art may be used to assess the stability of the pre-fusion conformation.

In some embodiments, a derivative of a LASV NP comprises a certain percent identity to a Lassa virus NP known to one of skill in the art (e.g.,, Lassa virus lineage II NP, such as Lassa virus/H. sapiens-wt/NGA/2018/IRR 013 NP). For example, a derivative of a LASV NP may have at least 80%, at least 85%, at least 90%, at least 95%, at least 96%, at least 97% or at least 98% identity to a Lassa virus NP known to one of skill in the art (e.g.,, Lassa virus lineage II NP, such as Lassa virus/H. sapiens-wt/NGA/2018/IRR 013 NP). A derivative of a LASV NP may have a certain number of amino acid mutations (e.g., insertions, deletions, and/or substitutions) relative to a Lassa virus NP known to one of skill in the art (e.g., Lassa virus lineage II NP, such as Lassa virus/H. sapiens-wt/NGA/2018/IRR 013 NP). For example, a derivative of a LASV NP thereof may comprise 1, 2, 3, 4, 5, 6, 7, 8, 9, 10 or more amino acid substitutions (e.g., conservative amino acid substitutions) relative to a Lassa virus NP known to one of skill in the art (e.g.,, Lassa virus lineage II NP, such as Lassa virus/H. sapiens-wt/NGA/2018/IRR 013 NP). In specific embodiments, the amino acid mutations (e.g., amino acid substitutions) inactivate the exonuclease function of the LASV NP. In some embodiments, a derivative of a LASV NP comprises amino acid substitutions at amino acid positions corresponding to amino acid positions 389 and 392 of Lassa virus/H. sapiens-wt/NGA/2018/IRR 013 NP. In some embodiments, a derivative of a LASV NP comprises the following: (1) an amino acid substitution to alanine at the amino acid position corresponding to amino acid position 389 of Lassa virus/H. sapiens-wt/NGA/2018/IRR 013 NP, and (2) an amino acid substitution to alanine at the amino acid position corresponding to amino acid position 392 of Lassa virus/H. sapiens-wt/NGA/2018/IRR 013 NP.

In some embodiments, provided herein is a polynucleotide or transgene comprising a nucleotide sequence encoding a LASV GP, wherein the ectodomain of the LASV GP comprises 1, 2, 3, 4, 5, 6, 7, 8 or more amino acid substitutions. In some embodiments, the amino acid substitutions are conservative amino acid substitutions. In some embodiments, the amino acid substitutions stabilize the pre-fusion conformation of the GP. In some embodiments, provided herein is a polynucleotide or transgene comprising a nucleotide sequence encoding a LASV GP, wherein the GP comprises amino acid substitutions at amino acid positions corresponding to amino acid positions 206, 328, and 359 of Lassa virus/H. sapiens-wt/NGA/2018/IRR 013 GP. In some embodiments, provided herein is a polynucleotide or transgene comprising a nucleotide sequence encoding a LASV GP, wherein the GP comprises the following: (1) an amino acid substitution to cysteine at the amino acid position corresponding to amino acid position 206 of Lassa virus/H. sapiens-wt/NGA/2018/IRR 013 GP, (2) an amino acid substitution to proline at the amino acid position corresponding to amino acid position 328 of Lassa virus/H. sapiens-wt/NGA/2018/IRR 013 GP, and (3) an amino acid substitution to cysteine at the amino acid position corresponding to amino acid position 359 of Lassa virus/H. sapiens-wt/NGA/2018/IRR 013 GP. In some embodiments, the LASV GP ectodomain comprises amino substitutions corresponding to those identified in SEQ ID NO: 12. The corresponding amino acid positions may be determined by aligning a Lassa virus GP with the GP of Lassa virus/H. sapiens-wt/NGA/2018/IRR 013.

In some embodiments, provided herein is a polynucleotide or transgene comprising a nucleotide sequence encoding a protein, wherein the protein comprises a LASV GP ectodomain (e.g., a LASV GP ectodomain described herein), wherein the LASV GP ectodomain comprises 1, 2, 3, 4, 5, 6, 7, 8 or more amino acid substitutions. In some embodiments, the amino acid substitutions are conservative amino acid substitutions. In some embodiments, the amino acid substitutions stabilize the pre-fusion conformation of the GP. In some embodiments, the amino acid substitutions are at amino acid positions corresponding to amino acid positions 206, 328, and 359 of Lassa virus/H. sapiens-wt/NGA/2018/IRR 013 GP. In some embodiments, the LASV GP ectodomain comprises the following amino acid substitutions: (1) an amino acid substitution to cysteine at the amino acid position corresponding to amino acid position 206 of Lassa virus/H. sapiens-wt/NGA/2018/IRR 013 GP, (2) an amino acid substitution to proline at the amino acid position corresponding to amino acid position 328 of Lassa virus/H. sapiens-wt/NGA/2018/IRR 013 GP, and (3) an amino acid substitution to cysteine at the amino acid position corresponding to amino acid position 359 of Lassa virus/H. sapiens-wt/NGA/2018/IRR 013 GP. In some embodiments, the LASV GP ectodomain comprises amino substitutions corresponding to those identified in SEQ ID NO: 12. The corresponding amino acid positions may be determined by aligning a Lassa virus GP with the GP of Lassa virus/H. sapiens-wt/NGA/2018/IRR 013.

In some embodiments, provided herein is a polynucleotide or transgene comprising the nucleotide sequence of SEQ ID NO:6 or 8. In some embodiments, provided herein is a polynucleotide or transgene comprising the nucleotide sequence of SEQ ID NO:6 or 8 without the signal sequence. In some embodiments, provided herein is a polynucleotide or transgene comprising the nucleotide sequence of SEQ ID NO:43 or 45. In some embodiments, provided herein is a polynucleotide or transgene comprising a nucleotide sequence that is at least 75%, at least 80%, at least 85%, or at least 90% identical to SEQ ID NO: 6 or 8. In some embodiments, provided herein is a polynucleotide or transgene comprising a nucleotide sequence that is at least 75%, at least 80%, at least 85%, or at least 90% identical to SEQ ID NO: 6 or 8 without the signal sequence. In some embodiments, provided herein is a polynucleotide or transgene comprising a nucleotide sequence that is at least 75%, at least 80%, at least 85%, or at least 90% identical to SEQ ID NO: 43 or 45. In some embodiments, provided herein is a polynucleotide or transgene comprising a nucleotide sequence that is at least 95%, at least 96%, at least 97%, at least 98%, or at least 99% identical to SEQ ID NO: 6 or 8. In some embodiments, provided herein is a polynucleotide or transgene comprising a nucleotide sequence that is at least 95%, at least 96%, at least 97%, at least 98%, or at least 99% identical to SEQ ID NO: 6 or 8 without the signal sequence. In some embodiments, provided herein is a polynucleotide or transgene comprising a nucleotide sequence that is at least 95%, at least 96%, at least 97%, at least 98%, or at least 99% identical to SEQ ID NO: 43 or 45.

In some embodiments, provided herein is a polynucleotide or transgene comprising a nucleotide sequence encoding the amino acid sequence of SEQ ID NO: 10, 12, 37, or 39. In some embodiments, provided herein is a polynucleotide or transgene comprising a nucleotide sequence encoding an amino acid sequence that is at least 80%, at least 81%, at least 82%, at least 83%, or at least 84% identical to the amino acid sequence of SEQ ID NO: 10, 12, 37, or 39. In some embodiments, provided herein is a polynucleotide or transgene comprising a nucleotide sequence encoding an amino acid sequence that is at least 85%, at least 86%, at least 87%, at least 88%, or at least 89% identical to the amino acid sequence of SEQ ID NO: 10, 12, 37, or 39. In some embodiments, provided herein is a polynucleotide or transgene comprising a nucleotide sequence encoding an amino acid sequence that is at least 90%, at least 91%, at least 92%, at least 93%, or at least 95% identical to the amino acid sequence of SEQ ID NO: 10, 12, 37, or 39. In some embodiments, provided herein is a polynucleotide or transgene comprising a nucleotide sequence encoding an amino acid sequence that is at least 80%, at least 85%, or at least 90% identical to the amino acid sequence of SEQ ID NO: 10, 12, 37, or 39. In some embodiments, provided herein is a polynucleotide or transgene comprising a nucleotide sequence encoding an amino acid sequence that is at least 95%, at least 96%, at least 97%, at least 98%, or at least 99% identical to the amino acid sequence of SEQ ID NO: 10, 12, 37, or 39. In some embodiments, the nucleotide sequence is codon optimized. See, e.g., Section 5.1.1, infra, regarding optimization.

In some embodiments, provided herein is a polynucleotide or transgene comprising a nucleotide sequence encoding a LASV NP, wherein the LASV NP comprises 1, 2, 3, 4, 5, 6, 7, 8 or more amino acid substitutions. In some embodiments, the amino acid substitutions are conservative amino acid substitutions. In some embodiments, the amino acid substitutions inhibit the exonuclease domain of the LASV NP. In some embodiments, provided herein is a polynucleotide or transgene comprising a nucleotide sequence encoding a LASV NP, wherein the NP comprises amino acid substitutions at amino acid positions corresponding to amino acid positions 389 and 392 of Lassa virus/H. sapiens-wt/NGA/2018/IRR 013 NP. In some embodiments, provided herein is a polynucleotide or transgene comprising a nucleotide sequence encoding a LASV NP comprises the following: (1) an amino acid substitution to alanine at the amino acid position corresponding to amino acid position 389 of Lassa virus/H. sapiens-wt/NGA/2018/IRR 013 NP, and (2) an amino acid substitution to alanine at the amino acid position corresponding to amino acid position 392 of Lassa virus/H. sapiens-wt/NGA/2018/IRR 013 NP. In some embodiments, the LASV NP ectodomain comprises amino substitutions corresponding to those identified in SEQ ID NO: 17. The corresponding amino acid positions may be determined by aligning a Lassa virus NP with the NP of Lassa virus/H. sapiens-wt/NGA/2018/IRR 013.

In some embodiments, provided herein is a polynucleotide or transgene comprising the nucleotide sequence of SEQ ID NO:14 or 15. In some embodiments, provided herein is a polynucleotide or transgene comprising a nucleotide sequence that is at least 75%, at least 80%, at least 85%, or at least 90% identical to SEQ ID NO: 14 or 15. In some embodiments, provided herein is a polynucleotide or transgene comprising a nucleotide sequence that is at least 95%, at least 96%, at least 97%, at least 98%, or at least 99% identical to SEQ ID NO: 14 or 15.

In some embodiments, provided herein is a polynucleotide or transgene comprising a nucleotide sequence encoding the amino acid sequence of SEQ ID NO: 16 or 17. In some embodiments, provided herein is a polynucleotide or transgene comprising a nucleotide sequence encoding an amino acid sequence that is at least 80%, at least 85%, or at least 90% identical to the amino acid sequence of SEQ ID NO: 16 or 17. In some embodiments, provided herein is a polynucleotide or transgene comprising a nucleotide sequence encoding an amino acid sequence that is at least 95%, at least 96%, at least 97%, at least 98%, or at least 99% identical to the amino acid sequence of SEQ ID NO: 16 or 17. In some embodiments, the nucleotide sequence is codon optimized. See, e.g., Section 5.1.1, infra, regarding optimization.

In some embodiments, provided herein is a polynucleotide or transgene comprising a nucleotide sequence encoding protein, wherein the protein comprises an amino acid sequence that is at least 80%, at least 81%, at least 82%, at least 83%, or at least 84% identical to the amino acid sequence of SEQ ID NO:36 or 42. In some embodiments, the protein comprises an amino acid sequence that is at least 85%, at least 86%, at least 87%, at least 88%, or at least 89% identical to the amino acid sequence of SEQ ID NO:36 or 42. In some embodiments, the protein comprises an amino acid sequence that is at least 90%, at least 91%, at least 92%, at least 93%, or at least 94% identical to the amino acid sequence of SEQ ID NO:36 or 42. In some embodiments, the protein comprises an amino acid sequence that is at least 95%, at least 96%, at least 97%, at least 98%, or at least 99% identical to the amino acid sequence of SEQ ID NO:36 or 42. In some embodiments, the protein comprises amino acid substitutions at amino acid positions corresponding to amino acid positions 206, 328, and 359 of Lassa virus/H. sapiens-wt/NGA/2018/IRR 013 GP. In some embodiments, the protein comprises the following: (1) an amino acid substitution to cysteine at the amino acid position corresponding to amino acid position 206 of Lassa virus/H. sapiens-wt/NGA/2018/IRR 013 GP, (2) an amino acid substitution to proline at the amino acid position corresponding to amino acid position 328 of Lassa virus/H. sapiens-wt/NGA/2018/IRR 013 GP, and (3) an amino acid substitution to cysteine at the amino acid position corresponding to amino acid position 359 of Lassa virus/H. sapiens-wt/NGA/2018/IRR 013 GP. In some embodiments, the amino acid substitutions stabilize the pre-fusion conformation of a LASV GP. In some embodiments, the protein comprises the amino acid sequence of SEQ ID NO: 36 or 42. In some embodiments, the nucleotide sequence is codon optimized. See, e.g., Section 5.1.1, infra, regarding optimization.

In some embodiments, provided herein is a polynucleotide or transgene comprising a nucleotide sequence encoding a protein, wherein the nucleotide sequence comprises a nucleic acid sequence that is at least 80%, at least 81%, at least 82%, at least 83%, or at least 84% identical to the nucleotide sequence of SEQ ID NO:34 or 48. In some embodiments, the nucleotide sequence comprises nucleic acid sequence that is at least 85%, at least 86%, at least 87%, at least 88%, or at least 89% identical to the nucleotide sequence of SEQ ID NO:34 or 48. In some embodiments, the nucleotide sequence comprises nucleic acid sequence that is at least 90%, at least 91%, at least 92%, at least 93%, or at least 94% identical to the nucleotide sequence of SEQ ID NO:34 or 48. In some embodiments, the nucleotide sequence comprises nucleic acid sequence that is at least 95%, at least 96%, at least 97%, at least 98%, or at least 99% identical to the nucleotide sequence of SEQ ID NO:34 or 48. In some embodiments, the protein comprises an amino acid sequence that is at least 85%, at least 86%, at least 87%, at least 88%, or at least 89% identical to the amino acid sequence of SEQ ID NO:36 or 42. In some embodiments, the protein comprises an amino acid sequence that is at least 90%, at least 91%, at least 92%, at least 93%, or at least 94% identical to the amino acid sequence of SEQ ID NO:36 or 42. In some embodiments, the protein comprises an amino acid sequence that is at least 95%, at least 96%, at least 97%, at least 98%, or at least 99% identical to the amino acid sequence of SEQ ID NO:36 or 42. In some embodiments, the protein comprises amino acid substitutions at amino acid positions corresponding to amino acid positions 206, 328, and 359 of Lassa virus/H. sapiens-wt/NGA/2018/IRR 013 GP. In some embodiments, the protein comprises the following: (1) an amino acid substitution to cysteine at the amino acid position corresponding to amino acid position 206 of Lassa virus/H. sapiens-wt/NGA/2018/IRR 013 GP, (2) an amino acid substitution to proline at the amino acid position corresponding to amino acid position 328 of Lassa virus/H. sapiens-wt/NGA/2018/IRR 013 GP, and (3) an amino acid substitution to cysteine at the amino acid position corresponding to amino acid position 359 of Lassa virus/H. sapiens-wt/NGA/2018/IRR 013 GP. In some embodiments, the amino acid substitutions stabilize the pre-fusion conformation of a LASV GP. In some embodiments, the nucleotide sequence comprises the nucleotide sequence of SEQ ID NO:34 or 48.

In some embodiments, provided herein is a polynucleotide or transgene comprising a nucleotide sequence encoding protein, wherein the protein comprises an amino acid sequence that is at least 80%, at least 81%, at least 82%, at least 83%, or at least 84% identical to the amino acid sequence of SEQ ID NO:35 or 41. In some embodiments, the protein comprises an amino acid sequence that is at least 85%, at least 86%, at least 87%, at least 88%, or at least 89% identical to the amino acid sequence of SEQ ID NO:35 or 41. In some embodiments, the protein comprises an amino acid sequence that is at least 90%, at least 91%, at least 92%, at least 93%, or at least 94% identical to the amino acid sequence of SEQ ID NO:35 or 41. In some embodiments, the protein comprises an amino acid sequence that is at least 95%, at least 96%, at least 97%, at least 98%, or at least 99% identical to the amino acid sequence of SEQ ID NO:35 or 41. In some embodiments, the protein comprises the amino acid sequence of SEQ ID NO:35 or 41. In some embodiments, the nucleotide sequence is codon optimized. See, e.g., Section 5.1.1, infra, regarding optimization.

In some embodiments, provided herein is a polynucleotide or transgene comprising a nucleotide sequence encoding a protein, wherein the nucleotide sequence comprises a nucleic acid sequence that is at least 80%, at least 81%, at least 82%, at least 83%, or at least 84% identical to the nucleotide sequence of SEQ ID NO:33 or 47. In some embodiments, the nucleotide sequence comprises nucleic acid sequence that is at least 85%, at least 86%, at least 87%, at least 88%, or at least 89% identical to the nucleotide sequence of SEQ ID NO: 33 or 47. In some embodiments, the nucleotide sequence comprises nucleic acid sequence that is at least 90%, at least 91%, at least 92%, at least 93%, or at least 94% identical to the nucleotide sequence of SEQ ID NO: 33 or 47. In some embodiments, the nucleotide sequence comprises nucleic acid sequence that is at least 95%, at least 96%, at least 97%, at least 98%, or at least 99% identical to the nucleotide sequence of SEQ ID NO: 33 or 47. In some embodiments, the nucleotide sequence comprises the nucleotide sequence of SEQ ID NO: 33 or 47.

In another embodiment, described herein are polynucleotides or transgenes comprising a nucleotide sequence encoding a chimeric Lassa virus glycoprotein, wherein the chimeric Lassa virus glycoprotein comprises a LASV GP ectodomain (e.g., a LASV GP ectodomain described herein) and NDV F protein transmembrane and cytoplasmic domains. In some embodiments, the ectodomain of LASV GP comprises the amino acid sequence of SEQ ID NO: 35, 36, 41 or 42. In some embodiments, the ectodomain of LASV GP comprises an amino acid sequence that is at least 80%, at least 85%, or at least 90% identical to SEQ ID NO: 35, 36, 41, or 42. In some embodiments, the ectodomain of LASV GP comprises an amino acid sequence that is at least 95%, at least 96%, at least 97%, at least 98%, or at least 99% identical to SEQ ID NO: 35, 36, 41, or 42. In some embodiments, the ectodomain of LASV GP is encoded by a nucleotide sequence comprising the sequence of SEQ ID NO: 33 or 34. In some embodiments, the ectodomain of LASV GP is encoded by a nucleotide sequence comprising a nucleic acid sequence that is at least 75%, at least 80%, at least 85%, or at least 90% identical to SEQ ID NO: 33 or 34. In some embodiments, the ectodomain of LASV GP is encoded by a nucleotide sequence comprising a nucleic acid sequence that is at least 75%, at least 80%, at least 85%, or at least 90% identical to SEQ ID NO: 47 or 48. In some embodiments, the ectodomain of LASV GP is encoded by a nucleotide sequence comprising a nucleic acid sequence that is at least 95%, at least 96%, at least 97%, at least 98%, or at least 99% identical to SEQ ID NO: 33 or 34. In some embodiments, the ectodomain of LASV GP is encoded by a nucleotide sequence comprising a nucleic acid sequence that is at least 95%, at least 96%, at least 97%, at least 98%, or at least 99% identical to SEQ ID NO:47 or 48.

In some embodiments, provided herein is a polynucleotide or transgene comprising a nucleotide sequence encoding a chimeric Lassa virus glycoprotein, wherein the chimeric Lassa virus glycoprotein comprises a LASV GP ectodomain (e.g., a LASV GP ectodomain described herein) and NDV F protein transmembrane and cytoplasmic domains, wherein the LASV GP ectodomain comprises 1, 2, 3, 4, 5, 6, 7, 8 or more amino acid substitutions. In some embodiments, the amino acid substitutions are conservative amino acid substitutions. In some embodiments, the amino acid substitutions stabilize the pre-fusion conformation of the GP. In some embodiments, the amino acid substitutions are at amino acid positions corresponding to amino acid positions 206, 328, and 359 of Lassa virus/H. sapiens-wt/NGA/2018/IRR 013 GP. In some embodiments, the LASV GP ectodomain comprises the following amino acid substitutions: (1) an amino acid substitution to cysteine at the amino acid position corresponding to amino acid position 206 of Lassa virus/H. sapiens-wt/NGA/2018/IRR 013 GP, (2) an amino acid substitution to proline at the amino acid position corresponding to amino acid position 328 of Lassa virus/H. sapiens-wt/NGA/2018/IRR 013 GP, and (3) an amino acid substitution to cysteine at the amino acid position corresponding to amino acid position 359 of Lassa virus/H. sapiens-wt/NGA/2018/IRR 013 GP. In some embodiments, the LASV GP ectodomain comprises amino substitutions corresponding to those identified in SEQ ID NO: 12. The corresponding amino acid positions may be determined by aligning a Lassa virus GP with the GP of Lassa virus/H. sapiens-wt/NGA/2018/IRR 013.

In specific embodiments, the entire NDV F protein transmembrane and cytoplasmic domains is included in a chimeric Lassa virus glycoprotein. In a specific embodiment, the NDV F protein transmembrane and cytoplasmic domains comprise the amino acid sequence of SEQ ID NO:5. In some embodiments, the entire NDV F protein transmembrane and cytoplasmic domains is not included in a chimeric Lassa virus glycoprotein. For example, a few amino acid residues (e.g., 1, 2, 3, 4, 5, 6, 7, 8, 9, 10, 11, 12, 13, 14, 15, 1-5, 1-10, or 5-15 amino acid residues) upstream to the NDV F protein transmembrane may be included in a chimeric Lassa virus glycoprotein and/or a few amino acid residues (e.g., 1-5, 1-10, or 5-15 amino acid residues) downstream of the NDV F protein cytoplasmic domain may be included in a chimeric Lassa virus glycoprotein. For example, a few amino acid residues (e.g., 1, 2, 3, 4, 5, or 1-5 amino acid residues) less than the entire NDV F protein transmembrane may be included in a chimeric Lassa virus glycoprotein and/or a few amino acid residues (e.g., 1, 2, 3, 4, 5, or 1-5 amino acid residues) less than the entire NDV F protein cytoplasmic domain may be included. In specific embodiments, described herein are polynucleotides or transgenes comprising a nucleotide sequence encoding a chimeric Lassa virus glycoprotein, wherein the chimeric Lassa virus glycoprotein comprises a LASV GP ectodomain (e.g., a LASV GP ectodomain described herein), a NDV F protein transmembrane domain plus or minus 1, 2, 3, 4, or 5 amino acid residues, and a NDV F protein cytoplasmic domain plus or minus 1, 2, 3, 4, or 5 amino acid residues. In specific embodiments, the entire transmembrane and cytoplasmic domains of the LASV GP are not present in the chimeric Lassa virus glycoprotein. In some embodiments, 1, 2, or 3 amino acid residues of the transmembrane domain and/or cytoplasmic domain of the LASV GP are present in the chimeric Lassa virus glycoprotein. The ectodomain, transmembrane and cytoplasmic domains of the LASV GP and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the LASV GP and NDV F protein. See, e.g., Table 2, infra, with the transmembrane and cytoplasmic domains of NDV F protein indicated. In specific embodiments, the LASV GP ectodomain is fused to the NDV F protein transmembrane and cytoplasmic domains via a linker. The linker may be any linker that does not interfere with folding of the ectodomain, function of the ectodomain or both. For example, the linker may be a glycine-serine linker or glycine linker. In some embodiments, the NDV F protein transmembrane and cytoplasmic domains are fused directly to the LASV GP ectodomain. In certain embodiments, the polynucleotide or transgene encoding the chimeric Lassa virus glycoprotein, or LASV GP ectodomain of the chimeric Lassa virus glycoprotein is codon optimized. See, e.g., Section 5.1.1, infra, for a discussion regarding codon optimization.

In specific embodiments, NDV F protein transmembrane and cytoplasmic domains of a chimeric Lassa virus glycoprotein may be from any NDV strain known in the art or described herein. For example, NDV F protein transmembrane and cytoplasmic domains of a chimeric Lassa virus glycoprotein may be from the NDV F protein of LaSota strain, Hitchner B1 strain, Fuller strain, Ulster strain, Roakin strain, or Komarov strain. In some embodiments, the NDV F protein transmembrane and cytoplasmic domains are from the NDV F protein of LaSota strain. In some embodiments, the NDV F protein transmembrane and cytoplasmic domains comprise the amino acid sequence of SEQ ID NO:5.

In some embodiments, provided herein is a polynucleotide or transgene comprising the nucleotide sequence of SEQ ID NO:7 or 9. In some embodiments, provided herein is a polynucleotide or transgene comprising the nucleotide sequence of SEQ ID NO:7 or 9 without the signal sequence. In some embodiments, provided herein is a polynucleotide or transgene comprising the nucleotide sequence of SEQ ID NO:44 or 46. In some embodiments, provided herein is a polynucleotide or transgene comprising a nucleotide sequence that is at least 75%, at least 80%, at least 85%, or at least 90% identical to SEQ ID NO: 7 or 9. In some embodiments, provided herein is a polynucleotide or transgene comprising a nucleotide sequence that is at least 75%, at least 80%, at least 85%, or at least 90% identical to SEQ ID NO: 7 or 9 without the signal sequence. In some embodiments, provided herein is a polynucleotide or transgene comprising a nucleotide sequence that is at least 75%, at least 80%, at least 85%, or at least 90% identical to SEQ ID NO:44 or 46. In some embodiments, provided herein is a polynucleotide or transgene comprising a nucleotide sequence that is at least 95%, at least 96%, at least 97%, at least 98%, or at least 99% identical to SEQ ID NO: 7 or 9. In some embodiments, provided herein is a polynucleotide or transgene comprising a nucleotide sequence that is at least 95%, at least 96%, at least 97%, at least 98%, or at least 99% identical to SEQ ID NO: 7 or 9 without the signal sequence. In some embodiments, provided herein is a polynucleotide or transgene comprising a nucleotide sequence that is at least 95%, at least 96%, at least 97%, at least 98%, or at least 99% identical to SEQ ID NO: 44 or 46.

In some embodiments, provided herein is a polynucleotide or transgene comprising a nucleotide sequence encoding the amino acid sequence of SEQ ID NO: 11, 13, 38, or 40. In some embodiments, provided herein is a polynucleotide or transgene comprising a nucleotide sequence encoding an amino acid sequence that is at least 80%, at least 85%, or at least 90% identical to the amino acid sequence of SEQ ID NO: 11, 13, 38, or 40. In some embodiments, provided herein is a polynucleotide or transgene comprising a nucleotide sequence encoding an amino acid sequence that is at least 95%, at least 96%, at least 97%, at least 98%, or at least 99% identical to the amino acid sequence of SEQ ID NO: 11, 13, 38, or 40.

In some embodiments, a polynucleotide or transgene described herein comprises the elements needed for a NDV transcriptional unit. In certain embodiments, a polynucleotide or transgene described herein comprises a NDV regulatory signal (e.g., gene end, intergenic, and/or gene start sequences) and a Kozak sequence. In some embodiments, a polynucleotide or transgene described herein comprises a NDV gene end sequence, a NDV gene start sequence, and a Kozak sequence. In some embodiments, a polynucleotide or transgene described herein comprises a NDV regulatory signal (e.g., gene end, intergenic, and/or gene start sequences), a Kozak sequence, and a restriction site(s) to facilitate cloning. In some embodiments, a polynucleotide or transgene described herein comprises a NDV regulatory signal (e.g., gene end, intergenic, and/or gene start sequences), a Kozak sequence, a restriction site(s) to facilitate cloning, and additional nucleotides in the non-coding region to ensure compliance with the rule of six. See, e.g., SEQ ID NOS: 18-21 for examples of a restriction sequence (SacII), a gene end sequence, a gene start sequence and a Kozak sequence that may be used. In a preferred embodiment, the polynucleotide or transgene complies with the rule of six.

In a specific embodiment, a transgene encoding a chimeric Lassa virus glycoprotein is one described in the Example (Section 6), infra. In a specific embodiment, a transgene comprises a nucleotide sequence described in Table 3 or 4, infra. In a specific embodiment, a transgene encodes a protein comprising an amino acid sequence described in Table 3, infra. In a specific embodiment, a transgene encoding a chimeric Lassa virus glycoprotein comprises an amino acid sequence described in Table 3, infra.

In a specific embodiment, a polynucleotide or transgene described herein is incorporated into the genome of any NDV type or strain (e.g., NDV LaSota strain). See., e.g., Section 5.2, for types and strains of NDV that may be used. In a specific embodiment, the NDV is one described herein (e.g., in Section 5.2.1). The polynucleotide or transgene may be incorporated between any two NDV transcription units (e.g., between the NDV P and M transcription units, between the NDV NP and P transcription units, or between the NDV HN and L transcription units).

In some embodiments, provided herein is a nucleic acid sequence comprising a polynucleotide or transgene described herein and (1) a nucleotide sequence coding for a NDV F transcription unit, (2) a nucleotide sequence coding for a NDV M transcription unit, (3) a nucleotide sequence coding for a NDV L transcription unit, (4) a nucleotide sequence coding for a NDV P transcription unit, (5) a nucleotide sequence coding for a NDV HN transcription unit, and (6) a nucleotide sequence coding for a NDV HN transcription unit. In some embodiments, provided herein is a nucleic acid sequence comprising a polynucleotide or transgene described herein and (1) a NDV F transcription unit, (2) a NDV M transcription unit, (3) a NDV L transcription unit, (4) a NDV P transcription unit, (5) a NDV HN transcription unit, and (6) a NDV HN transcription unit. The polynucleotide or transgene may be incorporated between any two NDV transcription units (e.g., between the NDV P and M transcription units, between the NDV NP and P transcription units, or between the NDV HN and L transcription units).

In some embodiments, provided herein is a protein described herein. In some embodiments, provided herein a protein (e.g., a chimeric Lassa virus glycoprotein) is one encoded by a polynucleotide or transgene described herein. In some embodiments, provided herein is a recombinant protein encoded by a transgene described herein, or a polynucleotide, nucleic acid sequence, or nucleotide sequence described herein. In a specific embodiment, a recombinant LASV GP or LASV NP is one described in Section 6, infra. In a specific embodiment, a chimeric Lassa virus glycoprotein is one described in Section 6, infra. In some embodiments, provided herein is a recombinant protein comprising (or consisting of) an amino acid described herein (e.g., in Table 3 or 4, infra). In a specific embodiment, a chimeric Lassa virus glycoprotein comprises an amino acid sequence described in Table 3, infra. In specific embodiments, the protein is a recombinant protein.

In some embodiments, a LASV GP or a derivative thereof, a chimeric Lassa virus glycoprotein, or a protein comprising a LASV GP ectodomain or a derivative thereof retains one, two, or more functions of a LASV GP (e.g., binding to the LASV cellular receptor (matriglycan, a linear carbohydrate present on α-dystroglycan) and/or pH-dependent fusion of the viral envelope with the cellular target membrane). In some embodiments, a LASV GP or a derivative thereof, a chimeric Lassa virus glycoprotein, or a protein comprising a LASV GP ectodomain or a derivative thereof does not retain all of the functions of a LASV GP (e.g., the protein only retains only one or two functions). In some embodiments, a LASV GP or a derivative thereof, a chimeric Lassa virus glycoprotein, or a protein comprising a LASV GP ectodomain or a derivative thereof does not retain any functions of a LASV GP. In specific embodiments, a LASV GP or a derivative thereof, a chimeric Lassa virus glycoprotein, or a protein comprising a LASV GP ectodomain or a derivative thereof forms trimers. In specific embodiments, a derivative of a LASV GP or a protein comprising a LASV GP ectodomain or a LASV GP or a derivative thereof, a chimeric Lassa virus glycoprotein, or a protein comprising a LASV GP ectodomain or a derivative thereof has a prefusion conformation.

In some embodiments, a LASV NP or a derivative thereof retains one, two, or more functions of a LASV NP (e.g., antagonizes cellular IFN). In some embodiments, a LASV NP or a derivative thereof does not retain all of the functions of a LASV NP (e.g., the protein only retains one or two functions). In some embodiments, a LASV NP or a derivative thereof does not retain the ability to antagonize cellular IFN. In specific embodiments, a LASV NP or a derivative thereof does not have the exonuclease function of LASV NP described herein. In some embodiments, a LASV NP or a derivative thereof does not retain any functions of a LASV NP.

In some embodiments, a LASV GP or chimeric Lassa virus glycoprotein encoded by a polynucleotide sequence or transgene described herein retains one, two, or more functions of a LASV GP (e.g., binding to the LASV cellular receptor (matriglycan, a linear carbohydrate present on α-dystroglycan) and/or pH-dependent fusion of the viral envelope with the cellular target membrane). In some embodiments, a LASV GP or chimeric Lassa virus glycoprotein encoded by a polynucleotide sequence or transgene described herein does not retain all of the functions of a LASV GP (e.g., the protein only retains only one or two functions). In some embodiments, a LASV GP or chimeric Lassa virus glycoprotein encoded by a polynucleotide sequence or transgene described herein does not retain any functions of a LASV GP. In specific embodiments, a LASV GP or chimeric Lassa virus glycoprotein encoded by a polynucleotide sequence or transgene described herein forms trimers. In specific embodiments, a LASV GP or chimeric Lassa virus glycoprotein encoded by a polynucleotide sequence or transgene described herein has a prefusion conformation.

In some embodiments, a derivative of a LASV GP or a protein comprising a LASV GP ectodomain or a derivative thereof encoded by a polynucleotide sequence or transgene described herein retains one, two, or more functions of a LASV GP (e.g., binding to the LASV cellular receptor (matriglycan, a linear carbohydrate present on α-dystroglycan) and/or pH-dependent fusion of the viral envelope with the cellular target membrane). In some embodiments, a derivative of a LASV GP or a protein comprising a LASV GP ectodomain or a derivative thereof encoded by a polynucleotide sequence or transgene described herein does not retain all of the functions of a LASV GP (e.g., the protein only retains only one or two functions). In some embodiments, a derivative of a LASV GP or a protein comprising a LASV GP ectodomain or a derivative thereof encoded by a polynucleotide sequence or transgene described herein does not retain any functions of a LASV GP. In specific embodiments, a derivative of a LASV GP or a protein comprising a LASV GP ectodomain or a derivative thereof forms encoded by a polynucleotide sequence or transgene described herein trimers. In specific embodiments, a derivative of a LASV GP or a protein comprising a LASV GP ectodomain or a derivative thereof encoded by a polynucleotide sequence or transgene described herein has a prefusion conformation.

In some embodiments, a LASV NP encoded by a polynucleotide sequence or transgene described herein retains one, two, or more functions of a LASV NP (e.g., antagonizes cellular IFN). In some embodiments, a LASV NP encoded by a polynucleotide sequence or transgene described herein does not retain all of the functions of a LASV NP (e.g., the protein only retains one or two functions). In some embodiments, a LASV NP encoded by a polynucleotide sequence or transgene described herein does not retain the ability to antagonize cellular IFN. In specific embodiments, a LASV NP encoded by a polynucleotide sequence or transgene described herein does not have the exonuclease function of LASV NP described herein. In some embodiments, a LASV NP encoded by a polynucleotide sequence or transgene described herein does not retain any functions of a LASV NP.

In some embodiments, a derivative of a LASV NP encoded by a polynucleotide sequence or transgene described herein retains one, two, or more functions of a LASV NP (e.g., antagonizes cellular IFN). In some embodiments, a derivative of a LASV NP encoded by a polynucleotide sequence or transgene described herein does not retain all of the functions of a LASV NP (e.g., the protein only retains one or two functions). In some embodiments, a derivative of a LASV NP encoded by a polynucleotide sequence or transgene described herein does not retain the ability to antagonize cellular IFN. In specific embodiments, a derivative of a LASV NP encoded by a polynucleotide sequence or transgene described herein does not have the exonuclease function of LASV NP described herein. In some embodiments, a derivative of a LASV NP encoded by a polynucleotide sequence or transgene described herein does not retain any functions of a LASV NP.

In some embodiments, provided herein is a vector (e.g., a plasmid or viral vector) comprising a transgene, polynucleotide, nucleic acid or nucleotide sequence described herein.

### 5.1.1 Codon Optimization

Any codon optimization technique known to one of skill in the art may be used to codon optimize polynucleotide encoding a LASV GP or a portion thereof (e.g., ectodomain thereof), a derivative of a LASV GP or a portion thereof (e.g., ectodomain thereof), or a chimeric Lassa virus glycoprotein. In addition, any codon optimization technique known to one of skill in the art may be used to codon optimize polynucleotide encoding a LASV NP or a derivative of a LASV NP. Methods of codon optimization are known in the art, e.g., the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety.

### 5.2 Recombinant NDV and Compositions

In specific embodiments, provided herein is a recombinant Newcastle disease virus (NDV) comprising a packaged genome, wherein the package genome comprises a polynucleotide or transgene described herein (e.g., in Section 5.1 or 6). In some embodiments, provided herein is a recombinant Newcastle disease virus (NDV) comprising a protein described herein. The NDV may be any NDV described herein (e.g., in Section 5.2.1 or 6) or known to one of skill in the art.

In some embodiments, provided herein is a recombinant Newcastle disease virus (NDV) comprising a protein that comprises the amino acid sequence of SEQ ID NO:10, 12, 37 or 39. In some embodiments, provided herein is a recombinant NDV comprising a protein that comprises an amino acid sequence that is at least 80%, at least 85%, or at least 90%, identical to the amino acid sequence of SEQ ID NO:10, 12, 37, or 39. In some embodiments, provided herein is a recombinant NDV comprising a protein that comprises an amino acid sequence that is at least 95%, at least 96%, at least 97%, at least 98%, or at least 99%, identical to the amino acid sequence of SEQ ID NO:10, 12, 37, or 39.

In some embodiments, provided herein is a recombinant Newcastle disease virus (NDV) comprising a protein that comprises the amino acid sequence of SEQ ID NO: 11, 13, 38, or 40. In some embodiments, provided herein is a recombinant NDV comprising a protein that comprises an amino acid sequence that is at least 80%, at least 85%, or at least 90%, identical to the amino acid sequence of SEQ ID NO: 11, 13, 38, or 40. In some embodiments, provided herein is a recombinant NDV comprising a protein that comprises an amino acid sequence that is at least 95%, at least 96%, at least 97%, at least 98%, or at least 99%, identical to the amino acid sequence of SEQ ID NO: 11, 13, 38, or 40.

In some embodiments, provided herein is a recombinant NDV comprising a protein, wherein the protein comprises the amino acid sequence of SEQ ID NO:35 or 41. In some embodiments, provided herein is a recombinant NDV comprising a protein, wherein the protein comprises an amino acid sequence that is at least 80%, at least 85%, or at least 90%, identical to the amino acid sequence of SEQ ID NO:35 or 41. In some embodiments, provided herein is a recombinant NDV comprising a protein, wherein the protein comprises an amino acid sequence that is at least 95%, at least 96%, at least 97%, at least 98% or at least 99%, identical to the amino acid sequence of SEQ ID NO:35 or 41. In some embodiments, provided herein is a recombinant NDV comprising a protein, wherein the protein comprises the amino acid sequence of SEQ ID NO:36 or 42. In some embodiments, provided herein is a recombinant NDV comprising a protein, wherein the protein comprises an amino acid sequence that is at least 80%, at least 85%, or at least 90%, identical to the amino acid sequence of SEQ ID NO:36 or 42. In some embodiments, provided herein is a recombinant NDV comprising a protein, wherein the protein comprises an amino acid sequence that is at least 95%, at least 96%, at least 97%, at least 98%, or at least 99%, identical to the amino acid sequence of SEQ ID NO:36 or 42.

In some embodiments, provided herein is a recombinant Newcastle disease virus (NDV) comprising a protein that comprises the amino acid sequence of SEQ ID NO: 16 or 17. In some embodiments, provided herein is a recombinant NDV comprising a protein that comprises an amino acid sequence that is at least 80%, at least 85%, or at least 90%, identical to the amino acid sequence of SEQ ID NO: 16 or 17. In some embodiments, provided herein is a recombinant NDV comprising a protein that comprises an amino acid sequence that is at least 95%, at least 96%, at least 97%, at least 98%, or at least 99%, identical to the amino acid sequence of SEQ ID NO: 16 or 17.

In some embodiments, provided herein is a recombinant Newcastle disease virus (NDV) comprising a packaged genome, wherein the package genome comprises a transgene comprising a polynucleotide sequence encoding protein, wherein the protein comprises the ectodomain of a Lassa virus glycoprotein, and wherein the ectodomain comprises the amino acid sequence of SEQ ID NO:35 or 41. In some embodiments, provided herein is a recombinant NDV comprising a packaged genome, wherein the package genome comprises a transgene comprising a polynucleotide sequence encoding a protein, wherein the protein comprises the ectodomain of a Lassa virus glycoprotein, and wherein the ectodomain comprises an amino acid sequence that is at least 80%, at least 85%, or at least 90%, identical to the amino acid sequence of SEQ ID NO:35 or 41. In some embodiments, the ectodomain comprises an amino acid sequence that is at least 95%, at least 96%, at least 97%, at least 98%, or at least 99%, identical to the amino acid sequence of SEQ ID NO:35 or 41.

In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain comprises the amino acid sequence of SEQ ID NO:35 or 41. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain comprises an amino acid sequence that is at least 80%, at least 85%, or at least 90%, identical to the amino acid sequence of SEQ ID NO:35 or 41. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain comprises an amino acid sequence that is at least 95%, at least 96%, at least 97%, at least 98%, or at least 99%, identical to the amino acid sequence of SEQ ID NO:35 or 41.

In some embodiments, provided herein is a recombinant Newcastle disease virus (NDV) comprising a packaged genome, wherein the package genome comprises a transgene comprising a polynucleotide sequence encoding a protein, wherein the protein comprises the ectodomain of a Lassa virus glycoprotein, and wherein the ectodomain comprises the amino acid sequence of SEQ ID NO:36 or 42. In some embodiments, provided herein is a recombinant NDV comprising a packaged genome, wherein the package genome comprises a transgene comprising a polynucleotide sequence encoding a protein, wherein the protein comprises the ectodomain of a Lassa virus glycoprotein, and wherein the ectodomain comprises an amino acid sequence that is at least 80%, at least 85%, or at least 90%, identical to the amino acid sequence of SEQ ID NO: 36 or 42. In some embodiments, the ectodomain comprises an amino acid sequence that is at least 95%, at least 96%, at least 97%, at least 98%, or at least 99%, identical to the amino acid sequence of SEQ ID NO: 36 or 42.

In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain comprises the amino acid sequence of SEQ ID NO:36 or 42. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain comprises an amino acid sequence that is at least 80%, at least 85%, or at least 90%, identical to the amino acid sequence of SEQ ID NO: 36 or 42. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain comprises an amino acid sequence that is at least 95%, at least 96%, at least 97%, at least 98%, or at least 99%, identical to the amino acid sequence of SEQ ID NO: 36 or 42.

In some embodiments, provided herein is a recombinant NDV comprising a packaged genome, wherein the package genome comprises a transgene comprising a polynucleotide sequence encoding a protein, wherein the protein comprises the ectodomain of a Lassa virus glycoprotein, and wherein the ectodomain is encoded by the nucleotide sequence of SEQ ID NO:33 or 34. In some embodiments, provided herein is a recombinant NDV comprising a packaged genome, wherein the package genome comprises a transgene comprising a polynucleotide sequence encoding a protein, wherein the protein comprises the ectodomain of a Lassa virus glycoprotein, and wherein the ectodomain is encoded by the nucleotide sequence of SEQ ID NO:33 or 34 without the signal sequence. In some embodiments, provided herein is a recombinant NDV comprising a packaged genome, wherein the package genome comprises a transgene comprising a polynucleotide sequence encoding a protein, wherein the protein comprises the ectodomain of a Lassa virus glycoprotein, and wherein the ectodomain is encoded by the nucleotide sequence of SEQ ID NO:47 or 48.

In some embodiments, provided herein is a recombinant Newcastle disease virus (NDV) comprising a packaged genome, wherein the package genome comprises a transgene comprising a polynucleotide sequence encoding a protein, wherein the protein comprises the ectodomain of a Lassa virus glycoprotein, and wherein the ectodomain is encoded by a nucleotide sequence that is at least 75%, at least 80%, at least 81%, at least 82%, at least 83%, at least 84%, or at least 85% identical to the nucleotide sequence of SEQ ID NO:33 or 34. In some embodiments, the ectodomain is encoded by a nucleotide sequence that is at least 86%, at least 87%, at least 88%, at least 89%, or at least 89% identical to the nucleotide sequence of SEQ ID NO:33 or 34. In some embodiments, the ectodomain is encoded by a nucleotide sequence that is at least 90%, at least 91%, at least 92%, at least 93%, or at least 94% identical to the nucleotide sequence of SEQ ID NO:33 or 34. In some embodiments, the ectodomain is encoded by a nucleotide sequence that is at least 95%, at least 96%, at least 97%, at least 98%, or at least 99% identical to the nucleotide sequence of SEQ ID NO:33 or 34. In some embodiments, the ectodomain is encoded by the nucleotide sequence of SEQ ID NO:33 or 34.

In some embodiments, provided herein is a recombinant Newcastle disease virus (NDV) comprising a packaged genome, wherein the package genome comprises a transgene comprising a polynucleotide sequence encoding a protein, wherein the protein comprises the ectodomain of a Lassa virus glycoprotein, and wherein the ectodomain is encoded by a nucleotide sequence that is at least 75%, at least 80%, at least 81%, at least 82%, at least 83%, at least 84%, or at least 85% identical to the nucleotide sequence of SEQ ID NO:33 or 34, without the signal peptide. In some embodiments, the ectodomain is encoded by a nucleotide sequence that is at least 86%, at least 87%, at least 88%, at least 89%, or at least 89% identical to the nucleotide sequence of SEQ ID NO:33 or 34. In some embodiments, the ectodomain is encoded by a nucleotide sequence that is at least 90%, at least 91%, at least 92%, at least 93%, or at least 94% identical to the nucleotide sequence of SEQ ID NO:33 or 34, without the signal peptide. In some embodiments, the ectodomain is encoded by a nucleotide sequence that is at least 95%, at least 96%, at least 97%, at least 98%, or at least 99% identical to the nucleotide sequence of SEQ ID NO:33 or 34, without the signal peptide. In some embodiments, the ectodomain is encoded by the nucleotide sequence of SEQ ID NO:33 or 34, without the signal peptide.

In some embodiments, provided herein is a recombinant Newcastle disease virus (NDV) comprising a packaged genome, wherein the package genome comprises a transgene comprising a polynucleotide sequence encoding a protein, wherein the protein comprises the ectodomain of a Lassa virus glycoprotein, and wherein the ectodomain is encoded by a nucleotide sequence that is at least 75%, at least 80%, at least 81%, at least 82%, at least 83%, at least 84%, or at least 85% identical to the nucleotide sequence of SEQ ID NO:47 or 48. In some embodiments, the ectodomain is encoded by a nucleotide sequence that is at least 86%, at least 87%, at least 88%, at least 89%, or at least 89% identical to the nucleotide sequence of SEQ ID NO: 47 or 48. In some embodiments, the ectodomain is encoded by a nucleotide sequence that is at least 90%, at least 91%, at least 92%, at least 93%, or at least 94% identical to the nucleotide sequence of SEQ ID NO: 47 or 48. In some embodiments, the ectodomain is encoded by a nucleotide sequence that is at least 95%, at least 96%, at least 97%, at least 98%, or at least 99% identical to the nucleotide sequence of SEQ ID NO: 47 or 48. In some embodiments, the ectodomain is encoded by the nucleotide sequence of SEQ ID NO:47 or 48.

In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by the nucleotide sequence of SEQ ID NO:33 or 34. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by the nucleotide sequence of SEQ ID NO:33 or 34 without the signal sequence. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by the nucleotide sequence of SEQ ID NO:47 or 48.

In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by a nucleotide sequence that is at least 75%, at least 80%, at least 85%, or at least 90% identical to the nucleotide sequence of SEQ ID NO:33 or 34. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by a nucleotide sequence that is at least 75%, at least 80%, at least 85%, or at least 90% identical to the nucleotide sequence of SEQ ID NO:33 or 34 without the signal sequence. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by a nucleotide sequence that is at least 75%, at least 80%, at least 85%, or at least 90% identical to the nucleotide sequence of SEQ ID NO:47 or 48.

In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by a nucleotide sequence that is at least 95%, at least 96%, at least 97%, at least 98%, or at least 99% identical to the nucleotide sequence of SEQ ID NO:33 or 34. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by a nucleotide sequence that is at least 95%, at least 96%, at least 97%, at least 98%, or at least 99% identical to the nucleotide sequence of SEQ ID NO:33 or 34 without the signal sequence. In some embodiments, provided herein is a recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by a nucleotide sequence that is at least 95%, at least 96%, at least 97%, at least 98%, or at least 99% identical to the nucleotide sequence of SEQ ID NO:47 or 48.

In another embodiment, described herein are recombinant NDV comprising a packaged genome, wherein the packaged genome comprises a transgene encoding a protein described herein. In another embodiment, described herein are recombinant NDV comprising a packaged genome, wherein the packaged genome comprises a transgene encoding a chimeric Lassa virus glycoprotein described herein. In a specific embodiment, the chimeric Lassa virus glycoprotein is expressed by cells infected with the recombinant NDV. In another specific embodiment, the chimeric Lassa virus glycoprotein is incorporated into the NDV virion. In another specific embodiment, the chimeric Lassa virus glycoprotein is expressed by cells infected with the recombinant NDV and the chimeric Lassa virus glycoprotein is incorporated into the NDV virion.

In a specific embodiment, a recombinant NDV is one described in the Example (Section 6), infra. In specific embodiments, a recombinant NDV described herein is replication competent. In other embodiments, a recombinant NDV described herein has been inactivated.

In some embodiments, the packaged genome of recombinant NDV encodes a LASV GP, LASV NP or chimeric Lassa virus glycoprotein described herein. In some embodiments, the packaged genome of recombinant NDV encodes a derivative of a LASV GP described herein, protein comprising a LASV GP ectodomain or a derivative thereof described herein, or a derivative of a LASV NP described herein. In certain embodiment, the genome of the recombinant NDV does not comprise a heterologous sequence encoding a heterologous protein other than the LASV GP, LASV NP or chimeric Lassa virus glycoprotein described herein. In certain embodiment, the genome of the recombinant NDV does not comprise a heterologous sequence encoding a heterologous protein other than a derivative of a LASV GP described herein, protein comprising a LASV GP ectodomain or a derivative thereof described herein, or a derivative of a LASV NP described herein. In a specific embodiment, a heterologous sequence encodes a protein that is not found associated with naturally-occurring NDV. In some embodiments, the genome of the recombinant NDV does not comprise a transgene other than a transgene encoding a LASV GP, LASV NP or chimeric Lassa virus glycoprotein described herein. In some embodiments, the genome of the recombinant NDV does not comprise a transgene other than a transgene encoding a derivative of a LASV GP described herein, protein comprising a LASV GP ectodomain or a derivative thereof described herein, or a derivative of a LASV NP described herein. In preferred embodiments, a recombinant NDV described herein comprises a packaged genome, wherein the genome comprises the genes found in NDV and a transgene encoding a LASV GP, LASV NP or chimeric Lassa virus glycoprotein described herein. In preferred embodiments, a recombinant NDV described herein comprises a packaged genome, wherein the genome comprises the genes found in NDV and a transgene encoding a derivative of a LASV GP described herein, protein comprising a LASV GP ectodomain or a derivative thereof described herein, or a derivative of a LASV NP described herein. In other words, the recombinant NDV encodes for both NDV F protein and the Lassa virus glycoprotein or a derivative thereof, a Lassa virus nucleoprotein or a derivative thereof, a protein comprising a LASV GP ectodomain or a derivative thereof described herein, or a chimeric Lassa virus glycoprotein described herein. In some embodiments, a recombinant NDV described herein comprises a packaged genome, wherein the genome comprises the genes found in NDV and a transgene encoding a LASV GP, LASV NP or chimeric Lassa virus glycoprotein described herein, but does not include any other transgenes. In some embodiments, a recombinant NDV described herein comprises a packaged genome, wherein the genome comprises the genes found in NDV and a transgene encoding a derivative of a LASV GP described herein, protein comprising a LASV GP ectodomain or a derivative thereof described herein, or a derivative of a LASV NP described herein, but does not include any other transgenes. In some embodiments, a recombinant NDV described herein comprises a packaged genome, wherein the genome comprises the genes found in NDV, a transgene encoding a LASV GP described herein, and a second transgene encoding a LASV GP signal peptide (e.g., one described below), but does not include any other transgenes.

In a specific embodiment, provided herein is a NDV virion comprising a LASV GP, LASV NP or chimeric Lassa virus glycoprotein described herein. In a specific embodiment, provided herein is a NDV virion comprising a derivative of a LASV GP described herein, protein comprising a LASV GP ectodomain or a derivative thereof described herein, or a derivative of a LASV NP described herein. See, e.g., Section 5.1 or 6 for examples of such a protein that may incorporated into the virion of a recombinant NDV. In a specific embodiment, the protein is one described in Section 5.1. In specific embodiments, the NDV virion is recombinantly produced.

In a specific embodiment, provided herein is a NDV virion comprising a LASV GP or chimeric Lassa virus glycoprotein described herein (e.g., Section 5.1 or 6). In a specific embodiment, provided herein is a NDV virion comprising a derivative of a LASV GP described herein, protein comprising a LASV GP ectodomain or a derivative thereof described herein, or a derivative of a LASV NP described herein (e.g., Section 5.1 or 6).

In a specific embodiment, a LASV GP or chimeric Lassa virus glycoprotein described herein is in a pre-fusion conformation. In a specific embodiment, a derivative of a LASV GP described herein, protein comprising a LASV GP ectodomain or a derivative thereof described herein is in a pre-fusion conformation. In some embodiments, a LASV GP or chimeric Lassa virus glycoprotein described herein is in a post-fusion conformation. In some embodiments, a derivative of a LASV GP described herein, protein comprising a LASV GP ectodomain or a derivative thereof described herein is in a post-fusion conformation.

A protein described herein may be isolated from a cell (e.g., a cell line or primary cell) or embryonated egg (e.g., embryonated chicken egg). An “isolated” protein is a protein which is substantially separated from other proteins. An “isolated” protein is one which is separated from other proteins which are present in the natural source of the protein. Moreover, an “isolated” protein can be substantially free of other cellular material, or culture medium when produced by recombinant techniques, or substantially free of chemical precursors or other chemicals when chemically synthesized.

### 5.2.1 NDV

Newcastle disease virus (NDV) is a member of the Avulavirus genus in the Paramyxoviridae family, which has been shown to infect a number of avian species (Alexander, DJ (1988). Newcastle disease, Newcastle disease virus—an avian paramyxovirus. Kluwer Academic Publishers: Dordrecht, The Netherlands. pp 1-22). NDV possesses a single-stranded RNA genome in negative sense and does not undergo recombination with the host genome or with other viruses (Alexander, DJ (1988). Newcastle disease, Newcastle disease virus—an avian paramyxovirus. Kluwer Academic Publishers: Dordrecht, The Netherlands. pp 1-22). The genomic RNA contains genes in the order of 3′-NP-P-M-F-HN-L-5′. Two additional proteins, V and W, are produced by NDV from the P gene by alternative mRNAs that are generated by RNA editing. The genomic RNA also contains a leader sequence at the 3′ end.

The structural elements of the virion include the virus envelope which is a lipid bilayer derived from the cell plasma membrane. The glycoprotein, hemagglutinin-neuraminidase (HN) protrudes from the envelope allowing the virus to contain both hemagglutinin (e.g., receptor binding/fusogenic) and neuraminidase activities. The fusion glycoprotein (F), which also interacts with the viral membrane, is first produced as an inactive precursor, then cleaved post-translationally to produce two disulfide linked polypeptides. The active F protein is involved in penetration of NDV into host cells by facilitating fusion of the viral envelope with the host cell plasma membrane. The matrix protein (M) is involved with viral assembly, and interacts with both the viral membrane as well as the nucleocapsid proteins.

The main protein subunit of the NDV nucleocapsid is the nucleocapsid protein (NP) which confers helical symmetry on the capsid. In association with the nucleocapsid are the P and L proteins. The phosphoprotein (P), which is subject to phosphorylation, is thought to play a regulatory role in transcription, and may also be involved in methylation, phosphorylation and polyadenylation. The L gene, which encodes an RNA-dependent RNA polymerase, is required for viral RNA synthesis together with the P protein. The L protein, which takes up nearly half of the coding capacity of the viral genome is the largest of the viral proteins, and plays an important role in both transcription and replication.

Any NDV type or strain may be serve as the “backbone” that is engineered to encode a polynucleotide or transgene described herein, including, but not limited to, naturally-occurring strains, variants or mutants, mutagenized viruses, reassortants and/or genetically engineered viruses. See, e.g., Section 5.1 and Section 6 for examples of a polynucleotides or transgenes. In a specific embodiment, a polynucleotide or transgene described herein is incorporated into the genome of a lentogenic NDV. In another specific embodiment, a polynucleotide or transgene described herein is incorporated into the genome of NDV strain LaSota. In another embodiment, a polynucleotide or transgene described herein is incorporated into the genome of NDV Hitchner B1 strain. In some embodiments, a lentogenic strain other than NDV Hitchner B1 strain is used as the backbone into which a nucleotide sequence may be incorporated. The polynucleotide or transgene described herein may be incorporated into the NDV genome between two transcription units (e.g., between the NDV M and P transcription units, between the NDV NP and P transcription units, or between the NDV HN and L transcription units).

In a specific embodiment, a NDV that is engineered to a polynucleotide or transgene described herein is a naturally-occurring strain. Specific examples of NDV strains include, but are not limited to, Hitchner B1 strain (see, e.g., GenBank No. AF309418 or NC_002617) and LaSota strain (see, e.g., GenBank Nos. AY845400, AF07761.1 and JF950510.1, and GI No. 56799463). In a specific embodiment, the NDV that is engineered to comprises a polynucleotide or transgene described herein is the Hitchner B1 strain. Table 1 provides cDNA sequences of the genome of certain NDV strains, which may be used in accordance with the provisions described herein. In another embodiment, the NDV that is engineered to comprise a polynucleotide or transgene described herein is a B1 strain as identified by GenBank No. AF309418 or NC_002617. In a specific embodiment, the nucleotide sequence of the Hitchner B1 genome comprises an RNA sequence corresponding to the negative sense of the cDNA sequence set forth in SEQ ID NO:2. In another specific embodiment, the NDV that is engineered to comprise a polynucleotide or transgene described herein is the LaSota strain. In another embodiment, the NDV that is engineered to comprise a polynucleotide or transgene described herein is a LaSota strain as identified by AY845400, AF07761.1 or JF950510.1. In a specific embodiment, the nucleotide sequence of the LaSota genome comprises an RNA sequence corresponding to the negative sense of the cDNA sequence set forth in SEQ ID NO:1. In another specific embodiment, the nucleotide sequence of the LaSota genome comprises an RNA sequence corresponding to the negative sense of the cDNA sequence set forth in SEQ ID NO:3. One skilled in the art will understand that the NDV genomic RNA sequence is an RNA sequence corresponding to the negative sense of a cDNA sequence encoding the NDV genome. Thus, any program that generates converts a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar). Accordingly, the nucleotide sequences provided in Tables 1-4, infra, may be readily converted to the negative-sense RNA sequence of the NDV genome by one of skill in the art.

In a specific embodiment, the NDV that is engineered to comprise a polynucleotide or transgene described herein comprises a genome encoding an NDV F protein in which a leucine amino acid residue at amino acid position 289 of NDV F protein is substituted for alanine (as described by, e.g., Sergel et al., 2000, Journal of Virology 74: 5101-5107). In another specific embodiment, the NDV that is engineered to comprise a polynucleotide or transgene described herein comprises a nucleotide sequence encoding an NDV F protein in which leucine at the amino acid position corresponding to amino acid residue 289 of LaSota NDV F protein is substituted for alanine. An alignment of NDV F proteins may be conducted to identify the amino acid position corresponding to amino acid residue 289 of LaSota NDV F protein. In another specific embodiment, the NDV that is engineered to comprise a polynucleotide or transgene described herein comprises a nucleotide sequence encoding an NDV F protein in which leucine at the amino acid residue 289 of LaSota NDV F protein is substituted for alanine. In another specific embodiment, the NDV that is engineered to comprise a polynucleotide or transgene described herein is of the LaSota strain (e.g., GenBank Accession Nos. AY845400, AF07761.1 or JF950510.1) and the genome of the LaSota strain encodes an NDV F protein in which a leucine amino acid residue at amino acid position 289 of NDV F protein is substituted for alanine. In another specific embodiment, the NDV that is engineered to comprise a polynucleotide or transgene described herein is of the LaSota strain (e.g., GenBank Accession Nos. AY845400, AF07761.1 or JF950510.1) and the genome of the LaSota strain comprises a nucleotide sequence encoding LaSota NDV F protein in which leucine at amino acid residue 289 of the NDV F protein (as counted by the LaSota strain F protein) is substituted for alanine. In another specific embodiment, the NDV that is engineered to comprise a polynucleotide or transgene described herein is of the Hitchner B1 strain (e.g., GenBank No. AF309418 or NC_002617) and the genome of the Hitchner B1 strain encodes an NDV F protein in which a leucine amino acid residue at amino acid position 289 of NDV F protein (as counted by the LaSota strain F protein) is substituted for alanine.

In some embodiments, the NDV that is engineered to comprise a polynucleotide or transgene described herein is of the Fuller strain. In certain embodiments, the NDV genome that is engineered to comprise a polynucleotide or transgene described herein is of the Ulster strain. In some embodiments, the NDV that is engineered to comprise a polynucleotide or transgene described herein is of the Roakin strain. In certain embodiments, the NDV that is engineered to comprise a polynucleotide or transgene described herein is of the Komarov strain. In some embodiments, the NDV that is engineered to comprise a polynucleotide or transgene described herein is of the Roakin strain. In certain embodiments, the NDV that is engineered to comprise a polynucleotide or transgene described herein is of the r73T-R1 16 virus.

In specific embodiments, the NDV that is engineered to comprise a polynucleotide or transgene described herein is not pathogenic in birds as assessed by a technique known to one of skill. In certain specific embodiments, the NDV that is engineered to comprise a polynucleotide or transgene described herein is not pathogenic as assessed by intracranial injection of 1-day-old chicks with the virus, and disease development and death as scored for 8 days. In some embodiments, the NDV that is engineered to comprise a polynucleotide or transgene described herein has an intracranial pathogenicity index of less than 0.7, less than 0.6, less than 0.5, less than 0.4, less than 0.3, less than 0.2 or less than 0.1. In certain embodiments, the NDV that is engineered to comprise a polynucleotide or transgene described herein has an intracranial pathogenicity index of zero. See, e.g., OIE Terrestrial Manual 2012, Chapter 2.3.14, entitled “Newcastle Disease (Infection With Newcastle Disease Virus) for a description of this assay, which is found at the following website www.oie.int/fileadmin/Home/eng/Health_standards/tahm/2.03.14_NEWCASTLE_DIS.pdf, which is incorporated herein by reference in its entirety.

In certain embodiments, the NDV that is engineered to comprise a polynucleotide or transgene described herein is a mesogenic strain that has been genetically engineered so as not be a considered pathogenic in birds as assessed by techniques known to one skilled in the art.

In preferred embodiments, the NDV that is engineered to comprise a polynucleotide or transgene described herein is non-pathogenic in humans. In preferred embodiments, the NDV that is engineered to comprise a polynucleotide or transgene described herein is non-pathogenic in human and avians. In certain embodiments, the NDV that is engineered to comprise a polynucleotide or transgene described herein is attenuated such that the NDV remains, at least partially, infectious and can replicate in vivo, but only generate low titers resulting in subclinical levels of infection that are non-pathogenic (see, e.g., Khattar et al., 2009, J. Virol. 83:7779-7782). Such attenuated NDVs may be especially suited for embodiments wherein the virus is administered to a subject in order to act as an immunogen, e.g., a live vaccine. The viruses may be attenuated by any method known in the art. In a specific embodiment, the genome of NDV comprises sequences necessary for infection and replication of the virus such that progeny is produced and the infection level is subclinical. In certain embodiments, NDV is attenuated by introducing one, two, or more mutations (e.g., amino acid substitutions) in the NDV V protein.

In a specific embodiment, provided herein is a nucleic acid sequence comprising: (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a polynucleotide or transgene described herein. In certain embodiments, the NDV transcription units are LaSota NDV transcription units. In a specific embodiment, provided herein is a nucleotide sequence comprising: (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a polynucleotide or transgene described herein, wherein the NDV F transcription unit encodes an NDV F protein with an amino acid substitution of leucine to alanine at the amino acid residue corresponding to amino acid position 289 of LaSota NDV F protein. In another specific embodiment, provided herein is a nucleotide sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a polynucleotide or transgene described herein, wherein the NDV F transcription unit encodes an NDV F protein with an amino acid substitution of leucine to alanine at amino acid position 289 of LaSota NDV F protein. In certain embodiments, the NDV transcription units are LaSota NDV transcription units. In certain embodiments, the nucleotide sequence is part of a vector (e.g., a plasmid). The vector may be a plasmid or a viral vector. In specific embodiments, the nucleic acid sequence is isolated.

In a specific embodiment, provided herein is a nucleic acid sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a polynucleotide or transgene described herein. In another specific embodiment, provided herein is a polynucleotide sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a polynucleotide or transgene described herein, wherein the NDV F comprises an amino acid substitution of leucine to alanine at the amino acid position corresponding to amino acid residue 289 of LaSota NDV F. In another specific embodiment, provided herein is a polynucleotide sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position 289 of LaSota NDV F. In certain embodiments, the NDV proteins are LaSota NDV proteins. In another specific embodiment, provided herein is a polynucleotide sequence comprising a nucleotide sequence of an NDV genome known in the art or described (see, e.g., Section 5.1 or the Example below; see also SEQ ID NO: 1, 2 or 3) and a transgene described herein. In certain embodiments, the nucleic acid sequence is part of a vector (e.g., a plasmid). The vector may be a plasmid or a viral vector. In a specific embodiment, the nucleic acid sequence is isolated.

### 5.3 Construction of NDV

The recombinant NDVs described herein (see, e.g., Sections 5.2 and 6) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. patent application Ser. No. 09/152,845; in International Patent Publications PCT WO 97/12032 published Apr. 3, 1997; WO 96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer a NDV described herein. Briefly, a complete cDNA of a NDV (e.g., the Hitchner B1 strain or LaSota strain) is constructed, inserted into a plasmid vector and engineered to contain a unique restriction site between two transcription units (e.g., the NDV P and M genes; the NDV NP and P genes; or the NDV HN and L genes). A nucleotide sequence encoding a heterologous amino acid sequence (e.g., a polynucleotide or transgene described herein, or other nucleotide sequence described herein) may be inserted into the viral genome at the unique restriction site. Alternatively, a nucleotide sequence encoding a heterologous amino acid sequence (e.g., a polynucleotide or transgene described herein, or other nucleotide sequence described herein) may be engineered into a NDV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety).

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribosomes to the RNA molecule and allow downstream translation in a cap independent manner. Briefly, a coding region of one protein is inserted downstream of the ORF of a second protein. The insertion is flanked by an IRES and any untranslated signal sequences necessary for proper expression and/or function. The insertion must not disrupt the open reading frame, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which are incorporated by reference herein in their entirety).

Methods for cloning recombinant NDV to encode a transgene and express a heterologous protein encoded by the transgene are known to one skilled in the art, such as, e.g., insertion of the transgene into a restriction site that has been engineered into the NDV genome, inclusion an appropriate signals in the transgene for recognition by the NDV RNA-dependent-RNA polymerase (e.g., sequences upstream of the open reading frame of the transgene that allow for the NDV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for NDV cloning; and inclusion of silent mutations to remove extraneous gene end and/or gene start sequences within the transgene. See, e.g., SEQ ID NO:18-21 for examples of a restriction site sequence, gene end sequence, gene start sequence, and Kozak sequence. Regarding the rule of six, one skilled in the art will understand that efficient replication of NDV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a recombinant NDV described herein, care should be taken to satisfy the “Rule of Six” for NDV cloning. Methods known to one skilled in the art to satisfy the Rule of Six for NDV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi:10.3791/50830 (2013) for a discussion of methods for cloning and rescuing of NDV (e.g., recombinant NDV), which is incorporated by reference herein in its entirety.

In some embodiments, a LASV GP protein signal sequence is provided in trans along with the recombinant NDV to a cell (e.g., an in vitro or ex vivo cell) or subject. In some embodiments, a vector comprising a nucleotide sequence encoding a LASV GP protein signal sequence is provided in trans along with the recombinant NDV to a cell (e.g., an in vitro or ex vivo cell).

In a specific embodiment, an NDV described herein (see, e.g., Section 5.2, and 6) may be generated according to a method described in Section 6, infra.

Techniques and procedures described or referenced herein include those that are generally well understood and/or commonly employed using conventional methodology by those skilled in the art, such as, for example, the widely utilized methodologies described in, e.g., Sambrook et al., Molecular Cloning: A Laboratory Manual (3d ed. 2001); Current Protocols in Molecular Biology (Ausubel et al. eds., 2003). Conventional methodologies well understood and/or commonly employed by those of skill in the art may be used to produced a protein described herein.

### 5.4 Propagation of Ndvs

The recombinant NDVs described herein (e.g., Sections 5.2 and 6) can be propagated in any substrate that allows the virus to grow to titers that permit the uses of the viruses described herein. In one embodiment, the substrate allows the recombinant NDVs described herein to grow to titers comparable to those determined for the corresponding wild-type viruses.

The recombinant NDVs described herein (e.g., Sections 5.2 and 6) may be grown in cells (e.g., avian cells, chicken cells, etc.) that are susceptible to infection by the viruses, embryonated eggs (e.g., chicken eggs or quail eggs) or animals (e.g., birds). Such methods are well known to those skilled in the art. In a specific embodiment, the recombinant NDVs described herein may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, the recombinant NDVs described herein may be propagated in cell lines, e.g., cancer cell lines such as HeLa cells, MCF7 cells, THP-1 cells, U87 cells, DU145 cells, Lncap cells, and T47D cells. In certain embodiments, the cells or cell lines (e.g., cancer cells or cancer cell lines) are obtained, derived, or obtained and derived from a human(s). In another embodiment, the recombinant NDVs described herein are propagated in interferon deficient systems or interferon (IFN) deficient substrates, such as, e.g., IFN deficient cells (e.g., IFN deficient cell lines) or IFN deficient embryonated eggs. In another embodiment, the recombinant NDVs described herein are propagated in chicken cells or embryonated chicken eggs. Representative chicken cells include, but are not limited to, chicken embryo fibroblasts and chicken embryo kidney cells. In a specific embodiment, the recombinant NDVs described herein are propagated in Vero cells. In another specific embodiment, the recombinant NDVs described herein are propagated in chicken eggs or quail eggs. In certain embodiments, a recombinant NDV virus described herein is first propagated in embryonated eggs and then propagated in cells (e.g., a cell line). In another specific embodiment, the recombinant NDVs described herein are propagated as described in Section 6, infra.

The recombinant NDVs described herein may be propagated in embryonated eggs (e.g., chicken embryonated eggs), e.g., from 6 to 14 days old, 6 to 12 days old, 6 to 10 days old, 6 to 9 days old, 6 to 8 days old, 8 to 10 day old, 9 to 11 days old, or 10 to 12 days old. In a specific embodiment, 10 day old embryonated chicken eggs are used to propagate the recombinant NDVs described herein. Young or immature embryonated eggs (e.g., chicken embryonated eggs) can be used to propagate the recombinant NDVs described herein. Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. The recombinant NDVs described herein can be propagated in different locations of the embryonated egg, e.g., the allantoic cavity (such as, e.g., the allantoic cavity of chicken embryonated eggs). For a detailed discussion on the growth and propagation viruses, see, e.g., U.S. Pat. Nos. 6,852,522 and 7,494,808, both of which are hereby incorporated by reference in their entireties.

In a specific embodiment, a virus is propagated as described in the Example below (e.g., Section 6).

For virus isolation, the recombinant NDVs described herein can be removed from embryonated eggs or cell culture and separated from cellular components, typically by well-known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography.

In a specific embodiment, virus isolation from allantoic fluid of an infected egg (e.g., a chicken egg) begins with harvesting allantoic fluid, which is clarified using a filtration system to remove cells and other large debris.

In a specific embodiment, provided herein is a cell (e.g., a cell line) or embryonated egg (e.g., a chicken embryonated egg) comprising a recombinant NDV described herein. In another specific embodiment, provided herein is a method for propagating a recombinant NDV described herein, the method comprising culturing a cell (e.g., a cell line) or embryonated egg (e.g., a chicken embryonated egg) infected with the recombinant NDV. In some embodiments, the method may further comprise isolating or purifying the recombinant NDV from the cell or embryonated egg. In a specific embodiment, provided herein is a method for propagating a recombinant NDV described herein, the method comprising (a) culturing a cell (e.g., a cell line) or embryonated egg infected with a recombinant NDV described herein; and (b) isolating the recombinant NDV from the cell or embryonated egg. The cell or embryonated egg may be one described herein or known to one of skill in the art. In some embodiments, the cell or embryonated egg is IFN deficient. The cell may be one described herein. In specific embodiments, the cell is in vitro or ex vivo. In specific embodiments, the cell(s) is isolated.

In a specific embodiment, provided herein is a method for producing a pharmaceutical composition (e.g., an immunogenic composition) comprising a recombinant NDV described herein, the method comprising (a) propagating a recombinant NDV described herein a cell (e.g., a cell line) or embryonated egg; and (b) isolating the recombinant NDV from the cell or embryonated egg. The method may further comprise adding the recombinant NDV to a container along with a pharmaceutically acceptable carrier.

In some embodiments, provided herein are cells (e.g., cell line) comprising a transgene, polynucleotide, nucleic acid sequence, or nucleotide sequence described herein. In some embodiments, provided herein are cells comprising a vector described herein. The cells may be transfected, transformed, or transduced with the transgene described herein, polynucleotide described herein, nucleic acid sequence described herein, vector described herein, or nucleotide sequence described herein. In specific embodiments, the cells are isolated. In some embodiments, the cells are cell lines. In some embodiments, the cells are primary cells. In some embodiments, the cells are in vitro or ex vivo. The cell(s) may be one described herein. For example, the cell(s) may be in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). The cell(s) may be a cell line(s), e.g., cancer cell lines such as HeLa cells, MCF7 cells, THP-1 cells, U87 cells, DU145 cells, Lncap cells, and T47D cells. The cells or cell lines (e.g., cancer cells or cancer cell lines) are obtained, derived, or obtained and derived from a human(s). The cell(s) may be Vero cells. In specific embodiments, the cell(s) is in vitro or ex vivo.

In some embodiments, provided herein is an embryonated egg comprising a polynucleotide herein or a transgene described herein. In some embodiments, provided herein is an embryonated egg comprising a vector described herein. In some embodiments, provided herein is an embryonated egg expressing a recombinant protein described herein. In some embodiments, the embryonated egg is a non-human egg. In some embodiments, the embryonated egg is ex vivo. In some embodiments, the embryonated egg is a non-human egg that is ex vivo. In some embodiments, the embryonated egg is a chicken egg or other avian egg. In some embodiments, the embryonated egg is a chicken egg that is about 8 to about 12 days old (e.g., 8, 9, 10 or 11 days old). The embryonated egg may be one described herein.

### 5.5 Compositions and Routes of Administration

Provided herein are compositions comprising a recombinant NDV described herein (e.g., Section 5.2, or 6). In a specific embodiment, the compositions are pharmaceutical compositions, such as immunogenic compositions (e.g., vaccine compositions). In some embodiment, provided herein are compositions (e.g., immunogenic compositions) comprising a polynucleotide or transgene described herein, a vector described herein, or a recombinant protein described herein (e.g., Section 5.1, or 6). In some embodiments, provided herein is an immunogenic composition comprising a recombinant protein described herein. In some embodiments, provided herein is an immunogenic composition comprising a polynucleotide described herein, a nucleotide sequence described herein, a transgene described herein, or a nucleic acid sequence described herein. In some embodiments, provided herein is a vector described herein. In a specific embodiment, provided herein are immunogenic compositions comprising a recombinant NDV described herein (e.g., Section 5.2, or 6). The compositions may be include a carrier or excipient. The compositions may or may not include an adjuvant. In some embodiments, an adjuvant is administered before, concomitantly with, or after administration of the composition. The compositions may or may not comprise one or more additional active agents (e.g., prophylactic or therapeutic agents). The compositions may be used in methods of inducing an immune response to LASV GP or LASV NP. The compositions may be used in methods for inducing an immune response to LASV or immunizing against LASV. The compositions may be used in methods for immunizing against a Lassa virus disease (e.g., Lassa fever or Lassa hemorrhagic fever). The compositions may be used in methods for preventing a Lassa virus disease (e.g., Lassa fever or Lassa hemorrhagic fever).

In one embodiment, an immunogenic composition comprises a recombinant NDV described herein (e.g., Section 5.2, or 6), in an admixture with a pharmaceutically acceptable carrier. In some embodiments, the immunogenic composition further comprises one or more additional prophylactic or therapeutic agents. In a specific embodiment, an immunogenic composition comprises an effective amount of a recombinant NDV described herein (e.g., Section 5.2, or 6), and optionally one or more additional prophylactic or therapeutic agents, in a pharmaceutically acceptable carrier. In some embodiments, the recombinant NDV (e.g., Section 5.2, or 6) is the only active ingredient included in the immunogenic composition. In some embodiments, an immunogenic composition comprises two recombinant NDV described herein (e.g., a recombinant NDV expressing a LASV GP or a chimeric LASV glycoprotein, and a recombinant NDV expressing a LASV NP, or a recombinant NDV comprising a LASV GP or a chimeric LASV glycoprotein, and a recombinant NDV comprising a LASV NP). In a particular embodiment, the immunogenic composition is a vaccine.

In a specific embodiment, administration of an immunogenic composition described herein to a subject (e.g., a human) generates neutralizing antibody (e.g., anti-LASV GP IgG or anti-LASV NP IgG). In certain embodiments, administration of an immunogenic composition described herein to a subject (e.g., a human) generates an immune response that provides some level of protection against developing a Lassa virus disease (e.g., Lassa fever or Lassa hemorrhagic fever).

In a specific embodiment, the recombinant NDV included in an immunogenic composition described herein is a live virus. In particular embodiments, the recombinant NDV included in a pharmaceutical composition described herein is an attenuated live virus. In some embodiments, the recombinant NDV included in an immunogenic composition described herein is inactivated. Any technique known to one of skill in the art may be used to inactivate a recombinant NDV described herein. For example, formalin or beta-propiolactone may be used to inactivate a recombinant NDV described herein. In a specific embodiment, the recombinant NDV included in a composition described herein is inactivated using 0.05% to 2% (e.g., 0.05%, 0.1%, 0.5%, 1%, or 2%) beta-Propiolactone, or another technique known to one of skill in the art.

In specific embodiments, an immunogenic composition described herein or a recombinant NDV described herein does not require frozen storage, which makes it difficult to transport and store in low-income countries. In specific embodiments, an immunogenic composition described herein or a recombinant NDV described herein may be stored at about 2° C. to about 8° C. (e.g., 4° C.).

The immunogenic compositions provided herein can be in any form that allows for the composition to be administered to a subject. In a specific embodiment, the pharmaceutical compositions are suitable for veterinary administration, human administration, or both. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, the immunogenic compositions are formulated to be suitable for the intended route of administration to a subject. For example, an immunogenic composition may be formulated to be suitable for parenteral, intravenous, intraarterial, intrapleural, inhalation, intranasal, intraperitoneal, oral, intradermal, colorectal, intraperitoneal, and intracranial administration. In one embodiment, an immunogenic composition may be formulated for intravenous, intraarterial, oral, intraperitoneal, intranasal, intratracheal, intrapleural, intracranial, subcutaneous, intramuscular, topical, or pulmonary administration. In a specific embodiment, an immunogenic composition may be formulated for intranasal administration. In certain embodiments, an immunogenic composition is formulated for a nasal spray. In another embodiment, an immunogenic composition may be formulated for intramuscular administration.

In a specific embodiment, an immunogenic composition comprising a recombinant NDV described herein (see, e.g., Sections 5.2, and 6) is formulated to be suitable for intranasal administration to the subject (e.g., human subject).

In a specific embodiment, an immunogenic composition comprising an inactivated recombinant NDV described herein may comprise an adjuvant. In certain embodiments, the compositions described herein comprise, or are administered in combination with, an adjuvant. The adjuvant for administration in combination with a composition described herein may be administered before, concomitantly with, or after administration of the composition. In specific embodiments, an inactivated virus immunogenic composition described herein comprises one or more adjuvants. In some embodiments, the term “adjuvant” refers to a compound that when administered in conjunction with or as part of a composition described herein augments, enhances and/or boosts the immune response to a recombinant NDV, but when the compound is administered alone does not generate an immune response to the virus. In some embodiments, the adjuvant generates an immune response to a recombinant NDV and does not produce an allergy or other adverse reaction. In some embodiments, a composition described herein (e.g., a live recombinant NDV composition) does not contain an adjuvant.

In certain embodiments, an immunogenic composition described herein comprises an effective amount of a recombinant NDV described herein. In specific embodiments, an effective amount of a recombinant NDV described herein is an amount of recombinant NDV to generate an immune response in a subject or a population of subjects. In specific embodiments, an effective amount of a recombinant NDV described herein is 10⁴ to 10¹² PFU or EID50.

In some embodiments, an immunogenic composition described herein comprises 1 to 15 micrograms of LASV GP, LASV-NP, or chimeric Lassa virus GP expressed by a recombinant NDV described herein. In some embodiments, an immunogenic composition described herein comprises 1 to 15 micrograms of a derivative of a LASV GP, a derivative of a LASV-NP, or a protein comprising a Lassa virus GP ectodomain or a derivative thereof described herein expressed by a recombinant NDV described herein.

In some embodiments, an immunogenic composition described herein comprises 1 to 15 micrograms of a LASV GP or a derivative thereof described herein, a LASV-NP or a derivative thereof described herein, a protein comprising a Lassa virus GP ectodomain or a derivative thereof described herein, or a chimeric Lassa virus glycoprotein described herein.

In some embodiments, an immunogenic composition described herein comprises 1 to 15 micrograms of inactivated recombinant NDV described herein.

In a specific embodiment, an immunogenic composition described herein may be stored at 2° to 8° C. (e.g., 4° C.).

### 5.6 Uses of Recombinant NDV and Compositions

The recombinant NDV(s) described herein or immunogenic composition described herein may be used to immunize a subject against Lassa virus, induce an immune response to LASV GP or LASV NP, or prevent a Lassa virus disease (e.g., Lassa fever or Lassa hemorrhagic fever). In a specific aspect, the recombinant NDV(s) described herein may be used to immunize a subject against Lassa virus lineage II, induce an immune response to a Lassa virus lineage II GP or NP, or prevent a Lassa virus disease (e.g., Lassa fever or Lassa hemorrhagic fever) caused by or associated with Lassa virus lineage II. In some embodiments, the recombinant NDV(s) described herein may be used to immunize a subject against Lassa virus lineage II disease. In a specific embodiment, the recombinant NDV(s) described herein may be used to immunize a subject against Lassa virus/H. sapiens-wt/NGA/2018/IRR 013, induce an immune response to Lassa virus/H. sapiens-wt/NGA/2018/IRR 013 GP or NP, or prevent a Lassa virus disease (e.g., Lassa fever or Lassa hemorrhagic fever) caused by or associated with Lassa virus/H. sapiens-wt/NGA/2018/IRR 013. In a specific embodiment, the recombinant NDV(s) described herein may be used to immunize a subject against Lassa virus/H. sapiens-wt/NGA/2018/IRR 013 disease.

In a specific aspect, an immunogenic composition described herein may be used to immunize a subject against Lassa virus lineage II disease, induce an immune response to a Lassa virus lineage II GP or NP, or prevent a Lassa virus disease (e.g., Lassa fever or Lassa hemorrhagic fever) caused by or associated with Lassa virus lineage II. In a specific embodiment, the recombinant NDV(s) described herein may be used to immunize a subject against Lassa virus/H. sapiens-wt/NGA/2018/IRR 013 disease, induce an immune response to Lassa virus/H. sapiens-wt/NGA/2018/IRR 013 GP or NP, or prevent a Lassa virus disease (e.g., Lassa fever or Lassa hemorrhagic fever) caused by or associated with Lassa virus/H. sapiens-wt/NGA/2018/IRR 013.

In one aspect, presented herein are methods for inducing an immune response to Lassa virus (e.g., a Lassa virus lineage II, or Lassa virus/H. sapiens-wt/NGA/2018/IRR 013) in a subject (e.g., a human subject), comprising administering to the subject (e.g., a human subject) a recombinant NDV described herein or an immunogenic composition described herein (e.g., an immunogenic composition comprising a recombinant NDV described herein). In one embodiment, presented herein is a method for inducing an immune response to LASV GP or LASV NP (e.g., a Lassa virus lineage II GP or NP, or Lassa virus/H. sapiens-wt/NGA/2018/IRR 013 GP or NP) in a subject (e.g., a human subject), comprising administering to the subject (e.g., a human subject) a recombinant NDV described herein or an immunogenic composition described herein, such as described in Section 5.5. In another embodiment, presented herein is a method for inducing an immune response to LASV GP or LASV NP (e.g., a Lassa virus lineage II GP or NP, or Lassa virus/H. sapiens-wt/NGA/2018/IRR 013 GP or NP) in a subject (e.g., a human subject), comprising administering to the subject (e.g., a human subject) an effective amount of a recombinant NDV described herein or an immunogenic composition described herein. See, e.g., Section 5.2 and 6 for recombinant NDV and Section 5.5 or 6 for immunogenic compositions. In a specific embodiment, the recombinant NDV is one described in Section 5.2 or 6, and the immunogenic composition is one described in Section 5.5 or 6.

In another aspect, presented herein are methods for immunizing a subject (e.g., a human subject) against Lassa virus (e.g., a Lassa virus lineage II, or Lassa virus/H. sapiens-wt/NGA/2018/IRR 013) comprising administering to the subject (e.g., a human subject) a recombinant NDV described herein or an immunogenic composition described herein (e.g., an immunogenic composition comprising a recombinant NDV described herein). In one embodiment, presented herein is a method for immunizing a subject (e.g., a human subject) against Lassa virus (e.g., a Lassa virus lineage II, or Lassa virus/H. sapiens-wt/NGA/2018/IRR 013), comprising administering to the subject (e.g., a human subject) a recombinant NDV described herein, or an immunogenic composition described herein. In another embodiment, presented herein is a method for immunizing a subject (e.g., a human subject) against Lassa virus (e.g., a Lassa virus lineage II, or Lassa virus/H. sapiens-wt/NGA/2018/IRR 013), comprising administering to the subject (e.g., a human subject) an effective amount of a recombinant NDV described herein, or an immunogenic composition described herein. In some embodiments, presented herein are methods for immunizing a subject (e.g., a human subject) against Lassa virus (e.g., a Lassa virus lineage II, or Lassa virus/H. sapiens-wt/NGA/2018/IRR 013) comprising administering to the subject (e.g., a human subject) a recombinant NDV described herein or an immunogenic composition described herein. See, e.g., Section 5.2 and 6 for recombinant NDV and Section 5.5 and 6 for compositions. In a specific embodiment, the recombinant NDV is one described in Section 5.2 or 6, and the immunogenic composition is one described in Section 5.5 or 6.

In another aspect, presented herein are methods for preventing a Lassa virus disease (e.g., Lassa fever or Lassa hemorrhagic fever) in a subject (e.g., a human subject), comprising administering to the subject (e.g., a human subject) a recombinant NDV described herein, or an immunogenic composition described herein (e.g., an immunogenic composition comprising a recombinant NDV described herein). In one embodiment, presented herein is a method for preventing a Lassa virus disease (e.g., Lassa fever or Lassa hemorrhagic fever) in a subject (e.g., a human subject), comprising administering to the subject (e.g., a human subject) a recombinant NDV described herein or an immunogenic composition described herein. In another embodiment, presented herein is a method for preventing a Lassa virus disease (e.g., Lassa fever or Lassa hemorrhagic fever) in a subject (e.g., a human subject), comprising administering to the subject (e.g., a human subject) an effective amount of a recombinant NDV described herein or an immunogenic composition described herein. In a specific embodiment, the recombinant NDV is one described in Section 5.2 or 6, and the immunogenic composition is one described in Section 5.5 or 6. The Lassa virus disease (e.g., Lassa fever) may be caused by or associated with a Lassa virus lineage II (e.g., Lassa virus/H. sapiens-wt/NGA/2018/IRR 013).

The recombinant NDV described herein may be administered to a subject in combination with one or more other therapies. The recombinant NDV and one or more other therapies may be administered by the same or different routes of administration to the subject. In a specific embodiment, the recombinant NDV is administered to a subject intranasally. See, e.g., Sections 5.2, and 6, infra for information regarding recombinant NDV, Section 5.6.2 for information regarding other therapies, and Section 5.5 and 6 for information regarding compositions and routes of administration.

The recombinant NDV and one or more additional therapies may be administered concurrently or sequentially to the subject. In certain embodiments, the recombinant NDV and one or more additional therapies are administered in the same composition. In other embodiments, the recombinant NDV and one or more additional therapies are administered in different compositions. The recombinant NDV and one or more other therapies may be administered by the same or different routes of administration to the subject. Any route known to one of skill in the art or described herein may be used to administer the recombinant NDV and one or more other therapies. In a specific embodiment, the recombinant NDV is administered intranasally or intramuscularly and the one or more other therapies are administered by the same or a different route. In a specific embodiment, the recombinant NDV is administered intranasally and the one or more other therapies is administered intravenously or orally.

An immunogenic composition described herein may be administered to a subject in combination with one or more other therapies. The immunogenic composition and one or more other therapies may be administered by the same or different routes of administration to the subject. In a specific embodiment, an immunogenic composition described herein is administered to a subject intranasally. See, e.g., Sections 5.5 and 6, infra for information regarding immunogenic compositions, Section 5.6.2 for information regarding other therapies, and Section 5.5 and 6 for information regarding routes of administration.

An immunogenic composition described herein and one or more additional therapies may be administered concurrently or sequentially to the subject. An immunogenic composition described herein and one or more other therapies may be administered by the same or different routes of administration to the subject. Any route known to one of skill in the art or described herein may be used to administer an immunogenic composition described herein and one or more other therapies. In a specific embodiment, an immunogenic composition described herein is administered intranasally or intramuscularly and the one or more other therapies are administered by the same or a different route. In a specific embodiment, an immunogenic composition described herein is administered intranasally and the one or more other therapies is administered intravenously or orally.

In some embodiments, two immunogenic compositions described herein are administered concurrently or sequentially to the subject. In some embodiments, three immunogenic compositions described herein are administered concurrently or sequentially to the subject. In some embodiments, three immunogenic compositions described herein are administered concurrently or sequentially to the subject. In some embodiments, four immunogenic compositions described herein are administered concurrently or sequentially to the subject.

In a specific embodiment, the immune response resulting from administration of a recombinant NDV described herein, or an immunogenic composition described herein provides some protection against a Lassa virus disease (e.g., Lassa fever or Lassa hemorrhagic fever) caused by or associated with Lassa virus (e.g., a Lassa virus lineage II, or Lassa virus/H. sapiens-wt/NGA/2018/IRR 013). In another specific embodiment, an antibody induced by a recombinant NDV described herein, or an immunogenic composition described herein binds to a LASV GP or LASV NP (e.g., a Lassa virus lineage II GP or NP, or Lassa virus/H. sapiens-wt/NGA/2018/IRR 013 GP or NP). In another specific embodiment, an antibody induced by a recombinant NDV described herein, or an immunogenic composition described herein may neutralize a LASV (e.g., a Lassa virus lineage II, or Lassa virus/H. sapiens-wt/NGA/2018/IRR 013), as assessed by an assay described herein or known to one of skill in the art. In some embodiments, the immune response resulting from administration of a recombinant NDV described herein, or an immunogenic composition described herein provides some protection against a Lassa virus disease (e.g., Lassa fever or Lassa hemorrhagic fever) caused by or associated with Lassa virus (e.g., a Lassa virus lineage II, or Lassa virus/H. sapiens-wt/NGA/2018/IRR 013), as assessed by an assay described herein or known to one of skill in the art.

In some embodiments, a recombinant NDV described herein or an immunogenic composition described herein, or a combination therapy described herein is administered to a patient to prevent the onset of one, two or more symptoms of a Lassa virus disease (e.g., Lassa fever or Lassa hemorrhagic fever). In a specific embodiment, the administration of a recombinant NDV described herein or an immunogenic composition described herein, or a combination therapy described herein to a subject prevents the onset or development of one, two or more symptoms of a Lassa virus disease (e.g., Lassa fever or Lassa hemorrhagic fever), or reduces the severity of one, two or more symptoms of a Lassa virus disease (e.g., Lassa fever or Lassa hemorrhagic fever). In a specific embodiment, the administration of a recombinant NDV described herein or an immunogenic composition described herein, or a combination therapy described herein to a subject prevents the onset or development of one, two or more symptoms of a Lassa virus disease (e.g., Lassa fever or Lassa hemorrhagic fever) and reduces the severity of one, two or more symptoms of a Lassa virus disease (e.g., Lassa fever or Lassa hemorrhagic fever). Symptoms of Lassa hemorrhagic fever may include fever, general malaise and weakness, headache, hemorrhaging (in gums, eyes, or nose, as examples), respiratory distress, repeated vomiting, facial swelling, pain in the chest, back, and abdomen, shock, hearing loss, tremors, encephalitis, and multi-organ failure.

In a specific embodiment, the administration of a recombinant NDV described herein, an immunogenic composition described herein, or a combination therapy described herein to a subject prevents hospitalization. In another specific embodiment, the administration of a recombinant NDV described herein or an immunogenic composition described herein, or a combination therapy described herein to a subject prevents a Lassa virus disease (e.g., Lassa fever or Lassa hemorrhagic fever). In another embodiment, the administration of a recombinant NDV described herein, an immunogenic composition described herein, or a combination therapy described herein to a subject reduces the length of hospitalization.

In another specific embodiment, the administration of a recombinant NDV described herein, or an immunogenic composition described herein to a subject induces LASV-specific T cells (e.g., cytotoxic T cells). In another specific embodiment, the administration of a recombinant NDV described herein, an immunogenic composition described herein, or a combination therapy described herein to a subject induces LASV-specific antibodies (e.g., neutralizing IgG antibodies) and LASV-specific T cells (e.g., cytotoxic T cells).

In some embodiments, a recombinant NDV described herein or a composition thereof, or a combination therapy described herein is administered to a subject predisposed or susceptible to of a Lassa virus disease (e.g., Lassa fever or Lassa hemorrhagic fever).

In certain embodiments, a recombinant NDV described herein, an immunogenic composition described herein, or a combination therapy described herein is administered to a human.

In some embodiments, a recombinant NDV described herein, or an immunogenic composition described herein is administered to a non-human subject (e.g., a mouse, rat, etc.) and the antibodies generated in response to the polypeptide are isolated. Hybridomas may be made and monoclonal antibodies produced as known to one of skill in the art. The antibodies may also be optimized. In some embodiments, the antibodies produced are humanized or chimerized. In certain embodiments, the non-human subject produces human antibodies. The antibodies produced using a recombinant NDV described herein, or immunogenic composition described herein may be optimized, using techniques known to one of skill in the art. In a specific embodiment, antibodies generated using a recombinant NDV described herein, or an immunogenic composition described herein may be used to prevent, treat or prevent and treat a Lassa virus disease (e.g., a Lassa fever).

In some embodiments, a recombinant NDV described herein, a LASV GP or a derivative thereof described herein, a LASV NP or a derivative thereof described herein, a protein comprising a LASV GP ectodomain or a derivative thereof described herein is used in an immunoassay (e.g., an ELISA assay) known to one of skill in the art or described herein to detect antibody specific for LASV GP or LASV NP. In one embodiment, method for detecting the presence of antibody specific to LASV GP or LASV NP, comprising contacting a specimen with the recombinant NDV described herein in an immunoassay (e.g., an ELISA). In another embodiment, method for detecting the presence of antibody specific to LASV GP or LASV NP, comprising contacting a specimen with a LASV GP or a derivative thereof described herein, a LASV NP or a derivative thereof, described herein a protein comprising a LASV GP ectodomain or a derivative thereof described herein in an immunoassay (e.g., an ELISA). In some embodiments, the specimen is a biological specimen. In a specific embodiment, the biological specimen is blood, plasma or sera from a subject (e.g., a human subject). In other embodiments, the specimen is an antibody or antisera.

### 5.6.1 Dosages and Frequency

The amount of a recombinant NDV or an immunogenic composition described herein, which will be effective in the prevention of a Lassa virus disease (e.g., Lassa fever or Lassa hemorrhagic fever) will depend on the route of administration, the general health of the subject, etc. Suitable dosage ranges of a recombinant NDV for administration are generally about 10⁴ to about 10¹² EID50, and can be administered to a subject once, twice, three, four or more times with intervals as often as needed. In some embodiments, a recombinant NDV described herein is administered to a subject (e.g., human) at a dose of 10⁴ to about 10¹² EID50. In some embodiments, a dose of about 10⁴ to about 10¹² EID50 of a composition comprising live recombinant NDV is administered to a subject (e.g., human).

In certain embodiments, a recombinant NDV described herein is administered to a subject (e.g., human) at a dose of 1 to 15 micrograms of LASV GP or LASV NP, or a chimeric Lassa virus glycoprotein. In some embodiments, a recombinant NDV described herein is administered to a subject (e.g., human) at a dose of 1 to 15 micrograms of a derivative of a LASV GP, a derivative of a LASV NP, or a protein comprising a LASV GP ectodomain or a derivative thereof.

In some embodiments, a LASV GP or a derivative thereof described herein, a LASV NP or a derivative thereof described herein, or a protein comprising a LASV GP ectodomain or a derivative thereof described herein is administered to a subject (e.g., human) at a dose of 1 to 15 micrograms.

In some embodiments, an immunogenic composition described herein is administered to a subject (e.g., human) at a dose of 10 to 100 micrograms of inactivated recombinant NDV described herein. In some embodiments, an immunogenic composition described herein is administered to a subject (e.g., human) at a dose of 10 to 100 micrograms of inactivated recombinant NDV described herein. In specific embodiments, an immunogenic composition described herein is administered to a subject (e.g., human) at a dose of 10 micrograms, 30 micrograms, or 100 micrograms of inactivated recombinant NDV described herein.

In certain embodiments, dosages of a recombinant NDV described herein, or a composition described herein similar to those currently being used in clinical trials for NDV are administered to a subject.

In certain embodiments, a recombinant NDV or an immunogenic composition described herein is administered to a subject as a single dose followed by a second dose 1 to 6 weeks, 1 to 5 weeks, 1 to 4 weeks, 1 to 3 weeks, 1 to 2 weeks, 6 to 12 weeks, 3 to 6 months, 6 to 9 months, 6 to 12 months, or 6 to 9 months later. In certain embodiments, a subject is administered one or more boosters. The recombinant NDV used for each booster may administered by the same or different routes.

In certain embodiments, a recombinant NDV or an immunogenic composition described herein is administered to a subject in combination with one or more additional therapies, such as a therapy described in Section 5.6.2, infra. The dosage of the other one or more additional therapies will depend upon various factors including, e.g., the therapy, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. Recommended doses for approved therapies can be found in the Physician's Desk Reference.

In certain embodiments, a recombinant NDV or an immunogenic composition described herein is administered to a subject concurrently with the administration of one or more additional therapies. In some embodiments, an immunogenic composition comprising recombinant NDV and a pharmaceutical composition comprising one or more additional therapies may be administered concurrently, or before or after each other.

### 5.6.2 Additional Therapies

Additional therapies that can be used in a combination with a recombinant NDV described herein or a composition thereof include, but are not limited to, acetaminophen, ibuprofen, throat lozenges, cough suppressants, inhalers, antivirals, monoclonal antibodies, and oxygen. In a specific embodiment, the additional therapy is a second recombinant NDV described herein.

Additional therapies that can be used in a combination with a composition described herein (e.g., an immunogenic composition described herein) include, but are not limited to, acetaminophen, ibuprofen, throat lozenges, cough suppressants, inhalers, antivirals, monoclonal antibodies, and oxygen. In some embodiments, the additional therapy is a second immunogenic composition described herein.

### 5.7 Biological Assays

In a specific embodiment, one, two or more of the assays described in Section 6 may be used to characterize a recombinant NDV described herein, a LASV GP described herein, a derivative of a LASV GP described herein, protein comprising a LASV GP or a derivative thereof described herein, a LASV NP described herein, a derivative of a LASV NP described herein, or a chimeric Lassa virus glycoprotein described herein. In another specific embodiment, assays known to one of skill in the art may be used to characterize immunoglobulin samples from a subject (e.g., a human subject) administered a recombinant NDV described herein or a composition described herein. For example, the IgG titer and microneutralization of IgG induced may be assessed as described herein or known to one of skill in the art. In some embodiments, a subject administered a recombinant NDV described herein or a composition described herein is assessed for anti-NDV antibodies as well as anti-LASV GP or anti-LASV NP antibodies.

### 5.7.1 In Vitro Viral Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of the recombinant NDVs described herein can be assessed by any method known in the art or described herein (e.g., in cell culture (e.g., cultures of BSTT7 or embryonated chicken cells) (see, e.g., Section 6). Viral titer may be determined by inoculating serial dilutions of a recombinant NDV described herein into cell cultures (e.g., BSTT7 or embryonated chicken cells), chick embryos (e.g., 9 to 11 day old embryonated eggs), or live non-human animals. After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50).

Incorporation of nucleotide sequences encoding a heterologous peptide or protein (e.g., a transgene into the genome of a recombinant NDV described herein can be assessed by any method known in the art or described herein (e.g., in cell culture, an animal model or viral culture in embryonated eggs)). For example, viral particles from cell culture of the allantoic fluid of embryonated eggs can be purified by centrifugation through a sucrose cushion and subsequently analyzed for protein expression by Western blotting using methods well known in the art. In a specific embodiment, a method described in Section 6, infra, is used to assess the incorporation of a transgene into the genome of a recombinant NDV.

Immunofluorescence-based approaches may also be used to detect virus and assess viral growth. Such approaches are well known to those of skill in the art, e.g., fluorescence microscopy and flow cytometry. Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

### 5.7.2 Interferon Assays

IFN induction and release induced by a recombinant NDV described a LASV GP described herein, a derivative of a LASV GP described herein, protein comprising a LASV GP or a derivative thereof described herein, a LASV NP described herein, a derivative of a LASV NP described herein, a chimeric Lassa virus glycoprotein described herein, or an immunogenic composition described herein may be determined using techniques known to one of skill in the art. For example, the amount of IFN induced in cells following infection with a recombinant NDV described herein or administration of an immunogenic composition described herein may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. Further, the induction and release of cytokines and/or interferon-stimulated genes may be determined by, e.g., an immunoassay or ELISPOT assay at the protein level and/or quantitative RT-PCR or northern blots at the RNA level.

### 5.7.3 Toxicity Studies

In some embodiments, the recombinant NDVs described herein or compositions thereof, a composition described herein, or combination therapies described herein are tested for cytotoxicity in mammalian, preferably human, cell lines. In some embodiments, the ToxLite assay is used to assess cytotoxicity.

Many assays well-known in the art can be used to assess viability of cells or cell lines following infection with a recombinant NDV described herein or composition thereof, or contact with a composition described herein, and, thus, determine the cytotoxicity of the recombinant NDV or composition thereof, or a composition described herein. For example, cell proliferation can be assayed by measuring Bromodeoxyuridine (BrdU) incorporation, (³H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc.). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, a recombinant NDV described herein or composition thereof does not kill healthy (i.e., non-cancerous) cells.

In specific embodiments, cell viability may be measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The recombinant NDVs described herein or compositions described herein, or combination therapies can be tested for in vivo toxicity in animal models. For example, animals are administered a range of pfu of a recombinant NDV described herein, and subsequently, the animals are monitored over time for various parameters, such as one, two or more of the following: lethality, weight loss or failure to gain weight, and levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and regimen in addition to dosages. See, e.g., the Examples, infra, for assays that may be used to assess toxicity.

### 5.7.4 Biological Activity Assays

The recombinant NDVs described herein or compositions described herein, or combination therapies described herein can be tested for biological activity using animal models for inhibiting a Lassa virus disease (e.g., Lassa fever), antibody response to the recombinant NDVs, etc. Such animal model systems include, but are not limited to, rats, mice, hamsters, cotton rats, chicken, cows, monkeys (e.g., African green monkey), pigs, dogs, rabbits, etc.

In a specific embodiment, the recombinant NDVs described herein, compositions described herein, or combination therapies described herein may be tested using animal models for the ability to induce a certain geometric mean titer of antibody(ies) that binds to LASV GP or LASV NP. An immunoassay, such as an ELISA, or known to one of skill in the art may be used to measure antibody titer. In another specific embodiment, the recombinant NDVs described herein, compositions described herein, or combination therapies described herein may be tested using animal models for the ability to induce antibodies that have neutralizing activity against LASV GP in a microneutralization assay. In certain embodiments, the recombinant NDVs described herein, or compositions described herein, or combination therapies described herein may be tested using animal models for the ability to induce a protective immune response. In some embodiments, the recombinant NDVs described herein, or compositions described herein, or combination therapies described herein may be tested using animal models such as described in Section 6, infra.

### 5.7.5 Expression of Transgene

Assays for testing the expression of a LASV GP or a derivative thereof, a LASV NP or a derivative thereof, a protein comprising a LASV GP ectodomain or a derivative thereof, or a chimeric Lassa virus glycoprotein in cells infected with a recombinant NDV comprising a packaged genome comprising a transgene that comprises a nucleotide sequence encoding a LASV GP or a derivative thereof described herein, a LASV NP or a derivative thereof described herein, a protein comprising a LASV GP ectodomain or a derivative thereof described herein, or a chimeric Lassa virus glycoprotein described herein may be conducted using any assay known in the art, such as, e.g., western blot, immunofluorescence, and ELISA, or any assay described herein. Also, assays for testing the expression of a LASV GP or a derivative thereof, a LASV NP or a derivative thereof, a protein comprising a LASV GP ectodomain or a derivative thereof, or a chimeric Lassa virus glycoprotein by cells include western blot, immunofluorescence, and ELISA, or any assay described herein or known to one of skill in the art.

In a specific aspect, ELISA is utilized to detect expression of a LASV GP or a derivative thereof, a LASV NP or a derivative thereof, or a chimeric Lassa virus glycoprotein in cells infected with a recombinant NDV comprising a packaged genome comprising a transgene that comprises a nucleotide sequence encoding a LASV GP or a derivative thereof described herein, a LASV NP or a derivative thereof described herein, or a chimeric Lassa virus glycoprotein described herein.

In one embodiment, a LASV GP or a derivative thereof, a LASV NP or a derivative thereof, or a chimeric Lassa virus glycoprotein encoded by a packaged genome of a recombinant NDV described herein is assayed for proper folding by testing its ability to bind specifically to an anti-LASV GP or anti-LASV NP using any assay for antibody-antigen interaction known in the art. In another embodiment, a LASV GP or a derivative thereof, a LASV NP or a derivative thereof, or a chimeric Lassa virus glycoprotein encoded by a packaged genome of a recombinant NDV described herein is assayed for proper folding by determination of the structure or conformation of the LASV GP or a derivative thereof, LASV NP or a derivative thereof, or chimeric Lassa virus glycoprotein, respectively using any method known in the art such as, e.g., NMR, X-ray crystallographic methods, or secondary structure prediction methods, e.g., circular dichroism. Additional assays assessing the conformation and antigenicity of a LASV GP or a derivative thereof, a LASV NP or a derivative thereof, or a chimeric Lassa virus glycoprotein may include, e.g., immunofluorescence microscopy, flow cytometry, western blot, and ELISA may be used.

In one embodiment, a protein comprising a LASV GP ectodomain or a derivative thereof is assayed for proper folding by testing its ability to bind specifically to an anti-LASV GP or anti-LASV NP using any assay for antibody-antigen interaction known in the art. In another embodiment, a protein comprising a LASV GP ectodomain or a derivative thereof is assayed for proper folding by determination of the structure or conformation of the protein using any method known in the art such as, e.g., NMR, X-ray crystallographic methods, or secondary structure prediction methods, e.g., circular dichroism. Additional assays assessing the conformation and antigenicity of a protein comprising a LASV GP ectodomain or a derivative thereof may include, e.g., immunofluorescence microscopy, flow cytometry, western blot, and ELISA may be used.

### 5.8 Kits

In one aspect, provided herein is a pharmaceutical pack or kit comprising one or more containers filled with one or more of the ingredients of a composition (e.g., an immunogenic composition) described herein. In a specific embodiment, provided herein is a pharmaceutical pack or kit comprising a container, wherein the container comprises a recombinant NDV described herein. In a specific embodiment, provided herein is a pharmaceutical pack or kit comprising a container, wherein the container comprises an immunogenic composition described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, provided herein is a kit comprising in one or more containers filled with one or more recombinant NDVs described herein. In another embodiment, provided herein is a kit comprising in one or more containers one or more transgenes described herein. In another embodiment, provided herein is a kit comprising in one or more containers a polynucleotide or a nucleic acid sequence described herein. In another embodiment, provided herein is a kit comprising in one or more containers one or more nucleotide sequences comprising the genome of NDV and a transgene described herein. In another embodiment, provided herein is a kit comprising, in a container, a vector comprising a polynucleotide described herein or a transgene described herein. In another embodiment, provided herein is a kit comprising, in a container, a vector comprising a nucleic acid sequence described herein.

In a specific embodiment, provided herein is a kit comprising, in a container, a nucleotide sequence comprising a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV F transcription unit encodes a NDV F protein comprising a leucine to alanine amino acid substitution at the amino residue corresponding to amino acid residue 289 of the LaSota NDV strain.

In a specific embodiment, provided herein is a kit comprising, in a container, a vector comprising a nucleotide sequence, wherein the nucleotide sequence comprises a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV F transcription unit encodes a NDV F protein comprising a leucine to alanine amino acid substitution at the amino residue corresponding to amino acid residue 289 of the LaSota NDV strain.

### 5.9 SEQUENCES

TABLE 1
CDNA OF GENOME OF NDV STRAINS

| | | SEQ ID |
|---|---|---|
| Description | Sequence | NO: |
| cDNA of | accaaacagagaatccgtgagttacgataaaaggcgaaggagcaattgaagtcgcacggg | SEQ ID |
| genomic | tagaaggtgtgaatctcgagtgcgagcccgaagcacaaactcgagaaagccttctgccaac | NO: 1 |
| sequence of | atgtcttccgtatttgatgagtacgaacagctcctcgcggctcagactcgccccaatggagct | |
| NDV strain | catggagggggagaaaaagggagtaccttaaaagtagacgtcccggtattcactcttaaca | |
| LaSota | gtgatgacccagaagatagatggagctttgtggtattctgcctccggattgctgttagcgaag | |
| | atgccaacaaaccactcaggcaaggtgctctcatatctcttttatgctcccactcacaggtaat | |
| | gaggaaccatgttgccCttgcagggaaacagaatgaagccacattggccgtgcttgagatt | |
| | gatggctttgccaacggcacgccccagttcaacaataggagtggagtgtctgaagagagag | |
| | cacagagatttgcgatgatagcaggatctctccctcgggcatgcagcaacggaaccccgttc | |
| | gtcacagccggggcCgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaGgaGgggcctggc | |
| | agctgctgcccaacgggtctccgaGgaGaccagcagcataGacatgcctactcaacaag | |
| | tcggagtcctcactgggcttagcgagggggggtcccaagctctacaaggcggatcgaata | |
| | gatcgcaagggcaaccagaagccggggatggggagacccaattcctggatctgatgaga | |
| | gcggtagcaaatagcatgagggaggcgccaaactctgcacagggcactccccaatcggg | |
| | gcctcccccaactcctgggccatcccaagataacgacaccgactgggggtattgatggaca | |
| | aaacccagcctgcttccacaaaaacatcccaatgccctcacccgtagtcgacccctcgatttg | |
| | cggctctatatgaccacaccctcaaacaaacatccccctctttcctccctccccctgctgtaca | |
| | actAcgTacgccctagataccacaggcacaatgcggctcactaacaatcaaaacagagc | |
| | cgagggaattagaaaaaagtacgggtagaagagggatattcagagatcagggcaagtctc | |
| | ccgagtctctgctctctcctctacctgatagaccaggacaaacatggccacctttacagatgc | |
| | agagatcgacgagctatttgagacaagtggaactgtcattgacaacataattacagcccagg | |
| | gtaaaccagcagagactgttggaaggagtgcaatcccacaaggcaagaccaaggtgctga | |
| | gcgcagcatgggagaagcatgggagcatccagccaccggccagtcaagacaaccccgat | |
| | cgacaggacagatctgacaaacaaccatccacacccgagcaaacgaccccgcatgacag | |
| | cccgccggccacatccgccgaccagccccccacccaggccacagacgaagccgtcgac | |
| | acacagCtcaggaccggagcaagcaactctctgctgttgatgcttgacaagctcagcaata | |
| | aatcgtccaatgctaaaaagggcccatggtcgagcccccaagaggggaatcaccaacgtc | |
| | cgactcaacagcaggggagtcaacccagtcgcggaaacagtcaggaaagaccgcagaa | |
| | ccaagtcaaggccgcccctggaaaccagggcacagacgtgaacacagcatatcatggac | |
| | aatgggaggagtcacaactatcagctggtgcaacccctcatgctctccgatcaaggcagag | |
| | ccaagacaatacccttgtatctgcggatcatgtccagccacctgtagactttgtgcaagcgat | |
| | gatgtctatgatggaggcgatatcacagagagtaagtaaggttgactatcagctagatcttgtc | |
| | ttgaaacagacatcctccatccctatgatgcggtccgaaatccaacagctgaaaacatctgtt | |
| | gcagtcatggaagccaacttgggaatgatgaagattctggatcccggttgtgccaacatttca | |
| | tctctgagtgatctacgggcagttgcccgatctcacccggttttagtttcaggccctggagacc | |
| | cctctccctatgtgacacaaggaggcgaaatggcacttaataaactttcgcaaccagtgcca | |
| | catccatctgaattgattaaacccgccactgcatgcgggcctgatataggagtggaaaagga | |
| | cactgtccgtgcattgatcatgtcacgcccaatgcacccgagttcttcagccaagctcctaag | |
| | caagttagatgcagccgggtcgatcgaggaaatcaggaaaatcaagcgccttgctctaaat | |
| | ggctaattactactgccacacgtagcgggtccctgtccactcggcatcacacggaatctgca | |
| | ccgagttcccccccgcGgacccaaggtccaactctccaageggcaatcctctctegcttcct | |
| | cagccccactgaatgAtcgcgtaaccgtaattaatctagctacatttaagattaagaaaaaat | |
| | acgggtagaattggagtgccccaattgtgccaagatggactcatctaggacaattgggctgt | |
| | actttgattctgcccattcttctagcaacctgttagcatttccgatcgtcctacaagAcacagga | |
| | gatgggaagaagcaaatcgccccgcaatataggatccagcgccttgacttgtggactgata | |
| | gtaaggaggactcagtattcatcaccacctatggattcatctttcaagttgggaatgaagaagc | |
| | cacCgtcggcatgatcgatgataaacccaagcgcgagttactttccgctgcgatgctctgcc | |
| | taggaagcgtcccaaataccggagaccttattgagctggcaagggcctgtctcactatgata | |
| | gtcacatgcaagaagagtgcaactaatactgagagaatggttttctcagtagtgcaggcacc | |
| | ccaagtgctgcaaagctgtagggttgtggcaaacaaatactcatcagtgaatgcagtcaagc | |
| | acgtgaaagcgccagagaagattcccgggagtggaaccctagaatacaaggtgaactttgt | |
| | ctccttgactgtggtaccgaagaGggatgtctacaagatcccagctgcagtattgaaggtttc | |
| | tggctcgagtctgtacaatcttgcgctcaatgtcactattaatgtggaggtagacccgaggagt | |
| | cctttggttaaatctCtgtctaagtctgacagcggatactatgctaacctcttcttgcatattgga | |
| | cttatgaccacTgtagataggaaggggaagaaagtgacatttgacaagctggaaaagaaa | |
| | ataaggagccttgatctatctgtcgggctcagtgatgtgctcgggccttccgtgttggtaaaag | |
| | caagaggtgcacggactaagcttttggcacctttcttctctagcagtgggacagcctgctatc | |
| | ccatagcaaatgcttctcctcaggggccaagatactctggagtcaaaccgcgtgcctgcgg | |
| | agcgttaaaatcattatccaagcaggtacccaacgcgctgtcgcagtgaccgccgaccacg | |
| | aggttacctctactaagctggagaaggggcacacccttgccaaatacaatccttttaagaaat | |
| | aagctgcgtctctgagattgcgctccgcccactcacccagatcatcatgacacaaaaaacta | |
| | atctgtcttgattatttacagttagtttacctgtctatcaagttagaaaaaacacgggtagaagatt | |
| | ctggatcccggttggcgccctccaggtgcaagatgggctccagaccttctaccaagaaccc | |
| | agcacctatgatgctgactatccgggttgcgctggtactgagttgcatctgtccggcaaactc | |
| | cattgatggcaggcctcttgcagctgcaggaattgtggttacaggagacaaagccgtcaaca | |
| | tatacacctcatcccagacaggatcaatcatagttaagctcctcccgaatctgcccaaggata | |
| | aggaggcatgtgcgaaagcccccttggatgcatacaacaggacattgaccactttgctcacc | |
| | ccccttggtgactctatccgtaggatacaagagtctgtgactacatctggaggggggagaca | |
| | ggggcgccttataggcgccattattggcggtgtggctcttggggttgcaactgccgcacaaa | |
| | taacagcggccgcagctctgatacaagccaaacaaaatgctgccaacatcctccgacttaaa | |
| | gagagcattgccgcaaccaatgaggctgtgcatgaggtcactgacggattatcgcaactag | |
| | cagtggcagttgggaagatgcagcagtttgttaatgaccaatttaataaaacagctcaggaat | |
| | tagactgcatcaaaattgcacagcaagttggtgtagagctcaacctgtacctaaccgaattga | |
| | ctacagtattcggaccacaaatcacttcacctgctttaaacaagctgactattcaggcactttac | |
| | aatctagctggtggaaatatggattacttattgactaagttaggtgtagggaacaatcaactca | |
| | gctcattaatcggtagcggcttaatcaccggtaaccctattctatacgactcacagactcaact | |
| | cttgggtatacaggtaactctaccttcagtcgggaacctaaataatatgcgtgccacctacttg | |
| | gaaaccttatccgtaagcacaaccaggggatttgcctcggcacttgtcccAaaagtggtga | |
| | cacaggtcggttctgtgatagaagaacttgacacctcatactgtatagaaactgacttagattta | |
| | tattgtacaagaatagtaacgttccctatgtcccctggtatttattcctgcttgagcggcaatacg | |
| | tcggcctgtatgtactcaaagaccgaaggcgcacttactacaccatacatgactatcaaaggt | |
| | tcagtcatcgccaactgcaagatgacaacatgtagatgtgtaaaccccccgggtatcatatcg | |
| | caaaactatggagaagccgtgtctctaatagataaacaatcatgcaatgttttatccttaggcg | |
| | ggataactttaaggctcagtggggaattcgatgtaacttatcagaagaatatctcaatacaaga | |
| | ttctcaagtaataataacaggcaatcttgatatctcaactgagcttgggaatgtcaacaactcg | |
| | atcagtaatgctttgaataagttagaggaaagcaacagaaaactagacaaagtcaatgtcaa | |
| | actgactagcacatctgctctcattacctatatcgttttgactatcatatctcttgtttttggtatac | |
| | ttagcctgattctagcatgctacctaatgtacaagcaaaaggcgcaacaaaagaccttattatgg | |
| | cttgggaataatactctagatcagatgagagccactacaaaaatgtgaacacagatgaggaa | |
| | cgaaggtttccctaatagtaatttgtgtgaaagttctggtagtctgtcagttcagagagttaaga | |
| | aaaaactaccggttgtagatgaccaaaggacgatatacgggtagaacggtaagagaggcc | |
| | gcccctcaattgcgagccaggcttcacaacctccgttctaccgcttcaccgacaacagtcctc | |
| | aatcatggaccgcgccgttagccaagttgcgttagagaatgatgaaagagaggcaaaaaat | |
| | acatggcgcttgatattccggattgcaatcttattcttaacagtagtgaccttggctatatctgta | |
| | gcctcccttttatatagcatgggggctagcacacctagcgatcttgtaggcataccgactagg | |
| | atttccagggcagaagaaaagattacatctacacttggttccaatcaagatgtagtagatagg | |
| | atatataagcaagtggcccttgagtctccgttggcattgttaaatactgagaccacaattatgaa | |
| | cgcaataacatctctctcttatcagattaatggagctgcaaacaacagtgggtggggggcac | |
| | ctatccatgacccagattatataggggggataggcaaagaactcattgtagatgatgctagtg | |
| | atgtcacatcattctatccctctgcatttcaagaacatctgaattttatcccggcgcctactacag | |
| | gatcaggttgcactcgaataccctcatttgacatgagtgctacccattactgctacacccataat | |
| | gtaatattgtctggatgcagagatcactcacattcatatcagtatttagcacttggtgtgctccg | |
| | gacatctgcaacagggagggtattcttttctactctgcgttccatcaacctggacgacaccca | |
| | aaatcggaagtcttgcagtgtgagtgcaactcccctgggttgtgatatgctgtgctcgaaagt | |
| | cacggagacagaggaagaagattataactcagctgtccctacgcggatggtacatgggag | |
| | gttagggttcgacggccagtaccacgaaaaggacctagatgtcacaacattattcggggact | |
| | gggtggccaactacccaggagtagggggtggatcttttattgacagccgcgtatggttctca | |
| | gtctacggagggttaaaacccaattcacccagtgacactgtacaggaagggaaatatgtgat | |
| | atacaagcgatacaatgacacatgcccagatgagcaagactaccagattcgaatggccaag | |
| | tcttcgtataagcctggacggtttggtgggaaacgcatacagcaggctatcttatctatcaagg | |
| | tgtcaacatccttaggcgaagacccggtactgactgtaccgcccaacacagtcacactcatg | |
| | ggggccgaaggcagaattctcacagtagggacatctcatttcttgtatcaacgagggtcatca | |
| | tacttctctcccgcgttattatatcctatgacagtcagcaacaaaacagccactcttcatagtcct | |
| | tatacattcaatgccttcactcggccaggtagtatcccttgccaggcttcagcaagatgcccca | |
| | actcgtgtgttactggagtctatacagatccatatcccctaatcttctatagaaaccacaccttg | |
| | cgaggggtattcgggacaatgcttgatggtgtacaagcaagacttaaccctgcgtctgcagt | |
| | attcgatagcacatcccgcagtcgcattactcgagtgagttcaagcagtaccaaagcagcat | |
| | acacaacatcaacttgttttaaagtggtcaagactaataagacctattgtctcagcattgctgaa | |
| | atatctaatactctcttcggagaattcagaatcgtcccgttactagttgagatcctcaaagatga | |
| | cggggttagagaagccaggtctggctagttgagtcaattataaaggagttggaaagatggca | |
| | ttgtatcacctatcttctgcgacatcaagaatcaaaccgaatgccggcgcgtgctcgaattcca | |
| | tgttgccagttgaccacaatcagccagtgctcatgcgatcagattaagccttgtcaAtaGtct | |
| | cttgattaagaaaaaatgtaagtggcaatgagatacaaggcaaaacagctcatggtTaaCa | |
| | atacgggtaggacatggcgagctccggtcctgaaagggcagagcatcagattatcctacca | |
| | gagTcacacctgtcttcaccattggtcaagcacaaactactctattactggaaattaactggg | |
| | ctaccgcttcctgatgaatgtgacttcgaccacctcattctcagccgacaatggaaaaaaata | |
| | cttgaatcggcctctcctgatactgagagaatgataaaactcggaagggcagtacaccaaac | |
| | tcttaaccacaattccagaataaccggagtgctccaccccaggtgtttagaaGaactggcta | |
| | atattgaggtcccagattcaaccaacaaatttcggaagattgagaagaagatccaaattcaca | |
| | acacgagatatggagaactgttcacaaggctgtgtacgcatatagagaagaaactgctggg | |
| | gtcatcttggtctaacaatgtcccccggtcagaggagttcagcagcattcgtacggatccggc | |
| | attctggtttcactcaaaatggtccacagccaagtttgcatggctccatataaaacagatccag | |
| | aggcatctgatggtggcagctaGgacaaggtctgcggccaacaaattggtgatgctaaccc | |
| | ataaggtaggccaagtctttgtcactcctgaacttgtcgttgtgacgcatacgaatgagaacaa | |
| | gttcacatgtcttacccaggaacttgtattgatgtatgcagatatgatggagggcagagatatg | |
| | gtcaacataatatcaaccacggcggtgcatctcagaagcttatcagagaaaattgatgacattt | |
| | tgcggttaatagacgctctggcaaaagacttgggtaatcaagtctacgatgttgtatcactaat | |
| | ggagggatttgcatacggagctgtccagctactcgagccgtcaggtacatttgcaggagattt | |
| | cttcgcattcaacctgcaggagcttaaagacattctaattggcctcctccccaatgatatagca | |
| | gaatccgtgactcatgcaatcgctactgtattctctggtttagaacagaatcaagcagctgaga | |
| | tgttgtgtctgttgcgtctgtggggtcacccactgcttgagtcccgtattgcagcaaaggcagt | |
| | caggagccaaatgtgcgcaccgaaaatggtagactttgatatgatccttcaggtactgtctttc | |
| | ttcaagggaacaatcatcaacgggtacagaaagaagaatgcaggtgtgtggccgcgagtc | |
| | aaagtggatacaatatatgggaaggtcattgggcaactacatgcagattcagcagagatttca | |
| | cacgatatcatgttgagagagtataagagtttatctgcacttgaatttgagccatgtatagaatat | |
| | gaccctgtcaccaacctgagcatgttcctaaaagacaaggcaatcgcacaccccaacgata | |
| | attggcttgcctcgtttaggcggaaccttctctccgaagaccagaagaaacatgtaaaagaa | |
| | gcaacttcgactaatcgcctcttgatagagtttttagagtcaaatgattttgatccatataaagag | |
| | atggaatatctgacgacccttgagtaccttagagatgacaatgtggcagtatcatactcgctca | |
| | aggagaaggaagtgaaagttaatggacggatcttcgctaagctgacaaagaagttaaggaa | |
| | ctgtcaggtgatggcggaagggatcctagccgatcagattgcacctttctttcagggaaatgg | |
| | agtcattcaggatagcatatccttgaccaagagtatgctagcgatgagtcaactgtcttttaaca | |
| | gcaataagaaacgtatcactgactgtaaagaaagagtatcttcaaaccgcaatcatgatccga | |
| | aaagcaagaaccgtcggagagttgcaaccttcataacaactgacctgcaaaagtactgtctt | |
| | aattggagatatcagacaatcaaattgttcgctcatgccatcaatcagttgatgggcctacctc | |
| | acttcttcgaatggattcacctaagactgatggacactacgatgttcgtaggagaccctttcaat | |
| | cctccaagtgaccctactgactgtgacctctcaagagtccctaatgatgacatatatattgtca | |
| | gtgccagagggggtatcgaaggattatgccagaagctatggacaatgatctcaattgctgca | |
| | atccaacttgctgcagctagatcgcattgtcgtgttgcctgtatggtacagggtgataatcaag | |
| | taatagcagtaacgagagaggtaagatcagacgactctccggagatggtgttgacacagttg | |
| | catcaagccagtgataatttcttcaaggaattaattcatgtcaatcatttgattggccataatttga | |
| | aggatcgtgaaaccatcaggtcagacacattcttcatatacagcaaacgaatcttcaaagatg | |
| | gagcaatcctcagtcaagtcctcaaaaattcatctaaattagtgctagtgtcaggtgatctcagt | |
| | gaaaacaccgtaatgtcctgtgccaacattgcctctactgtagcacggctatgcgagaacgg | |
| | gcttcccaaagacttctgttactatttaaactatataatgagttgtgtgcagacatactttgactct | |
| | gagttctccatcaccaacaattcgcaccccgatcttaatcagtcgtggattgaggacatctcttt | |
| | tgtgcactcatatgttctgactcctgcccaattagggggactgagtaaccttcaatactcaagg | |
| | ctctacactagaaatatcggtgacccggggactactgcttttgcagagatcaagcgactaga | |
| | agcagtgggattactgagtcctaacattatgactaatatcttaactaggccgcctgggaatgg | |
| | agattgggccagtctgtgcaacgacccatactctttcaattttgagactgttgcaagcccaaat | |
| | attgttcttaagaaacatacgcaaagagtcctatttgaaacttgttcaaatcccttattgtctgga | |
| | gtgcacacagaggataatgaggcagaagagaaggcattggctgaattcttgcttaatcaaga | |
| | ggtgattcatccccgcgttgcgcatgccatcatggaggcaagctctgtaggtaggagaaag | |
| | caaattcaagggcttgttgacacaacaaacaccgtaattaagattgcgcttactaggaggcca | |
| | ttaggcatcaagaggctgatgcggatagtcaattattctagcatgcatgcaatgctgtttagag | |
| | acgatgttttttcctccagtagatccaaccaccccttagtctcttctaatatgtgttctctgacact | |
| | ggcagactatgcacggaatagaagctggtcacctttgacgggaggcaggaaaatactgggt | |
| | gtatctaatcctgatacgatagaactcgtagagggtgagattcttagtgtaagcggagggtgt | |
| | acaagatgtgacagcggagatgaacaatttacttggttccatcttccaagcaatatagaattga | |
| | ccgatgacaccagcaagaatcctccgatgagggtaccatatctcgggtcaaagacacagga | |
| | gaggagagctgcctcacttgcaaaaatagctcatatgtcgccacatgtaaaggctgccctaa | |
| | gggcatcatccgtgttgatctgggcttatggggataatgaagtaaattggactgctgctcttac | |
| | gattgcaaaatctcggtgtaatgtaaacttagagtatcttcggttactgtcccctttacccacgg | |
| | ctgggaatcttcaacatagactagatgatggtataactcagatgacattcacccctgcatctct | |
| | ctacaggGtgtcaccttacattcacatatccaatgattctcaaaggctgttcactgaagaagg | |
| | agtcaaagaggggaatgtggtttaccaacagatcatgctcttgggtttatctctaatcgaatcg | |
| | atctttccaatgacaacaaccaggacatatgatgagatcacactgcacctacatagtaaattta | |
| | gttgctgtatcagagaagcacctgttgcggttcctttcgagctacttggggtggtaccggaact | |
| | gaggacagtgacctcaaataagtttatgtatgatcctagccctgtatcggagggagactttgc | |
| | gagacttgacttagctatcttcaagagttatgagcttaatctggagtcatatcccacgatagag | |
| | ctaatgaacattctttcaatatccagcgggaagttgattggccagtctgtggtttcttatgatgaa | |
| | gatacctccataaagaatgacgccataatagtgtatgacaatacccgaaattggatcagtgaa | |
| | gctcagaattcagatgtggtccgcctatttgaatatgcagcacttgaagtgctcctcgactgttc | |
| | ttaccaactctattacctgagagtaagaggcctGgacaatattgtcttatatatgggtgatttata | |
| | caagaatatgccaggaattctactttccaacattgcagctacaatatctcatcccgtcattcattc | |
| | aaggttacatgcagtgggcctggtcaaccatgacggatcacaccaacttgcagatacggatt | |
| | ttatcgaaatgtctgcaaaactattagtatcttgcacccgacgtgtgatctccggcttatattcag | |
| | gaaataagtatgatctgctgttcccatctgtcttagatgataacctgaatgagaagatgcttcag | |
| | ctgatatcccggttatgctgtctgtacacggtactctttgctacaacaagagaaatcccgaaaa | |
| | taagaggcttaactgcagaagagaaatgttcaatactcactgagtatttactgtcggatgctgt | |
| | gaaaccattacttagccccgatcaagtgagctctatcatgtctcctaacataattacattcccag | |
| | ctaatctgtactacatgtctcggaagagcctcaatttgatcagggaaagggaggacagggat | |
| | actatcctggcgttgttgttcccccaagagccattattagagttcccttctgtgcaagatattggt | |
| | gctcgagtgaaagatccattcacccgacaacctgcggcatttttgcaagagttagatttgagt | |
| | gctccagcaaggtatgacgcattcacacttagtcagattcatcctgaactcacatctccaaatc | |
| | cggaggaagactacttagtacgatacttgttcagagggatagggactgcatcttcctcttggta | |
| | taaggcatctcatctcctttctgtacccgaggtaagatgtgcaagacacgggaactccttatac | |
| | ttagctgaagggagcggagccatcatgagtcttctcgaactgcatgtaccacatgaaactatc | |
| | tattacaatacgctcttttcaaatgagatgaaccccccgcaacgacatttcgggccgacccca | |
| | actcagtttttgaattcggttgtttataggaatctacaggcggaggtaacatgcaaagatggatt | |
| | tgtccaagagttccgtccattatggagagaaaatacagaggaaagCgacctgacctcagat | |
| | aaagTagtggggtatattacatctgcagtgccctacagatctgtatcattgctgcattgtgaca | |
| | ttgaaattcctccagggtccaatcaaagcttactagatcaactagctatcaatttatctctgattg | |
| | ccatgcattctgtaagggagggcggggtagtaatcatcaaagtgttgtatgcaatgggatact | |
| | actttcatctactcatgaacttgtttgctccgtgttccacaaaaggatatattctctctaatggttat | |
| | gcatgtcgaggagatatggagtgttacctggtatttgtcatgggttacctgggcgggcctaca | |
| | tttgtacatgaggtggtgaggatggcGaaaactctggtgcagcggcacggtacgctTttgt | |
| | ctaaatcagatgagatcacactgaccaggttattcacctcacagcggcagcgtgtgacagac | |
| | atcctatccagtcctttaccaagattaataaagtacttgaggaagaatattgacactgcgctgat | |
| | tgaagccgggggacagcccgtccgtccattctgtgcggagagtctggtgagcacgctagc | |
| | gaacataactcagataacccagatCatcgctagtcacattgacacagttatccggtctgtgat | |
| | atatatggaagctgagggtgatctcgctgacacagtatttctatttaccccttacaatctctctac | |
| | tgacgggaaaaagaggacatcacttaAacagtgcacgagacagatcctagaggttacaat | |
| | actaggtcttagagtcgaaaatctcaataaaataggcgatataatcagcctagtgcttaaagg | |
| | catgatctccatggaggaccttatcccactaaggacatacttgaagcatagtacctgccctaa | |
| | atatttgaaggctgtcctaggtattaccaaactcaaagaaatgtttacagacacttctgtaCtgt | |
| | acttgactcgtgctcaacaaaaattctacatgaaaactataggcaatgcagtcaaaggatatta | |
| | cagtaactgtgactcttaacgaaaatcacatattaataggctccttttttggccaattgtattcttgt | |
| | tgatttaatcatattatgttagaaaaaagttgaaccctgactccttaggactcgaattcgaactca | |
| | aataaatgtcttaaaaaaaggttgcgcacaattattcttgagtgtagtctcgtcattcaccaaatc | |
| | tttgtttggt | |
| cDNA of | ACCAAACAGAGAATCCGTAAGTTACGATAAAAGGCGA | SEQ ID |
| genomic | AGGAGCAATTGAAGTCGCACGGGTAGAAGGTGTGAATC | NO: 2 |
| sequence of | TCGAGTGCGAGCCCGAAGCACAAACTCGAGGAAGCCTT | |
| NDV strain | CTGCCAACATGTCTTCCGTATTCGACGAGTACGAACAG | |
| Hitchner B1 | CTCCTCGCGGCTCAGACTCGCCCCAATGGAGCTCATGG | |
| | AGGGGGGGAGAAAGGGAGTACCTTAAAAGTAGACGTC | |
| | CCGGTATTCACTCTTAACAGTGATGACCCAGAAGATAG | |
| | GTGGAGCTTTGTGGTATTCTGCCTCCGGATTGCTGTTAG | |
| | CGAAGATGCCAACAAACCACTCAGGCAAGGTGCTCTCA | |
| | TATCTCTTTTATGCTCCCACTCACAGGTAATGAGGAACC | |
| | ATGTTGCCCTTGCAGGGAAACAGAATGAAGCCACATTG | |
| | GCCGTGCTTGAGATTGATGGCTTTGCCAACGGCACGCC | |
| | CCAGTTCAACAATAGGAGTGGAGTGTCTGAAGAGAGAG | |
| | CACAGAGATTTGCGATGATAGCAGGATCTCTCCCTCGG | |
| | GCATGCAGCAACGGCACCCCGTTCGTCACAGCCGGGGC | |
| | TGAAGATGATGCACCAGAAGACATCACCGATACCCTGG | |
| | AGAGGATCCTCTCTATCCAGGCTCAAGTATGGGTCACA | |
| | GTAGCAAAAGCCATGACTGCGTATGAGACTGCAGATGA | |
| | GTCGGAAACAAGGCGAATCAATAAGTATATGCAGCAAG | |
| | GCAGGGTCCAAAAGAAATACATCCTCTACCCCGTATGC | |
| | AGGAGCACAATCCAACTCACGATCAGACAGTCTCTTGC | |
| | AGTCCGCATCTTTTTGGTTAGCGAGCTCAAGAGAGGCC | |
| | GCAACACGGCAGGTGGTACCTCTACTTATTATAACCTA | |
| | GTAGGGGACGTAGACTCATATATCAGGAATACCGGGCT | |
| | TACTGCATTCTTCTTGACACTCAAGTACGGAATCAACAC | |
| | CAAGACATCAGCCCTTGCACTTAGTAGCCTCTCAGGCG | |
| | ACATCCAGAAGATGAAGCAGCTCATGCGTTTGTATCGG | |
| | ATGAAAGGAGATAATGCGCCGTACATGACATTACTTGG | |
| | TGATAGTGACCAGATGAGCTTTGCGCCTGCCGAGTATG | |
| | CACAACTTTACTCCTTTGCCATGGGTATGGCATCAGTCC | |
| | TAGATAAAGGTACTGGGAAATACCAATTTGCCAGGGAC | |
| | TTTATGAGCACATCATTCTGGAGACTTGGAGTAGAGTA | |
| | CGCTCAGGCTCAGGGAAGTAGCATTAACGAGGATATGG | |
| | CTGCCGAGCTAAAGCTAACCCCGGCAGCAAGGAGGGGC | |
| | CTGGCAGCTGCTGCCCAACGAGTCTCCGAGGTGACCAG | |
| | CAGCATAGACATGCCTACTCAACAAGTCGGAGTCCTCA | |
| | CTGGGCTTAGCGAGGGGGGATCCCAAGCCCTACAAGGC | |
| | GGATCGAATAGATCGCAAGGGCAACCAGAAGCCGGGG | |
| | ATGGGGAGACCCAATTCCTGGATCTGATGAGAGCGGTA | |
| | GCAAATAGCATGAGGGAGGCGCCAAACTCTGCACAGG | |
| | GCACTCCCCAATCGGGGCCTCCCCCAACTCCTGGGCCAT | |
| | CCCAAGATAACGACACCGACTGGGGGTATTGATTGACA | |
| | AAACCCAGCCTGCTTCTACAAGAACATCCCAATGCTCTC | |
| | ACCCGTAGTCGACCCCTCGATTTGCGGCTCTATATGACC | |
| | ACACCCTCAAACAAACATCCCCCTCTTTCCTCCCTCCCC | |
| | CTGCTGTACAACTCCGCACGCCCTAGATACCACAGGCA | |
| | CACCGCGGCTCACTAACAATCAAAACAGAGCCGAGGGA | |
| | ATTAGAAAAAAGTACGGGTAGAAGAGGGATATTCAGA | |
| | GATCAGGGCAAGTCTCCCGAGTCTCTGCTCTCTCCTCTA | |
| | CCTGATAGACCAGGACAAACATGGCCACCTTTACAGAT | |
| | GCAGAGATCGACGAGCTATTTGAGACAAGTGGAACTGT | |
| | CATTGACAACATAATTACAGCCCAGGGTAAACCAGCAG | |
| | AGACTGTTGGAAGGAGTGCAATCCCACAGGGCAAGACC | |
| | AAGGTGCTGAGCGCAGCATGGGAGAAGCATGGGAGCA | |
| | TCCAGCCACCGGCCAGTCAAGACAACCTCGATCGACAG | |
| | GACAGATCTGACAAACAACCATCCACACCCGAGCAAAC | |
| | GACCCCGCACGACAGCCCGCCGGCCACATCCGCTGACC | |
| | AGCCCCCCACCCAGGCCACAGACGAAGCCGTCGACACA | |
| | CAGCTCAGGACCGGAGCAAGCAACTCTCTGCTGTTGAT | |
| | GCTTGACAAGCTCAGCAATAAATCGTCCAATGCTAAAA | |
| | AGGGCCCATGGTCGAGCCCCCAAGAGGGGAATCACCAA | |
| | CGTCCGACTCAACAGCAGGGGAGTCAACCCAGTCGCGG | |
| | AAACAGCCAGGAAAGACTGCAGAACCAAGTCAAGGCC | |
| | GCCCCTGGAAACCAGGGCACAGACGTGAACACAGCATA | |
| | TCATGGACAATGGGAGGAGTCACAACTATCAGCTGGTG | |
| | CAACCCCTCATGCTCTCCGATCAAGGCAGAGCCAAGAC | |
| | AATACCCTTGTATCTGCGGATCATGTCCAGCCACCTGTA | |
| | GACTTTGTGCAAGCGATGATGTCTATGATGGGGGCGAT | |
| | ATCACAGAGAGTAAGTAAGGTTGACTATCAGCTAGATC | |
| | TTGTCTTGAAACAGACATCCTCCATCCCTATGATGCGGT | |
| | CCGAAATCCAACAGCTGAAAACATCTGTTGCAGTCATG | |
| | GAAGCCAACTTGGGAATGATGAAGATTCTGGATCCCGG | |
| | TTGTGCCAACATTTCATCTCTGAGTGATCTACGGGCAGT | |
| | TGCCCGATCTCACCCGGTTTTAGTTTCAGGCCCTGGAGA | |
| | CCCATCTCCCTATGTGATACAAGGAGGCGAAATGGCAC | |
| | TTAATAAACTTTCGCAACCAGTGCCACATCCATCTGAAT | |
| | TGATTAAACCCGCCACTGCATGCGGGCCTGATATAGGA | |
| | GTGGAGAGGGACACTGTCCGTGCATTGATCATGTCACG | |
| | CCCAATGCACCCGAGTTCTTCAGCCAAGCTCCTAAGCA | |
| | AGTTAGATGCAGCCGGGTCGATCGAGGAAATCAGGAAA | |
| | ATCAAGCGCCTTGCTCTAAATGGCTAATTACTACTGCCA | |
| | CACGTAGCGGGTCCCTGTCCACTCGGCATCACACGGAA | |
| | TCTGCACCGAGTTCCCCCCCGCAGACCCAAGGTCCAAC | |
| | TCTAGAAGCGGCAATCCTCTCTCGCTTCCTCAGCCCCAC | |
| | TGAATGATCGCGTAACCGTAATTAATCTAGCTACATTAA | |
| | GGATTAAGAAAAAATACGGGTAGAATTGGAGTGCCCCA | |
| | ATTGTGCCAAGATGGACTCATCTAGGACAATTGGGCTG | |
| | TACTTTGATTCTGCCCATTCTTCTAGCAACCTGTTAGCA | |
| | TTTCCGATCGTCCTACAAGACACAGGAGATGGGAAGAA | |
| | GCAAATCGCCCCGCAATATAGGATCCAGCGCCTTGACT | |
| | CGTGGACTGATAGTAAGGAAGACTCAGTATTCATCACC | |
| | ACCTATGGATTCATCTTTCAAGTTGGGAATGAGGAAGC | |
| | CACTGTCGGCATGATCGATGATAAACCCAAGCGCGAGT | |
| | TACTTTCCGCTGCGATGCTCTGCCTAGGAAGCGTCCCAA | |
| | ATACCGGAGACCTTGTTGAGCTGGCAAGGGCCTGTCTC | |
| | ACTATGATGGTCACATGCAAGAAGAGTGCAACTAATAC | |
| | TGAGAGAATGGTTTTCTCAGTAGTGCAGGCACCCCAAG | |
| | TGCTGCAAAGCTGTAGGGTTGTGGCAAATAAATACTCA | |
| | TCAGTGAATGCAGTCAAGCACGTGAAAGCGCCAGAGAA | |
| | GATCCCCGGGAGTGGAACCCTAGAATACAAGGTGAACT | |
| | TTGTCTCCTTGACTGTGGTACCGAAGAAGGATGTCTACA | |
| | AGATCCCAGCTGCAGTATTGAAGATTTCTGGCTCGAGTC | |
| | TGTACAATCTTGCGCTCAATGTCACTATTAATGTGGAGG | |
| | TAGACCCGAGGAGTCCTTTGGTTAAATCTCTGTCTAAGT | |
| | CTGACAGCGGATACTATGCTAACCTCTTCTTGCATATTG | |
| | GACTTATGACCACCGTAGATAGGAAGGGGAAGAAAGT | |
| | GACATTTGACAAGCTGGAAAAGAAAATAAGGAGCCTTG | |
| | ATCTATCTGTCGGGCTCAGTGATGTGCTCGGGCCTTCCG | |
| | TGTTGGTAAAAGCAAGAGGTGCACGGACTAAGCTTTTG | |
| | GCACCTTTCTTCTCTAGCAGTGGGACAGCCTGCTATCCC | |
| | ATAGCAAATGCTTCTCCTCAGGTGGCCAAGATACTCTG | |
| | GAGTCAAACCGCGTGCCTGCGGAGCGTTAAAATCATTA | |
| | TCCAAGCAGGTACCCAACGCGCTGTCGCAGTGACCGCT | |
| | GACCACGAGGTTACCTCTACTAAGCTGGAGAAGGGGCA | |
| | CACCCTTGCCAAATACAATCCTTTTAAGAAATAAGCTGC | |
| | GTCTCTGAGATTGCGCTCCGCCCACTCACCCAGATCATC | |
| | ATGACACAAAAAACTAATCTGTCTTGATTATTTACAGTT | |
| | AGTTTACCTGTCCATCAAGTTAGAAAAAACACGGGTAG | |
| | AAGATTCTGGATCCCGGTTGGCGCCCTCCAGGTGCAGG | |
| | ATGGGCTCCAGACCTTCTACCAAGAACCCAGCACCTAT | |
| | GATGCTGACTATCCGGGTCGCGCTGGTACTGAGTTGCAT | |
| | CTGCCCGGCAAACTCCATTGATGGCAGGCCTCTTGCAG | |
| | CTGCAGGAATTGTGGTTACAGGAGACAAAGCAGTCAAC | |
| | ATATACACCTCATCCCAGACAGGATCAATCATAGTTAA | |
| | GCTCCTCCCGAATCTGCCCAAGGATAAGGAGGCATGTG | |
| | CGAAAGCCCCCTTGGATGCATACAACAGGACATTGACC | |
| | ACTTTGCTCACCCCCCTTGGTGACTCTATCCGTAGGATA | |
| | CAAGAGTCTGTGACTACATCTGGAGGGGGGAGACAGGG | |
| | GCGCCTTATAGGCGCCATTATTGGCGGTGTGGCTCTTGG | |
| | GGTTGCAACTGCCGCACAAATAACAGCGGCCGCAGCTC | |
| | TGATACAAGCCAAACAAAATGCTGCCAACATCCTCCGA | |
| | CTTAAAGAGAGCATTGCCGCAACCAATGAGGCTGTGCA | |
| | TGAGGTCACTGACGGATTATCGCAACTAGCAGTGGCAG | |
| | TTGGGAAGATGCAGCAGTTTGTTAATGACCAATTTAAT | |
| | AAAACAGCTCAGGAATTAGACTGCATCAAAATTGCACA | |
| | GCAAGTTGGTGTAGAGCTCAACCTGTACCTAACCGAAT | |
| | TGACTACAGTATTCGGACCACAAATCACTTCACCTGCCT | |
| | TAAACAAGCTGACTATTCAGGCACTTTACAATCTAGCTG | |
| | GTGGGAATATGGATTACTTATTGACTAAGTTAGGTATA | |
| | GGGAACAATCAACTCAGCTCATTAATCGGTAGCGGCTT | |
| | AATCACCGGTAACCCTATTCTATACGACTCACAGACTCA | |
| | ACTCTTGGGTATACAGGTAACTCTACCTTCAGTCGGGAA | |
| | CCTAAATAATATGCGTGCCACCTACTTGGAAACCTTATC | |
| | CGTAAGCACAACCAGGGGATTTGCCTCGGCACTTGTCC | |
| | CAAAAGTGGTGACACAGGTCGGTTCTGTGATAGAAGAA | |
| | CTTGACACCTCATACTGTATAGAAACTGACTTAGATTTA | |
| | TATTGTACAAGAATAGTAACGTTCCCTATGTCCCCTGGT | |
| | ATTTACTCCTGCTTGAGCGGCAATACATCGGCCTGTATG | |
| | TACTCAAAGACCGAAGGCGCACTTACTACACCATATAT | |
| | GACTATCAAAGGCTCAGTCATCGCTAACTGCAAGATGA | |
| | CAACATGTAGATGTGTAAACCCCCCGGGTATCATATCG | |
| | CAAAACTATGGAGAAGCCGTGTCTCTAATAGATAAACA | |
| | ATCATGCAATGTTTTATCCTTAGGCGGGATAACTTTAAG | |
| | GCTCAGTGGGGAATTCGATGTAACTTATCAGAAGAATA | |
| | TCTCAATACAAGATTCTCAAGTAATAATAACAGGCAAT | |
| | CTTGATATCTCAACTGAGCTTGGGAATGTCAACAACTCG | |
| | ATCAGTAATGCTTTGAATAAGTTAGAGGAAAGCAACAG | |
| | AAAACTAGACAAAGTCAATGTCAAACTGACCAGCACAT | |
| | CTGCTCTCATTACCTATATCGTTTTGACTATCATATCTCT | |
| | TGTTTTTGGTATACTTAGCCTGATTCTAGCATGCTACCT | |
| | AATGTACAAGCAAAAGGCGCAACAAAAGACCTTATTAT | |
| | GGCTTGGGAATAATACCCTAGATCAGATGAGAGCCACT | |
| | ACAAAAATGTGAACACAGATGAGGAACGAAGGTTTCCC | |
| | TAATAGTAATTTGTGTGAAAGTTCTGGTAGTCTGTCAGT | |
| | TCGGAGAGTTAAGAAAAAACTACCGGTTGTAGATGACC | |
| | AAAGGACGATATACGGGTAGAACGGTAAGAGAGGCCG | |
| | CCCCTCAATTGCGAGCCAGACTTCACAACCTCCGTTCTA | |
| | CCGCTTCACCGACAACAGTCCTCAATCATGGACCGCGC | |
| | CGTTAGCCAAGTTGCGTTAGAGAATGATGAAAGAGAGG | |
| | CAAAAAATACATGGCGCTTGATATTCCGGATTGCAATC | |
| | TTATTCTTAACAGTAGTGACCTTGGCTATATCTGTAGCC | |
| | TCCCTTTTATATAGCATGGGGGCTAGCACACCTAGCGAT | |
| | CTTGTAGGCATACCGACTAGGATTTCCAGGGCAGAAGA | |
| | AAAGATTACATCTACACTTGGTTCCAATCAAGATGTAGT | |
| | AGATAGGATATATAAGCAAGTGGCCCTTGAGTCTCCAT | |
| | TGGCATTGTTAAATACTGAGACCACAATTATGAACGCA | |
| | ATAACATCTCTCTCTTATCAGATTAATGGAGCTGCAAAC | |
| | AACAGCGGGTGGGGGGCACCTATTCATGACCCAGATTA | |
| | TATAGGGGGGATAGGCAAAGAACTCATTGTAGATGATG | |
| | CTAGTGATGTCACATCATTCTATCCCTCTGCATTTCAAG | |
| | AACATCTGAATTTTATCCCGGCGCCTACTACAGGATCAG | |
| | GTTGCACTCGAATACCCTCATTTGACATGAGTGCTACCC | |
| | ATTACTGCTACACCCATAATGTAATATTGTCTGGATGCA | |
| | GAGATCACTCACACTCACATCAGTATTTAGCACTTGGTG | |
| | TGCTCCGGACATCTGCAACAGGGAGGGTATTCTTTTCTA | |
| | CTCTGCGTTCCATCAACCTGGACGACACCCAAAATCGG | |
| | AAGTCTTGCAGTGTGAGTGCAACTCCCCTGGGTTGTGAT | |
| | ATGCTGTGCTCGAAAGCCACGGAGACAGAGGAAGAAG | |
| | ATTATAACTCAGCTGTCCCTACGCGGATGGTACATGGG | |
| | AGGTTAGGGTTCGACGGCCAATATCACGAAAAGGACCT | |
| | AGATGTCACAACATTATTCGGGGACTGGGTGGCCAACT | |
| | ACCCAGGAGTAGGGGGTGGATCTTTTATTGACAGCCGC | |
| | GTATGGTTCTCAGTCTACGGAGGGTTAAAACCCAATAC | |
| | ACCCAGTGACACTGTACAGGAAGGGAAATATGTGATAT | |
| | ACAAGCGATACAATGACACATGCCCAGATGAGCAAGAC | |
| | TACCAGATTCGAATGGCCAAGTCTTCGTATAAGCCTGG | |
| | ACGGTTTGGTGGGAAACGCATACAGCAGGCTATCTTAT | |
| | CTATCAAAGTGTCAACATCCTTAGGCGAAGACCCGGTA | |
| | CTGACTGTACCGCCCAACACAGTCACACTCATGGGGGC | |
| | CGAAGGCAGAATTCTCACAGTAGGGACATCCCATTTCT | |
| | TGTATCAGCGAGGGTCATCATACTTCTCTCCCGCGTTAT | |
| | TATATCCTATGACAGTCAGCGACAAAACAGCCACTCTT | |
| | CATAGTCCTTATACATTCAATGCCTTCACTCGGCCAGGT | |
| | AGTATCCCTTGCCAGGCTTCAGCAAGATGCCCCAACTC | |
| | GTGTGTTACTGGAGTCTATACAGATCCATATCCCCTAAT | |
| | CTTCTATAGAAACCACACCTTGCGAGGGGTATTCGGGA | |
| | CAATGCTTGATGGTGAACAAGCAAGACTTAACCCTGCG | |
| | TCTGCAGTATTCGATAGCACATCCCGCAGTCGCATAACT | |
| | CGAGTGAGTTCAAGCAGCATCAAAGCAGCATACACAAC | |
| | ATCAACTTGTTTTAAAGTGGTCAAGACCAATAAGACCT | |
| | ATTGTCTCAGCATTGCTGAAATATCTAATACTCTCTTCG | |
| | GAGAATTCAGAATCGTCCCGTTACTAGTTGAGATCCTCA | |
| | AAGATGACGGGGTTAGAGAAGCCAGGTCTGGCTAGTTG | |
| | AGTCAACTATGAAAGAGTTGGAAAGATGGCATTGTATC | |
| | ACCTATCTTCTGCGACATCAAGAATCAAACCGAATGCC | |
| | GGCGCGTGCTCGAATTCCATGTCGCCAGTTGACCACAA | |
| | TCAGCCAGTGCTCATGCGATCAGATTAAGCCTTGTCAAT | |
| | AGTCTCTTGATTAAGAAAAAATGTAAGTGGCAATGAGA | |
| | TACAAGGCAAAACAGCTCACGGTAAATAATACGGGTAG | |
| | GACATGGCGAGCTCCGGTCCTGAAAGGGCAGAGCATCA | |
| | GATTATCCTACCAGAGTCACACCTGTCTTCACCATTGGT | |
| | CAAGCACAAACTACTCTATTATTGGAAATTAACTGGGC | |
| | TACCGCTTCCTGATGAATGTGACTTCGACCACCTCATTC | |
| | TCAGCCGACAATGGAAAAAAATACTTGAATCGGCCTCT | |
| | CCTGATACTGAGAGAATGATAAAACTCGGAAGGGCAGT | |
| | ACACCAAACTCTTAACCACAATTCCAGAATAACCGGAG | |
| | TACTCCACCCCAGGTGTTTAGAAGAACTGGCTAATATTG | |
| | AGGTCCCTGATTCAACCAACAAATTTCGGAAGATTGAG | |
| | AAGAAGATCCAAATTCACAACACGAGATATGGAGAACT | |
| | GTTCACAAGGCTGTGTACGCATATAGAGAAGAAACTGC | |
| | TGGGGTCATCTTGGTCTAACAATGTCCCCCGGTCAGAG | |
| | GAGTTCAGCAGCATTCGTACGGATCCGGCATTCTGGTTT | |
| | CACTCAAAATGGTCCACAGCCAAGTTTGCATGGCTCCA | |
| | TATAAAACAGATCCAGAGGCATCTGATTGTGGCAGCTA | |
| | GGACAAGGTCTGCGGCCAACAAATTGGTGATGCTAACC | |
| | CATAAGGTAGGCCAAGTCTTTGTCACTCCTGAACTTGTT | |
| | GTTGTGACGCATACGAATGAGAACAAGTTCACATGTCT | |
| | TACCCAGGAACTTGTATTGATGTATGCAGATATGATGG | |
| | AGGGCAGAGATATGGTCAACATAATATCAACCACGGCG | |
| | GTGCATCTCAGAAGCTTATCAGAGAAAATTGATGACAT | |
| | TTTGCGGTTAATAGACGCTCTGGCAAAAGACTTGGGTA | |
| | ATCAAGTCTACGATGTTGTATCACTAATGGAGGGATTTG | |
| | CATACGGAGCTGTCCAGCTACTCGAGCCGTCAGGTACA | |
| | TTTGCGGGAGATTTCTTCGCATTCAACCTGCAGGAGCTT | |
| | AAAGACATTCTAATTGGCCTCCTCCCCAATGATATAGCA | |
| | GAATCCGTGACTCATGCAATCGCTACTGTATTCTCTGGT | |
| | TTAGAACAGAATCAAGCAGCTGAGATGTTGTGCCTGTT | |
| | GCGTCTGTGGGGTCACCCACTGCTTGAGTCCCGTATTGC | |
| | AGCAAAGGCAGTCAGGAGCCAAATGTGCGCACCGAAA | |
| | ATGGTAGACTTTGATATGATCCTTCAGGTACTGTCTTTC | |
| | TTCAAGGGAACAATCATCAACGGATACAGAAAGAAGA | |
| | ATGCAGGTGTGTGGCCGCGAGTCAAAGTGGATACAATA | |
| | TATGGGAAGGTCATTGGGCAACTACATGCAGATTCAGC | |
| | AGAGATTTCACACGATATCATGTTGAGAGAGTATAAGA | |
| | GTTTATCTGCACTTGAATTTGAGCCATGTATAGAATACG | |
| | ACCCTGTCACTAACCTGAGCATGTTCCTAAAAGACAAG | |
| | GCAATCGCACACCCCAACGATAATTGGCTTGCCTCGTTT | |
| | AGGCGGAACCTTCTCTCCGAAGACCAGAAGAAACATGT | |
| | AAAGGAAGCGACTTCGACTAACCGCCTCTTGATAGAGT | |
| | TTTTAGAGTCAAATGATTTTGATCCATATAAAGAGATGG | |
| | AATATCTGACGACCCTTGAGTACCTTAGAGATGACAAT | |
| | GTGGCAGTATCATACTCGCTCAAAGAGAAGGAAGTGAA | |
| | AGTTAATGGACGGATCTTCGCTAAGCTGACAAAGAAGT | |
| | TAAGGAACTGTCAGGTGATGGCGGAAGGGATCCTAGCC | |
| | GATCAGATTGCACCTTTCTTTCAGGGAAATGGAGTCATT | |
| | CAGGATAGCATATCCTTGACCAAGAGTATGCTAGCGAT | |
| | GAGTCAACTGTCTTTTAACAGCAATAAGAAACGTATCA | |
| | CTGACTGTAAAGAAAGAGTATGTTCAAACCGCAATCAT | |
| | GATCCGAAAAGCAAGAACCGTCGGAGAGTTGCAACCTT | |
| | CATAACAACTGACCTGCAAAAGTACTGTCTTAATTGGA | |
| | GATATCAGACGATCAAATTGTTCGCTCATGCCATCAATC | |
| | AGTTGATGGGCCTACCTCATTTCTTCGAGTGGATTCACC | |
| | TAAGACTGATGGACACTACGATGTTCGTAGGAGACCCT | |
| | TTCAATCCTCCAAGTGACCCTACTGACTGTGACCTCTCA | |
| | AGAGTCCCTAATGATGACATATATATTGTCAGTGCCAG | |
| | AGGGGGTATCGAAGGATTATGCCAGAAGCTATGGACAA | |
| | TGATCTCAATTGCTGCAATCCAACTTGCTGCAGCTAGAT | |
| | CGCATTGTCGTGTTGCCTGTATGGTACAGGGTGATAATC | |
| | AAGTAATAGCAGTAACGAGAGAGGTAAGATCAGATGA | |
| | CTCTCCGGAGATGGTGTTGACACAGTTGCATCAAGCCA | |
| | GTGATAATTTCTTCAAGGAATTAATCCATGTCAATCATT | |
| | TGATTGGCCATAATTTGAAGGATCGTGAAACCATCAGG | |
| | TCAGACACATTCTTCATATACAGCAAACGAATCTTCAA | |
| | AGATGGAGCAATCCTCAGTCAAGTCCTCAAAAATTCAT | |
| | CTAAATTAGTGCTAGTGTCAGGTGATCTCAGTGAAAAC | |
| | ACCGTAATGTCCTGTGCCAACATTGCCTCTACTGTAGCA | |
| | CGGCTATGCGAGAACGGGCTTCCCAAAGACTTCTGTTA | |
| | CTATTTAAACTATATAATGAGTTGTGTGCAGACATACTT | |
| | TGACTCTGAGTTCTCCATCACCAACAATTCGCACCCCGA | |
| | TCTTAATCAGTCGTGGATTGAGGACATCTCTTTTGTGCA | |
| | CTCATATGTTCTGACTCCTGCCCAATTAGGGGGACTGAG | |
| | TAACCTTCAATACTCAAGGCTCTACACTAGAAATATCG | |
| | GTGACCCGGGGACTACTGCTTTTGCAGAGATCAAGCGA | |
| | CTAGAAGCAGTGGGACTACTGAGTCCTAACATTAGGAC | |
| | TAATATCTTAACTAGGCCGCCTGGGAATGGAGATTGGG | |
| | CCAGTCTGTGCAACGACCCATACTCTTTCAATTTTGAGA | |
| | CTGTTGCAAGCCCAAACATTGTTCTTAAGAAACATACG | |
| | CAAAGAGTCCTATTTGAAACTTGTTCAAATCCCTTATTG | |
| | TCTGGAGTGCACACAGAGGATAATGAGGCAGAAGAGA | |
| | AGGCATTGGCTGAATTCTTGCTTAATCAAGAGGTGATTC | |
| | ATCCCCGCGTTGCGCATGCCATCATGGAGGCAAGCTCT | |
| | GTAGGTAGGAGAAAGCAAATTCAAGGGCTTGTTGACAC | |
| | AACAAACACTGTAATTAAGATTGCGCTTACTAGGAGGC | |
| | CATTAGGCATCAAGAGGCTGATGCGGATAGTCAATTAT | |
| | TCTAGCATGCATGCAATGCTGTTTAGAGACGATGTTTTT | |
| | TCCTCTAGTAGATCCAACCACCCCTTAGTCTCTTCTAAT | |
| | ATGTGTTCTCTGACACTGGCAGACTATGCACGGAATAG | |
| | AAGCTGGTCACCTTTGACGGGAGGCAGGAAAATACTGG | |
| | GTGTATCTAATCCTGATACGATAGAACTCGTAGAGGGT | |
| | GAGATTCTTAGTGTAAGCGGAGGGTGTACAAGATGTGA | |
| | CAGCGGAGATGAACAATTTACTTGGTTCCATCTTCCAAG | |
| | CAATATAGAATTGACCGATGACACCAGCAAGAATCCTC | |
| | CGATGAGGGTACCATATCTCGGGTCAAAGACACAGGAG | |
| | AGGAGAGCTGCCTCACTTGCGAAAATAGCTCATATGTC | |
| | GCCACATGTGAAGGCTGCCCTAAGGGCATCATCCGTGT | |
| | TGATCTGGGCTTATGGGGATAATGAAGTAAATTGGACT | |
| | GCTGCTCTTACGATTGCAAAATCTCGGTGTAATGTAAAC | |
| | TTAGAGTATCTTCGGTTACTGTCCCCTTTACCCACGGCT | |
| | GGGAATCTTCAACATAGACTAGATGATGGTATAACTCA | |
| | GATGACATTCACCCCTGCATCTCTCTACAGGGTGTCACC | |
| | TTACATTCACATATCCAATGATTCTCAAAGGCTGTTCAC | |
| | TGAAGAAGGAGTCAAAGAGGGGAATGTGGTTTACCAAC | |
| | AGATCATGCTCTTGGGTTTATCTCTAATCGAATCGATCT | |
| | TTCCAATGACAACAACCAGAACATATGATGAGATCACA | |
| | CTGCACCTACATAGTAAATTTAGTTGCTGTATCAGGGAA | |
| | GCACCTGTTGCGGTTCCTTTCGAGCTACTTGGGGGGCA | |
| | CCGGAACTGAGGACAGTGACCTCAAATAAGTTTATGTA | |
| | TGATCCTAGCCCTGTATCGGAGGGAGACTTTGCGAGAC | |
| | TTGACTTAGCTATCTTCAAGAGTTATGAGCTTAATCTGG | |
| | AGTCATATCCCACGATAGAGCTAATGAACATTCTTTCAA | |
| | TATCCAGCGGGAAGTTGATTGGCCAGTCTGTGGTTTCTT | |
| | ATGATGAAGATACCTCCATAAAGAATGATGCCATAATA | |
| | GTGTATGACAATACCCGAAATTGGATCAGTGAAGCTCA | |
| | GAATTCAGATGTGGTCCGCCTATTTGAATATGCAGCACT | |
| | TGAAGTGCTCCTCGACTGTTCTTACCAACTCTATTACCT | |
| | GAGAGTAAGAGACCTAGACAATATTGTCTTATATATGG | |
| | GTGATTTATACAAGAATATGCCAGGAATTCTACTTTCCA | |
| | ACATTGCAGCTACAATATCTCATCCTGTCATTCATTCAA | |
| | GGTTACATGCAGTGGGCCTGGTCAACCATGACGGATCA | |
| | CACCAACTTGCAGATACGGATTTTATCGAAATGTCTGCA | |
| | AAACTGTTAGTATCTTGCACCCGACGTGTGATCTCCGGC | |
| | TTATATTCAGGAAATAAGTATGATCTGCTGTTCCCATCT | |
| | GTCTTAGATGATAACCTGAATGAGAAGATGCTTCAGCT | |
| | GATATCCCGGTTATGCTGTCTGTACACGGTACTCTTTGC | |
| | TACAACAAGAGAAATCCCGAAAATAAGAGGCTTAACTG | |
| | CAGAAGAGAAATGTTCAATACTCACTGAGTATTTACTG | |
| | TCGGATGCTGTGAAACCATTACTTAGCCCCGATCAAGT | |
| | GAGCTCTATCATGTCTCCTAACATAATTACATTCCCAGC | |
| | TAATCTGTACTACATGTCTCGGAAGAGCCTCAATTTGAT | |
| | CAGGGAAAGGGAGGACAGGGATACTATCCTGGCGTTGT | |
| | TGTTCCCCCAAGAGCCATTATTAGAGTTCCCTTCTGTGC | |
| | AAGATATTGGTGCTCGAGTGAAAGATCCATTCACCCGA | |
| | CAACCTGCGGCATTTTTGCAAGAGTTAGATTTGAGTGCT | |
| | CCAGCAAGGTATGACGCATTCACACTTAGTCAGATTCA | |
| | TCCTGAACTCACATCTCCAAATCCGGAGGAAGACTACT | |
| | TAGTACGATACTTGTTCAGAGGGATAGGGACTGCATCT | |
| | TCCTCTTGGTATAAGGCATCCCATCTCCTTTCTGTACCC | |
| | GAGGTAAGATGTGCAAGACACGGGAACTCCTTATACTT | |
| | GGCTGAAGGAAGCGGAGCCATCATGAGTCTTCTTGAAC | |
| | TGCATGTACCACATGAAACTATCTATTACAATACGCTCT | |
| | TTTCAAATGAGATGAACCCCCCGCAACGACATTTCGGG | |
| | CCGACCCCAACTCAGTTTTTGAATTCGGTTGTTTATAGG | |
| | AATCTACAGGCGGAGGTAACATGCAAGGATGGATTTGT | |
| | CCAAGAGTTCCGTCCATTATGGAGAGAAAATACAGAGG | |
| | AAAGTGACCTGACCTCAGATAAAGCAGTGGGGTATATT | |
| | ACATCTGCAGTACCCTACAGATCTGTATCATTGCTGCAT | |
| | TGTGACATTGAAATTCCTCCAGGGTCCAATCAAAGCTTA | |
| | CTAGATCAACTAGCTATCAATTTATCTCTGATTGCCATG | |
| | CATTCTGTAAGGGAGGGGGGGGTAGTAATCATCAAAGT | |
| | GTTGTATGCAATGGGATACTACTTTCATCTACTCATGAA | |
| | CTTGTTTGCTCCGTGTTCCACAAAAGGATATATTCTCTC | |
| | TAATGGTTATGCATGTCGAGGGGATATGGAGTGTTACC | |
| | TGGTATTTGTCATGGGTTACCTGGGCGGGCCTACATTTG | |
| | TACATGAGGTGGTGAGGATGGCAAAAACTCTGGTGCAG | |
| | CGGCACGGTACGCTTTTGTCTAAATCAGATGAGATCAC | |
| | ACTGACCAGGTTATTCACCTCACAGCGGCAGCGTGTGA | |
| | CAGACATCCTATCCAGTCCTTTACCAAGATTAATAAAGT | |
| | ACTTGAGGAAGAATATTGACACTGCGCTGATTGAAGCC | |
| | GGGGGACAGCCCGTCCGTCCATTCTGTGCGGAGAGTCT | |
| | GGTGAGCACGCTAGCGAACATAACTCAGATAACCCAGA | |
| | TCATCGCTAGTCACATTGACACAGTCATCCGGTCTGTGA | |
| | TATATATGGAAGCTGAGGGTGATCTCGCTGACACAGTA | |
| | TTTCTATTTACCCCTTACAATCTCTCTACTGACGGGAAA | |
| | AAGAGGACATCACTTAAACAGTGCACGAGACAGATCCT | |
| | AGAGGTTACAATACTAGGTCTTAGAGTCGAAAATCTCA | |
| | ATAAAATAGGCGATATAATCAGCCTAGTGCTTAAAGGC | |
| | ATGATCTCCATGGAGGACCTTATCCCACTAAGGACATA | |
| | CTTGAAGCATAGTACCTGCCCTAAATATTTGAAGGCTGT | |
| | CCTAGGTATTACCAAACTCAAAGAAATGTTTACAGACA | |
| | CTTCTGTACTGTACTTGACTCGTGCTCAACAAAAATTCT | |
| | ACATGAAAACTATAGGCAATGCAGTCAAAGGATATTAC | |
| | AGTAACTGTGACTCCTAACGAAAATCACATATTAATAG | |
| | GCTCCTTTTTTGGCCAATTGTATTCTTGTTGATTTAATTA | |
| | TATTATGTTAGAAAAAAGTTGAACTCTGACTCCTTAGGA | |
| | CTCGAATTCGAACTCAAATAAATGTCTTTAAAAAAGGT | |
| | TGCGCACAATTATTCTTGAGTGTAGTCTCGTCATTCACC | |
| | AAATCTTTGTTTGGT | |
| cDNA of | ACCAAACAGAGAATCCGTGAGTTACGATAAAAGGCGA | SEQ ID |
| genomic | AGGAGCAATTGAAGTCGCACGGGTAGAAGGTGTGAATC | NO: 3 |
| sequence of | TCGAGTGCGAGCCCGAAGCACAAACTCGAGAAAGCCTT | |
| NDV strain | CTGCCAACATGTCTTCCGTATTTGATGAGTACGAACAGC | |
| LaSota (L289A | TCCTCGCGGCTCAGACTCGCCCCAATGGAGCTCATGGA | |
| mutation) | GGGGGAGAAAAAGGGAGTACCTTAAAAGTAGACGTCC | |
| | CGGTATTCACTCTTAACAGTGATGACCCAGAAGATAGA | |
| | TGGAGCTTTGTGGTATTCTGCCTCCGGATTGCTGTTAGC | |
| | GAAGATGCCAACAAACCACTCAGGCAAGGTGCTCTCAT | |
| | ATCTCTTTTATGCTCCCACTCACAGGTAATGAGGAACCA | |
| | TGTTGCCCTTGCAGGGAAACAGAATGAAGCCACATTGG | |
| | CCGTGCTTGAGATTGATGGCTTTGCCAACGGCACGCCCC | |
| | AGTTCAACAATAGGAGTGGAGTGTCTGAAGAGAGAGCA | |
| | CAGAGATTTGCGATGATAGCAGGATCTCTCCCTCGGGC | |
| | ATGCAGCAACGGAACCCCGTTCGTCACAGCCGGGGCCG | |
| | AAGATGATGCACCAGAAGACATCACCGATACCCTGGAG | |
| | AGGATCCTCTCTATCCAGGCTCAAGTATGGGTCACAGT | |
| | AGCAAAAGCCATGACTGCGTATGAGACTGCAGATGAGT | |
| | CGGAAACAAGGCGAATCAATAAGTATATGCAGCAAGG | |
| | CAGGGTCCAAAAGAAATACATCCTCTACCCCGTATGCA | |
| | GGAGCACAATCCAACTCACGATCAGACAGTCTCTTGCA | |
| | GTCCGCATCTTTTTGGTTAGCGAGCTCAAGAGAGGCCG | |
| | CAACACGGCAGGTGGTACCTCTACTTATTATAACCTGGT | |
| | AGGGGACGTAGACTCATACATCAGGAATACCGGGCTTA | |
| | CTGCATTCTTCTTGACACTCAAGTACGGAATCAACACCA | |
| | AGACATCAGCCCTTGCACTTAGTAGCCTCTCAGGCGAC | |
| | ATCCAGAAGATGAAGCAGCTCATGCGTTTGTATCGGAT | |
| | GAAAGGAGATAATGCGCCGTACATGACATTACTTGGTG | |
| | ATAGTGACCAGATGAGCTTTGCGCCTGCCGAGTATGCA | |
| | CAACTTTACTCCTTTGCCATGGGTATGGCATCAGTCCTA | |
| | GATAAAGGTACTGGGAAATACCAATTTGCCAGGGACTT | |
| | TATGAGCACATCATTCTGGAGACTTGGAGTAGAGTACG | |
| | CTCAGGCTCAGGGAAGTAGCATTAACGAGGATATGGCT | |
| | GCCGAGCTAAAGCTAACCCCAGCAGCAAGGAGGGGCCT | |
| | GGCAGCTGCTGCCCAACGGGTCTCCGAGGAGACCAGCA | |
| | GCATAGACATGCCTACTCAACAAGTCGGAGTCCTCACT | |
| | GGGCTTAGCGAGGGGGGGTCCCAAGCTCTACAAGGCGG | |
| | ATCGAATAGATCGCAAGGGCAACCAGAAGCCGGGGAT | |
| | GGGGAGACCCAATTCCTGGATCTGATGAGAGCGGTAGC | |
| | AAATAGCATGAGGGAGGCGCCAAACTCTGCACAGGGC | |
| | ACTCCCCAATCGGGGCCTCCCCCAACTCCTGGGCCATCC | |
| | CAAGATAACGACACCGACTGGGGGTATTGATGGACAAA | |
| | ACCCAGCCTGCTTCCACAAAAACATCCCAATGCCCTCA | |
| | CCCGTAGTCGACCCCTCGATTTGCGGCTCTATATGACCA | |
| | CACCCTCAAACAAACATCCCCCTCTTTCCTCCCTCCCCC | |
| | TGCTGTACAACTACGTACGCCCTAGATACCACAGGCAC | |
| | AATGCGGCTCACTAACAATCAAAACAGAGCCGAGGGA | |
| | ATTAGAAAAAAGTACGGGTAGAAGAGGGATATTCAGA | |
| | GATCAGGGCAAGTCTCCCGAGTCTCTGCTCTCTCCTCTA | |
| | CCTGATAGACCAGGACAAACATGGCCACCTTTACAGAT | |
| | GCAGAGATCGACGAGCTATTTGAGACAAGTGGAACTGT | |
| | CATTGACAACATAATTACAGCCCAGGGTAAACCAGCAG | |
| | AGACTGTTGGAAGGAGTGCAATCCCACAAGGCAAGACC | |
| | AAGGTGCTGAGCGCAGCATGGGAGAAGCATGGGAGCA | |
| | TCCAGCCACCGGCCAGTCAAGACAACCCCGATCGACAG | |
| | GACAGATCTGACAAACAACCATCCACACCCGAGCAAAC | |
| | GACCCCGCATGACAGCCCGCCGGCCACATCCGCCGACC | |
| | AGCCCCCCACCCAGGCCACAGACGAAGCCGTCGACACA | |
| | CAGCTCAGGACCGGAGCAAGCAACTCTCTGCTGTTGAT | |
| | GCTTGACAAGCTCAGCAATAAATCGTCCAATGCTAAAA | |
| | AGGGCCCATGGTCGAGCCCCCAAGAGGGGAATCACCAA | |
| | CGTCCGACTCAACAGCAGGGGAGTCAACCCAGTCGCGG | |
| | AAACAGTCAGGAAAGACCGCAGAACCAAGTCAAGGCC | |
| | GCCCCTGGAAACCAGGGCACAGACGTGAACACAGCATA | |
| | TCATGGACAATGGGAGGAGTCACAACTATCAGCTGGTG | |
| | CAACCCCTCATGCTCTCCGATCAAGGCAGAGCCAAGAC | |
| | AATACCCTTGTATCTGCGGATCATGTCCAGCCACCTGTA | |
| | GACTTTGTGCAAGCGATGATGTCTATGATGGAGGCGAT | |
| | ATCACAGAGAGTAAGTAAGGTTGACTATCAGCTAGATC | |
| | TTGTCTTGAAACAGACATCCTCCATCCCTATGATGCGGT | |
| | CCGAAATCCAACAGCTGAAAACATCTGTTGCAGTCATG | |
| | GAAGCCAACTTGGGAATGATGAAGATTCTGGATCCCGG | |
| | TTGTGCCAACATTTCATCTCTGAGTGATCTACGGGCAGT | |
| | TGCCCGATCTCACCCGGTTTTAGTTTCAGGCCCTGGAGA | |
| | CCCCTCTCCCTATGTGACACAAGGAGGCGAAATGGCAC | |
| | TTAATAAACTTTCGCAACCAGTGCCACATCCATCTGAAT | |
| | TGATTAAACCCGCCACTGCATGCGGGCCTGATATAGGA | |
| | GTGGAAAAGGACACTGTCCGTGCATTGATCATGTCACG | |
| | CCCAATGCACCCGAGTTCTTCAGCCAAGCTCCTAAGCA | |
| | AGTTAGATGCAGCCGGGTCGATCGAGGAAATCAGGAAA | |
| | ATCAAGCGCCTTGCTCTAAATGGCTAATTACTACTGCCA | |
| | CACGTAGCGGGTCCCTGTCCACTCGGCATCACACGGAA | |
| | TCTGCACCGAGTTCCCCCCCGCGGACCCAAGGTCCAAC | |
| | TCTCCAAGCGGCAATCCTCTCTCGCTTCCTCAGCCCCAC | |
| | TGAATGATCGCGTAACCGTAATTAATCTAGCTACATTTA | |
| | AGATTAAGAAAAAATACGGGTAGAATTGGAGTGCCCCA | |
| | ATTGTGCCAAGATGGACTCATCTAGGACAATTGGGCTG | |
| | TACTTTGATTCTGCCCATTCTTCTAGCAACCTGTTAGCA | |
| | TTTCCGATCGTCCTACAAGACACAGGAGATGGGAAGAA | |
| | GCAAATCGCCCCGCAATATAGGATCCAGCGCCTTGACT | |
| | TGTGGACTGATAGTAAGGAGGACTCAGTATTCATCACC | |
| | ACCTATGGATTCATCTTTCAAGTTGGGAATGAAGAAGC | |
| | CACCGTCGGCATGATCGATGATAAACCCAAGCGCGAGT | |
| | TACTTTCCGCTGCGATGCTCTGCCTAGGAAGCGTCCCAA | |
| | ATACCGGAGATCTTATTGAGCTGGCAAGGGCCTGTCTC | |
| | ACTATGATAGTCACATGCAAGAAGAGTGCAACTAATAC | |
| | TGAGAGAATGGTTTTCTCAGTAGTGCAGGCACCCCAAG | |
| | TGCTGCAAAGCTGTAGGGTTGTGGCAAACAAATACTCA | |
| | TCAGTGAATGCAGTCAAGCACGTGAAAGCGCCAGAGAA | |
| | GATTCCCGGGAGTGGAACCCTAGAATACAAGGTGAACT | |
| | TTGTCTCCTTGACTGTGGTACCGAAGAGGGATGTCTACA | |
| | AGATCCCAGCTGCAGTATTGAAGGTTTCTGGCTCGAGTC | |
| | TGTACAATCTTGCGCTCAATGTCACTATTAATGTGGAGG | |
| | TAGACCCGAGGAGTCCTTTGGTTAAATCTCTGTCTAAGT | |
| | CTGACAGCGGATACTATGCTAACCTCTTCTTGCATATTG | |
| | GACTTATGACCACTGTAGATAGGAAGGGGAAGAAAGTG | |
| | ACATTTGACAAGCTGGAAAAGAAAATAAGGAGCCTTGA | |
| | TCTATCTGTCGGGCTCAGTGATGTGCTCGGGCCTTCCGT | |
| | GTTGGTAAAAGCAAGAGGTGCACGGACTAAGCTTTTGG | |
| | CACCTTTCTTCTCTAGCAGTGGGACAGCCTGCTATCCCA | |
| | TAGCAAATGCTTCTCCTCAGGTGGCCAAGATACTCTGG | |
| | AGTCAAACCGCGTGCCTGCGGAGCGTTAAAATCATTAT | |
| | CCAAGCAGGTACCCAACGCGCTGTCGCAGTGACCGCCG | |
| | ACCACGAGGTTACCTCTACTAAGCTGGAGAAGGGGCAC | |
| | ACCCTTGCCAAATACAATCCTTTTAAGAAATAAGCTGC | |
| | GTCTCTGAGATTGCGCTCCGCCCACTCACCCAGATCATC | |
| | ATGACACAAAAAACTAATCTGTCTTGATTATTTACAGTT | |
| | AGTTTACCTGTCTATCAAGTTAGAAAAAACACGGGTAG | |
| | AAGATTCTGGATCCCGGTTGGCGCCCTCCAGGTGCAAG | |
| | ATGGGCTCCAGACCTTCTACCAAGAACCCAGCACCTAT | |
| | GATGCTGACTATCCGGGTTGCGCTGGTACTGAGTTGCAT | |
| | CTGTCCGGCAAACTCCATTGATGGCAGGCCTCTTGCAGC | |
| | TGCAGGAATTGTGGTTACAGGAGACAAAGCAGTCAACA | |
| | TATACACCTCATCCCAGACAGGATCAATCATAGTTAAG | |
| | CTCCTCCCGAATCTGCCCAAGGATAAGGAGGCATGTGC | |
| | GAAAGCCCCCTTGGATGCATACAACAGGACATTGACCA | |
| | CTTTGCTCACCCCCCTTGGTGACTCTATCCGTAGGATAC | |
| | AAGAGTCTGTGACTACATCTGGAGGGCGGAGACAGAGG | |
| | CGCCTTATAGGCGCCATTATTGGCGGTGTGGCTCTTGGG | |
| | GTTGCAACTGCCGCACAAATAACAGCGGCCGCAGCTCT | |
| | GATACAAGCCAAACAAAATGCTGCCAACATCCTCCGAC | |
| | TTAAAGAGAGCATTGCCGCAACCAATGAGGCTGTGCAT | |
| | GAGGTCACTGACGGATTATCGCAACTAGCAGTGGCAGT | |
| | TGGGAAGATGCAGCAGTTTGTTAATGACCAATTTAATA | |
| | AAACAGCTCAGGAATTAGACTGCATCAAAATTGCACAG | |
| | CAAGTTGGTGTAGAGCTCAACCTGTACCTAACCGAATT | |
| | GACTACAGTATTCGGACCACAAATCACTTCACCTGCTTT | |
| | AAACAAGCTGACTATTCAGGCACTTTACAATCTAGCTG | |
| | GTGGAAATATGGATTACTTATTGACTAAGTTAGGTGTA | |
| | GGGAACAATCAACTCAGCTCATTAATCGGTAGCGGCTT | |
| | AATCACCGGTAACCCTATTCTATACGACTCACAGACTCA | |
| | ACTCTTGGGTATACAGGTAACTGCCCCTTCAGTCGGGA | |
| | ACCTAAATAATATGCGTGCCACCTACTTGGAAACCTTAT | |
| | CCGTAAGCACAACCAGGGGATTTGCCTCGGCACTTGTC | |
| | CCAAAAGTGGTGACACAGGTCGGTTCTGTGATAGAAGA | |
| | ACTTGACACCTCATACTGTATAGAAACTGACTTAGATTT | |
| | ATATTGTACAAGAATAGTAACGTTCCCTATGTCCCCTGG | |
| | TATTTATTCCTGCTTGAGCGGCAATACGTCGGCCTGTAT | |
| | GTACTCAAAGACCGAAGGCGCACTTACTACACCATACA | |
| | TGACTATCAAAGGTTCAGTCATCGCCAACTGCAAGATG | |
| | ACAACATGTAGATGTGTAAACCCCCCGGGTATCATATC | |
| | GCAAAACTATGGAGAAGCCGTGTCTCTAATAGATAAAC | |
| | AATCATGCAATGTTTTATCCTTAGGCGGGATAACTTTAA | |
| | GGCTCAGTGGGGAATTCGATGTAACTTATCAGAAGAAT | |
| | ATCTCAATACAAGATTCTCAAGTAATAATAACAGGCAA | |
| | TCTTGATATCTCAACTGAGCTTGGGAATGTCAACAACTC | |
| | GATCAGTAATGCTTTGAATAAGTTAGAGGAAAGCAACA | |
| | GAAAACTAGACAAAGTCAATGTCAAACTGACTAGCACA | |
| | TCTGCTCTCATTACCTATATCGTTTTGACTATCATATCTC | |
| | TTGTTTTTGGTATACTTAGCCTGATTCTAGCATGCTACCT | |
| | AATGTACAAGCAAAAGGCGCAACAAAAGACCTTATTAT | |
| | GGCTTGGGAATAATACTCTAGATCAGATGAGAGCCACT | |
| | ACAAAAATGTGAACACAGATGAGGAACGAAGGTTTCCC | |
| | TAATAGTAATTTGTGTGAAAGTTCTGGTAGTCTGTCAGT | |
| | TCAGAGAGTTAAGAAAAAACTACCGGTTGTAGATGACC | |
| | AAAGGACGATATACGGGTAGAACGGTAAGAGAGGCCG | |
| | CCCCTCAATTGCGAGCCAGGCTTCACAACCTCCGTTCTA | |
| | CCGCTTCACCGACAACAGTCCTCAATCATGGACCGCGC | |
| | CGTTAGCCAAGTTGCGTTAGAGAATGATGAAAGAGAGG | |
| | CAAAAAATACATGGCGCTTGATATTCCGGATTGCAATC | |
| | TTATTCTTAACAGTAGTGACCTTGGCTATATCTGTAGCC | |
| | TCCCTTTTATATAGCATGGGGGCTAGCACACCTAGCGAT | |
| | CTTGTAGGCATACCGACTAGGATTTCCAGGGCAGAAGA | |
| | AAAGATTACATCTACACTTGGTTCCAATCAAGATGTAGT | |
| | AGATAGGATATATAAGCAAGTGGCCCTTGAGTCTCCGT | |
| | TGGCATTGTTAAATACTGAGACCACAATTATGAACGCA | |
| | ATAACATCTCTCTCTTATCAGATTAATGGAGCTGCAAAC | |
| | AACAGTGGGTGGGGGGCACCTATCCATGACCCAGATTA | |
| | TATAGGGGGGATAGGCAAAGAACTCATTGTAGATGATG | |
| | CTAGTGATGTCACATCATTCTATCCCTCTGCATTTCAAG | |
| | AACATCTGAATTTTATCCCGGCGCCTACTACAGGATCAG | |
| | GTTGCACTCGAATACCCTCATTTGACATGAGTGCTACCC | |
| | ATTACTGCTACACCCATAATGTAATATTGTCTGGATGCA | |
| | GAGATCACTCACATTCATATCAGTATTTAGCACTTGGTG | |
| | TGCTCCGGACATCTGCAACAGGGAGGGTATTCTTTTCTA | |
| | CTCTGCGTTCCATCAACCTGGACGACACCCAAAATCGG | |
| | AAGTCTTGCAGTGTGAGTGCAACTCCCCTGGGTTGTGAT | |
| | ATGCTGTGCTCGAAAGTCACGGAGACAGAGGAAGAAG | |
| | ATTATAACTCAGCTGTCCCTACGCGGATGGTACATGGG | |
| | AGGTTAGGGTTCGACGGCCAGTACCACGAAAAGGACCT | |
| | AGATGTCACAACATTATTCGGGGACTGGGTGGCCAACT | |
| | ACCCAGGAGTAGGGGGTGGATCTTTTATTGACAGCCGC | |
| | GTATGGTTCTCAGTCTACGGAGGGTTAAAACCCAATTC | |
| | ACCCAGTGACACTGTACAGGAAGGGAAATATGTGATAT | |
| | ACAAGCGATACAATGACACATGCCCAGATGAGCAAGAC | |
| | TACCAGATTCGAATGGCCAAGTCTTCGTATAAGCCTGG | |
| | ACGGTTTGGTGGGAAACGCATACAGCAGGCTATCTTAT | |
| | CTATCAAGGTGTCAACATCCTTAGGCGAAGACCCGGTA | |
| | CTGACTGTACCGCCCAACACAGTCACACTCATGGGGGC | |
| | CGAAGGCAGAATTCTCACAGTAGGGACATCTCATTTCTT | |
| | GTATCAACGAGGGTCATCATACTTCTCTCCCGCGTTATT | |
| | ATATCCTATGACAGTCAGCAACAAAACAGCCACTCTTC | |
| | ATAGTCCTTATACATTCAATGCCTTCACTCGGCCAGGTA | |
| | GTATCCCTTGCCAGGCTTCAGCAAGATGCCCCAACTCGT | |
| | GTGTTACTGGAGTCTATACAGATCCATATCCCCTAATCT | |
| | TCTATAGAAACCACACCTTGCGAGGGGTATTCGGGACA | |
| | ATGCTTGATGGTGTACAAGCAAGACTTAACCCTGCGTCT | |
| | GCAGTATTCGATAGCACATCCCGCAGTCGCATTACTCG | |
| | AGTGAGTTCAAGCAGTACCAAAGCAGCATACACAACAT | |
| | CAACTTGTTTTAAAGTGGTCAAGACTAATAAGACCTATT | |
| | GTCTCAGCATTGCTGAAATATCTAATACTCTCTTCGGAG | |
| | AATTCAGAATCGTCCCGTTACTAGTTGAGATCCTCAAAG | |
| | ATGACGGGGTTAGAGAAGCCAGGTCTGGCTAGTTGAGT | |
| | CAATTATAAAGGAGTTGGAAAGATGGCATTGTATCACC | |
| | TATCTTCTGCGACATCAAGAATCAAACCGAATGCCGGC | |
| | GCGTGCTCGAATTCCATGTTGCCAGTTGACCACAATCAG | |
| | CCAGTGCTCATGCGATCAGATTAAGCCTTGTCAATAGTC | |
| | TCTTGATTAAGAAAAAATGTAAGTGGCAATGAGATACA | |
| | AGGCAAAACAGCTCATGGTTAACAATACGGGTAGGACA | |
| | TGGCGAGCTCCGGTCCTGAAAGGGCAGAGCATCAGATT | |
| | ATCCTACCAGAGTCACACCTGTCTTCACCATTGGTCAAG | |
| | CACAAACTACTCTATTACTGGAAATTAACTGGGCTACC | |
| | GCTTCCTGATGAATGTGACTTCGACCACCTCATTCTCAG | |
| | CCGACAATGGAAAAAAATACTTGAATCGGCCTCTCCTG | |
| | ATACTGAGAGAATGATAAAACTCGGAAGGGCAGTACAC | |
| | CAAACTCTTAACCACAATTCCAGAATAACCGGAGTGCT | |
| | CCACCCCAGGTGTTTAGAAGAACTGGCTAATATTGAGG | |
| | TCCCAGATTCAACCAACAAATTTCGGAAGATTGAGAAG | |
| | AAGATCCAAATTCACAACACGAGATATGGAGAACTGTT | |
| | CACAAGGCTGTGTACGCATATAGAGAAGAAACTGCTGG | |
| | GGTCATCTTGGTCTAACAATGTCCCCCGGTCAGAGGAG | |
| | TTCAGCAGCATTCGTACGGATCCGGCATTCTGGTTTCAC | |
| | TCAAAATGGTCCACAGCCAAGTTTGCATGGCTCCATAT | |
| | AAAACAGATCCAGAGGCATCTGATGGTGGCAGCTAGGA | |
| | CAAGGTCTGCGGCCAACAAATTGGTGATGCTAACCCAT | |
| | AAGGTAGGCCAAGTCTTTGTCACTCCTGAACTTGTCGTT | |
| | GTGACGCATACGAATGAGAACAAGTTCACATGTCTTAC | |
| | CCAGGAACTTGTATTGATGTATGCAGATATGATGGAGG | |
| | GCAGAGATATGGTCAACATAATATCAACCACGGCGGTG | |
| | CATCTCAGAAGCTTATCAGAGAAAATTGATGACATTTT | |
| | GCGGTTAATAGACGCTCTGGCAAAAGACTTGGGTAATC | |
| | AAGTCTACGATGTTGTATCACTAATGGAGGGATTTGCAT | |
| | ACGGAGCTGTCCAGCTACTCGAGCCGTCAGGTACATTT | |
| | GCAGGAGATTTCTTCGCATTCAACCTGCAGGAGCTTAA | |
| | AGACATTCTAATTGGCCTCCTCCCCAATGATATAGCAGA | |
| | ATCCGTGACTCATGCAATCGCTACTGTATTCTCTGGTTT | |
| | AGAACAGAATCAAGCAGCTGAGATGTTGTGTCTGTTGC | |
| | GTCTGTGGGGTCACCCACTGCTTGAGTCCCGTATTGCAG | |
| | CAAAGGCAGTCAGGAGCCAAATGTGCGCACCGAAAAT | |
| | GGTAGACTTTGATATGATCCTTCAGGTACTGTCTTTCTT | |
| | CAAGGGAACAATCATCAACGGGTACAGAAAGAAGAAT | |
| | GCAGGTGTGTGGCCGCGAGTCAAAGTGGATACAATATA | |
| | TGGGAAGGTCATTGGGCAACTACATGCAGATTCAGCAG | |
| | AGATTTCACACGATATCATGTTGAGAGAGTATAAGAGT | |
| | TTATCTGCACTTGAATTTGAGCCATGTATAGAATATGAC | |
| | CCTGTCACCAACCTGAGCATGTTCCTAAAAGACAAGGC | |
| | AATCGCACACCCCAACGATAATTGGCTTGCCTCGTTTAG | |
| | GCGGAACCTTCTCTCCGAAGACCAGAAGAAACATGTAA | |
| | AAGAAGCAACTTCGACTAATCGCCTCTTGATAGAGTTTT | |
| | TAGAGTCAAATGATTTTGATCCATATAAAGAGATGGAA | |
| | TATCTGACGACCCTTGAGTACCTTAGAGATGACAATGT | |
| | GGCAGTATCATACTCGCTCAAGGAGAAGGAAGTGAAAG | |
| | TTAATGGACGGATCTTCGCTAAGCTGACAAAGAAGTTA | |
| | AGGAACTGTCAGGTGATGGCGGAAGGGATCCTAGCCGA | |
| | TCAGATTGCACCTTTCTTTCAGGGAAATGGAGTCATTCA | |
| | GGATAGCATATCCTTGACCAAGAGTATGCTAGCGATGA | |
| | GTCAACTGTCTTTTAACAGCAATAAGAAACGTATCACT | |
| | GACTGTAAAGAAAGAGTATCTTCAAACCGCAATCATGA | |
| | TCCGAAAAGCAAGAACCGTCGGAGAGTTGCAACCTTCA | |
| | TAACAACTGACCTGCAAAAGTACTGTCTTAATTGGAGA | |
| | TATCAGACAATCAAATTGTTCGCTCATGCCATCAATCAG | |
| | TTGATGGGCCTACCTCACTTCTTCGAATGGATTCACCTA | |
| | AGACTGATGGACACTACGATGTTCGTAGGAGACCCTTT | |
| | CAATCCTCCAAGTGACCCTACTGACTGTGACCTCTCAAG | |
| | AGTCCCTAATGATGACATATATATTGTCAGTGCCAGAG | |
| | GGGGTATCGAAGGATTATGCCAGAAGCTATGGACAATG | |
| | ATCTCAATTGCTGCAATCCAACTTGCTGCAGCTAGATCG | |
| | CATTGTCGTGTTGCCTGTATGGTACAGGGTGATAATCAA | |
| | GTAATAGCAGTAACGAGAGAGGTAAGATCAGACGACTC | |
| | TCCGGAGATGGTGTTGACACAGTTGCATCAAGCCAGTG | |
| | ATAATTTCTTCAAGGAATTAATTCATGTCAATCATTTGA | |
| | TTGGCCATAATTTGAAGGATCGTGAAACCATCAGGTCA | |
| | GACACATTCTTCATATACAGCAAACGAATCTTCAAAGA | |
| | TGGAGCAATCCTCAGTCAAGTCCTCAAAAATTCATCTA | |
| | AATTAGTGCTAGTGTCAGGTGATCTCAGTGAAAACACC | |
| | GTAATGTCCTGTGCCAACATTGCCTCTACTGTAGCACGG | |
| | CTATGCGAGAACGGGCTTCCCAAAGACTTCTGTTACTAT | |
| | TTAAACTATATAATGAGTTGTGTGCAGACATACTTTGAC | |
| | TCTGAGTTCTCCATCACCAACAATTCGCACCCCGATCTT | |
| | AATCAGTCGTGGATTGAGGACATCTCTTTTGTGCACTCA | |
| | TATGTTCTGACTCCTGCCCAATTAGGGGGACTGAGTAAC | |
| | CTTCAATACTCAAGGCTCTACACTAGAAATATCGGTGA | |
| | CCCGGGGACTACTGCTTTTGCAGAGATCAAGCGACTAG | |
| | AAGCAGTGGGATTACTGAGTCCTAACATTATGACTAAT | |
| | ATCTTAACTAGGCCGCCTGGGAATGGAGATTGGGCCAG | |
| | TCTGTGCAACGACCCATACTCTTTCAATTTTGAGACTGT | |
| | TGCAAGCCCAAATATTGTTCTTAAGAAACATACGCAAA | |
| | GAGTCCTATTTGAAACTTGTTCAAATCCCTTATTGTCTG | |
| | GAGTGCACACAGAGGATAATGAGGCAGAAGAGAAGGC | |
| | ATTGGCTGAATTCTTGCTTAATCAAGAGGTGATTCATCC | |
| | CCGCGTTGCGCATGCCATCATGGAGGCAAGCTCTGTAG | |
| | GTAGGAGAAAGCAAATTCAAGGGCTTGTTGACACAACA | |
| | AACACCGTAATTAAGATTGCGCTTACTAGGAGGCCATT | |
| | AGGCATCAAGAGGCTGATGCGGATAGTCAATTATTCTA | |
| | GCATGCATGCAATGCTGTTTAGAGACGATGTTTTTTCCT | |
| | CCAGTAGATCCAACCACCCCTTAGTCTCTTCTAATATGT | |
| | GTTCTCTGACACTGGCAGACTATGCACGGAATAGAAGC | |
| | TGGTCACCTTTGACGGGAGGCAGGAAAATACTGGGTGT | |
| | ATCTAATCCTGATACGATAGAACTCGTAGAGGGTGAGA | |
| | TTCTTAGTGTAAGCGGAGGGTGTACAAGATGTGACAGC | |
| | GGAGATGAACAATTTACTTGGTTCCATCTTCCAAGCAAT | |
| | ATAGAATTGACCGATGACACCAGCAAGAATCCTCCGAT | |
| | GAGGGTACCATATCTCGGGTCAAAGACACAGGAGAGG | |
| | AGAGCTGCCTCACTTGCAAAAATAGCTCATATGTCGCC | |
| | ACATGTAAAGGCTGCCCTAAGGGCATCATCCGTGTTGA | |
| | TCTGGGCTTATGGGGATAATGAAGTAAATTGGACTGCT | |
| | GCTCTTACGATTGCAAAATCTCGGTGTAATGTAAACTTA | |
| | GAGTATCTTCGGTTACTGTCCCCTTTACCCACGGCTGGG | |
| | AATCTTCAACATAGACTAGATGATGGTATAACTCAGAT | |
| | GACATTCACCCCTGCATCTCTCTACAGGGTGTCACCTTA | |
| | CATTCACATATCCAATGATTCTCAAAGGCTGTTCACTGA | |
| | AGAAGGAGTCAAAGAGGGGAATGTGGTTTACCAACAG | |
| | ATCATGCTCTTGGGTTTATCTCTAATCGAATCGATCTTT | |
| | CCAATGACAACAACCAGGACATATGATGAGATCACACT | |
| | GCACCTACATAGTAAATTTAGTTGCTGTATCAGAGAAG | |
| | CACCTGTTGCGGTTCCTTTCGAGCTACTTGGGGGGTAC | |
| | CGGAACTGAGGACAGTGACCTCAAATAAGTTTATGTAT | |
| | GATCCTAGCCCTGTATCGGAGGGAGACTTTGCGAGACT | |
| | TGACTTAGCTATCTTCAAGAGTTATGAGCTTAATCTGGA | |
| | GTCATATCCCACGATAGAGCTAATGAACATTCTTTCAAT | |
| | ATCCAGCGGGAAGTTGATTGGCCAGTCTGTGGTTTCTTA | |
| | TGATGAAGATACCTCCATAAAGAATGACGCCATAATAG | |
| | TGTATGACAATACCCGAAATTGGATCAGTGAAGCTCAG | |
| | AATTCAGATGTGGTCCGCCTATTTGAATATGCAGCACTT | |
| | GAAGTGCTCCTCGACTGTTCTTACCAACTCTATTACCTG | |
| | AGAGTAAGAGGCCTGGACAATATTGTCTTATATATGGG | |
| | TGATTTATACAAGAATATGCCAGGAATTCTACTTTCCAA | |
| | CATTGCAGCTACAATATCTCATCCCGTCATTCATTCAAG | |
| | GTTACATGCAGTGGGCCTGGTCAACCATGACGGATCAC | |
| | ACCAACTTGCAGATACGGATTTTATCGAAATGTCTGCA | |
| | AAACTATTAGTATCTTGCACCCGACGTGTGATCTCCGGC | |
| | TTATATTCAGGAAATAAGTATGATCTGCTGTTCCCATCT | |
| | GTCTTAGATGATAACCTGAATGAGAAGATGCTTCAGCT | |
| | GATATCCCGGTTATGCTGTCTGTACACGGTACTCTTTGC | |
| | TACAACAAGAGAAATCCCGAAAATAAGAGGCTTAACTG | |
| | CAGAAGAGAAATGTTCAATACTCACTGAGTATTTACTG | |
| | TCGGATGCTGTGAAACCATTACTTAGCCCCGATCAAGT | |
| | GAGCTCTATCATGTCTCCTAACATAATTACATTCCCAGC | |
| | TAATCTGTACTACATGTCTCGGAAGAGCCTCAATTTGAT | |
| | CAGGGAAAGGGAGGACAGGGATACTATCCTGGCGTTGT | |
| | TGTTCCCCCAAGAGCCATTATTAGAGTTCCCTTCTGTGC | |
| | AAGATATTGGTGCTCGAGTGAAAGATCCATTCACCCGA | |
| | CAACCTGCGGCATTTTTGCAAGAGTTAGATTTGAGTGCT | |
| | CCAGCAAGGTATGACGCATTCACACTTAGTCAGATTCA | |
| | TCCTGAACTCACATCTCCAAATCCGGAGGAAGACTACT | |
| | TAGTACGATACTTGTTCAGAGGGATAGGGACTGCATCT | |
| | TCCTCTTGGTATAAGGCATCTCATCTCCTTTCTGTACCC | |
| | GAGGTAAGATGTGCAAGACACGGGAACTCCTTATACTT | |
| | AGCTGAAGGGAGCGGAGCCATCATGAGTCTTCTCGAAC | |
| | TGCATGTACCACATGAAACTATCTATTACAATACGCTCT | |
| | TTTCAAATGAGATGAACCCCCCGCAACGACATTTCGGG | |
| | CCGACCCCAACTCAGTTTTTGAATTCGGTTGTTTATAGG | |
| | AATCTACAGGCGGAGGTAACATGCAAAGATGGATTTGT | |
| | CCAAGAGTTCCGTCCATTATGGAGAGAAAATACAGAGG | |
| | AAAGCGACCTGACCTCAGATAAAGTAGTGGGGTATATT | |
| | ACATCTGCAGTGCCCTACAGATCTGTATCATTGCTGCAT | |
| | TGTGACATTGAAATTCCTCCAGGGTCCAATCAAAGCTTA | |
| | CTAGATCAACTAGCTATCAATTTATCTCTGATTGCCATG | |
| | CATTCTGTAAGGGAGGGGGGGGTAGTAATCATCAAAGT | |
| | GTTGTATGCAATGGGATACTACTTTCATCTACTCATGAA | |
| | CTTGTTTGCTCCGTGTTCCACAAAAGGATATATTCTCTC | |
| | TAATGGTTATGCATGTCGAGGAGATATGGAGTGTTACC | |
| | TGGTATTTGTCATGGGTTACCTGGGCGGGCCTACATTTG | |
| | TACATGAGGTGGTGAGGATGGCGAAAACTCTGGTGCAG | |
| | CGGCACGGTACGCTTTTGTCTAAATCAGATGAGATCAC | |
| | ACTGACCAGGTTATTCACCTCACAGCGGCAGCGTGTGA | |
| | CAGACATCCTATCCAGTCCTTTACCAAGATTAATAAAGT | |
| | ACTTGAGGAAGAATATTGACACTGCGCTGATTGAAGCC | |
| | GGGGGACAGCCCGTCCGTCCATTCTGTGCGGAGAGTCT | |
| | GGTGAGCACGCTAGCGAACATAACTCAGATAACCCAGA | |
| | TCATCGCTAGTCACATTGACACAGTTATCCGGTCTGTGA | |
| | TATATATGGAAGCTGAGGGTGATCTCGCTGACACAGTA | |
| | TTTCTATTTACCCCTTACAATCTCTCTACTGACGGGAAA | |
| | AAGAGGACATCACTTAAACAGTGCACGAGACAGATCCT | |
| | AGAGGTTACAATACTAGGTCTTAGAGTCGAAAATCTCA | |
| | ATAAAATAGGCGATATAATCAGCCTAGTGCTTAAAGGC | |
| | ATGATCTCCATGGAGGACCTTATCCCACTAAGGACATA | |
| | CTTGAAGCATAGTACCTGCCCTAAATATTTGAAGGCTGT | |
| | CCTAGGTATTACCAAACTCAAAGAAATGTTTACAGACA | |
| | CTTCTGTACTGTACTTGACTCGTGCTCAACAAAAATTCT | |
| | ACATGAAAACTATAGGCAATGCAGTCAAAGGATATTAC | |
| | AGTAACTGTGACTCTTAACGAAAATCACATATTAATAG | |
| | GCTCCTTTTTTGGCCAATTGTATTCTTGTTGATTTAATCA | |
| | TATTATGTTAGAAAAAAGTTGAACCCTGACTCCTTAGG | |
| | ACTCGAATTCGAACTCAAATAAATGTCTTAAAAAAAGG | |
| | TTGCGCACAATTATTCTTGAGTGTAGTCTCGTCATTCAC | |
| | CAAATCTTTGTTTGGT | |

TABLE 2
NDV LaSota F protein

| | | SEQ ID |
|---|---|---|
| Description | Sequence | NO: |
| Amino acid | MGSRPSTKNPAPMTLTIRVALVLSCICPANSIDGRPLAAAG | SEQ ID |
| sequence of F | IVVTGDKAVNIYTSSQTGSIIVKLLPNLPKDKEACAKAPLD | NO: 4 |
| protein of NDV | AYNRTLTTLLTPLGDSIRRIQESVTTSGGGRQGRLIGAIIG | |
| strain LaSota | GVALGVATAAQITAAAALIQAKQNAANILRLKESIAATNEA | |
| (transmembrane | VHEVTDGLSQLAVAVGKMQQFVNDQFNKTAQELDCIKIAQQ | |
| domain is | VGVELNLYLTELTTVFGPQITSPALNKLTIQALYNLAGGNM | |
| underlined and | DYLLTKLGVGNNQLSSLIGSGLITGNPILYDSQTQLLGIQV | |
| cytoplasmic | TLPSVGNLNNMRATYLETLSVSTTRGFASALVPKVVTQVGS | |
| domain is in | VIEELDTSYCIETDLDLYCTRIVTFPMSPGIYSCLSGNTSA | |
| bold) | CMYSKTEGALTTPYMTIKGSVIANCKMTTCRCVNPPGIISQ | |
| | NYGEAVSLIDKQSCNVLSLGGITLRLSGEFDVTYQKNISIQ | |
| | DSQVIITGNLDISTELGNVNNSISNALNKLEESNRKLDKVN | |
| | VKLTSTSALITYIVLTIISLVFGILSLILACYLMYKQKAQQ | |
| | KTLLWLGNNTLDQMRATTKM | |
| Amino acid | LITYIVLTIISLVFGILSLILACYLMYKQKAQQKTLLWLGN | SEQ ID |
| sequence of | NTLDQMRATTKM | NO: 5 |
| transmembrane | | |
| and cytoplasmic | | |
| domains of F | | |
| protein of NDV | | |
| strain LaSota | | |

TABLE 3
LASV GP NUCLEOTIDE AND PROTEIN SEQUENCES

| | | SEQ ID |
|---|---|---|
| Description | Sequence | NO: |
| Nucleotide | ATGGGCCAGATCATCACCTTCTTCCAGGAGGTGCCCCA | SEQ ID |
| sequence of the | CGTGATCGAGGAGGTGATGAACATCGTGCTGATCGCCC | NO: 6 |
| codon optimized | TGAGCCTGCTGGCCATCCTGAAGGGCGTGTACAACGTG | |
| LASV GP (strain | GCCACCTGCGGCCTGTTCGGCCTGATCAGCTTCCTGCT | |
| LASV/H. sapiens- | GCTGTGCGGCAGAAGCTGCAGCGTGACCTACAAGGGCG | |
| wt/NGA/2018/IRR | TGTACGAGCTGCAGACCCTGGAGCTGGACATGGCCAAC | |
| 013) | CTGAACATGACCATGCCCCTGAGCTGCACCAAGAACAG | |
| | CAGCCACCACTACATCATGGTGGGCAACGAGACCGGCC | |
| | TGGAGCTGACCCTGACCAACACCAGCATCATCAACCAC | |
| | AAGTTCTGCAACCTGAGCGACGCCCACAAGAGAAACCT | |
| | GTACAACCACGCCCTGATGAGCATCATCAGCACCTTCC | |
| | ACCTGAGCATCCCCAACTTCAACCAGTACGAGGCCATG | |
| | AGCTGCGACTTCAACGGCGGCAAGATCAGCGTGCAGTA | |
| | CAACCTGAGCCACGCCTACGCCGTGGACGCCGCCAACC | |
| | ACTGCGGCACCATCGCCAACGGCGTGCTGCAGACCTTC | |
| | ATGAGAATGGCCTGGGGCGGCAGCTACATCGCCCTGGA | |
| | CAGCGGCAAGGGCAACTGGGACTGCATCATGACCAGCT | |
| | ACCAGTACCTGATCATCCAGAACACCACCTGGGAGGAC | |
| | CACTGCCAGTTCAGCAGACCCAGCCCCATCGGCTACCT | |
| | GGGCCTGCTGAGCCAGAGAACCAGAGACATCTACATCA | |
| | GCAGAAGACTGCTGGGCACCTTCACCTGGACCCTGAGC | |
| | GACAGCGAGGGCAACGAGGCCCCCGGCGGCTACTGCCT | |
| | GACCAGATGGATGCTGATCGAGGCCGAGCTGAAGTGCT | |
| | TCGGCAACACCGCCATCGCCAAGTGCAACGAGAAGCAC | |
| | GACGAGGAGTTCTGCGACATGCTGAGACTGTTCGACTT | |
| | CAACAAGCAGGCCATCGAGAGACTGAAGGCCGAGGCCC | |
| | AGATGAGCATCCAGCTGATCAACAAGGCCGTGAACGCC | |
| | CTGATCAACGACCAGCTGATCATGAAGAACCACCTGAG | |
| | AGACATCATGGGCATCCCCTACTGCAACTACAGCAAGT | |
| | ACTGGTACCTGAACCACACCGTGACCGGCAGAACCAGC | |
| | CTGCCCAGATGCTGGCTGGTGAGCAACGGCAGCTACCT | |
| | GAACGAGACCCACTTCAGCGACGACATCGAGCAGCAGG | |
| | CCGACAACATGATCACCGAGCTGCTGCAGAAGGAGTAC | |
| | ATGGACAGACAGGGCAAGACCCCCCTGGGCCTGGTGGA | |
| | CCTGTTCGTGTTCAGCACCAGCTTCTACCTGATCAGCA | |
| | TCTTCCTGCACCTGGTGAAGATCCCCACCCACAGACAC | |
| | ATCGTGGGCAGACCCTGCCCCAAGCCCCACAGACTGAA | |
| | CCACATGGGCATCTGCAGCTGCGGCCTGTACAAGCACC | |
| | CCGGCGTGCCCGTGAAGTGGAAGAGATGA | |
| Nucleotide | ATGGGCCAGATCATCACCTTCTTCCAGGAGGTGCCCCA | SEQ ID |
| sequence of the | CGTGATCGAGGAGGTGATGAACATCGTGCTGATCGCCC | NO: 7 |
| LASV GP | TGAGCCTGCTGGCCATCCTGAAGGGCGTGTACAACGTG | |
| chimera. (Codon | GCCACCTGCGGCCTGTTCGGCCTGATCAGCTTCCTGCT | |
| optimized | GCTGTGCGGCAGAAGCTGCAGCGTGACCTACAAGGGCG | |
| ectodomain of the | TGTACGAGCTGCAGACCCTGGAGCTGGACATGGCCAAC | |
| GP from strain | CTGAACATGACCATGCCCCTGAGCTGCACCAAGAACAG | |
| LASV/H. sapiens- | CAGCCACCACTACATCATGGTGGGCAACGAGACCGGCC | |
| wt/NGA/2018/IRR | TGGAGCTGACCCTGACCAACACCAGCATCATCAACCAC | |
| 013) fused to | AAGTTCTGCAACCTGAGCGACGCCCACAAGAGAAACCT | |
| the transmembrane | GTACAACCACGCCCTGATGAGCATCATCAGCACCTTCC | |
| and cytoplasmic | ACCTGAGCATCCCCAACTTCAACCAGTACGAGGCCATG | |
| regions of the F | AGCTGCGACTTCAACGGCGGCAAGATCAGCGTGCAGTA | |
| protein of NDV). | CAACCTGAGCCACGCCTACGCCGTGGACGCCGCCAACC | |
| Transmembrane | ACTGCGGCACCATCGCCAACGGCGTGCTGCAGACCTTC | |
| and cytoplasmic | ATGAGAATGGCCTGGGGCGGCAGCTACATCGCCCTGGA | |
| regions of the F | CAGCGGCAAGGGCAACTGGGACTGCATCATGACCAGCT | |
| protein of NDV | ACCAGTACCTGATCATCCAGAACACCACCTGGGAGGAC | |
| are underlined. | CACTGCCAGTTCAGCAGACCCAGCCCCATCGGCTACCT | |
| | GGGCCTGCTGAGCCAGAGAACCAGAGACATCTACATCA | |
| | GCAGAAGACTGCTGGGCACCTTCACCTGGACCCTGAGC | |
| | GACAGCGAGGGCAACGAGGCCCCCGGCGGCTACTGCCT | |
| | GACCAGATGGATGCTGATCGAGGCCGAGCTGAAGTGCT | |
| | TCGGCAACACCGCCATCGCCAAGTGCAACGAGAAGCAC | |
| | GACGAGGAGTTCTGCGACATGCTGAGACTGTTCGACTT | |
| | CAACAAGCAGGCCATCGAGAGACTGAAGGCCGAGGCCC | |
| | AGATGAGCATCCAGCTGATCAACAAGGCCGTGAACGCC | |
| | CTGATCAACGACCAGCTGATCATGAAGAACCACCTGAG | |
| | AGACATCATGGGCATCCCCTACTGCAACTACAGCAAGT | |
| | ACTGGTACCTGAACCACACCGTGACCGGCAGAACCAGC | |
| | CTGCCCAGATGCTGGCTGGTGAGCAACGGCAGCTACCT | |
| | GAACGAGACCCACTTCAGCGACGACATCGAGCAGCAGG | |
| | CCGACAACATGATCACCGAGCTGCTGCAGAAGGAGTAC | |
| | ATGGACAGACAGGGCAAGACCCCCGTTAACCTCATTAC | |
| | CTATATCGTTTTGACTATCATATCTCTTGTTTTTGGTA | |
| | TACTTAGCCTGATTCTAGCATGCTACCTAATGTACAAG | |
| | CAAAAGGCGCAACAAAAGACCTTATTATGGCTTGGGAA | |
| | TAATACCCTAGATCAGATGAGAGCCACTACAAAAATGT | |
| | GA | |
| Nucleotide | ATGGGCCAGATCATCACCTTCTTCCAGGAGGTGCCCCA | SEQ ID |
| sequence of the | CGTGATCGAGGAGGTGATGAACATCGTGCTGATCGCCC | NO: 8 |
| codon optimized | TGAGCCTGCTGGCCATCCTGAAGGGCGTGTACAACGTG | |
| LASV GP 1 Pro | GCCACCTGCGGCCTGTTCGGCCTGATCAGCTTCCTGCT | |
| (Codon optimized | GCTGTGCGGCAGAAGCTGCAGCGTGACCTACAAGGGCG | |
| full length | TGTACGAGCTGCAGACCCTGGAGCTGGACATGGCCAAC | |
| sequence of | CTGAACATGACCATGCCCCTGAGCTGCACCAAGAACAG | |
| LASV strain | CAGCCACCACTACATCATGGTGGGCAACGAGACCGGCC | |
| LASV/H. sapiens- | TGGAGCTGACCCTGACCAACACCAGCATCATCAACCAC | |
| wt/NGA/2018/IRR | AAGTTCTGCAACCTGAGCGACGCCCACAAGAGAAACCT | |
| 013 glycoprotein | GTACAACCACGCCCTGATGAGCATCATCAGCACCTTCC | |
| with 3 amino acid | ACCTGAGCATCCCCAACTTCAACCAGTACGAGGCCATG | |
| mutations to | AGCTGCGACTTCAACGGCGGCAAGATCAGCGTGCAGTA | |
| stabilize the | CAACCTGAGCCACGCCTACGCCGTGGACGCCGCCAACC | |
| pre-fusion | ACTGCGGCACCATCGCCAACGGCGTGCTGCAGACCTTC | |
| conformation). | ATGAGAATGGCCTGGGGCGGCAGCTACATCGCCCTGGA | |
| Mutated codons | CAGCGGCTGCGGCAACTGGGACTGCATCATGACCAGCT | |
| are underlined | ACCAGTACCTGATCATCCAGAACACCACCTGGGAGGAC | |
| | CACTGCCAGTTCAGCAGACCCAGCCCCATCGGCTACCT | |
| | GGGCCTGCTGAGCCAGAGAACCAGAGACATCTACATCA | |
| | GCAGAAGACTGCTGGGCACCTTCACCTGGACCCTGAGC | |
| | GACAGCGAGGGCAACGAGGCCCCCGGCGGCTACTGCCT | |
| | GACCAGATGGATGCTGATCGAGGCCGAGCTGAAGTGCT | |
| | TCGGCAACACCGCCATCGCCAAGTGCAACGAGAAGCAC | |
| | GACGAGGAGTTCTGCGACATGCTGAGACTGTTCGACTT | |
| | CAACAAGCAGGCCATCGAGAGACTGAAGGCCCCGGCCC | |
| | AGATGAGCATCCAGCTGATCAACAAGGCCGTGAACGCC | |
| | CTGATCAACGACCAGCTGATCATGAAGAACCACCTGAG | |
| | AGACATCATGTGTATCCCCTACTGCAACTACAGCAAGT | |
| | ACTGGTACCTGAACCACACCGTGACCGGCAGAACCAGC | |
| | CTGCCCAGATGCTGGCTGGTGAGCAACGGCAGCTACCT | |
| | GAACGAGACCCACTTCAGCGACGACATCGAGCAGCAGG | |
| | CCGACAACATGATCACCGAGCTGCTGCAGAAGGAGTAC | |
| | ATGGACAGACAGGGCAAGACCCCCCTGGGCCTGGTGGA | |
| | CCTGTTCGTGTTCAGCACCAGCTTCTACCTGATCAGCA | |
| | TCTTCCTGCACCTGGTGAAGATCCCCACCCACAGACAC | |
| | ATCGTGGGCAGACCCTGCCCCAAGCCCCACAGACTGAA | |
| | CCACATGGGCATCTGCAGCTGCGGCCTGTACAAGCACC | |
| | CCGGCGTGCCCGTGAAGTGGAAGAGATGA | |
| Nucleotide | ATGGGCCAGATCATCACCTTCTTCCAGGAGGTGCCCCA | SEQ ID |
| sequence of the | CGTGATCGAGGAGGTGATGAACATCGTGCTGATCGCCC | NO: 9 |
| LASV GP 1 Pro | TGAGCCTGCTGGCCATCCTGAAGGGCGTGTACAACGTG | |
| chimera (Codon | GCCACCTGCGGCCTGTTCGGCCTGATCAGCTTCCTGCT | |
| optimized | GCTGTGCGGCAGAAGCTGCAGCGTGACCTACAAGGGCG | |
| ectodomain of | TGTACGAGCTGCAGACCCTGGAGCTGGACATGGCCAAC | |
| LASV strain | CTGAACATGACCATGCCCCTGAGCTGCACCAAGAACAG | |
| LASV/H. sapiens- | CAGCCACCACTACATCATGGTGGGCAACGAGACCGGCC | |
| wt/NGA/2018/IRR | TGGAGCTGACCCTGACCAACACCAGCATCATCAACCAC | |
| 013 glycoprotein | AAGTTCTGCAACCTGAGCGACGCCCACAAGAGAAACCT | |
| with 3 amino acid | GTACAACCACGCCCTGATGAGCATCATCAGCACCTTCC | |
| mutations to | ACCTGAGCATCCCCAACTTCAACCAGTACGAGGCCATG | |
| stabilize the | AGCTGCGACTTCAACGGCGGCAAGATCAGCGTGCAGTA | |
| pre-fusion | CAACCTGAGCCACGCCTACGCCGTGGACGCCGCCAACC | |
| conformation | ACTGCGGCACCATCGCCAACGGCGTGCTGCAGACCTTC | |
| fused to the | ATGAGAATGGCCTGGGGCGGCAGCTACATCGCCCTGGA | |
| transmembrane | CAGCGGCTGCGGCAACTGGGACTGCATCATGACCAGCT | |
| and cytoplasmic | ACCAGTACCTGATCATCCAGAACACCACCTGGGAGGAC | |
| regions of the F | CACTGCCAGTTCAGCAGACCCAGCCCCATCGGCTACCT | |
| protein of NDV). | GGGCCTGCTGAGCCAGAGAACCAGAGACATCTACATCA | |
| Mutated codons | GCAGAAGACTGCTGGGCACCTTCACCTGGACCCTGAGC | |
| underlined. | GACAGCGAGGGCAACGAGGCCCCCGGCGGCTACTGCCT | |
| Transmembrane | GACCAGATGGATGCTGATCGAGGCCGAGCTGAAGTGCT | |
| and cytoplasmic | TCGGCAACACCGCCATCGCCAAGTGCAACGAGAAGCAC | |
| regions of the F | GACGAGGAGTTCTGCGACATGCTGAGACTGTTCGACTT | |
| protein of NDV | CAACAAGCAGGCCATCGAGAGACTGAAGGCCCCGGCCC | |
| are underlined. | AGATGAGCATCCAGCTGATCAACAAGGCCGTGAACGCC | |
| | CTGATCAACGACCAGCTGATCATGAAGAACCACCTGAG | |
| | AGACATCATGTGTATCCCCTACTGCAACTACAGCAAGT | |
| | ACTGGTACCTGAACCACACCGTGACCGGCAGAACCAGC | |
| | CTGCCCAGATGCTGGCTGGTGAGCAACGGCAGCTACCT | |
| | GAACGAGACCCACTTCAGCGACGACATCGAGCAGCAGG | |
| | CCGACAACATGATCACCGAGCTGCTGCAGAAGGAGTAC | |
| | ATGGACAGACAGGGCAAGACCCCCGTTAACCTCATTAC | |
| | CTATATCGTTTTGACTATCATATCTCTTGTTTTTGGTA | |
| | TACTTAGCCTGATTCTAGCATGCTACCTAATGTACAAG | |
| | CAAAAGGCGCAACAAAAGACCTTATTATGGCTTGGGAA | |
| | TAATACCCTAGATCAGATGAGAGCCACTACAAAAATGT | |
| | GA | |
| Amino acid | MGQIITFFQEVPHVIEEVMNIVLIALSLLAILKGVYNV | SEQ ID |
| sequence of | ATCGLFGLISFLLLCGRSCSVTYKGVYELQTLELDMAN | NO: 10 |
| LASV GP (strain | LNMTMPLSCTKNSSHHYIMVGNETGLELTLTNTSIINH | |
| LASV/H. sapiens- | KFCNLSDAHKRNLYNHALMSIISTFHLSIPNFNQYEAM | |
| wt/NGA/2018/IRR | SCDENGGKISVQYNLSHAYAVDAANHCGTIANGVLQTF | |
| 013. The signal | MRMAWGGSYIALDSGKGNWDCIMTSYQYLIIQNTTWED | |
| sequence is | HCQFSRPSPIGYLGLLSQRTRDIYISRRLLGTFTWTLS | |
| in bold. | DSEGNEAPGGYCLTRWMLIEAELKCFGNTAIAKCNEKH | |
| | DEEFCDMLRLFDFNKQAIERLKAEAQMSIQLINKAVNA | |
| | LINDQLIMKNHLRDIMGIPYCNYSKYWYLNHTVTGRTS | |
| | LPRCWLVSNGSYLNETHFSDDIEQQADNMITELLQKEY | |
| | MDRQGKTPLGLVDLFVFSTSFYLISIFLHLVKIPTHRH | |
| | IVGRPCPKPHRLNHMGICSCGLYKHPGVPVKWKR | |
| Amino acid | MGQIITFFQEVPHVIEEVMNIVLIALSLLAILKGVYNV | SEQ ID |
| sequence of | ATCGLFGLISFLLLCGRSCSVTYKGVYELQTLELDMAN | NO: 11 |
| LASV GP chimera | LNMTMPLSCTKNSSHHYIMVGNETGLELTLTNTSIINH | |
| (ectodomain of | KFCNLSDAHKRNLYNHALMSIISTFHLSIPNFNQYEAM | |
| the GP from | SCDENGGKISVQYNLSHAYAVDAANHCGTIANGVLQTF | |
| strain LASV/ | MRMAWGGSYIALDSGKGNWDCIMTSYQYLIIQNTTWED | |
| H. sapiens- | HCQFSRPSPIGYLGLLSQRTRDIYISRRLLGTFTWTLS | |
| wt/NGA/2018/IRR | DSEGNEAPGGYCLTRWMLIEAELKCFGNTAIAKCNEKH | |
| 013 fused to | DEEFCDMLRLFDFNKQAIERLKAEAQMSIQLINKAVNA | |
| the transmembrane | LINDQLIMKNHLRDIMGIPYCNYSKYWYLNHTVTGRTS | |
| and cytoplasmic | LPRCWLVSNGSYLNETHFSDDIEQQADNMITELLQKEY | |
| regions of the F | MDRQGKTPVNLITYIVLTIISLVFGILSLILACYLMYK | |
| protein of NDV). | QKAQQKTLLWLGNNTLDQMRATTKM | |
| Transmembrane | | |
| and cytoplasmic | | |
| regions of the F | | |
| protein of NDV | | |
| are underlined. | | |
| The signal | | |
| sequence is in | | |
| bold. | | |
| Amino acid | MGQIITFFQEVPHVIEEVMNIVLIALSLLAILKGVYNV | SEQ ID |
| sequence of | ATCGLFGLISFLLLCGRSCSVTYKGVYELQTLELDMAN | NO: 12 |
| LASV GP 1 Pro | LNMTMPLSCTKNSSHHYIMVGNETGLELTLTNTSIINH | |
| (full length | KFCNLSDAHKRNLYNHALMSIISTFHLSIPNFNQYEAM | |
| sequence of | SCDENGGKISVQYNLSHAYAVDAANHCGTIANGVLQTF | |
| LASV strain | MRMAWGGSYIALDSGCGNWDCIMTSYQYLIIQNTTWED | |
| LASV/H. sapiens- | HCQFSRPSPIGYLGLLSQRTRDIYISRRLLGTFTWTLS | |
| wt/NGA/2018/IRR | DSEGNEAPGGYCLTRWMLIEAELKCFGNTAIAKCNEKH | |
| 013 glycoprotein | DEEFCDMLRLFDENKQAIERLKAPAQMSIQLINKAVNA | |
| with 3 amino acid | LINDQLIMKNHLRDIMCIPYCNYSKYWYLNHTVTGRTS | |
| mutations to | LPRCWLVSNGSYLNETHFSDDIEQQADNMITELLQKEY | |
| stabilize the | MDRQGKTPLGLVDLFVFSTSFYLISIFLHLVKIPTHRH | |
| pre-fusion | IVGRPCPKPHRLNHMGICSCGLYKHPGVPVKWKR | |
| conformation). | | |
| Mutated amino | | |
| acids are | | |
| underlined The | | |
| signal sequence | | |
| is in bold. | | |
| Amino acid | MGQIITFFQEVPHVIEEVMNIVLIALSLLAILKGVYNV | SEQ ID |
| sequence of | ATCGLFGLISFLLLCGRSCSVTYKGVYELQTLELDMAN | NO: 13 |
| LASV GP 1 Pro | LNMTMPLSCTKNSSHHYIMVGNETGLELTLTNTSIINH | |
| chimera | KFCNLSDAHKRNLYNHALMSIISTFHLSIPNFNQYEAM | |
| (ectodomain of | SCDENGGKISVQYNLSHAYAVDAANHCGTIANGVLQTF | |
| LASV strain | MRMAWGGSYIALDSGCGNWDCIMTSYQYLIIQNTTWED | |
| LASV/H. sapiens- | HCQFSRPSPIGYLGLLSQRTRDIYISRRLLGTFTWTLS | |
| wt/NGA/2018/IRR | DSEGNEAPGGYCLTRWMLIEAELKCFGNTAIAKCNEKH | |
| 013 glycoprotein | DEEFCDMLRLFDENKQAIERLKAPAQMSIQLINKAVNA | |
| with 3 amino acid | LINDQLIMKNHLRDIMCIPYCNYSKYWYLNHTVTGRTS | |
| mutations to | LPRCWLVSNGSYLNETHFSDDIEQQADNMITELLQKEY | |
| stabilize the | MDRQGKTPVNLITYIVLTIISLVFGILSLILACYLMYK | |
| pre-fusion | QKAQQKTLLWLGNNTLDQMRATTKM | |
| conformation | | |
| fused to the | | |
| transmembrane | | |
| and cytoplasmic | | |
| regions of the F | | |
| protein of NDV). | | |
| Mutated amino | | |
| acids underlined. | | |
| Transmembrane | | |
| and cytoplasmic | | |
| regions of the F | | |
| protein of NDV | | |
| are underlined. | | |
| The signal | | |
| sequence is in | | |
| bold. | | |
| Nucleotide | ATGGGCCAGATCATCACCTTCTTCCAGGAGGTGCCCCA | SEQ ID |
| sequence of the | CGTGATCGAGGAGGTGATGAACATCGTGCTGATCGCCC | NO: 33 |
| ectodomain of | TGAGCCTGCTGGCCATCCTGAAGGGCGTGTACAACGTG | |
| LASV GP | GCCACCTGCGGCCTGTTCGGCCTGATCAGCTTCCTGCT | |
| chimera. (Codon | GCTGTGCGGCAGAAGCTGCAGCGTGACCTACAAGGGCG | |
| optimized | TGTACGAGCTGCAGACCCTGGAGCTGGACATGGCCAAC | |
| ectodomain of the | CTGAACATGACCATGCCCCTGAGCTGCACCAAGAACAG | |
| GP from strain | CAGCCACCACTACATCATGGTGGGCAACGAGACCGGCC | |
| LASV/H. sapiens- | TGGAGCTGACCCTGACCAACACCAGCATCATCAACCAC | |
| wt/NGA/2018/IRR | AAGTTCTGCAACCTGAGCGACGCCCACAAGAGAAACCT | |
| 013) | GTACAACCACGCCCTGATGAGCATCATCAGCACCTTCC | |
| | ACCTGAGCATCCCCAACTTCAACCAGTACGAGGCCATG | |
| | AGCTGCGACTTCAACGGCGGCAAGATCAGCGTGCAGTA | |
| | CAACCTGAGCCACGCCTACGCCGTGGACGCCGCCAACC | |
| | ACTGCGGCACCATCGCCAACGGCGTGCTGCAGACCTTC | |
| | ATGAGAATGGCCTGGGGCGGCAGCTACATCGCCCTGGA | |
| | CAGCGGCAAGGGCAACTGGGACTGCATCATGACCAGCT | |
| | ACCAGTACCTGATCATCCAGAACACCACCTGGGAGGAC | |
| | CACTGCCAGTTCAGCAGACCCAGCCCCATCGGCTACCT | |
| | GGGCCTGCTGAGCCAGAGAACCAGAGACATCTACATCA | |
| | GCAGAAGACTGCTGGGCACCTTCACCTGGACCCTGAGC | |
| | GACAGCGAGGGCAACGAGGCCCCCGGCGGCTACTGCCT | |
| | GACCAGATGGATGCTGATCGAGGCCGAGCTGAAGTGCT | |
| | TCGGCAACACCGCCATCGCCAAGTGCAACGAGAAGCAC | |
| | GACGAGGAGTTCTGCGACATGCTGAGACTGTTCGACTT | |
| | CAACAAGCAGGCCATCGAGAGACTGAAGGCCGAGGCCC | |
| | AGATGAGCATCCAGCTGATCAACAAGGCCGTGAACGCC | |
| | CTGATCAACGACCAGCTGATCATGAAGAACCACCTGAG | |
| | AGACATCATGGGCATCCCCTACTGCAACTACAGCAAGT | |
| | ACTGGTACCTGAACCACACCGTGACCGGCAGAACCAGC | |
| | CTGCCCAGATGCTGGCTGGTGAGCAACGGCAGCTACCT | |
| | GAACGAGACCCACTTCAGCGACGACATCGAGCAGCAGG | |
| | CCGACAACATGATCACCGAGCTGCTGCAGAAGGAGTAC | |
| | ATGGACAGACAGGGCAAGACCCCCGTTAAC | |
| Nucleotide | ATGGGCCAGATCATCACCTTCTTCCAGGAGGTGCCCCA | SEQ ID |
| sequence of the | CGTGATCGAGGAGGTGATGAACATCGTGCTGATCGCCC | NO: 34 |
| ectodomain of | TGAGCCTGCTGGCCATCCTGAAGGGCGTGTACAACGTG | |
| LASV GP 1 Pro | GCCACCTGCGGCCTGTTCGGCCTGATCAGCTTCCTGCT | |
| chimera (Codon | GCTGTGCGGCAGAAGCTGCAGCGTGACCTACAAGGGCG | |
| optimized | TGTACGAGCTGCAGACCCTGGAGCTGGACATGGCCAAC | |
| ectodomain of | CTGAACATGACCATGCCCCTGAGCTGCACCAAGAACAG | |
| LASV strain | CAGCCACCACTACATCATGGTGGGCAACGAGACCGGCC | |
| LASV/H. sapiens- | TGGAGCTGACCCTGACCAACACCAGCATCATCAACCAC | |
| wt/NGA/2018/IRR | AAGTTCTGCAACCTGAGCGACGCCCACAAGAGAAACCT | |
| 013 glycoprotein | GTACAACCACGCCCTGATGAGCATCATCAGCACCTTCC | |
| with 3 amino acid | ACCTGAGCATCCCCAACTTCAACCAGTACGAGGCCATG | |
| mutations to | AGCTGCGACTTCAACGGCGGCAAGATCAGCGTGCAGTA | |
| stabilize the | CAACCTGAGCCACGCCTACGCCGTGGACGCCGCCAACC | |
| pre-fusion | ACTGCGGCACCATCGCCAACGGCGTGCTGCAGACCTTC | |
| conformation). | ATGAGAATGGCCTGGGGCGGCAGCTACATCGCCCTGGA | |
| Mutated codons | CAGCGGCTGCGGCAACTGGGACTGCATCATGACCAGCT | |
| underlined. | ACCAGTACCTGATCATCCAGAACACCACCTGGGAGGAC | |
| | CACTGCCAGTTCAGCAGACCCAGCCCCATCGGCTACCT | |
| | GGGCCTGCTGAGCCAGAGAACCAGAGACATCTACATCA | |
| | GCAGAAGACTGCTGGGCACCTTCACCTGGACCCTGAGC | |
| | GACAGCGAGGGCAACGAGGCCCCCGGCGGCTACTGCCT | |
| | GACCAGATGGATGCTGATCGAGGCCGAGCTGAAGTGCT | |
| | TCGGCAACACCGCCATCGCCAAGTGCAACGAGAAGCAC | |
| | GACGAGGAGTTCTGCGACATGCTGAGACTGTTCGACTT | |
| | CAACAAGCAGGCCATCGAGAGACTGAAGGCCCCGGCCC | |
| | AGATGAGCATCCAGCTGATCAACAAGGCCGTGAACGCC | |
| | CTGATCAACGACCAGCTGATCATGAAGAACCACCTGAG | |
| | AGACATCATGTGTATCCCCTACTGCAACTACAGCAAGT | |
| | ACTGGTACCTGAACCACACCGTGACCGGCAGAACCAGC | |
| | CTGCCCAGATGCTGGCTGGTGAGCAACGGCAGCTACCT | |
| | GAACGAGACCCACTTCAGCGACGACATCGAGCAGCAGG | |
| | CCGACAACATGATCACCGAGCTGCTGCAGAAGGAGTAC | |
| | ATGGACAGACAGGGCAAGACCCCCGTTAAC | |
| Amino acid | MGQIITFFQEVPHVIEEVMNIVLIALSLLAILKGVYNV | SEQ ID |
| sequence of the | ATCGLFGLISFLLLCGRSCSVTYKGVYELQTLELDMAN | NO: 35 |
| ectodomain of | LNMTMPLSCTKNSSHHYIMVGNETGLELTLTNTSIINH | |
| LASV GP chimera | KFCNLSDAHKRNLYNHALMSIISTFHLSIPNFNQYEAM | |
| (ectodomain of | SCDENGGKISVQYNLSHAYAVDAANHCGTIANGVLQTF | |
| the GP from | MRMAWGGSYIALDSGKGNWDCIMTSYQYLIIQNTTWED | |
| strain LASV/ | HCQFSRPSPIGYLGLLSQRTRDIYISRRLLGTFTWTLS | |
| H. sapiens- | DSEGNEAPGGYCLTRWMLIEAELKCFGNTAIAKCNEKH | |
| wt/NGA/2018/IRR | DEEFCDMLRLFDENKQAIERLKAEAQMSIQLINKAVNA | |
| 013). The | LINDQLIMKNHLRDIMGIPYCNYSKYWYLNHTVTGRTS | |
| signal sequence | LPRCWLVSNGSYLNETHFSDDIEQQADNMITELLQKEY | |
| is bold. | MDRQGKTPVN | |
| Amino acid | MGQIITFFQEVPHVIEEVMNIVLIALSLLAILKGVYNV | SEQ ID |
| sequence of | ATCGLFGLISFLLLCGRSCSVTYKGVYELQTLELDMAN | NO: 36 |
| ectodomain of | LNMTMPLSCTKNSSHHYIMVGNETGLELTLTNTSIINH | |
| LASV GP 1 Pro | KFCNLSDAHKRNLYNHALMSIISTFHLSIPNFNQYEAM | |
| chimera | SCDENGGKISVQYNLSHAYAVDAANHCGTIANGVLQTF | |
| (ectodomain of | MRMAWGGSYIALDSGCGNWDCIMTSYQYLIIQNTTWED | |
| LASV strain | HCQFSRPSPIGYLGLLSQRTRDIYISRRLLGTFTWTLS | |
| LASV/H. sapiens- | DSEGNEAPGGYCLTRWMLIEAELKCFGNTAIAKCNEKH | |
| wt/NGA/2018/IRR | DEEFCDMLRLFDENKQAIERLKAPAQMSIQLINKAVNA | |
| 013 glycoprotein | LINDQLIMKNHLRDIMCIPYCNYSKYWYLNHTVTGRTS | |
| with 3 amino acid | LPRCWLVSNGSYLNETHFSDDIEQQADNMITELLQKEY | |
| mutations to | MDRQGKTPVN | |
| stabilize the | | |
| pre-fusion | | |
| conformation). | | |
| Mutated amino | | |
| acids underlined. | | |
| The signal | | |
| sequence is in | | |
| bold. | | |
| Amino acid | VTYKGVYELQTLELDMANLNMTMPLSCTKNSSHHYIMV | SEQ ID |
| sequence of | GNETGLELTLTNTSIINHKFCNLSDAHKRNLYNHALMS | NO: 37 |
| LASV GP (strain | IISTFHLSIPNFNQYEAMSCDENGGKISVQYNLSHAYA | |
| LASV/H. sapiens- | VDAANHCGTIANGVLQTFMRMAWGGSYIALDSGKGNWD | |
| wt/NGA/2018/IRR | CIMTSYQYLIIQNTTWEDHCQFSRPSPIGYLGLLSQRT | |
| 013 without the | RDIYISRRLLGTFTWTLSDSEGNEAPGGYCLTRWMLIE | |
| signal sequence. | AELKCFGNTAIAKCNEKHDEEFCDMLRLFDFNKQAIER | |
| | LKAEAQMSIQLINKAVNALINDQLIMKNHLRDIMGIPY | |
| | CNYSKYWYLNHTVTGRTSLPRCWLVSNGSYLNETHFSD | |
| | DIEQQADNMITELLQKEYMDRQGKTPLGLVDLFVFSTS | |
| | FYLISIFLHLVKIPTHRHIVGRPCPKPHRLNHMGICSC | |
| | GLYKHPGVPVKWKR | |
| Amino acid | VTYKGVYELQTLELDMANLNMTMPLSCTKNSSHHYIMV | SEQ ID |
| sequence of | GNETGLELTLTNTSIINHKFCNLSDAHKRNLYNHALMS | NO: 38 |
| LASV GP | IISTFHLSIPNFNQYEAMSCDENGGKISVQYNLSHAYA | |
| chimera without | VDAANHCGTIANGVLQTFMRMAWGGSYIALDSGKGNWD | |
| the signal | CIMTSYQYLIIQNTTWEDHCQFSRPSPIGYLGLLSQRT | |
| sequence | RDIYISRRLLGTFTWTLSDSEGNEAPGGYCLTRWMLIE | |
| (ectodomain of | AELKCFGNTAIAKCNEKHDEEFCDMLRLFDFNKQAIER | |
| the GP from | LKAEAQMSIQLINKAVNALINDQLIMKNHLRDIMGIPY | |
| strain LASV/ | CNYSKYWYLNHTVTGRTSLPRCWLVSNGSYLNETHFSD | |
| H. sapiens- | DIEQQADNMITELLQKEYMDRQGKTPVNLITYIVLTII | |
| wt/NGA/2018/IRR | SLVFGILSLILACYLMYKQKAQQKTLLWLGNNTLDQMR | |
| 013 fused to | ATTKM | |
| the transmembrane | | |
| and cytoplasmic | | |
| regions of the F | | |
| protein of NDV). | | |
| Transmembrane | | |
| and cytoplasmic | | |
| regions of the F | | |
| protein of NDV | | |
| are underlined. | | |
| Amino acid | VTYKGVYELQTLELDMANLNMTMPLSCTKNSSHHYIMV | SEQ ID |
| sequence of | GNETGLELTLTNTSIINHKFCNLSDAHKRNLYNHALMS | NO: 39 |
| LASV GP 1 Pro | IISTFHLSIPNFNQYEAMSCDFNGGKISVQYNLSHAYA | |
| without the | VDAANHCGTIANGVLQTFMRMAWGGSYIALDSGCGNWD | |
| signal sequence | CIMTSYQYLIIQNTTWEDHCQFSRPSPIGYLGLLSQRT | |
| (full length | RDIYISRRLLGTFTWTLSDSEGNEAPGGYCLTRWMLIE | |
| sequence of LASV | AELKCFGNTAIAKCNEKHDEEFCDMLRLFDFNKQAIER | |
| strain LASV/ | LKAPAQMSIQLINKAVNALINDQLIMKNHLRDIMCIPY | |
| H. sapiens- | CNYSKYWYLNHTVTGRTSLPRCWLVSNGSYLNETHFSD | |
| wt/NGA/2018/IRR | DIEQQADNMITELLQKEYMDRQGKTPLGLVDLFVFSTS | |
| 013 glycoprotein | FYLISIFLHLVKIPTHRHIVGRPCPKPHRLNHMGICSC | |
| with 3 amino acid | GLYKHPGVPVKWKR | |
| mutations to | | |
| stabilize the | | |
| pre-fusion | | |
| conformation). | | |
| Mutated amino | | |
| acids are | | |
| underlined | | |
| Amino acid | VTYKGVYELQTLELDMANLNMTMPLSCTKNSSHHYIMV | SEQ ID |
| sequence of | GNETGLELTLTNTSIINHKFCNLSDAHKRNLYNHALMS | NO: 40 |
| LASV GP 1 Pro | IISTFHLSIPNFNQYEAMSCDENGGKISVQYNLSHAYA | |
| chimera without | VDAANHCGTIANGVLQTFMRMAWGGSYIALDSGCGNWD | |
| the signal | CIMTSYQYLIIQNTTWEDHCQFSRPSPIGYLGLLSQRT | |
| sequence | RDIYISRRLLGTFTWTLSDSEGNEAPGGYCLTRWMLIE | |
| (ectodomain of | AELKCFGNTAIAKCNEKHDEEFCDMLRLFDFNKQAIER | |
| LASV strain | LKAPAQMSIQLINKAVNALINDQLIMKNHLRDIMCIPY | |
| LASV/H. sapiens- | CNYSKYWYLNHTVTGRTSLPRCWLVSNGSYLNETHFSD | |
| wt/NGA/2018/IRR | DIEQQADNMITELLQKEYMDRQGKTPVNLITYIVLTII | |
| 013 glycoprotein | SLVFGILSLILACYLMYKQKAQQKTLLWLGNNTLDQMR | |
| with 3 amino acid | ATTKM | |
| mutations to | | |
| stabilize the | | |
| pre-fusion | | |
| conformation | | |
| fused to the | | |
| transmembrane | | |
| and cytoplasmic | | |
| regions of the F | | |
| protein of NDV). | | |
| Mutated amino | | |
| acids underlined. | | |
| Transmembrane | | |
| and cytoplasmic | | |
| regions of the F | | |
| protein of NDV | | |
| are underlined. | | |
| Amino acid | VTYKGVYELQTLELDMANLNMTMPLSCTKNSSHHYIMV | SEQ ID |
| sequence of the | GNETGLELTLTNTSIINHKFCNLSDAHKRNLYNHALMS | NO: 41 |
| ectodomain of | IISTFHLSIPNFNQYEAMSCDENGGKISVQYNLSHAYA | |
| LASV GP | VDAANHCGTIANGVLQTFMRMAWGGSYIALDSGKGNWD | |
| chimera without | CIMTSYQYLIIQNTTWEDHCQFSRPSPIGYLGLLSQRT | |
| signal sequence | RDIYISRRLLGTFTWTLSDSEGNEAPGGYCLTRWMLIE | |
| (ectodomain of | AELKCFGNTAIAKCNEKHDEEFCDMLRLFDFNKQAIER | |
| the GP from | LKAEAQMSIQLINKAVNALINDQLIMKNHLRDIMGIPY | |
| strain LASV/ | CNYSKYWYLNHTVTGRTSLPRCWLVSNGSYLNETHFSD | |
| H. sapiens- | DIEQQADNMITELLQKEYMDRQGKTPVN | |
| wt/NGA/2018/ | | |
| IRR 013). | | |
| Amino acid | VTYKGVYELQTLELDMANLNMTMPLSCTKNSSHHYIMV | SEQ ID |
| sequence of | GNETGLELTLTNTSIINHKFCNLSDAHKRNLYNHALMS | NO: 42 |
| ectodomain of | IISTFHLSIPNFNQYEAMSCDENGGKISVQYNLSHAYA | |
| LASV GP 1 Pro | VDAANHCGTIANGVLQTFMRMAWGGSYIALDSGCGNWD | |
| chimera without | CIMTSYQYLIIQNTTWEDHCQFSRPSPIGYLGLLSQRT | |
| signal sequence | RDIYISRRLLGTFTWTLSDSEGNEAPGGYCLTRWMLIE | |
| (ectodomain of | AELKCFGNTAIAKCNEKHDEEFCDMLRLFDFNKQAIER | |
| LASV strain | LKAPAQMSIQLINKAVNALINDQLIMKNHLRDIMCIPY | |
| LASV/H. sapiens- | CNYSKYWYLNHTVTGRTSLPRCWLVSNGSYLNETHFSD | |
| wt/NGA/2018/IRR | DIEQQADNMITELLQKEYMDRQGKTPVN | |
| 013 glycoprotein | | |
| with 3 amino acid | | |
| mutations to | | |
| stabilize the | | |
| pre-fusion | | |
| conformation). | | |
| Mutated amino | | |
| acids underlined. | | |
| Nucleotide | GTGACCTACAAGGGCGTGTACGAGCTGCAGACCCTGGA | SEQ ID |
| counterpart of | GCTGGACATGGCCAACCTGAACATGACCATGCCCCTGA | NO: 43 |
| SEQ ID NO: 37 | GCTGCACCAAGAACAGCAGCCACCACTACATCATGGTG | |
| | GGCAACGAGACCGGCCTGGAGCTGACCCTGACCAACAC | |
| | CAGCATCATCAACCACAAGTTCTGCAACCTGAGCGACG | |
| | CCCACAAGAGAAACCTGTACAACCACGCCCTGATGAGC | |
| | ATCATCAGCACCTTCCACCTGAGCATCCCCAACTTCAA | |
| | CCAGTACGAGGCCATGAGCTGCGACTTCAACGGCGGCA | |
| | AGATCAGCGTGCAGTACAACCTGAGCCACGCCTACGCC | |
| | GTGGACGCCGCCAACCACTGCGGCACCATCGCCAACGG | |
| | CGTGCTGCAGACCTTCATGAGAATGGCCTGGGGCGGCA | |
| | GCTACATCGCCCTGGACAGCGGCAAGGGCAACTGGGAC | |
| | TGCATCATGACCAGCTACCAGTACCTGATCATCCAGAA | |
| | CACCACCTGGGAGGACCACTGCCAGTTCAGCAGACCCA | |
| | GCCCCATCGGCTACCTGGGCCTGCTGAGCCAGAGAACC | |
| | AGAGACATCTACATCAGCAGAAGACTGCTGGGCACCTT | |
| | CACCTGGACCCTGAGCGACAGCGAGGGCAACGAGGCCC | |
| | CCGGCGGCTACTGCCTGACCAGATGGATGCTGATCGAG | |
| | GCCGAGCTGAAGTGCTTCGGCAACACCGCCATCGCCAA | |
| | GTGCAACGAGAAGCACGACGAGGAGTTCTGCGACATGC | |
| | TGAGACTGTTCGACTTCAACAAGCAGGCCATCGAGAGA | |
| | CTGAAGGCCGAGGCCCAGATGAGCATCCAGCTGATCAA | |
| | CAAGGCCGTGAACGCCCTGATCAACGACCAGCTGATCA | |
| | TGAAGAACCACCTGAGAGACATCATGGGCATCCCCTAC | |
| | TGCAACTACAGCAAGTACTGGTACCTGAACCACACCGT | |
| | GACCGGCAGAACCAGCCTGCCCAGATGCTGGCTGGTGA | |
| | GCAACGGCAGCTACCTGAACGAGACCCACTTCAGCGAC | |
| | GACATCGAGCAGCAGGCCGACAACATGATCACCGAGCT | |
| | GCTGCAGAAGGAGTACATGGACAGACAGGGCAAGACCC | |
| | CCCTGGGCCTGGTGGACCTGTTCGTGTTCAGCACCAGC | |
| | TTCTACCTGATCAGCATCTTCCTGCACCTGGTGAAGAT | |
| | CCCCACCCACAGACACATCGTGGGCAGACCCTGCCCCA | |
| | AGCCCCACAGACTGAACCACATGGGCATCTGCAGCTGC | |
| | GGCCTGTACAAGCACCCCGGCGTGCCCGTGAAGTGGAA | |
| | GAGATGA | |
| Nucleotide | GTGACCTACAAGGGCGTGTACGAGCTGCAGACCCTGGA | SEQ ID |
| counterpart of | GCTGGACATGGCCAACCTGAACATGACCATGCCCCTGA | NO: 44 |
| SEQ ID NO: 38 | GCTGCACCAAGAACAGCAGCCACCACTACATCATGGTG | |
| | GGCAACGAGACCGGCCTGGAGCTGACCCTGACCAACAC | |
| | CAGCATCATCAACCACAAGTTCTGCAACCTGAGCGACG | |
| | CCCACAAGAGAAACCTGTACAACCACGCCCTGATGAGC | |
| | ATCATCAGCACCTTCCACCTGAGCATCCCCAACTTCAA | |
| | CCAGTACGAGGCCATGAGCTGCGACTTCAACGGCGGCA | |
| | AGATCAGCGTGCAGTACAACCTGAGCCACGCCTACGCC | |
| | GTGGACGCCGCCAACCACTGCGGCACCATCGCCAACGG | |
| | CGTGCTGCAGACCTTCATGAGAATGGCCTGGGGCGGCA | |
| | GCTACATCGCCCTGGACAGCGGCAAGGGCAACTGGGAC | |
| | TGCATCATGACCAGCTACCAGTACCTGATCATCCAGAA | |
| | CACCACCTGGGAGGACCACTGCCAGTTCAGCAGACCCA | |
| | GCCCCATCGGCTACCTGGGCCTGCTGAGCCAGAGAACC | |
| | AGAGACATCTACATCAGCAGAAGACTGCTGGGCACCTT | |
| | CACCTGGACCCTGAGCGACAGCGAGGGCAACGAGGCCC | |
| | CCGGCGGCTACTGCCTGACCAGATGGATGCTGATCGAG | |
| | GCCGAGCTGAAGTGCTTCGGCAACACCGCCATCGCCAA | |
| | GTGCAACGAGAAGCACGACGAGGAGTTCTGCGACATGC | |
| | TGAGACTGTTCGACTTCAACAAGCAGGCCATCGAGAGA | |
| | CTGAAGGCCGAGGCCCAGATGAGCATCCAGCTGATCAA | |
| | CAAGGCCGTGAACGCCCTGATCAACGACCAGCTGATCA | |
| | TGAAGAACCACCTGAGAGACATCATGGGCATCCCCTAC | |
| | TGCAACTACAGCAAGTACTGGTACCTGAACCACACCGT | |
| | GACCGGCAGAACCAGCCTGCCCAGATGCTGGCTGGTGA | |
| | GCAACGGCAGCTACCTGAACGAGACCCACTTCAGCGAC | |
| | GACATCGAGCAGCAGGCCGACAACATGATCACCGAGCT | |
| | GCTGCAGAAGGAGTACATGGACAGACAGGGCAAGACCC | |
| | CCGTTAACCTCATTACCTATATCGTTTTGACTATCATA | |
| | TCTCTTGTTTTTGGTATACTTAGCCTGATTCTAGCATG | |
| | CTACCTAATGTACAAGCAAAAGGCGCAACAAAAGACCT | |
| | TATTATGGCTTGGGAATAATACCCTAGATCAGATGAGA | |
| | GCCACTACAAAAATGTGA | |
| Nucleotide | GTGACCTACAAGGGCGTGTACGAGCTGCAGACCCTGGA | SEQ ID |
| counterpart of | GCTGGACATGGCCAACCTGAACATGACCATGCCCCTGA | NO: 45 |
| SEQ ID NO: 39 | GCTGCACCAAGAACAGCAGCCACCACTACATCATGGTG | |
| | GGCAACGAGACCGGCCTGGAGCTGACCCTGACCAACAC | |
| | CAGCATCATCAACCACAAGTTCTGCAACCTGAGCGACG | |
| | CCCACAAGAGAAACCTGTACAACCACGCCCTGATGAGC | |
| | ATCATCAGCACCTTCCACCTGAGCATCCCCAACTTCAA | |
| | CCAGTACGAGGCCATGAGCTGCGACTTCAACGGCGGCA | |
| | AGATCAGCGTGCAGTACAACCTGAGCCACGCCTACGCC | |
| | GTGGACGCCGCCAACCACTGCGGCACCATCGCCAACGG | |
| | CGTGCTGCAGACCTTCATGAGAATGGCCTGGGGCGGCA | |
| | GCTACATCGCCCTGGACAGCGGCTGCGGCAACTGGGAC | |
| | TGCATCATGACCAGCTACCAGTACCTGATCATCCAGAA | |
| | CACCACCTGGGAGGACCACTGCCAGTTCAGCAGACCCA | |
| | GCCCCATCGGCTACCTGGGCCTGCTGAGCCAGAGAACC | |
| | AGAGACATCTACATCAGCAGAAGACTGCTGGGCACCTT | |
| | CACCTGGACCCTGAGCGACAGCGAGGGCAACGAGGCCC | |
| | CCGGCGGCTACTGCCTGACCAGATGGATGCTGATCGAG | |
| | GCCGAGCTGAAGTGCTTCGGCAACACCGCCATCGCCAA | |
| | GTGCAACGAGAAGCACGACGAGGAGTTCTGCGACATGC | |
| | TGAGACTGTTCGACTTCAACAAGCAGGCCATCGAGAGA | |
| | CTGAAGGCCCCGGCCCAGATGAGCATCCAGCTGATCAA | |
| | CAAGGCCGTGAACGCCCTGATCAACGACCAGCTGATCA | |
| | TGAAGAACCACCTGAGAGACATCATGTGTATCCCCTAC | |
| | TGCAACTACAGCAAGTACTGGTACCTGAACCACACCGT | |
| | GACCGGCAGAACCAGCCTGCCCAGATGCTGGCTGGTGA | |
| | GCAACGGCAGCTACCTGAACGAGACCCACTTCAGCGAC | |
| | GACATCGAGCAGCAGGCCGACAACATGATCACCGAGCT | |
| | GCTGCAGAAGGAGTACATGGACAGACAGGGCAAGACCC | |
| | CCCTGGGCCTGGTGGACCTGTTCGTGTTCAGCACCAGC | |
| | TTCTACCTGATCAGCATCTTCCTGCACCTGGTGAAGAT | |
| | CCCCACCCACAGACACATCGTGGGCAGACCCTGCCCCA | |
| | AGCCCCACAGACTGAACCACATGGGCATCTGCAGCTGC | |
| | GGCCTGTACAAGCACCCCGGCGTGCCCGTGAAGTGGAA | |
| | GAGATGA | |
| Nucleotide | GTGACCTACAAGGGCGTGTACGAGCTGCAGACCCTGGA | SEQ ID |
| counterpart of | GCTGGACATGGCCAACCTGAACATGACCATGCCCCTGA | NO: 46 |
| SEQ ID NO: 40 | GCTGCACCAAGAACAGCAGCCACCACTACATCATGGTG | |
| | GGCAACGAGACCGGCCTGGAGCTGACCCTGACCAACAC | |
| | CAGCATCATCAACCACAAGTTCTGCAACCTGAGCGACG | |
| | CCCACAAGAGAAACCTGTACAACCACGCCCTGATGAGC | |
| | ATCATCAGCACCTTCCACCTGAGCATCCCCAACTTCAA | |
| | CCAGTACGAGGCCATGAGCTGCGACTTCAACGGCGGCA | |
| | AGATCAGCGTGCAGTACAACCTGAGCCACGCCTACGCC | |
| | GTGGACGCCGCCAACCACTGCGGCACCATCGCCAACGG | |
| | CGTGCTGCAGACCTTCATGAGAATGGCCTGGGGCGGCA | |
| | GCTACATCGCCCTGGACAGCGGCTGCGGCAACTGGGAC | |
| | TGCATCATGACCAGCTACCAGTACCTGATCATCCAGAA | |
| | CACCACCTGGGAGGACCACTGCCAGTTCAGCAGACCCA | |
| | GCCCCATCGGCTACCTGGGCCTGCTGAGCCAGAGAACC | |
| | AGAGACATCTACATCAGCAGAAGACTGCTGGGCACCTT | |
| | CACCTGGACCCTGAGCGACAGCGAGGGCAACGAGGCCC | |
| | CCGGCGGCTACTGCCTGACCAGATGGATGCTGATCGAG | |
| | GCCGAGCTGAAGTGCTTCGGCAACACCGCCATCGCCAA | |
| | GTGCAACGAGAAGCACGACGAGGAGTTCTGCGACATGC | |
| | TGAGACTGTTCGACTTCAACAAGCAGGCCATCGAGAGA | |
| | CTGAAGGCCCCGGCCCAGATGAGCATCCAGCTGATCAA | |
| | CAAGGCCGTGAACGCCCTGATCAACGACCAGCTGATCA | |
| | TGAAGAACCACCTGAGAGACATCATGTGTATCCCCTAC | |
| | TGCAACTACAGCAAGTACTGGTACCTGAACCACACCGT | |
| | GACCGGCAGAACCAGCCTGCCCAGATGCTGGCTGGTGA | |
| | GCAACGGCAGCTACCTGAACGAGACCCACTTCAGCGAC | |
| | GACATCGAGCAGCAGGCCGACAACATGATCACCGAGCT | |
| | GCTGCAGAAGGAGTACATGGACAGACAGGGCAAGACCC | |
| | CCGTTAACCTCATTACCTATATCGTTTTGACTATCATA | |
| | TCTCTTGTTTTTGGTATACTTAGCCTGATTCTAGCATG | |
| | CTACCTAATGTACAAGCAAAAGGCGCAACAAAAGACCT | |
| | TATTATGGCTTGGGAATAATACCCTAGATCAGATGAGA | |
| | GCCACTACAAAAATGTGA | |
| Nucleotide | GTGACCTACAAGGGCGTGTACGAGCTGCAGACCCTGGA | SEQ ID |
| counterpart of | GCTGGACATGGCCAACCTGAACATGACCATGCCCCTGA | NO: 47 |
| SEQ ID NO: 41 | GCTGCACCAAGAACAGCAGCCACCACTACATCATGGTG | |
| | GGCAACGAGACCGGCCTGGAGCTGACCCTGACCAACAC | |
| | CAGCATCATCAACCACAAGTTCTGCAACCTGAGCGACG | |
| | CCCACAAGAGAAACCTGTACAACCACGCCCTGATGAGC | |
| | ATCATCAGCACCTTCCACCTGAGCATCCCCAACTTCAA | |
| | CCAGTACGAGGCCATGAGCTGCGACTTCAACGGCGGCA | |
| | AGATCAGCGTGCAGTACAACCTGAGCCACGCCTACGCC | |
| | GTGGACGCCGCCAACCACTGCGGCACCATCGCCAACGG | |
| | CGTGCTGCAGACCTTCATGAGAATGGCCTGGGGCGGCA | |
| | GCTACATCGCCCTGGACAGCGGCAAGGGCAACTGGGAC | |
| | TGCATCATGACCAGCTACCAGTACCTGATCATCCAGAA | |
| | CACCACCTGGGAGGACCACTGCCAGTTCAGCAGACCCA | |
| | GCCCCATCGGCTACCTGGGCCTGCTGAGCCAGAGAACC | |
| | AGAGACATCTACATCAGCAGAAGACTGCTGGGCACCTT | |
| | CACCTGGACCCTGAGCGACAGCGAGGGCAACGAGGCCC | |
| | CCGGCGGCTACTGCCTGACCAGATGGATGCTGATCGAG | |
| | GCCGAGCTGAAGTGCTTCGGCAACACCGCCATCGCCAA | |
| | GTGCAACGAGAAGCACGACGAGGAGTTCTGCGACATGC | |
| | TGAGACTGTTCGACTTCAACAAGCAGGCCATCGAGAGA | |
| | CTGAAGGCCGAGGCCCAGATGAGCATCCAGCTGATCAA | |
| | CAAGGCCGTGAACGCCCTGATCAACGACCAGCTGATCA | |
| | TGAAGAACCACCTGAGAGACATCATGGGCATCCCCTAC | |
| | TGCAACTACAGCAAGTACTGGTACCTGAACCACACCGT | |
| | GACCGGCAGAACCAGCCTGCCCAGATGCTGGCTGGTGA | |
| | GCAACGGCAGCTACCTGAACGAGACCCACTTCAGCGAC | |
| | GACATCGAGCAGCAGGCCGACAACATGATCACCGAGCT | |
| | GCTGCAGAAGGAGTACATGGACAGACAGGGCAAGACCC | |
| | CCGTTAAC | |
| Nucleotide | GTGACCTACAAGGGCGTGTACGAGCTGCAGACCCTGGA | SEQ ID |
| counterpart of | GCTGGACATGGCCAACCTGAACATGACCATGCCCCTGA | NO: 48 |
| SEQ ID NO: 42 | GCTGCACCAAGAACAGCAGCCACCACTACATCATGGTG | |
| | GGCAACGAGACCGGCCTGGAGCTGACCCTGACCAACAC | |
| | CAGCATCATCAACCACAAGTTCTGCAACCTGAGCGACG | |
| | CCCACAAGAGAAACCTGTACAACCACGCCCTGATGAGC | |
| | ATCATCAGCACCTTCCACCTGAGCATCCCCAACTTCAA | |
| | CCAGTACGAGGCCATGAGCTGCGACTTCAACGGCGGCA | |
| | AGATCAGCGTGCAGTACAACCTGAGCCACGCCTACGCC | |
| | GTGGACGCCGCCAACCACTGCGGCACCATCGCCAACGG | |
| | CGTGCTGCAGACCTTCATGAGAATGGCCTGGGGCGGCA | |
| | GCTACATCGCCCTGGACAGCGGCTGCGGCAACTGGGAC | |
| | TGCATCATGACCAGCTACCAGTACCTGATCATCCAGAA | |
| | CACCACCTGGGAGGACCACTGCCAGTTCAGCAGACCCA | |
| | GCCCCATCGGCTACCTGGGCCTGCTGAGCCAGAGAACC | |
| | AGAGACATCTACATCAGCAGAAGACTGCTGGGCACCTT | |
| | CACCTGGACCCTGAGCGACAGCGAGGGCAACGAGGCCC | |
| | CCGGCGGCTACTGCCTGACCAGATGGATGCTGATCGAG | |
| | GCCGAGCTGAAGTGCTTCGGCAACACCGCCATCGCCAA | |
| | GTGCAACGAGAAGCACGACGAGGAGTTCTGCGACATGC | |
| | TGAGACTGTTCGACTTCAACAAGCAGGCCATCGAGAGA | |
| | CTGAAGGCCCCGGCCCAGATGAGCATCCAGCTGATCAA | |
| | CAAGGCCGTGAACGCCCTGATCAACGACCAGCTGATCA | |
| | TGAAGAACCACCTGAGAGACATCATGTGTATCCCCTAC | |
| | TGCAACTACAGCAAGTACTGGTACCTGAACCACACCGT | |
| | GACCGGCAGAACCAGCCTGCCCAGATGCTGGCTGGTGA | |
| | GCAACGGCAGCTACCTGAACGAGACCCACTTCAGCGAC | |
| | GACATCGAGCAGCAGGCCGACAACATGATCACCGAGCT | |
| | GCTGCAGAAGGAGTACATGGACAGACAGGGCAAGACCC | |
| | CCGTTAAC | |

TABLE 4
LASV NP NUCLEOTIDE PROTEIN SEQUENCES

| | | SEQ ID |
|---|---|---|
| Description | Sequence | NO: |
| Nucleotide | ATGAGCGTGAGCAAGGAGGTGAAGAGCTTCCTGTGGAC | SEQ ID |
| sequence of the | CCAGAGCCTGAGAAGAGAGCTGAGCGGCTACTGCAGCA | NO: 14 |
| codon optimized | ACATCAAGCTGCAGGTGGTGAAGGACGCCCAGGCCCTG | |
| LASV NP (strain | CTGCACGGCCTGGACTTCAGCGAGGTGAGCAACGTGCA | |
| LASV/H. sapiens- | GAGACTGATGAGAAAGCAGAAGAGAGACGACGGCGACC | |
| wt/NGA/2018/IRR | TGAAGAGACTGAGAGACCTGAACCAGGCCGTGAACAAC | |
| 013) | CTGGTGGAGCTGAAGAGCACCCAGCAGAAGAGCGTGCT | |
| | GAGAGTGGGCACCCTGACCAGCGACGACCTGCTGATCC | |
| | TGGCCGCCGACCTGGAGAAGCTGAAGAGCAAGGTGATC | |
| | AGAACCGAGAGACCCCTGAGCAGCGGCGTGTACATGGG | |
| | CAACCTGAGCACCCAGCAGCTGGAGCAGAGAAAGGCCC | |
| | TGCTGAGCATGATCGGCATGGTGGGCGGCGCCCAGGGC | |
| | ACCCAGCCCGGCAGAGACGGCGTGGTGAGAGTGTGGGA | |
| | CGTGAAGAACCCCGACCTGCTGAACAACCAGTTCGGCA | |
| | CCATGCCCAGCCTGACCCTGGCCTGCCTGACCAAGCAG | |
| | GGCCAGGTGGACCTGAACGACGTGGTGCTGGCCCTGAC | |
| | CGACCTGGGCCTGATCTACACCGCCAAGTACCCCAACA | |
| | GCAGCGACCTGGACAGACTGAGCCAGAGCCACCCCATC | |
| | CTGAACATGGTGGACACCAAGAAGAGCAGCCTGAACAT | |
| | CAGCGGCTACAACTTCAGCCTGGGCGCCGCCGTGAAGG | |
| | CCGGCGCCTGCATGCTGGACGGCGGCAACATGCTGGAG | |
| | ACCATCAAGGTGACCCCCCAGACCATGGACGGCATCCT | |
| | GAAGAGCATCCTGAAGGTGAAGAGAAGCCTGGGCATGT | |
| | TCGTGAGCGACACCCCCGGCGAGAGAAACCCCTACGAG | |
| | AACATCCTGTACAAGATCTGCCTGAGCGGCGACGGCTG | |
| | GCCCTACATCGCCAGCAGAACCAGCATCGTGGGCAGAG | |
| | CCTGGGAGAACACCACCGTGGACCTGGAGAGCAACGGC | |
| | AAGCCCCAGAAGGTGGGCACCGCCGGCAGCAACAAGAG | |
| | CCTGCAGAGCGCCGGCTTCCCCACCGGCCTGACCTACA | |
| | GCCAGCTGATGACCCTGAAGGACAGCATGATGCAGCTG | |
| | GACCCCAGCGCCAAGACCTGGATCGACATCGAGGGCAG | |
| | ACCCGAGGACCCCGTGGAGATCGCCCTGTACCAGCCCA | |
| | TGAGCGGCTGCTACATCCACTTCTTCAGAGAGCCCACC | |
| | GACCTGAAGCAGTTCAAGCAGGACGCCAAGTACAGCCA | |
| | CGGCATCGACGTGGCCGACCTGTTCAGCGCCCAGCCCG | |
| | GCCTGACCAGCGCCGTGATCGAGGCCCTGCCCAGAAAC | |
| | ATGGTGCTGACCTGCCAGGGCAGCGACGACATCAAGAA | |
| | GCTGCTGGACAGCCAGGGCAGAAGAGACATCAAGCTGA | |
| | TCGACATCAGCCTGAACAAGGTGGACAGCAGAAAGTTC | |
| | GAGAACGCCGTGTGGGACCAGTGCAAGGACCTGTGCCA | |
| | CATGCACACCGGCGTGGTGGTGGAGAAGAAGAAGAGAG | |
| | GCGGCAAGGAGGAGATCACCCCCCACTGCGCCCTGATG | |
| | GACTGCATCATGTTCGACGCCGCCGTGAGCGGCGGCCT | |
| | GAACATCCCCGTGCTGAGAGCCGTGCTGCCCAAGGACA | |
| | TGGTGTTCAGAACCAGCAGCCCCAAGGTGGTGCTGTGA | |
| Nucleotide | ATGAGCGTGAGCAAGGAGGTGAAGAGCTTCCTGTGGAC | SEQ ID |
| sequence of the | CCAGAGCCTGAGAAGAGAGCTGAGCGGCTACTGCAGCA | NO: 15 |
| codon optimized | ACATCAAGCTGCAGGTGGTGAAGGACGCCCAGGCCCTG | |
| LASV NP ExON | CTGCACGGCCTGGACTTCAGCGAGGTGAGCAACGTGCA | |
| KO (strain | GAGACTGATGAGAAAGCAGAAGAGAGACGACGGCGACC | |
| LASV/H. sapiens- | TGAAGAGACTGAGAGACCTGAACCAGGCCGTGAACAAC | |
| wt/NGA/2018/IRR | CTGGTGGAGCTGAAGAGCACCCAGCAGAAGAGCGTGCT | |
| 013 with 2 | GAGAGTGGGCACCCTGACCAGCGACGACCTGCTGATCC | |
| amino acid | TGGCCGCCGACCTGGAGAAGCTGAAGAGCAAGGTGATC | |
| mutations). | AGAACCGAGAGACCCCTGAGCAGCGGCGTGTACATGGG | |
| Mutated codons | CAACCTGAGCACCCAGCAGCTGGAGCAGAGAAAGGCCC | |
| underlined. | TGCTGAGCATGATCGGCATGGTGGGCGGCGCCCAGGGC | |
| | ACCCAGCCCGGCAGAGACGGCGTGGTGAGAGTGTGGGA | |
| | CGTGAAGAACCCCGACCTGCTGAACAACCAGTTCGGCA | |
| | CCATGCCCAGCCTGACCCTGGCCTGCCTGACCAAGCAG | |
| | GGCCAGGTGGACCTGAACGACGTGGTGCTGGCCCTGAC | |
| | CGACCTGGGCCTGATCTACACCGCCAAGTACCCCAACA | |
| | GCAGCGACCTGGACAGACTGAGCCAGAGCCACCCCATC | |
| | CTGAACATGGTGGACACCAAGAAGAGCAGCCTGAACAT | |
| | CAGCGGCTACAACTTCAGCCTGGGCGCCGCCGTGAAGG | |
| | CCGGCGCCTGCATGCTGGACGGCGGCAACATGCTGGAG | |
| | ACCATCAAGGTGACCCCCCAGACCATGGACGGCATCCT | |
| | GAAGAGCATCCTGAAGGTGAAGAGAAGCCTGGGCATGT | |
| | TCGTGAGCGACACCCCCGGCGAGAGAAACCCCTACGAG | |
| | AACATCCTGTACAAGATCTGCCTGAGCGGCGACGGCTG | |
| | GCCCTACATCGCCAGCAGAACCAGCATCGTGGGCAGAG | |
| | CCTGGGAGAACACCACCGTGGACCTGGAGAGCAACGGC | |
| | AAGCCCCAGAAGGTGGGCACCGCCGGCAGCAACAAGAG | |
| | CCTGCAGAGCGCCGGCTTCCCCaccGGCCTGACCTACA | |
| | GCCAGCTGATGACCCTGAAGGACAGCATGATGCAGCTG | |
| | GACCCCAGCGCCAAGACCTGGATCGCCATCGAGGCCAG | |
| | ACCCGAGGACCCCGTGGAGATCGCCCTGTACCAGCCCA | |
| | TGAGCGGCTGCTACATCCACTTCTTCAGAGAGCCCACC | |
| | GACCTGAAGCAGTTCAAGCAGGACGCCAAGTACAGCCA | |
| | CGGCATCGACGTGGCCGACCTGTTCAGCGCCCAGCCCG | |
| | GCCTGACCAGCGCCGTGATCGAGGCCCTGCCCAGAAAC | |
| | ATGGTGCTGACCTGCCAGGGCAGCGACGACATCAAGAA | |
| | GCTGCTGGACAGCCAGGGCAGAAGAGACATCAAGCTGA | |
| | TCGACATCAGCCTGAACAAGGTGGACAGCAGAAAGTTC | |
| | GAGAACGCCGTGTGGGACCAGTGCAAGGACCTGTGCCA | |
| | CATGCACACCGGCGTGGTGGTGGAGAAGAAGAAGAGAG | |
| | GCGGCAAGGAGGAGATCACCCCCCACTGCGCCCTGATG | |
| | GACTGCATCATGTTCGACGCCGCCGTGAGCGGCGGCCT | |
| | GAACATCCCCGTGCTGAGAGCCGTGCTGCCCAAGGACA | |
| | TGGTGTTCAGAACCAGCAGCCCCAAGGTGGTGCTGTGA | |
| Amino acid | MSVSKEVKSFLWTQSLRRELSGYCSNIKLQVVKDAQAL | SEQ ID |
| sequence of the | LHGLDFSEVSNVQRLMRKQKRDDGDLKRLRDLNQAVNN | NO: 16 |
| LASV NP (strain | LVELKSTQQKSVLRVGTLTSDDLLILAADLEKLKSKVI | |
| LASV/H. sapiens- | RTERPLSSGVYMGNLSTQQLEQRKALLSMIGMVGGAQG | |
| wt/NGA/2018/IRR | TQPGRDGVVRVWDVKNPDLLNNQFGTMPSLTLACLTKQ | |
| 013) | GQVDLNDVVLALTDLGLIYTAKYPNSSDLDRLSQSHPI | |
| | LNMVDTKKSSLNISGYNFSLGAAVKAGACMLDGGNMLE | |
| | TIKVTPQTMDGILKSILKVKRSLGMFVSDTPGERNPYE | |
| | NILYKICLSGDGWPYIASRTSIVGRAWENTTVDLESNG | |
| | KPQKVGTAGSNKSLQSAGFPTGLTYSQLMTLKDSMMQL | |
| | DPSAKTWIDIEGRPEDPVEIALYQPMSGCYIHFFREPT | |
| | DLKQFKQDAKYSHGIDVADLFSAQPGLTSAVIEALPRN | |
| | MVLTCQGSDDIKKLLDSQGRRDIKLIDISLNKVDSRKF | |
| | ENAVWDQCKDLCHMHTGVVVEKKKRGGKEEITPHCALM | |
| | DCIMFDAAVSGGLNIPVLRAVLPKDMVFRTSSPKVVL | |
| Amino acid | MSVSKEVKSFLWTQSLRRELSGYCSNIKLQVVKDAQAL | SEQ ID |
| sequence of the | LHGLDFSEVSNVQRLMRKQKRDDGDLKRLRDLNQAVNN | NO: 17 |
| LASV NP ExON | LVELKSTQQKSVLRVGTLTSDDLLILAADLEKLKSKVI | |
| KO (strain | RTERPLSSGVYMGNLSTQQLEQRKALLSMIGMVGGAQG | |
| LASV/H. sapiens- | TQPGRDGVVRVWDVKNPDLLNNQFGTMPSLTLACLTKQ | |
| wt/NGA/2018/IRR | GQVDLNDVVLALTDLGLIYTAKYPNSSDLDRLSQSHPI | |
| 013 with 2 | LNMVDTKKSSLNISGYNFSLGAAVKAGACMLDGGNMLE | |
| amino acid | TIKVTPQTMDGILKSILKVKRSLGMFVSDTPGERNPYE | |
| mutations. | NILYKICLSGDGWPYIASRTSIVGRAWENTTVDLESNG | |
| Mutated amino | KPQKVGTAGSNKSLQSAGFPTGLTYSQLMTLKDSMMQL | |
| acids underlined. | DPSAKTWIAIEARPEDPVEIALYQPMSGCYIHFFREPT | |
| | DLKQFKQDAKYSHGIDVADLFSAQPGLTSAVIEALPRN | |
| | MVLTCQGSDDIKKLLDSQGRRDIKLIDISLNKVDSRKF | |
| | ENAVWDQCKDLCHMHTGVVVEKKKRGGKEEITPHCALM | |
| | DCIMFDAAVSGGLNIPVLRAVLPKDMVFRTSSPKVVL | |

TABLE 5
OTHER SEQUENCES

| | | SEQ |
|---|---|---|
| Description | Sequence | ID NO: |
| SacII | CCGCGG | SEQ ID |
| Restriction | | NO: 18 |
| Sequence | | |
| NDV Gene End | TTAGAAAAAA | SEQ ID |
| Sequence | | NO: 19 |
| NDV Gene Start | ACGGGTAGAA | SEQ ID |
| Sequence | | NO: 20 |
| Kozak Sequence | CCGCCACC | SEQ ID |
| | | NO: 21 |
| Signal Sequence | MGQIITFFQE | SEQ ID |
| of the LASV GP | VPHVIEEVMN | NO: 22 |
| protein | IVLIALSLLA | |
| | ILKGVYNVAT | |
| | CGLFGLISFL | |
| | LLCGRSCS | |
| Peptide derived | KSFLWTQSL | SEQ ID |
| from the LASV NP | | NO: 23 |
| sequence | | |
| Peptide derived | QAVNNLVEL | SEQ ID |
| from the LASV NP | | NO: 24 |
| sequence | | |
| Peptide derived | LTYSQLMTL | SEQ ID |
| from the LASV NP | | NO: 25 |
| sequence | | |
| Peptide derived | YQPMSGCYI | SEQ ID |
| from the LASV NP | | NO: 26 |
| sequence | | |
| Peptide derived | SGGLNIPVL | SEQ ID |
| from the LASV NP | | NO: 27 |
| sequence | | |
| Peptide derived | QIITFFQEV | SEQ ID |
| from the LASV GP | | NO: 28 |
| sequence | | |
| Peptide derived | ANLNMTMPL | SEQ ID |
| from the LASV GP | | NO: 29 |
| sequence | | |
| Peptide derived | IINHKFCNL | SEQ ID |
| from the LASV GP | | NO: 30 |
| sequence | | |
| Peptide derived | NALINDQLI | SEQ ID |
| from the LASV GP | | NO: 31 |
| sequence | | |
| Peptide derived | CNYSKYWYL | SEQ ID |
| from the LASV GP | | NO: 32 |
| sequence | | |

### 5.10 Embodiments

1. A recombinant protein comprising a derivative of the ectodomain of a Lassa virus glycoprotein, wherein the derivative of the ectodomain comprises an amino acid sequence that is at least 90% identical to the amino acid sequence of SEQ ID NO:42 or 36, and wherein the derivative of the ectodomain comprises: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP.

2. The recombinant protein of embodiment 1, wherein the derivative of the ectodomain comprises an amino acid sequence that is at least 95% identical to the amino acid sequence of SEQ ID NO: 42 or 36.

3. The recombinant protein of embodiment 1, wherein the derivative of the ectodomain comprises the amino acid sequence of SEQ ID NO: 42 or 36.

4. A recombinant protein comprising a derivative of the ectodomain of a Lassa virus glycoprotein, wherein the derivative of the ectodomain comprises the amino acid sequence of a Lassa virus glycoprotein ectodomain and amino acid substitutions resulting in: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP.

5. The recombinant protein of any one of embodiments 1 to 4, which further comprises the transmembrane and cytoplasmic domains of NDV F protein.

6. The recombinant protein of embodiment 5, wherein the NDV F protein is of the LaSota strain.

7. The recombinant protein of embodiment 5, wherein the embodiments 1 to 4, wherein the transmembrane and cytoplasmic domains of NDV F protein comprise the amino acid sequence of SEQ ID NO:5.

8. The recombinant NDV of any one of embodiments 5 to 7, wherein the derivative of the ectodomain is linked directly to the transmembrane of the NDV F protein.

9. The recombinant NDV of any one of embodiments 5 to 7, wherein the derivative of the ectodomain is linked to the transmembrane of the NDV F protein by a linker.

10. A recombinant protein comprising an amino acid sequence that is at least 90% identical to the amino acid sequence of SEQ ID NO:39, 40, 12, or 13, wherein the protein comprises: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP.

11. The recombinant protein of embodiment 10, wherein the amino acid sequence is at least 95% identical to the amino acid sequence of SEQ ID NO:39, 40, 12, or 13.

12. The recombinant protein of embodiment 10, wherein the protein comprises the amino acid sequence of SEQ ID NO:39, 40, 12, or 13.

13. A recombinant protein comprising a derivative of a Lassa virus nucleoprotein, wherein the derivative comprises an amino acid sequence that is at least 90% identical to the amino acid sequence of SEQ ID NO:17, wherein the derivative comprises alanine at amino acid positions 389 and 392 of Lassa virus/H. sapiens-wt/NGA/2018/IRR 013 nucleoprotein (NP).

14. The recombinant protein of embodiment 13, wherein the derivative comprises an amino acid sequence that is at least 95% identical to the amino acid sequence of SEQ ID NO:17.

15. The recombinant protein of embodiment 13, wherein the derivative comprises the amino acid sequence of SEQ ID NO:17.

16. A polynucleotide comprises a nucleotide sequence encoding the recombinant protein of any one of embodiments 1 to 15.

17. A polynucleotide comprising the nucleotide sequence of SEQ ID NO:6, 7, 8, 9, 14, 15, 43, 44, 45, 46, 47, or 48, or a nucleotide sequence that is at least 80% identical to the nucleotide sequence of SEQ ID NO: 6, 7, 8, 9, 14, 15, 43, 44, 45, 46, 47, or 48.

18. The polynucleotide of embodiment 17, which comprises a nucleotide sequence that is at least 80% identical to the nucleotide sequence of SEQ ID NO: 6, 7, 8, 9, 14, 15, 43, 44, 45, 46, 47, or 48.

19. The polynucleotide of embodiment 17, which comprises a nucleotide sequence that is at least 85% identical to the nucleotide sequence of SEQ ID NO: 6, 7, 8, 9, 14, 15, 43, 44, 45, 46, 47, or 48.

20. The polynucleotide of embodiment 17, which comprises a nucleotide sequence that is at least 90% identical to the nucleotide sequence of SEQ ID NO: 6, 7, 8, 9, 14, 15, 43, 44, 45, 46, 47, or 48.

21. The polynucleotide of embodiment 17, which comprises a nucleotide sequence that is at least 95% identical to the nucleotide sequence of SEQ ID NO: 6, 7, 8, 9, 14, 15, 43, 44, 45, 46, 47, or 48.

22. The polynucleotide of embodiment 17, which comprises the nucleotide sequence of SEQ ID NO: 6, 7, 8, 9, 14, 15, 43, 44, 45, 46, 47, or 48.

23. A polynucleotide comprising the corresponding negative RNA sense of the nucleotide sequence of SEQ ID NO: 6, 7, 8, 9, 14, 15, 43, 44, 45, 46, 47, or 48 or the corresponding negative RNA sense of a nucleotide sequence that is at least 80% identical to the nucleotide sequence of SEQ ID NO: 6, 7, 8, 9, 14, 15, 43, 44, 45, 46, 47, or 48.

24. The polynucleotide of embodiment 23, which comprises the corresponding negative RNA sense of a nucleotide sequence that is at least 80% identical to the nucleotide sequence of SEQ ID NO: 6, 7, 8, 9, 14, 15, 43, 44, 45, 46, 47, or 48.

25. The polynucleotide of embodiment 23, which comprises the corresponding negative RNA sense of a nucleotide sequence that is at least 85% identical to the nucleotide sequence of SEQ ID NO: 6, 7, 8, 9, 14, 15, 43, 44, 45, 46, 47, or 48.

26. The polynucleotide of embodiment 23, which comprises the corresponding negative RNA sense of a nucleotide sequence that is at least 90% identical to the nucleotide sequence of SEQ ID NO: 6, 7, 8, 9, 14, 15, 43, 44, 45, 46, 47, or 48.

27. The polynucleotide of embodiment 23, which comprises the corresponding negative RNA sense of a nucleotide sequence that is at least 95% identical to the nucleotide sequence of SEQ ID NO: 6, 7, 8, 9, 14, 15, 43, 44, 45, 46, 47, or 48.

28. The polynucleotide of embodiment 23, which comprises the corresponding negative RNA sense of the nucleotide sequence of SEQ ID NO: 6, 7, 8, 9, 14, 15, 43, 44, 45, 46, 47, or 48.

29. The polynucleotide of any one of embodiments 16 or 23 to 28, which further comprises an NDV regulatory sequence.

30. The polynucleotide sequence of any one of embodiments 16 or 23 to 28, which further comprises an NDV gene start sequence.

31. The polynucleotide sequence of any one of embodiments 16, 23 to 28, or 30, which further comprises an NDV gene end sequence.

32. The polynucleotide of any one of embodiments 16 or 23 to 31, which further comprises a Kozak sequence.

33. The polynucleotide of any one of embodiments 16 or 23 to 32, which further comprises a restriction site.

34. The polynucleotide of any one of embodiments 16 to 22, which further comprises an NDV regulatory sequence, a Kozak sequence, a restriction site, or a combination thereof.

35. A nucleotide sequence comprising the polynucleotide of any one of embodiments 16 to 22, or 34, and (1) a nucleotide sequence coding for a NDV F transcription unit, (2) a nucleotide sequence coding for a NDV M transcription unit, (3) a nucleotide sequence coding for a NDV L transcription unit, (4) a nucleotide sequence coding for a NDV P transcription unit, (5) a nucleotide sequence coding for a NDV HN transcription unit, and (6) a nucleotide sequence coding for a NDV HN transcription unit.

36. A nucleotide sequence comprising the polynucleotide of any one of embodiments 16 or 23 to 33, and (1) a NDV F transcription unit, (2) a NDV M transcription unit, (3) a NDV L transcription unit, (4) a NDV P transcription unit, (5) a NDV HN transcription unit, and (6) a NDV HN transcription unit.

37. A vector comprising the polynucleotide of any one of embodiments 16 to 34.

38. A vector comprising the nucleotide sequence of embodiment 35 or 36.

39. A recombinant Newcastle disease virus (NDV) comprising a packaged genome, wherein the package genome comprises the polynucleotide of any one of embodiments 16 or 23 to 33.

40. A recombinant Newcastle disease virus (NDV) comprising a packaged genome, wherein the package genome comprises a polynucleotide sequence encoding the recombinant protein of any one of embodiments 1 to 15.

41. A recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain comprises the amino acid sequence of SEQ ID NO:41 or 35, or an amino acid sequence that is at least 90% identical to the amino acid sequence of SEQ ID NO:41 or 35.

42. The recombinant NDV of embodiment 41, wherein the ectodomain comprises an amino acid sequence that is at least 95% identical to the amino acid sequence of SEQ ID NO: 41 or 35.

43. A recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises a derivative of the ectodomain of a Lassa virus glycoprotein and the transmembrane and cytoplasmic domains of NDV F protein, wherein the derivative comprises the amino acid sequence of SEQ ID NO:42 or 36, or an amino acid sequence that is at least 90% identical to the amino acid sequence of SEQ ID NO:42 or 36.

44. The recombinant NDV of embodiment 43, wherein the ectodomain comprises an amino acid sequence that is at least 95% identical to the amino acid sequence of SEQ ID NO:42 or 36.

45. The recombinant NDV of any one of embodiments 41 to 44, wherein the NDV F protein is of the LaSota strain.

46. The recombinant NDV of any one of embodiments 41 to 44, wherein the transmembrane and cytoplasmic domain of NDV F protein comprise the amino acid sequence of SEQ ID NO:5.

47. The recombinant NDV of any one of embodiments 41 to 46, wherein the ectodomain or derivative of the ectodomain is linked directly to the transmembrane of the NDV F protein.

48. The recombinant NDV of any one of embodiments 41 to 46, wherein the ectodomain or derivative of the ectodomain is linked to the transmembrane of the NDV F protein by a linker.

49. A recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by the nucleotide sequence of SEQ ID NO: 47 or 33, or a nucleotide sequence that is at least 80% identical to the nucleotide sequence of SEQ ID NO: 47 or 33.

50. The recombinant NDV of embodiment 49, wherein the ectodomain is encoded by a nucleotide sequence that is at least 85% identical to the nucleotide sequence of SEQ ID NO: 47 or 33.

51. The recombinant NDV of embodiment 49, wherein the ectodomain is encoded by a nucleotide sequence that is at least 90% identical to the nucleotide sequence of SEQ ID NO: 47 or 33.

52. A recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises a derivative of the ectodomain of a Lassa virus glycoprotein and the transmembrane and cytoplasmic domains of NDV F protein, wherein the derivative of the ectodomain is encoded by the nucleotide sequence of SEQ ID NO: 48 or 34, or a nucleotide sequence that is at least 80% identical to the nucleotide sequence of SEQ ID NO: 48 or 34.

53. The recombinant NDV of embodiment 52, wherein the derivative of the ectodomain is encoded by a nucleotide sequence that is at least 85% identical to the nucleotide sequence of SEQ ID NO: 48 or 34.

54. The recombinant NDV of embodiment 52, wherein the derivative of the ectodomain is encoded by a nucleotide sequence that is at least 90% identical to the nucleotide sequence of SEQ ID NO: 48 or 34.

55. The recombinant NDV of any one of embodiments 49 to 54, wherein the NDV F protein is of the LaSota strain.

56. The recombinant NDV of any one of embodiments 49 to 54, wherein the transmembrane and cytoplasmic domain of NDV F protein comprise the amino acid sequence of SEQ ID NO:5.

57. The recombinant NDV of any one of embodiments 49 to 56, wherein the ectodomain or derivative of the ectodomain is directly linked to the transmembrane of the NDV F protein.

58. The recombinant NDV of any one of embodiments 49 to 56, wherein the ectodomain or derivative of the ectodomain is linked to the transmembrane of the NDV F protein by a linker.

59. A recombinant Newcastle disease virus (NDV) comprising the protein of any one of embodiments 1 to 15.

60. A recombinant Newcastle disease virus (NDV) comprising a protein comprising the amino acid sequence of SEQ ID NO:37, 39, 10, or 12, or an amino acid sequence that is at least 90% identical to the amino acid sequence of SEQ ID NO:37, 39, 10, or 12.

61. The recombinant NDV of embodiment 60, wherein the protein comprises an amino acid sequence that is at least 95% identical to the amino acid sequence of SEQ ID NO:37, 39, 10, or 12.

62. A recombinant Newcastle disease virus (NDV) comprising a protein comprising the amino acid sequence of SEQ ID NO: 38, 40, 11, or 13, or an amino acid sequence that is at least 90% identical to the amino acid sequence of SEQ ID NO: 38, 40, 11, or 13.

63. The recombinant NDV of embodiment 62, wherein the protein comprises an amino acid sequence that is at least 90% identical to the amino acid sequence of SEQ ID NO: 38, 40, 11, or 13.

64. A recombinant Newcastle disease virus (NDV) comprising a protein comprising the amino acid sequence of SEQ ID NO:16 or 17, or an amino acid sequence that is at least 90% identical to the amino acid sequence of SEQ ID NO:16 or 17.

65. The recombinant NDV of embodiment 64, wherein the protein comprises an amino acid sequence that is at least 90% identical to the amino acid sequence of SEQ ID NO:16 or 17.

66. A recombinant Newcastle disease virus (NDV) comprising a chimeric Lassa virus glycoprotein that comprises the ectodomain of a Lassa virus glycoprotein and the transmembrane and cytoplasmic domains of NDV F protein, wherein the ectodomain comprises the amino acid sequence of SEQ ID NO:41 or 35, or an amino acid sequence that is at least 90% identical to the amino acid sequence of SEQ ID NO:41 or 35.

67. The recombinant NDV of embodiment 66, wherein the ectodomain comprises an amino acid sequence that is at least 95% identical to the amino acid sequence of SEQ ID NO:41 or 35.

68. A recombinant Newcastle disease virus (NDV) comprising a chimeric Lassa virus glycoprotein, wherein the chimeric Lassa virus glycoprotein comprises a derivative of the ectodomain of a Lassa virus glycoprotein and the transmembrane and cytoplasmic domains of NDV F protein, wherein the derivative comprises the amino acid sequence of SEQ ID NO:42 or 36, or an amino acid sequence that is at least 90% identical to the amino acid sequence of SEQ ID NO:42 or 36.

69. The recombinant NDV of embodiment 68, wherein the derivative comprises an amino acid sequence that is at least 95% identical to the amino acid sequence of SEQ ID NO:42 or 36.

70. The recombinant NDV of any one of embodiments 66 to 69, wherein the NDV F protein is of the LaSota strain.

71. The recombinant NDV of any one of embodiments 66 to 69, wherein the transmembrane and cytoplasmic domain of NDV F protein comprise the amino acid sequence of SEQ ID NO:5.

72. The recombinant NDV of any one of embodiments 66 to 71, wherein the ectodomain or derivative of the ectodomain is directly linked to the transmembrane of the NDV F protein.

73. The recombinant NDV of any one of embodiments 66 to 71, wherein the ectodomain or derivative of the ectodomain is linked to the transmembrane of the NDV F protein by a linker.

74. An immunogenic composition comprising the recombinant protein of any one of embodiments 1 to 15.

75. An immunogenic composition comprising the polynucleotide of any one of embodiments 16 to 34.

76. An immunogenic composition comprising the nucleotide sequence of embodiment 35 or 36.

77. An immunogenic composition comprising the vector of embodiment 37 or 38.

78. An immunogenic composition comprising the recombinant NDV of any one of embodiments 39 to 73.

79. The immunogenic composition of embodiment 78, wherein the recombinant NDV is inactivated.

80. A method for immunizing against Lassa virus disease, comprising administering the immunogenic composition of any one of embodiments 74 to 79 to a subject.

81. A method for inducing an immune response against Lassa virus, comprising administering the immunogenic composition of any one of embodiments 74 to 79 to a subject.

82. A method for preventing Lassa virus disease, comprising administering the immunogenic composition of any one of embodiments 74 to 79 to a subject.

83. The method of any one of embodiments 80 to 82, wherein the subject is a human subject.

84. An in vitro or ex vivo cell comprising the recombinant NDV of any one of embodiments 39 to 73, the recombinant protein of any one of embodiments 1 to 15, the polynucleotide of any one of embodiments 16 to 34, the nucleotide sequence of embodiment 35 or 36, or the vector of embodiment 37 or 38.

85. An in vitro or ex vivo cell expressing the recombinant protein of any one of embodiments 1 to 15.

86. An ex vivo embryonated egg comprising the recombinant NDV of any one of embodiments 39 to 73, the recombinant protein of any one of embodiments 1 to 15, the polynucleotide of any one of embodiments 16 to 34, the nucleotide sequence of embodiment 35 or 36, or the vector of embodiment 37 or 38.

87. A kit comprising a container containing the recombinant NDV of any one of embodiments 39 to 73, the recombinant protein of any one of embodiments 1 to 15, the polynucleotide of any one of embodiments 16 to 34, the nucleotide sequence of embodiment 35 or 36, or the vector of embodiment 37 or 38.

### 6. EXAMPLES

### 6.1 Example 1: Recombinant NDV Expressing LASC GPC and NP

This example describes the design, generation and characterization of several recombinant NDVs expressing different versions of the glycoprotein (GP) and NP of a LASV strain representative of the lineage currently circulating in Nigeria (Lineage II) [4]. The recombinant viruses have been confirmed to contain the inserted genes and express the LASV proteins. Further, a mouse model of intranasal infection was used to confirm the induction of specific antibodies and cellular responses against the LASV proteins expressed by our recombinant NDVs.

### 6.1.1 Generation of Recombinant NDVs (Strain LaSota/L289A) Expressing Different Versions of LASV Lineage II GPC and NP (Strain LASV/H. sapiens-Wt/NGA/2018/IRR 013)

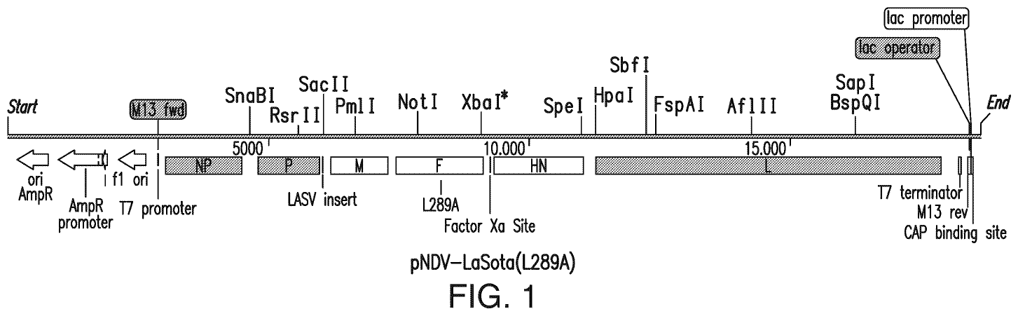

Synthetic and codon optimized sequences encoding the LASV glycoprotein complex (GPC) and nucleoprotein (NP) proteins were used to create recombinant NDV transcriptional units expressing different versions of these proteins. Transcriptional units contain: a restriction site (SacII), NDV regulatory sequences (Gene End, Intergenic and Gene Start), a Kozak sequence for optimal translation, the codon optimized open reading frame, and a second restriction site (SacII). The transcriptional unit was inserted in a plasmid containing the full-length cDNA of the NDV genome of strain LaSota/L289A (NDV LaSota strain in which the F protein comprises an amino acid substitution of L289A), at a unique SacII restriction site located between the viral genes encoding the phosphoprotein (P) and the matrix protein (M) (FIG. 1).

Four different transcriptional units, each comprising a different GPC gene, were generated. The four different GPC genes comprise:
A codon optimized full-length version of the LASV lineage II glycoprotein (GP) of strain LASV/H. sapiens-wt/NGA/2018/IRR 013, hereafter named “LASV GP” (SEQ ID NO:6). The recombinant NDV comprising the transcriptional unit that comprises LASV GP is referred to hereafter as “rNDV-LASV GP”.
A nucleotide sequence encoding a chimeric GP, wherein the chimeric GP comprises the ectodomain of the codon optimized LASV lineage II GP of strain LASV/H. sapiens-wt/NGA/2018/IRR 013 and the transmembrane and cytoplasmic domains of the F protein of NDV, hereafter named “LASV GP chimera” (SEQ ID NO:7). In other words, the transmembrane and cytoplasmic domains of LASV lineage II GP were replaced with the transmembrane and cytoplasmic domains of NDV F protein. The recombinant NDV comprising the transcriptional unit that comprises LASV GP chimera is referred to hereafter as “rNDV-LASV GP chimera”.
A codon optimized full-length LASV lineage II GP of strain LASV/H. sapiens-wt/NGA/2018/IRR 013 incorporating different mutations that stabilize the prefusion conformation, hereafter named “LASV GP 1 Pro” (SEQ ID NO:8). The recombinant NDV comprising the transcriptional unit that comprises LASV GP 1 Pro is referred to as “rNDV-LASV GP 1 Pro”.
A nucleotide sequence encoding a chimeric GP, wherein the chimeric GP comprises the ectodomain of the codon optimized of LASV GP 1 Pro and the transmembrane and cytoplasmic domains of the F protein of NDV, hereafter named “LASV GP 1 Pro chimera” (SEQ ID NO:9). In other words, the transmembrane and cytoplasmic domains of LASV GP 1 Pro were replaced with the transmembrane and cytoplasmic domains of NDV F protein. The recombinant NDV comprising the transcriptional unit that comprises LASV GP 1 Pro is referred to hereinafter as “rNDV-LASV GP 1 Pro chimera”.

Two different transcriptional units, each comprising a different version of the NP gene were generated. The two different NP genes comprise:
A codon optimized full-length version of the LASV lineage II NP of strain LASV/H. sapiens-wt/NGA/2018/IRR 013, hereafter named “LASV NP” (SEQ ID NO:14). The recombinant NDV comprising the transcriptional unit that comprises LASV NP is referred to as “rNDV-LASV NP”.
A codon optimized full-length version of the LASV lineage II NP of strain LASV/H. sapiens-wt/NGA/2018/IRR 013 in which the exonuclease domain has been inhibited, hereafter named “LASV NP ExoN KO” (SEQ ID NO:15). The recombinant NDV comprising the transcriptional unit that comprises LASV NP ExoN KO is referred to as “rNDV-LASV NP ExoN KO”.

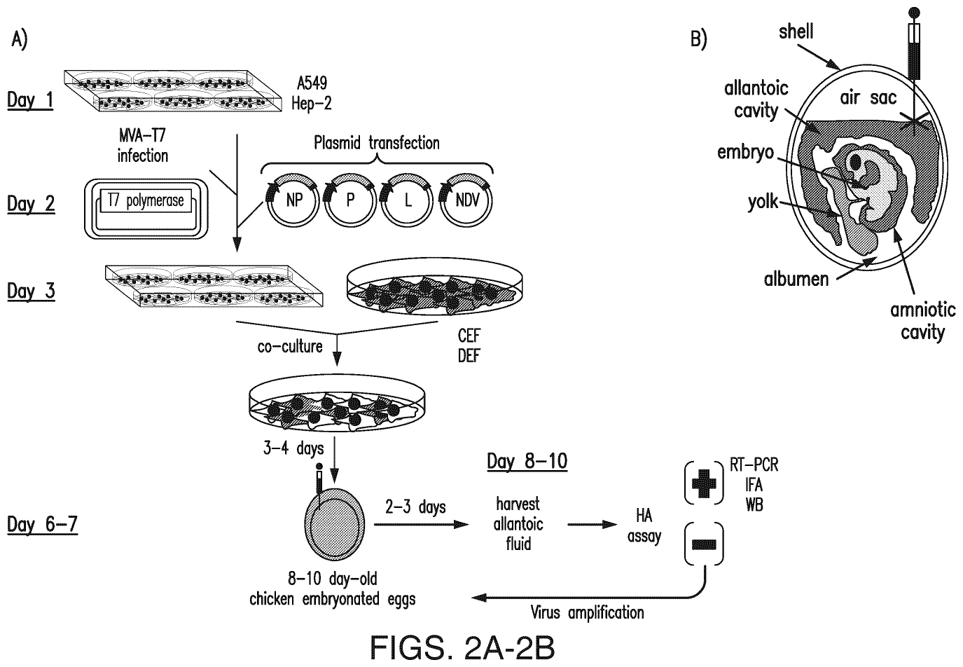

The rescue plasmids were confirmed by sequencing the insert and then used to rescue infectious recombinant NDVs following a well stablished protocol (FIG. 2). See, e.g., Ayllon et al., J Vis Exp. 2013; (80): 50830, for a description of the procedure to rescue recombinant NDV.

### 6.1.2 Characterization of the Rescued Viruses in Tissue Culture to Confirm the Expression of the LASV Proteins

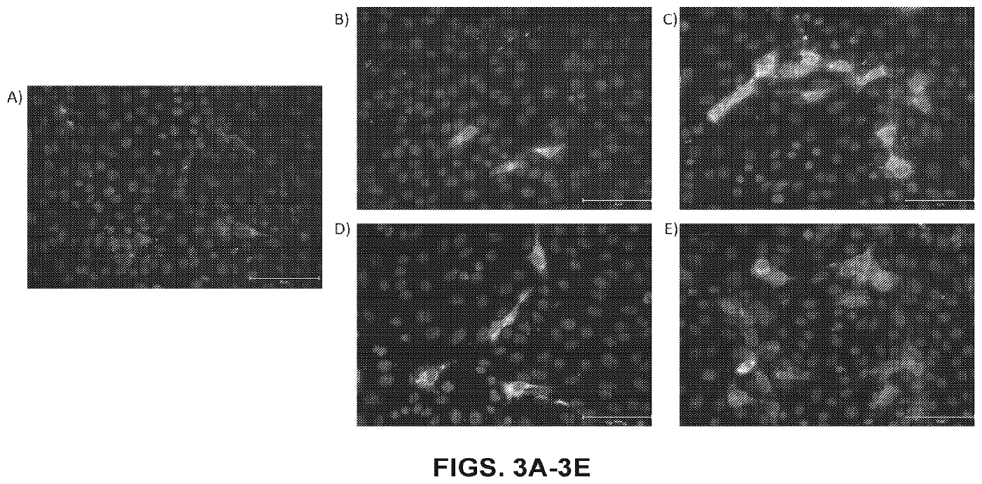

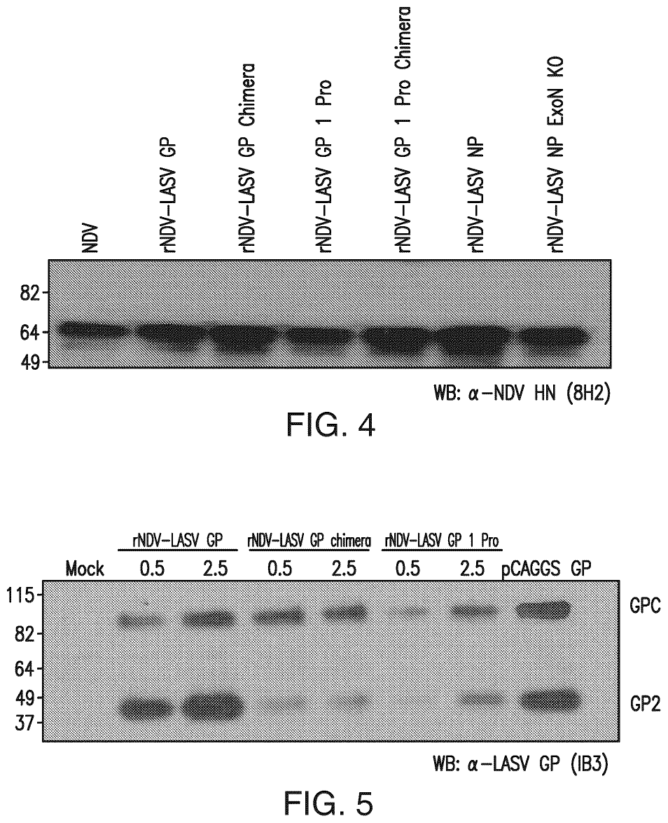

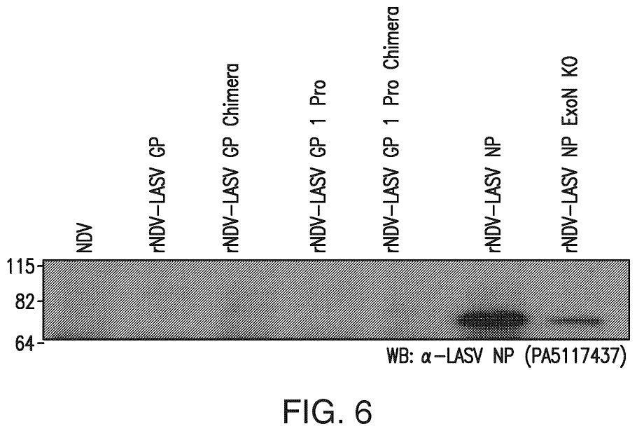

Specific antibodies were used to confirm the expression of the different LASV antigens, as well as the viral vector, in infected Vero cells at 24 hours post infection. The presence of LASV GP was confirmed by Immunofluorescence (IFA) (FIGS. 3A-3E) and western blot (WB) (FIG. 5). The expression of LASV NP (FIG. 6) and NDV hemagglutinin-neuraminidase (HN) (FIG. 4) were also confirmed by WB in concentrated viruses.

### 6.1.3 In Vivo Characterization of rNDV-LASV Candidates to Show Induction of LASV Specific Immune Responses

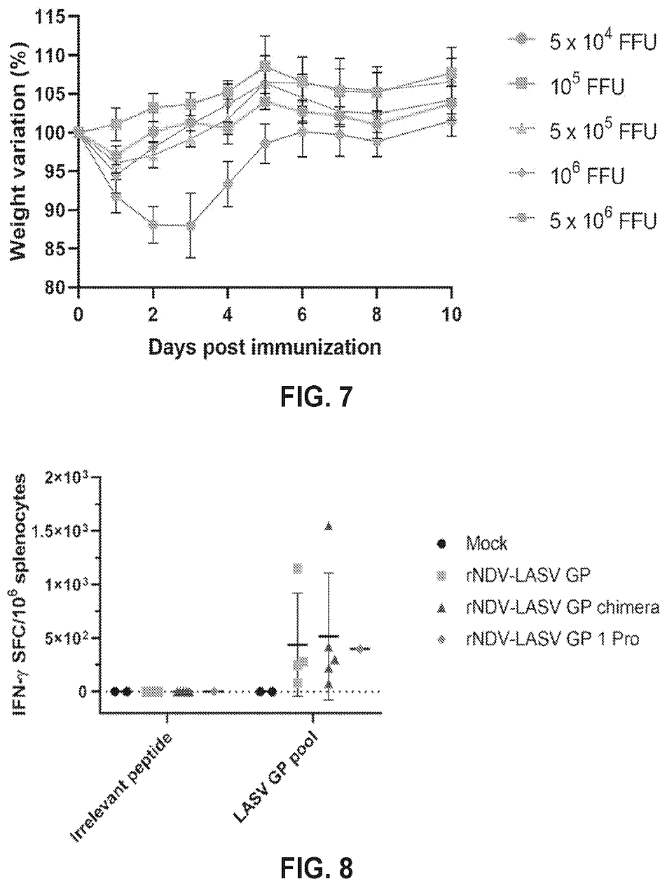

C57B1/6 female mice were used to study the safety and immunogenicity of recombinant NDVs, following a prime-boost regimen. Groups of mice were intranasally vaccinated with different doses (5×10⁴ FFU; 10⁵ FFU; 5×10⁵ FFU; 10⁶ FFU; or 5×10⁶ FFU) of attenuated live rNDV-LASV candidate viruses and boosted with the same dose, following a 3-week interval prime-boost vaccination regimen. Blood samples were collected for serology at different time points to quantify total serum IgGs. Blood was collected before each of the vaccination doses and at the end of the experiment. The cellular response was analyzed by quantifying IFN-7 secreting cells and the cytotoxic potential of T cells by harvesting spleens 10 days after the boost. The morbidity in type I Interferon receptor knockout (IFNAR^{-/-}) mice (C57BL/6 background) was studied. This initial assay was implemented because the challenge model for LASV, which is performed in a BSL-4 laboratory, uses a bone marrow chimera generated from this mouse line [5]. IFNAR^{-/-} mice that were vaccinated with different doses (5×10⁴ FFU; 10⁵ FFU; 5×10⁵ FFU; 10⁶ FFU; or 5×10⁶ FFU) of attenuated live rNDV-LASV candidate viruses were monitored (% weight variation), until complete recovery. As shown in FIG. 7, the highest viral dose (5×10⁶ FFU) induced a temporary weight loss (superior to 10%), but all the mice survived and recovered to initial body weight, corroborating the rNDV-LASV candidate vaccine safety.

The cellular immune response was characterized using enzyme-linked immunospot (ELISpot). Spleens were collected at 10 days post-immunization to quantify LASV-specific T cells. Isolated splenocytes were stimulated with either an irrelevant peptide pool or a LASV GP-derived peptide pool, QIITFFQEV (SEQ ID NO: 28), ANLNMTMPL (SEQ ID NO: 29), IINHKFCNL (SEQ ID NO: 30), NALINDQLI (SEQ ID NO: 31), and CNYSKYWYL (SEQ ID NO:32)) (GP-specific), which was predicted in-silico using the primary sequence of the GP. FIG. 8 shows the detection of specific IFN-γ producing cells (IFN-γ SFC splenocytes) in all the mice immunized with rNDV-LASV expressing 3 different versions of the GP (rNDV-LASV GP; rNDV-LASV GP chimera; rNDV-LASV GP 1 Pro).

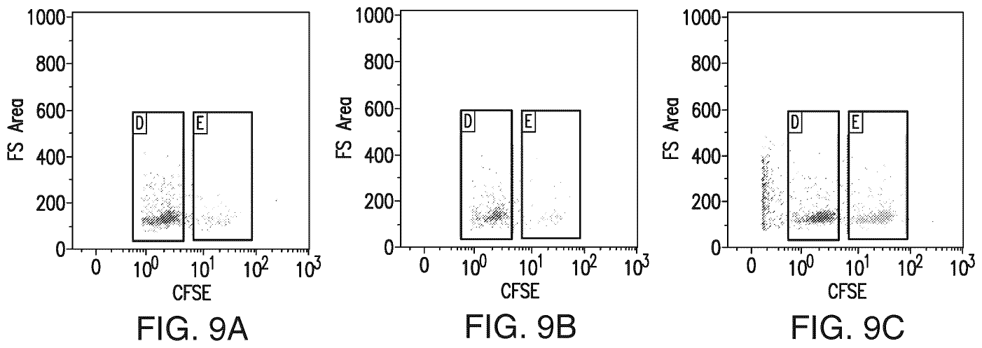

The cytotoxic potential of CD8 and NK cells was assessed by cytotoxic T Lymphocyte (CTL) assay. Spleens were harvested from C57BL/6J female donor mice to monitor T-cell mediated cytotoxicity. The cells were stimulated with either an irrelevant peptide pool (see insert D of FIGS. 9A-9C) or a LASV GP-derived peptide pool (IINHKFCNL (SEQ ID NO:30), NALINDQLI (SEQ ID NO:31); see insert E of FIGS. 9A-9C). After staining with different concentrations of CFSE, both populations of cells were mixed and injected via retro-orbital route into C57BL/6J female recipient mice, previously vaccinated. The spleens of these recipient mice were harvested to assess the killing activity of cytotoxic T cells by flow cytometry. FIGS. 9A-9C show a strong CTL response in mice vaccinated with the vaccine candidates rNDV-LASV GP (FIG. 9A) and rNDV-LASV GP 1 Pro (FIG. 9B), compared to NDV (FIG. 9C), with a killing activity of more than 80%.

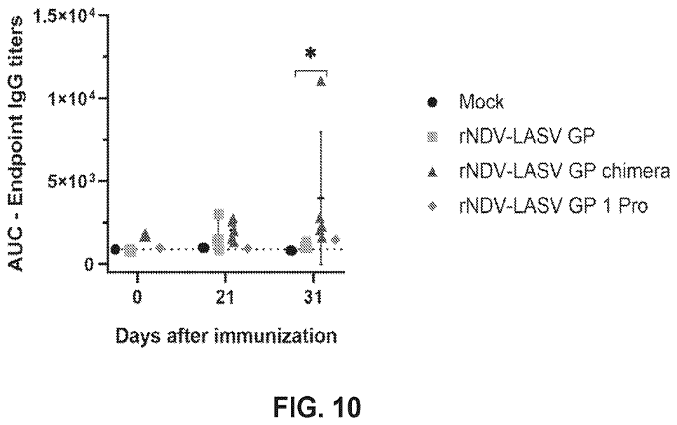

The LASV specific humoral response was assessed by enzyme-linked immunosorbent assay (ELISA) to determine the strongest LASV GP rNDV vaccine candidate. Blood was collected at days 0, 21 and 31 from mice intranasally vaccinated with either rNDV-LASV GP, rNDV-LASV GP chimera, or rNDV-LASV GP 1 Pro, following a 3 week-interval prime-boost regimen, for serological analysis to quantify total serum LASV GP IgG titers after the vaccine prime and boost. High titers of LASV-GP specific immunoglobulin G (IgG) were detected in the serum of mice vaccinated with rNDV-LASV GP chimera (FIG. 10).

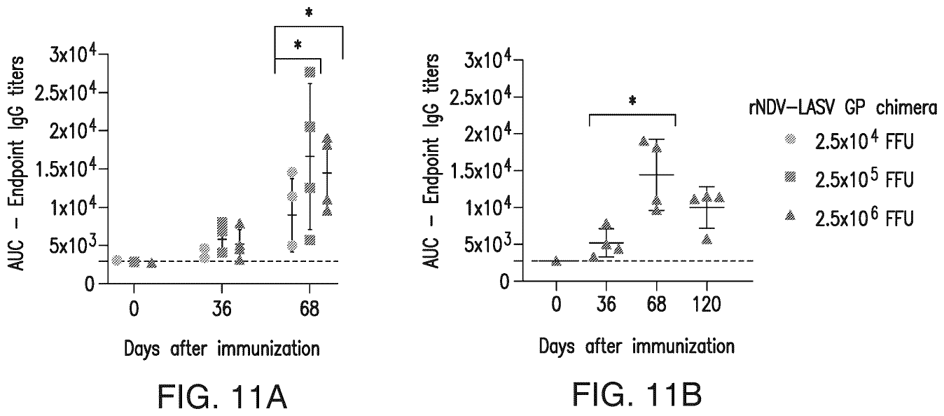

Characterization of the time- and dose-dependent response of antibody titers of mice vaccinated with rNDV-LASV GP chimera was performed. Blood was collected at days 0, 36 and 68 from mice intranasally vaccinated with either 2.5×10⁴ FFU, 2.5×10⁵ FFU, or 2.5×10⁶ FFU of rNDV-LASV GP chimera, following a 5 week-interval prime-boost regimen, for serological analysis to quantify total serum LASV GP IgG titers after the vaccine prime and boost. FIG. 11A shows a resulting 2-fold increase after the prime and 6-fold increase after the boost. The long-term humoral immunity for mice intranasally vaccinated with 2.5×10⁶ FFU of rNDV-LASV GP chimera was also studied. A significant increase was observed in antibody titers after the vaccine boost with no remarkable IgG loss over time (FIG. 11B).

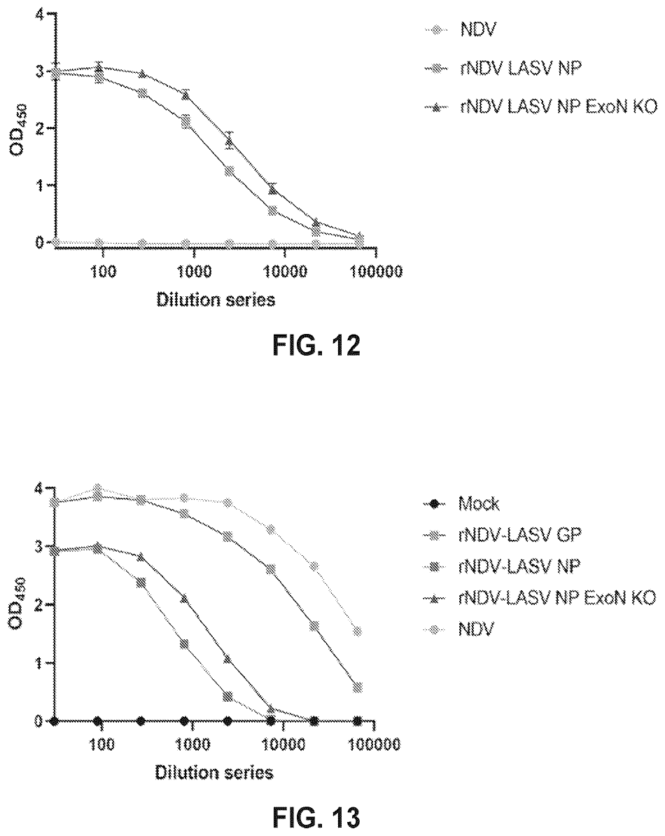

Serum LASV NP (FIG. 12) and NDV NP (FIG. 13) specific IgGs were quantified by ELISA in vaccinated C57BL/6J female mice. LASV NP-specific antibodies against both rNDV LASV NP and rNDV LASV NP ExoN K were detected in high titers after only one dose (the prime dose), which corroborate the appropriate expression of the NP after vaccination (FIG. 12). NDV NP-specific antibodies were detected after both prime and prime-boost vaccination regimens (FIG. 13), which was a confirmatory control for proper intranasal vaccine administration in mice.

### 6.1.4 Summary

Recombinant NDV viruses expressing different GP and NP proteins of LASV lineage II (i.e., rNDV-LASV GP; rNDV-LASV GP chimera; rNDV-LASV GP 1 Pro; rNDV-LASV GP 1 Pro chimera; rNDV-LASV NP; rNDV-LASV NP ExoN KO) were successfully rescued. Proper insertion of the sequence of the transcriptional units encoding the LASV GP and NP proteins in the genome of the rescued viruses was confirmed by Sanger sequencing. rNDV-LASV vaccine candidates were characterized in vitro and the expression of the proteins was confirmed by IFA and WB. The rNDV-LASV vaccines were shown to be safe in IFNAR^{-/-} (C57B1/6 background) mice. Vaccine approaches aim at the induction of neutralizing antibodies against the viral glycoprotein complex (GPC) and/or the induction of specific cytotoxic T lymphocytes (CTL) against epitopes in the GPC and the nucleoprotein (NP). Studies on human survivors of LASF indicate that a strong CTL response is important for protection [6, 7]. The immunogenicity of the rNDV-LASV vaccines was assessed in C57B1/6 mice to study the humoral and cellular immune responses. rNDV-LASV vaccination was shown to induce serum LASV GP-specific antibodies. rNDV-LASV vaccination resulted in a strong cellular immune response, as shown by ELISpot and CTL assays. Finally, the presence of LASV GP specific cytotoxic T cells was detected, which is crucial for a subject's recovery from LASF.

### 6.1.5 References Cited in the Background and Example 1

1. Asogun, D. A., Günther, S., Akpede, G. O., Ihekweazu, C. & Zumla, A. Lassa Fever: Epidemiology, Clinical Features, Diagnosis, Management and Prevention. Infect. Dis. Clin. North Am. 33, 933-951 (2019).
2. Salami, K., Gouglas, D., Schmaljohn, C., Saville, M. & Tornieporth, N. A review of Lassa fever vaccine candidates. Curr. Opin. Virol. 37, 105-111 (2019).
3. Salam, A. P. et al. Time to reconsider the role of ribavirin in Lassa fever. PLoS Negl. Trop. Dis. 15, e0009522-e0009522 (2021).
4. U., E. D. et al. Phylogeography of Lassa Virus in Nigeria. J. Virol. 93, e00929-19 (2022).
5. Oestereich, L. et al. Efficacy of Favipiravir Alone and in Combination With Ribavirin in a Lethal, Immunocompetent Mouse Model of Lassa Fever. J. Infect. Dis. 213, 934-938 (2016).
6. Mantlo, E., Paessler, S. & Huang, C. Differential Immune Responses to Hemorrhagic Fever-Causing Arenaviruses. Vaccines 7, 138 (2019).
7. Perdomo-Celis, F., Salvato, M. S., Medina-Moreno, S. & Zapata, J. C. T-Cell Response to Viral Hemorrhagic Fevers. Vaccines 7, (2019).

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A recombinant protein comprising a derivative of the ectodomain of a Lassa virus glycoprotein, wherein the derivative of the ectodomain comprises an amino acid sequence that is at least 90% identical to the amino acid sequence of SEQ ID NO:42 or 36, and wherein the derivative of the ectodomain comprises: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP.

2. The recombinant protein of claim 1, wherein the derivative of the ectodomain comprises an amino acid sequence that is at least 95% identical to the amino acid sequence of SEQ ID NO: 42 or 36.

3. The recombinant protein of claim 1, wherein the derivative of the ectodomain comprises the amino acid sequence of SEQ ID NO: 42 or 36.

4. A recombinant protein comprising a derivative of the ectodomain of a Lassa virus glycoprotein, wherein the derivative of the ectodomain comprises the amino acid sequence of a Lassa virus glycoprotein ectodomain and amino acid substitutions resulting in: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP.

5. The recombinant protein of any one of claims 1 to 4, which further comprises the transmembrane and cytoplasmic domains of NDV F protein.

6. The recombinant protein of claim 5, wherein the NDV F protein is of the LaSota strain.

7. The recombinant protein of claim 5, wherein the claims 1 to 4, wherein the transmembrane and cytoplasmic domains of NDV F protein comprise the amino acid sequence of SEQ ID NO:5.

8. The recombinant NDV of any one of claims 5 to 7, wherein the derivative of the ectodomain is linked directly to the transmembrane of the NDV F protein.

9. The recombinant NDV of any one of claims 5 to 7, wherein the derivative of the ectodomain is linked to the transmembrane of the NDV F protein by a linker.

10. A recombinant protein comprising an amino acid sequence that is at least 90% identical to the amino acid sequence of SEQ ID NO:39, 40, 12, or 13, wherein the protein comprises: (a) cysteine at the amino acid position corresponding to amino acid position 206 of Lassa virus/H. sapiens-wt/NGA/2018/IRR 013 glycoprotein (GP), (b) proline at the amino acid position corresponding to amino acid position 328 of Lassa virus/H. sapiens-wt/NGA/2018/IRR 013 GP, and (c) cysteine at the amino acid position corresponding to amino acid position 359 of Lassa virus/H. sapiens-wt/NGA/2018/IRR 013 GP.

11. The recombinant protein of claim 10, wherein the amino acid sequence is at least 95% identical to the amino acid sequence of SEQ ID NO:39, 40, 12, or 13.

12. The recombinant protein of claim 10, wherein the protein comprises the amino acid sequence of SEQ ID NO:39, 40, 12, or 13.

13. A recombinant protein comprising a derivative of a Lassa virus nucleoprotein, wherein the derivative comprises an amino acid sequence that is at least 90% identical to the amino acid sequence of SEQ ID NO:17, wherein the derivative comprises alanine at amino acid positions 389 and 392 of Lassa virus/H. sapiens-wt/NGA/2018/IRR 013 nucleoprotein (NP).

14. The recombinant protein of claim 13, wherein the derivative comprises an amino acid sequence that is at least 95% identical to the amino acid sequence of SEQ ID NO:17.

15. The recombinant protein of claim 13, wherein the derivative comprises the amino acid sequence of SEQ ID NO:17.

16. A polynucleotide comprises a nucleotide sequence encoding the recombinant protein of any one of claims 1 to 15.

17. A polynucleotide comprising the nucleotide sequence of SEQ ID NO:6, 7, 8, 9, 14, 15, 43, 44, 45, 46, 47, or 48, or a nucleotide sequence that is at least 80% identical to the nucleotide sequence of SEQ ID NO: 6, 7, 8, 9, 14, 15, 43, 44, 45, 46, 47, or 48.

18. The polynucleotide of claim 17, which comprises a nucleotide sequence that is at least 80% identical to the nucleotide sequence of SEQ ID NO: 6, 7, 8, 9, 14, 15, 43, 44, 45, 46, 47, or 48.

19. The polynucleotide of claim 17, which comprises a nucleotide sequence that is at least 85% identical to the nucleotide sequence of SEQ ID NO: 6, 7, 8, 9, 14, 15, 43, 44, 45, 46, 47, or 48.

20. The polynucleotide of claim 17, which comprises a nucleotide sequence that is at least 90% identical to the nucleotide sequence of SEQ ID NO: 6, 7, 8, 9, 14, 15, 43, 44, 45, 46, 47, or 48.

21. The polynucleotide of claim 17, which comprises a nucleotide sequence that is at least 95% identical to the nucleotide sequence of SEQ ID NO: 6, 7, 8, 9, 14, 15, 43, 44, 45, 46, 47, or 48.

22. The polynucleotide of claim 17, which comprises the nucleotide sequence of SEQ ID NO: 6, 7, 8, 9, 14, 15, 43, 44, 45, 46, 47, or 48.

23. A polynucleotide comprising the corresponding negative RNA sense of the nucleotide sequence of SEQ ID NO: 6, 7, 8, 9, 14, 15, 43, 44, 45, 46, 47, or 48 or the corresponding negative RNA sense of a nucleotide sequence that is at least 80% identical to the nucleotide sequence of SEQ ID NO: 6, 7, 8, 9, 14, 15, 43, 44, 45, 46, 47, or 48.

24. The polynucleotide of claim 23, which comprises the corresponding negative RNA sense of a nucleotide sequence that is at least 80% identical to the nucleotide sequence of SEQ ID NO: 6, 7, 8, 9, 14, 15, 43, 44, 45, 46, 47, or 48.

25. The polynucleotide of claim 23, which comprises the corresponding negative RNA sense of a nucleotide sequence that is at least 85% identical to the nucleotide sequence of SEQ ID NO: 6, 7, 8, 9, 14, 15, 43, 44, 45, 46, 47, or 48.

26. The polynucleotide of claim 23, which comprises the corresponding negative RNA sense of a nucleotide sequence that is at least 90% identical to the nucleotide sequence of SEQ ID NO: 6, 7, 8, 9, 14, 15, 43, 44, 45, 46, 47, or 48.

27. The polynucleotide of claim 23, which comprises the corresponding negative RNA sense of a nucleotide sequence that is at least 95% identical to the nucleotide sequence of SEQ ID NO: 6, 7, 8, 9, 14, 15, 43, 44, 45, 46, 47, or 48.

28. The polynucleotide of claim 23, which comprises the corresponding negative RNA sense of the nucleotide sequence of SEQ ID NO: 6, 7, 8, 9, 14, 15, 43, 44, 45, 46, 47, or 48.

29. The polynucleotide of any one of claims 16 or 23 to 28, which further comprises an NDV regulatory sequence.

30. The polynucleotide sequence of any one of claims 16 or 23 to 28, which further comprises an NDV gene start sequence.

31. The polynucleotide sequence of any one of claims 16, 23 to 28, or 30, which further comprises an NDV gene end sequence.

32. The polynucleotide of any one of claims 16 or 23 to 31, which further comprises a Kozak sequence.

33. The polynucleotide of any one of claims 16 or 23 to 32, which further comprises a restriction site.

34. The polynucleotide of any one of claims 16 to 22, which further comprises an NDV regulatory sequence, a Kozak sequence, a restriction site, or a combination thereof.

35. A nucleotide sequence comprising the polynucleotide of any one of claims 16 to 22, or 34, and (1) a nucleotide sequence coding for a NDV F transcription unit, (2) a nucleotide sequence coding for a NDV M transcription unit, (3) a nucleotide sequence coding for a NDV L transcription unit, (4) a nucleotide sequence coding for a NDV P transcription unit, (5) a nucleotide sequence coding for a NDV HN transcription unit, and (6) a nucleotide sequence coding for a NDV HN transcription unit.

36. A nucleotide sequence comprising the polynucleotide of any one of claims 16 or 23 to 33, and (1) a NDV F transcription unit, (2) a NDV M transcription unit, (3) a NDV L transcription unit, (4) a NDV P transcription unit, (5) a NDV HN transcription unit, and (6) a NDV HN transcription unit.

37. A vector comprising the polynucleotide of any one of claims 16 to 34.

38. A vector comprising the nucleotide sequence of claim 35 or 36.

39. A recombinant Newcastle disease virus (NDV) comprising a packaged genome, wherein the package genome comprises the polynucleotide of any one of claims 16 or 23 to 33.

40. A recombinant Newcastle disease virus (NDV) comprising a packaged genome, wherein the package genome comprises a polynucleotide sequence encoding the recombinant protein of any one of claims 1 to 15.

41. A recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain comprises the amino acid sequence of SEQ ID NO:41 or 35, or an amino acid sequence that is at least 90% identical to the amino acid sequence of SEQ ID NO:41 or 35.

42. The recombinant NDV of claim 41, wherein the ectodomain comprises an amino acid sequence that is at least 95% identical to the amino acid sequence of SEQ ID NO: 41 or 35.

43. A recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises a derivative of the ectodomain of a Lassa virus glycoprotein and the transmembrane and cytoplasmic domains of NDV F protein, wherein the derivative comprises the amino acid sequence of SEQ ID NO:42 or 36, or an amino acid sequence that is at least 90% identical to the amino acid sequence of SEQ ID NO:42 or 36.

44. The recombinant NDV of claim 43, wherein the ectodomain comprises an amino acid sequence that is at least 95% identical to the amino acid sequence of SEQ ID NO:42 or 36.

45. The recombinant NDV of any one of claims 41 to 44, wherein the NDV F protein is of the LaSota strain.

46. The recombinant NDV of any one of claims 41 to 44, wherein the transmembrane and cytoplasmic domain of NDV F protein comprise the amino acid sequence of SEQ ID NO:5.

47. The recombinant NDV of any one of claims 41 to 46, wherein the ectodomain or derivative of the ectodomain is linked directly to the transmembrane of the NDV F protein.

48. The recombinant NDV of any one of claims 41 to 46, wherein the ectodomain or derivative of the ectodomain is linked to the transmembrane of the NDV F protein by a linker.

49. A recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises the ectodomain of a Lassa virus glycoprotein and the transmembrane and cytoplasmic domains of NDV F protein, wherein the ectodomain is encoded by the nucleotide sequence of SEQ ID NO: 47 or 33, or a nucleotide sequence that is at least 80% identical to the nucleotide sequence of SEQ ID NO: 47 or 33.

50. The recombinant NDV of claim 49, wherein the ectodomain is encoded by a nucleotide sequence that is at least 85% identical to the nucleotide sequence of SEQ ID NO: 47 or 33.

51. The recombinant NDV of claim 49, wherein the ectodomain is encoded by a nucleotide sequence that is at least 90% identical to the nucleotide sequence of SEQ ID NO: 47 or 33.

52. A recombinant Newcastle disease virus (NDV) comprising a packaged genome, wherein the package genome comprises a transgene comprising a polynucleotide sequence encoding a chimeric Lassa virus glycoprotein, wherein the chimeric Lassa virus glycoprotein comprises a derivative of the ectodomain of a Lassa virus glycoprotein and the transmembrane and cytoplasmic domains of NDV F protein, wherein the derivative of the ectodomain is encoded by the nucleotide sequence of SEQ ID NO: 48 or 34, or a nucleotide sequence that is at least 80% identical to the nucleotide sequence of SEQ ID NO: 48 or 34.

53. The recombinant NDV of claim 52, wherein the derivative of the ectodomain is encoded by a nucleotide sequence that is at least 85% identical to the nucleotide sequence of SEQ ID NO: 48 or 34.

54. The recombinant NDV of claim 52, wherein the derivative of the ectodomain is encoded by a nucleotide sequence that is at least 90% identical to the nucleotide sequence of SEQ ID NO: 48 or 34.

55. The recombinant NDV of any one of claims 49 to 54, wherein the NDV F protein is of the LaSota strain.

56. The recombinant NDV of any one of claims 49 to 54, wherein the transmembrane and cytoplasmic domain of NDV F protein comprise the amino acid sequence of SEQ ID NO:5.

57. The recombinant NDV of any one of claims 49 to 56, wherein the ectodomain or derivative of the ectodomain is directly linked to the transmembrane of the NDV F protein.

58. The recombinant NDV of any one of claims 49 to 56, wherein the ectodomain or derivative of the ectodomain is linked to the transmembrane of the NDV F protein by a linker.

59. A recombinant Newcastle disease virus (NDV) comprising the protein of any one of claims 1 to 15.

60. A recombinant Newcastle disease virus (NDV) comprising a protein comprising the amino acid sequence of SEQ ID NO:37, 39, 10, or 12, or an amino acid sequence that is at least 90% identical to the amino acid sequence of SEQ ID NO:37, 39, 10, or 12.

61. The recombinant NDV of claim 60, wherein the protein comprises an amino acid sequence that is at least 95% identical to the amino acid sequence of SEQ ID NO:37, 39, 10, or 12.

62. A recombinant Newcastle disease virus (NDV) comprising a protein comprising the amino acid sequence of SEQ ID NO: 38, 40, 11, or 13, or an amino acid sequence that is at least 90% identical to the amino acid sequence of SEQ ID NO: 38, 40, 11, or 13.

63. The recombinant NDV of claim 62, wherein the protein comprises an amino acid sequence that is at least 90% identical to the amino acid sequence of SEQ ID NO: 38, 40, 11, or 13.

64. A recombinant Newcastle disease virus (NDV) comprising a protein comprising the amino acid sequence of SEQ ID NO:16 or 17, or an amino acid sequence that is at least 90% identical to the amino acid sequence of SEQ ID NO:16 or 17.

65. The recombinant NDV of claim 64, wherein the protein comprises an amino acid sequence that is at least 90% identical to the amino acid sequence of SEQ ID NO:16 or 17.

66. A recombinant Newcastle disease virus (NDV) comprising a chimeric Lassa virus glycoprotein that comprises the ectodomain of a Lassa virus glycoprotein and the transmembrane and cytoplasmic domains of NDV F protein, wherein the ectodomain comprises the amino acid sequence of SEQ ID NO:41 or 35, or an amino acid sequence that is at least 90% identical to the amino acid sequence of SEQ ID NO:41 or 35.

67. The recombinant NDV of claim 66, wherein the ectodomain comprises an amino acid sequence that is at least 95% identical to the amino acid sequence of SEQ ID NO:41 or 35.

68. A recombinant Newcastle disease virus (NDV) comprising a chimeric Lassa virus glycoprotein, wherein the chimeric Lassa virus glycoprotein comprises a derivative of the ectodomain of a Lassa virus glycoprotein and the transmembrane and cytoplasmic domains of NDV F protein, wherein the derivative comprises the amino acid sequence of SEQ ID NO:42 or 36, or an amino acid sequence that is at least 90% identical to the amino acid sequence of SEQ ID NO:42 or 36.

69. The recombinant NDV of claim 68, wherein the derivative comprises an amino acid sequence that is at least 95% identical to the amino acid sequence of SEQ ID NO:42 or 36.

70. The recombinant NDV of any one of claims 66 to 69, wherein the NDV F protein is of the LaSota strain.

71. The recombinant NDV of any one of claims 66 to 69, wherein the transmembrane and cytoplasmic domain of NDV F protein comprise the amino acid sequence of SEQ ID NO:5.

72. The recombinant NDV of any one of claims 66 to 71, wherein the ectodomain or derivative of the ectodomain is directly linked to the transmembrane of the NDV F protein.

73. The recombinant NDV of any one of claims 66 to 71, wherein the ectodomain or derivative of the ectodomain is linked to the transmembrane of the NDV F protein by a linker.

74. An immunogenic composition comprising the recombinant protein of any one of claims 1 to 15.

75. An immunogenic composition comprising the polynucleotide of any one of claims 16 to 34.

76. An immunogenic composition comprising the nucleotide sequence of claim 35 or 36.

77. An immunogenic composition comprising the vector of claim 37 or 38.

78. An immunogenic composition comprising the recombinant NDV of any one of claims 39 to 73.

79. The immunogenic composition of claim 78, wherein the recombinant NDV is inactivated.

80. A method for immunizing against Lassa virus disease, comprising administering the immunogenic composition of any one of claims 74 to 79 to a subject.

81. A method for inducing an immune response against Lassa virus, comprising administering the immunogenic composition of any one of claims 74 to 79 to a subject.

82. A method for preventing Lassa virus disease, comprising administering the immunogenic composition of any one of claims 74 to 79 to a subject.

83. The method of any one of claims 80 to 82, wherein the subject is a human subject.

84. An in vitro or ex vivo cell comprising the recombinant NDV of any one of claims 39 to 73, the recombinant protein of any one of claims 1 to 15, the polynucleotide of any one of claims 16 to 34, the nucleotide sequence of claim 35 or 36, or the vector of claim 37 or 38.

85. An in vitro or ex vivo cell expressing the recombinant protein of any one of claims 1 to 15.

86. An ex vivo embryonated egg comprising the recombinant NDV of any one of claims 39 to 73, the recombinant protein of any one of claims 1 to 15, the polynucleotide of any one of claims 16 to 34, the nucleotide sequence of claim 35 or 36, or the vector of claim 37 or 38.

87. A kit comprising a container containing the recombinant NDV of any one of claims 39 to 73, the recombinant protein of any one of claims 1 to 15, the polynucleotide of any one of claims 16 to 34, the nucleotide sequence of claim 35 or 36, or the vector of claim 37 or 38.


SEQUENCE LISTING (48 sequences)

SEQ ID NO: 1 - DNA, 15186 bp, synthetic construct
  source 1..15186: mol_type = other DNA; note = cDNA of genomic sequence of NDV strain LaSota
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 2 - DNA, 15186 bp, synthetic construct
  source 1..15186: mol_type = other DNA; note = cDNA of genomic sequence of NDV strain Hitchner B1
accaaacagagaatccgtaagttacgataaaaggcgaaggagcaattgaagtcgcacgggtagaaggtgtgaatctcgagtgcgagcccgaagcacaaactcgaggaagccttctgccaacatgtcttccgtattcgacgagtacgaacagctcctcgcggctcagactcgccccaatggagctcatggagggggggagaaagggagtaccttaaaagtagacgtcccggtattcactcttaacagtgatgacccagaagataggtggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggcaccccgttcgtcacagccggggctgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctagtaggggacgtagactcatatatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccggcagcaaggaggggcctggcagctgctgcccaacgagtctccgaggtgaccagcagcatagacatgcctactcaacaagtcggagtcctcactgggcttagcgaggggggatcccaagccctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgattgacaaaacccagcctgcttctacaagaacatcccaatgctctcacccgtagtcgacccctcgatttgcggctctatatgaccacaccctcaaacaaacatccccctctttcctccctccccctgctgtacaactccgcacgccctagataccacaggcacaccgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacagggcaagaccaaggtgctgagcgcagcatgggagaagcatgggagcatccagccaccggccagtcaagacaacctcgatcgacaggacagatctgacaaacaaccatccacacccgagcaaacgaccccgcacgacagcccgccggccacatccgctgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagccaggaaagactgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatgggggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccatctccctatgtgatacaaggaggcgaaatggcacttaataaactttcgcaaccagtgccacatccatctgaattgattaaacccgccactgcatgcgggcctgatataggagtggagagggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctagaagcggcaatcctctctcgcttcctcagccccactgaatgatcgcgtaaccgtaattaatctagctacattaaggattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgactcgtggactgatagtaaggaagactcagtattcatcaccacctatggattcatctttcaagttgggaatgaggaagccactgtcggcatgatcgatgataaacccaagcgcgagttactttccgctgcgatgctctgcctaggaagcgtcccaaataccggagaccttgttgagctggcaagggcctgtctcactatgatggtcacatgcaagaagagtgcaactaatactgagagaatggttttctcagtagtgcaggcaccccaagtgctgcaaagctgtagggttgtggcaaataaatactcatcagtgaatgcagtcaagcacgtgaaagcgccagagaagatccccgggagtggaaccctagaatacaaggtgaactttgtctccttgactgtggtaccgaagaaggatgtctacaagatcccagctgcagtattgaagatttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgctgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtccatcaagttagaaaaaacacgggtagaagattctggatcccggttggcgccctccaggtgcaggatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagttaagaaaaaactaccggttgtagatgaccaaaggacgatatacgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcgacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgattaagaaaaaatgtaagtggcaatgagatacaaggcaaaacagctcacggtaaataatacgggtaggacatggcgagctccggtcctgaaagggcagagcatcagattatcctaccagagtcacacctgtcttcaccattggtcaagcacaaactactctattattggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtactccaccccaggtgtttagaagaactggctaatattgaggtccctgattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgattgtggcagctaggacaaggtctgcggccaacaaattggtgatgctaacccataaggtaggccaagtctttgtcactcctgaacttgttgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcgggagatttcttcgcattcaacctgcaggagcttaaagacattctaattggcctcctccccaatgatatagcagaatccgtgactcatgcaatcgctactgtattctctggtttagaacagaatcaagcagctgagatgttgtgcctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacggatacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatacgaccctgtcactaacctgagcatgttcctaaaagacaaggcaatcgcacaccccaacgataattggcttgcctcgtttaggcggaaccttctctccgaagaccagaagaaacatgtaaaggaagcgacttcgactaaccgcctcttgatagagtttttagagtcaaatgattttgatccatataaagagatggaatatctgacgacccttgagtaccttagagatgacaatgtggcagtatcatactcgctcaaagagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatgttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggactactgagtcctaacattaggactaatatcttaactaggccgcctgggaatggagattgggccagtctgtgcaacgacccatactctttcaattttgagactgttgcaagcccaaacattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacactgtaattaagattgcgcttactaggaggccattaggcatcaagaggctgatgcggatagtcaattattctagcatgcatgcaatgctgtttagagacgatgttttttcctctagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcgaaaatagctcatatgtcgccacatgtgaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccagaacatatgatgagatcacactgcacctacatagtaaatttagttgctgtatcagggaagcacctgttgcggttcctttcgagctacttggggtggcaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctcgactgttcttaccaactctattacctgagagtaagagacctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagtcatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggt

SEQ ID NO: 3 - DNA, 15186 bp, synthetic construct
  source 1..15186: mol_type = other DNA; note = cDNA of genomic sequence of NDV strain LaSota (L289A mutation)
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagatcttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 4 - protein, 553 aa, synthetic construct
  source 1..553: note = Amino acid sequence of F protein of NDV strain LaSota
MGSRPSTKNPAPMTLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGVGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 5 - protein, 53 aa, synthetic construct
  source 1..53: note = Amino acid sequence of transmembrane and cytoplasmic domains of F protein of NDV strain LaSota
LITYIVLTIISLVFGILSLILACYLMYKQKAQQKTLLWLGNNTLDQMRATTKM

SEQ ID NO: 6 - DNA, 1473 bp, synthetic construct
  source 1..1473: mol_type = other DNA; note = Nucleotide sequence of the codon optimized LASV GP (strain LASV/H. sapiens-wt/NGA/2018/IRR 013)
atgggccagatcatcaccttcttccaggaggtgccccacgtgatcgaggaggtgatgaacatcgtgctgatcgccctgagcctgctggccatcctgaagggcgtgtacaacgtggccacctgcggcctgttcggcctgatcagcttcctgctgctgtgcggcagaagctgcagcgtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggcaagggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccgaggcccagatgagcatccagctgatcaacaaggccgtgaacgccctgatcaacgaccagctgatcatgaagaaccacctgagagacatcatgggcatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccctgggcctggtggacctgttcgtgttcagcaccagcttctacctgatcagcatcttcctgcacctggtgaagatccccacccacagacacatcgtgggcagaccctgccccaagccccacagactgaaccacatgggcatctgcagctgcggcctgtacaagcaccccggcgtgcccgtgaagtggaagagatga

SEQ ID NO: 7 - DNA, 1446 bp, synthetic construct
  source 1..1446: mol_type = other DNA; note = Nucleotide sequence of the LASV GP chimera (Codon optimized ectodomain of the GP from strain LASV/H. sapiens-wt/NGA/2018/IRR 013) fused to the transmembrane and cytoplasmic regions of the F protein of NDV)
atgggccagatcatcaccttcttccaggaggtgccccacgtgatcgaggaggtgatgaacatcgtgctgatcgccctgagcctgctggccatcctgaagggcgtgtacaacgtggccacctgcggcctgttcggcctgatcagcttcctgctgctgtgcggcagaagctgcagcgtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggcaagggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccgaggcccagatgagcatccagctgatcaacaaggccgtgaacgccctgatcaacgaccagctgatcatgaagaaccacctgagagacatcatgggcatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccgttaacctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtga

SEQ ID NO: 8 - DNA, 1473 bp, synthetic construct
  source 1..1473: mol_type = other DNA; note = Nucleotide sequence of the codon optimized LASV GP 1 Pro (Codon optimized full length sequence of LASV strain LASV/H. sapiens-wt/NGA/2018/IRR 013 glycoprotein with 3 amino acid mutations to stabilize the pre-fusion conformation)
atgggccagatcatcaccttcttccaggaggtgccccacgtgatcgaggaggtgatgaacatcgtgctgatcgccctgagcctgctggccatcctgaagggcgtgtacaacgtggccacctgcggcctgttcggcctgatcagcttcctgctgctgtgcggcagaagctgcagcgtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggctgcggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccccggcccagatgagcatccagctgatcaacaaggccgtgaacgccctgatcaacgaccagctgatcatgaagaaccacctgagagacatcatgtgtatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccctgggcctggtggacctgttcgtgttcagcaccagcttctacctgatcagcatcttcctgcacctggtgaagatccccacccacagacacatcgtgggcagaccctgccccaagccccacagactgaaccacatgggcatctgcagctgcggcctgtacaagcaccccggcgtgcccgtgaagtggaagagatga

SEQ ID NO: 9 - DNA, 1446 bp, synthetic construct
  source 1..1446: mol_type = other DNA; note = Nucleotide sequence of the LASV GP 1 Pro chimera (Codon optimized ectodomain of LASV strain LASV/H. sapiens-wt/NGA/2018/IRR 013 glycoprotein with 3 amino acid mutations to stabilize the pre-fusion conformation fused to the transmembrane and cytoplasmic regions of the F protein of NDV)
atgggccagatcatcaccttcttccaggaggtgccccacgtgatcgaggaggtgatgaacatcgtgctgatcgccctgagcctgctggccatcctgaagggcgtgtacaacgtggccacctgcggcctgttcggcctgatcagcttcctgctgctgtgcggcagaagctgcagcgtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggctgcggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccccggcccagatgagcatccagctgatcaacaaggccgtgaacgccctgatcaacgaccagctgatcatgaagaaccacctgagagacatcatgtgtatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccgttaacctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtga

SEQ ID NO: 10 - protein, 490 aa, synthetic construct
  source 1..490: note = Amino acid sequence of LASV GP (strain LASV/H. sapiens-wt/NGA/2018/IRR 013)
MGQIITFFQEVPHVIEEVMNIVLIALSLLAILKGVYNVATCGLFGLISFLLLCGRSCSVTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGKGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAEAQMSIQLINKAVNALINDQLIMKNHLRDIMGIPYCNYSKYWYLNHTVTGRTSLPRCWLVSNGSYLNETHFSDDIEQQADNMITELLQKEYMDRQGKTPLGLVDLFVFSTSFYLISIFLHLVKIPTHRHIVGRPCPKPHRLNHMGICSCGLYKHPGVPVKWKR

SEQ ID NO: 11 - protein, 481 aa, synthetic construct
  source 1..481: note = Amino acid sequence of LASV GP chimera (ectodomain of the GP from strain LASV/H. sapiens-wt/NGA/2018/IRR 013 fused to the transmembrane and cytoplasmic regions of the F protein of NDV)
MGQIITFFQEVPHVIEEVMNIVLIALSLLAILKGVYNVATCGLFGLISFLLLCGRSCSVTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGKGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAEAQMSIQLINKAVNALINDQLIMKNHLRDIMGIPYCNYSKYWYLNHTVTGRTSLPRCWLVSNGSYLNETHFSDDIEQQADNMITELLQKEYMDRQGKTPVNLITYIVLTIISLVFGILSLILACYLMYKQKAQQKTLLWLGNNTLDQMRATTKM

SEQ ID NO: 12 - protein, 490 aa, synthetic construct
  source 1..490: note = Amino acid sequence of LASV GP 1 Pro (full length sequence of LASV strain LASV/H. sapiens-wt/NGA/2018/IRR 013 glycoprotein with 3 amino acid mutations to stabilize the pre-fusion conformation)
MGQIITFFQEVPHVIEEVMNIVLIALSLLAILKGVYNVATCGLFGLISFLLLCGRSCSVTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGCGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAPAQMSIQLINKAVNALINDQLIMKNHLRDIMCIPYCNYSKYWYLNHTVTGRTSLPRCWLVSNGSYLNETHFSDDIEQQADNMITELLQKEYMDRQGKTPLGLVDLFVFSTSFYLISIFLHLVKIPTHRHIVGRPCPKPHRLNHMGICSCGLYKHPGVPVKWKR

SEQ ID NO: 13 - protein, 481 aa, synthetic construct
  source 1..481: note = Amino acid sequence of LASV GP 1 Pro chimera (ectodomain of LASV strain LASV/H. sapiens-wt/NGA/2018/IRR 013 glycoprotein with 3 amino acid mutations to stabilize the pre-fusion conformation fused to the transmembrane and cytoplasmic regions of the F protein of NDV)
MGQIITFFQEVPHVIEEVMNIVLIALSLLAILKGVYNVATCGLFGLISFLLLCGRSCSVTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGCGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAPAQMSIQLINKAVNALINDQLIMKNHLRDIMCIPYCNYSKYWYLNHTVTGRTSLPRCWLVSNGSYLNETHFSDDIEQQADNMITELLQKEYMDRQGKTPVNLITYIVLTIISLVFGILSLILACYLMYKQKAQQKTLLWLGNNTLDQMRATTKM

SEQ ID NO: 14 - DNA, 1710 bp, synthetic construct
  source 1..1710: mol_type = other DNA; note = Nucleotide sequence of the codon optimized LASV NP (strain LASV/H. sapiens-wt/NGA/2018/IRR 013)
atgagcgtgagcaaggaggtgaagagcttcctgtggacccagagcctgagaagagagctgagcggctactgcagcaacatcaagctgcaggtggtgaaggacgcccaggccctgctgcacggcctggacttcagcgaggtgagcaacgtgcagagactgatgagaaagcagaagagagacgacggcgacctgaagagactgagagacctgaaccaggccgtgaacaacctggtggagctgaagagcacccagcagaagagcgtgctgagagtgggcaccctgaccagcgacgacctgctgatcctggccgccgacctggagaagctgaagagcaaggtgatcagaaccgagagacccctgagcagcggcgtgtacatgggcaacctgagcacccagcagctggagcagagaaaggccctgctgagcatgatcggcatggtgggcggcgcccagggcacccagcccggcagagacggcgtggtgagagtgtgggacgtgaagaaccccgacctgctgaacaaccagttcggcaccatgcccagcctgaccctggcctgcctgaccaagcagggccaggtggacctgaacgacgtggtgctggccctgaccgacctgggcctgatctacaccgccaagtaccccaacagcagcgacctggacagactgagccagagccaccccatcctgaacatggtggacaccaagaagagcagcctgaacatcagcggctacaacttcagcctgggcgccgccgtgaaggccggcgcctgcatgctggacggcggcaacatgctggagaccatcaaggtgaccccccagaccatggacggcatcctgaagagcatcctgaaggtgaagagaagcctgggcatgttcgtgagcgacacccccggcgagagaaacccctacgagaacatcctgtacaagatctgcctgagcggcgacggctggccctacatcgccagcagaaccagcatcgtgggcagagcctgggagaacaccaccgtggacctggagagcaacggcaagccccagaaggtgggcaccgccggcagcaacaagagcctgcagagcgccggcttccccaccggcctgacctacagccagctgatgaccctgaaggacagcatgatgcagctggaccccagcgccaagacctggatcgacatcgagggcagacccgaggaccccgtggagatcgccctgtaccagcccatgagcggctgctacatccacttcttcagagagcccaccgacctgaagcagttcaagcaggacgccaagtacagccacggcatcgacgtggccgacctgttcagcgcccagcccggcctgaccagcgccgtgatcgaggccctgcccagaaacatggtgctgacctgccagggcagcgacgacatcaagaagctgctggacagccagggcagaagagacatcaagctgatcgacatcagcctgaacaaggtggacagcagaaagttcgagaacgccgtgtgggaccagtgcaaggacctgtgccacatgcacaccggcgtggtggtggagaagaagaagagaggcggcaaggaggagatcaccccccactgcgccctgatggactgcatcatgttcgacgccgccgtgagcggcggcctgaacatccccgtgctgagagccgtgctgcccaaggacatggtgttcagaaccagcagccccaaggtggtgctgtga

SEQ ID NO: 15 - DNA, 1710 bp, synthetic construct
  source 1..1710: mol_type = other DNA; note = Nucleotide sequence of the codon optimized LASV NP ExoN KO (strain LASV/H. sapiens-wt/NGA/2018/IRR 013 with 2 amino acid mutations)
atgagcgtgagcaaggaggtgaagagcttcctgtggacccagagcctgagaagagagctgagcggctactgcagcaacatcaagctgcaggtggtgaaggacgcccaggccctgctgcacggcctggacttcagcgaggtgagcaacgtgcagagactgatgagaaagcagaagagagacgacggcgacctgaagagactgagagacctgaaccaggccgtgaacaacctggtggagctgaagagcacccagcagaagagcgtgctgagagtgggcaccctgaccagcgacgacctgctgatcctggccgccgacctggagaagctgaagagcaaggtgatcagaaccgagagacccctgagcagcggcgtgtacatgggcaacctgagcacccagcagctggagcagagaaaggccctgctgagcatgatcggcatggtgggcggcgcccagggcacccagcccggcagagacggcgtggtgagagtgtgggacgtgaagaaccccgacctgctgaacaaccagttcggcaccatgcccagcctgaccctggcctgcctgaccaagcagggccaggtggacctgaacgacgtggtgctggccctgaccgacctgggcctgatctacaccgccaagtaccccaacagcagcgacctggacagactgagccagagccaccccatcctgaacatggtggacaccaagaagagcagcctgaacatcagcggctacaacttcagcctgggcgccgccgtgaaggccggcgcctgcatgctggacggcggcaacatgctggagaccatcaaggtgaccccccagaccatggacggcatcctgaagagcatcctgaaggtgaagagaagcctgggcatgttcgtgagcgacacccccggcgagagaaacccctacgagaacatcctgtacaagatctgcctgagcggcgacggctggccctacatcgccagcagaaccagcatcgtgggcagagcctgggagaacaccaccgtggacctggagagcaacggcaagccccagaaggtgggcaccgccggcagcaacaagagcctgcagagcgccggcttccccaccggcctgacctacagccagctgatgaccctgaaggacagcatgatgcagctggaccccagcgccaagacctggatcgccatcgaggccagacccgaggaccccgtggagatcgccctgtaccagcccatgagcggctgctacatccacttcttcagagagcccaccgacctgaagcagttcaagcaggacgccaagtacagccacggcatcgacgtggccgacctgttcagcgcccagcccggcctgaccagcgccgtgatcgaggccctgcccagaaacatggtgctgacctgccagggcagcgacgacatcaagaagctgctggacagccagggcagaagagacatcaagctgatcgacatcagcctgaacaaggtggacagcagaaagttcgagaacgccgtgtgggaccagtgcaaggacctgtgccacatgcacaccggcgtggtggtggagaagaagaagagaggcggcaaggaggagatcaccccccactgcgccctgatggactgcatcatgttcgacgccgccgtgagcggcggcctgaacatccccgtgctgagagccgtgctgcccaaggacatggtgttcagaaccagcagccccaaggtggtgctgtga

SEQ ID NO: 16 - protein, 569 aa, synthetic construct
  source 1..569: note = Amino acid sequence of the LASV NP (strain LASV/H. sapiens-wt/NGA/2018/IRR 013)
MSVSKEVKSFLWTQSLRRELSGYCSNIKLQVVKDAQALLHGLDFSEVSNVQRLMRKQKRDDGDLKRLRDLNQAVNNLVELKSTQQKSVLRVGTLTSDDLLILAADLEKLKSKVIRTERPLSSGVYMGNLSTQQLEQRKALLSMIGMVGGAQGTQPGRDGVVRVWDVKNPDLLNNQFGTMPSLTLACLTKQGQVDLNDVVLALTDLGLIYTAKYPNSSDLDRLSQSHPILNMVDTKKSSLNISGYNFSLGAAVKAGACMLDGGNMLETIKVTPQTMDGILKSILKVKRSLGMFVSDTPGERNPYENILYKICLSGDGWPYIASRTSIVGRAWENTTVDLESNGKPQKVGTAGSNKSLQSAGFPTGLTYSQLMTLKDSMMQLDPSAKTWIDIEGRPEDPVEIALYQPMSGCYIHFFREPTDLKQFKQDAKYSHGIDVADLFSAQPGLTSAVIEALPRNMVLTCQGSDDIKKLLDSQGRRDIKLIDISLNKVDSRKFENAVWDQCKDLCHMHTGVVVEKKKRGGKEEITPHCALMDCIMFDAAVSGGLNIPVLRAVLPKDMVFRTSSPKVVL

SEQ ID NO: 17 - protein, 569 aa, synthetic construct
  source 1..569: note = Amino acid sequence of the LASV NP ExoN KO (strain LASV/H. sapiens-wt/NGA/2018/IRR 013 with 2 amino acid mutations)
MSVSKEVKSFLWTQSLRRELSGYCSNIKLQVVKDAQALLHGLDFSEVSNVQRLMRKQKRDDGDLKRLRDLNQAVNNLVELKSTQQKSVLRVGTLTSDDLLILAADLEKLKSKVIRTERPLSSGVYMGNLSTQQLEQRKALLSMIGMVGGAQGTQPGRDGVVRVWDVKNPDLLNNQFGTMPSLTLACLTKQGQVDLNDVVLALTDLGLIYTAKYPNSSDLDRLSQSHPILNMVDTKKSSLNISGYNFSLGAAVKAGACMLDGGNMLETIKVTPQTMDGILKSILKVKRSLGMFVSDTPGERNPYENILYKICLSGDGWPYIASRTSIVGRAWENTTVDLESNGKPQKVGTAGSNKSLQSAGFPTGLTYSQLMTLKDSMMQLDPSAKTWIAIEARPEDPVEIALYQPMSGCYIHFFREPTDLKQFKQDAKYSHGIDVADLFSAQPGLTSAVIEALPRNMVLTCQGSDDIKKLLDSQGRRDIKLIDISLNKVDSRKFENAVWDQCKDLCHMHTGVVVEKKKRGGKEEITPHCALMDCIMFDAAVSGGLNIPVLRAVLPKDMVFRTSSPKVVL

SEQ ID NO: 18 - skipped

SEQ ID NO: 19 - DNA, 10 bp, synthetic construct
  source 1..10: mol_type = other DNA; note = NDV Gene End Sequence
ttagaaaaaa

SEQ ID NO: 20 - DNA, 10 bp, synthetic construct
  source 1..10: mol_type = other DNA; note = NDV Gene Start Sequence
acgggtagaa

SEQ ID NO: 21 - skipped

SEQ ID NO: 22 - protein, 58 aa, synthetic construct
  source 1..58: note = Signal Sequence of the LASV GP protein
MGQIITFFQEVPHVIEEVMNIVLIALSLLAILKGVYNVATCGLFGLISFLLLCGRSCS

SEQ ID NO: 23 - protein, 9 aa, synthetic construct
  source 1..9: note = Peptide derived from the LASV NP sequence
KSFLWTQSL

SEQ ID NO: 24 - protein, 9 aa, synthetic construct
  source 1..9: note = Peptide derived from the LASV NP sequence
QAVNNLVEL

SEQ ID NO: 25 - protein, 9 aa, synthetic construct
  source 1..9: note = Peptide derived from the LASV NP sequence
LTYSQLMTL

SEQ ID NO: 26 - protein, 9 aa, synthetic construct
  source 1..9: note = Peptide derived from the LASV NP sequence
YQPMSGCYI

SEQ ID NO: 27 - protein, 9 aa, synthetic construct
  source 1..9: note = Peptide derived from the LASV NP sequence
SGGLNIPVL

SEQ ID NO: 28 - protein, 9 aa, synthetic construct
  source 1..9: note = Peptide derived from the LASV GP sequence
QIITFFQEV

SEQ ID NO: 29 - protein, 9 aa, synthetic construct
  source 1..9: note = Peptide derived from the LASV GP sequence
ANLNMTMPL

SEQ ID NO: 30 - protein, 9 aa, synthetic construct
  source 1..9: note = Peptide derived from the LASV GP sequence
IINHKFCNL

SEQ ID NO: 31 - protein, 9 aa, synthetic construct
  source 1..9: note = Peptide derived from the LASV GP sequence
NALINDQLI

SEQ ID NO: 32 - protein, 9 aa, synthetic construct
  source 1..9: note = Peptide derived from the LASV GP sequence
CNYSKYWYL

SEQ ID NO: 33 - DNA, 1284 bp, synthetic construct
  source 1..1284: mol_type = other DNA; note = Nucleotide sequence of the ectodomain of LASV GP chimera. (Codon optimized ectodomain of the GP from strain LASV/H. sapiens-wt/NGA/2018/IRR 013)
atgggccagatcatcaccttcttccaggaggtgccccacgtgatcgaggaggtgatgaacatcgtgctgatcgccctgagcctgctggccatcctgaagggcgtgtacaacgtggccacctgcggcctgttcggcctgatcagcttcctgctgctgtgcggcagaagctgcagcgtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggcaagggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccgaggcccagatgagcatccagctgatcaacaaggccgtgaacgccctgatcaacgaccagctgatcatgaagaaccacctgagagacatcatgggcatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccgttaac

SEQ ID NO: 34 - DNA, 1284 bp, synthetic construct
  source 1..1284: mol_type = other DNA; note = Nucleotide sequence of the ectodomain of LASV GP 1 Pro chimera (Codon optimized ectodomain of LASV strain LASV/H. sapiens-wt/NGA/2018/IRR 013 glycoprotein with 3 amino acid mutations to stabilize the pre-fusion conformation)
atgggccagatcatcaccttcttccaggaggtgccccacgtgatcgaggaggtgatgaacatcgtgctgatcgccctgagcctgctggccatcctgaagggcgtgtacaacgtggccacctgcggcctgttcggcctgatcagcttcctgctgctgtgcggcagaagctgcagcgtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggctgcggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccccggcccagatgagcatccagctgatcaacaaggccgtgaacgccctgatcaacgaccagctgatcatgaagaaccacctgagagacatcatgtgtatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccgttaac

SEQ ID NO: 35 - protein, 428 aa, synthetic construct
  source 1..428: note = Amino acid sequence of the ectodomain of LASV GP chimera (ectodomain of the GP from strain LASV/H. sapiens-wt/NGA/2018/IRR 013)
MGQIITFFQEVPHVIEEVMNIVLIALSLLAILKGVYNVATCGLFGLISFLLLCGRSCSVTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGKGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAEAQMSIQLINKAVNALINDQLIMKNHLRDIMGIPYCNYSKYWYLNHTVTGRTSLPRCWLVSNGSYLNETHFSDDIEQQADNMITELLQKEYMDRQGKTPVN

SEQ ID NO: 36 - protein, 428 aa, synthetic construct
  source 1..428: note = Amino acid sequence of ectodomain of LASV GP 1 Pro chimera (ectodomain of LASV strain LASV/H. sapiens-wt/NGA/2018/IRR 013 glycoprotein with 3 amino acid mutations to stabilize the pre-fusion conformation)
MGQIITFFQEVPHVIEEVMNIVLIALSLLAILKGVYNVATCGLFGLISFLLLCGRSCSVTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGCGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAPAQMSIQLINKAVNALINDQLIMKNHLRDIMCIPYCNYSKYWYLNHTVTGRTSLPRCWLVSNGSYLNETHFSDDIEQQADNMITELLQKEYMDRQGKTPVN

SEQ ID NO: 37 - protein, 432 aa, synthetic construct
  source 1..432: note = Amino acid sequence of LASV GP (strain LASV/H. sapiens-wt/NGA/2018/IRR 013 without the signal sequence)
VTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGKGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAEAQMSIQLINKAVNALINDQLIMKNHLRDIMGIPYCNYSKYWYLNHTVTGRTSLPRCWLVSNGSYLNETHFSDDIEQQADNMITELLQKEYMDRQGKTPLGLVDLFVFSTSFYLISIFLHLVKIPTHRHIVGRPCPKPHRLNHMGICSCGLYKHPGVPVKWKR

SEQ ID NO: 38 - protein, 423 aa, synthetic construct
  source 1..423: note = Amino acid sequence of LASV GP chimera without the signal sequence (ectodomain of the GP from strain LASV/H. sapiens-wt/NGA/2018/IRR 013 fused to the transmembrane and cytoplasmic regions of the F protein of NDV)
VTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGKGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAEAQMSIQLINKAVNALINDQLIMKNHLRDIMGIPYCNYSKYWYLNHTVTGRTSLPRCWLVSNGSYLNETHFSDDIEQQADNMITELLQKEYMDRQGKTPVNLITYIVLTIISLVFGILSLILACYLMYKQKAQQKTLLWLGNNTLDQMRATTKM

SEQ ID NO: 39 - protein, 432 aa, synthetic construct
  source 1..432: note = Amino acid sequence of LASV GP 1 Pro without the signal sequence (full length sequence of LASV strain LASV/H. sapiens-wt/NGA/2018/IRR 013 glycoprotein with 3 amino acid mutations to stabilize the pre-fusion conformation)
VTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGCGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAPAQMSIQLINKAVNALINDQLIMKNHLRDIMCIPYCNYSKYWYLNHTVTGRTSLPRCWLVSNGSYLNETHFSDDIEQQADNMITELLQKEYMDRQGKTPLGLVDLFVFSTSFYLISIFLHLVKIPTHRHIVGRPCPKPHRLNHMGICSCGLYKHPGVPVKWKR

SEQ ID NO: 40 - protein, 423 aa, synthetic construct
  source 1..423: note = Amino acid sequence of LASV GP 1 Pro chimera without the signal sequence (ectodomain of LASV strain LASV/H. sapiens-wt/NGA/2018/IRR 013 glycoprotein with 3 amino acid mutations to stabilize the pre-fusion conformation fused to the transmembrane and cytoplasmic regions of the F protein of NDV)
VTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGCGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAPAQMSIQLINKAVNALINDQLIMKNHLRDIMCIPYCNYSKYWYLNHTVTGRTSLPRCWLVSNGSYLNETHFSDDIEQQADNMITELLQKEYMDRQGKTPVNLITYIVLTIISLVFGILSLILACYLMYKQKAQQKTLLWLGNNTLDQMRATTKM

SEQ ID NO: 41 - protein, 370 aa, synthetic construct
  source 1..370: note = Amino acid sequence of the ectodomain of LASV GP chimera without signal sequence (ectodomain of the GP from strain LASV/H. sapiens-wt/NGA/2018/IRR 013)
VTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGKGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAEAQMSIQLINKAVNALINDQLIMKNHLRDIMGIPYCNYSKYWYLNHTVTGRTSLPRCWLVSNGSYLNETHFSDDIEQQADNMITELLQKEYMDRQGKTPVN

SEQ ID NO: 42 - protein, 370 aa, synthetic construct
  source 1..370: note = Amino acid sequence of ectodomain of LASV GP 1 Pro chimera without signal sequence (ectodomain of LASV strain LASV/H. sapiens-wt/NGA/2018/IRR 013 glycoprotein with 3 amino acid mutations to stabilize the pre-fusion conformation)
VTYKGVYELQTLELDMANLNMTMPLSCTKNSSHHYIMVGNETGLELTLTNTSIINHKFCNLSDAHKRNLYNHALMSIISTFHLSIPNFNQYEAMSCDFNGGKISVQYNLSHAYAVDAANHCGTIANGVLQTFMRMAWGGSYIALDSGCGNWDCIMTSYQYLIIQNTTWEDHCQFSRPSPIGYLGLLSQRTRDIYISRRLLGTFTWTLSDSEGNEAPGGYCLTRWMLIEAELKCFGNTAIAKCNEKHDEEFCDMLRLFDFNKQAIERLKAPAQMSIQLINKAVNALINDQLIMKNHLRDIMCIPYCNYSKYWYLNHTVTGRTSLPRCWLVSNGSYLNETHFSDDIEQQADNMITELLQKEYMDRQGKTPVN

SEQ ID NO: 43 - DNA, 1299 bp, synthetic construct
  source 1..1299: mol_type = other DNA; note = Nucleotide counterpart of SEQ ID NO: 37
gtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggcaagggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccgaggcccagatgagcatccagctgatcaacaaggccgtgaacgccctgatcaacgaccagctgatcatgaagaaccacctgagagacatcatgggcatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccctgggcctggtggacctgttcgtgttcagcaccagcttctacctgatcagcatcttcctgcacctggtgaagatccccacccacagacacatcgtgggcagaccctgccccaagccccacagactgaaccacatgggcatctgcagctgcggcctgtacaagcaccccggcgtgcccgtgaagtggaagagatga

SEQ ID NO: 44 - DNA, 1272 bp, synthetic construct
  source 1..1272: mol_type = other DNA; note = Nucleotide counterpart of SEQ ID NO: 38
gtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggcaagggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccgaggcccagatgagcatccagctgatcaacaaggccgtgaacgccctgatcaacgaccagctgatcatgaagaaccacctgagagacatcatgggcatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccgttaacctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtga

SEQ ID NO: 45 - DNA, 1299 bp, synthetic construct
  source 1..1299: mol_type = other DNA; note = Nucleotide counterpart of SEQ ID NO: 39
gtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggctgcggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccccggcccagatgagcatccagctgatcaacaaggccgtgaacgccctgatcaacgaccagctgatcatgaagaaccacctgagagacatcatgtgtatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccctgggcctggtggacctgttcgtgttcagcaccagcttctacctgatcagcatcttcctgcacctggtgaagatccccacccacagacacatcgtgggcagaccctgccccaagccccacagactgaaccacatgggcatctgcagctgcggcctgtacaagcaccccggcgtgcccgtgaagtggaagagatga

SEQ ID NO: 46 - DNA, 1272 bp, synthetic construct
  source 1..1272: mol_type = other DNA; note = Nucleotide counterpart of SEQ ID NO: 40
gtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggctgcggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccccggcccagatgagcatccagctgatcaacaaggccgtgaacgccctgatcaacgaccagctgatcatgaagaaccacctgagagacatcatgtgtatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccgttaacctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtga

SEQ ID NO: 47 - DNA, 1110 bp, synthetic construct
  source 1..1110: mol_type = other DNA; note = Nucleotide counterpart of SEQ ID NO: 41
gtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggcaagggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccgaggcccagatgagcatccagctgatcaacaaggccgtgaacgccctgatcaacgaccagctgatcatgaagaaccacctgagagacatcatgggcatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccgttaac

SEQ ID NO: 48 - DNA, 1110 bp, synthetic construct
  source 1..1110: mol_type = other DNA; note = Nucleotide counterpart of SEQ ID NO: 42
gtgacctacaagggcgtgtacgagctgcagaccctggagctggacatggccaacctgaacatgaccatgcccctgagctgcaccaagaacagcagccaccactacatcatggtgggcaacgagaccggcctggagctgaccctgaccaacaccagcatcatcaaccacaagttctgcaacctgagcgacgcccacaagagaaacctgtacaaccacgccctgatgagcatcatcagcaccttccacctgagcatccccaacttcaaccagtacgaggccatgagctgcgacttcaacggcggcaagatcagcgtgcagtacaacctgagccacgcctacgccgtggacgccgccaaccactgcggcaccatcgccaacggcgtgctgcagaccttcatgagaatggcctggggcggcagctacatcgccctggacagcggctgcggcaactgggactgcatcatgaccagctaccagtacctgatcatccagaacaccacctgggaggaccactgccagttcagcagacccagccccatcggctacctgggcctgctgagccagagaaccagagacatctacatcagcagaagactgctgggcaccttcacctggaccctgagcgacagcgagggcaacgaggcccccggcggctactgcctgaccagatggatgctgatcgaggccgagctgaagtgcttcggcaacaccgccatcgccaagtgcaacgagaagcacgacgaggagttctgcgacatgctgagactgttcgacttcaacaagcaggccatcgagagactgaaggccccggcccagatgagcatccagctgatcaacaaggccgtgaacgccctgatcaacgaccagctgatcatgaagaaccacctgagagacatcatgtgtatcccctactgcaactacagcaagtactggtacctgaaccacaccgtgaccggcagaaccagcctgcccagatgctggctggtgagcaacggcagctacctgaacgagacccacttcagcgacgacatcgagcagcaggccgacaacatgatcaccgagctgctgcagaaggagtacatggacagacagggcaagacccccgttaac